SEQUENCE LISTING (743 sequences)

SEQ ID NO: 1 - protein, 48 aa, synthetic construct
  VARIANT 48: note = X can be any amino acid
MIPGGLSEAKPATPEIQEIVDKVKPQLEEKTGETYGKLEAVQYKTQVX

SEQ ID NO: 2 - protein, 24 aa, synthetic construct
GTNYYIKVRAGDNKYMHLKVFKSL

SEQ ID NO: 3 - protein, 21 aa, synthetic construct
EDLVLTGYQVDKNKDDELTGF

SEQ ID NO: 4 - protein, 95 aa, synthetic construct
  VARIANT 4: note = X can be any amino acid
  VARIANT 49: note = X residue can be repeated one or more times
  VARIANT 71..75: note = X can be any amino acid
  VARIANT 74: note = X residue can be repeated one or more times
  VARIANT 77: note = X can be any amino acid
  VARIANT 48..50: note = X can be any amino acid
MIPXGLSEAKPATPEIQEIVDKVKPQLEEKTGETYGKLEAVQYKTQVXXXTNYYIKVRAGDNKYMHLKVFXXXXXDXVLTGYQVDKNKDDELTGF

SEQ ID NO: 5 - protein, 95 aa, synthetic construct
  VARIANT 49: note = X residue can be repeated one or more times
  VARIANT 74: note = X residue can be repeated one or more times
  VARIANT 49: note = X can be any amino acid
  VARIANT 74: note = X can be any amino acid
MIPGGLSEAKPATPEIQEIVDKVKPQLEEKTGETYGKLEAVQYKTQVDXGTNYYIKVRAGDNKYMHLKVFKSLXEDLVLTGYQVDKNKDDELTGF

SEQ ID NO: 6 - protein, 9 aa, synthetic construct
HDVFYDQKA

SEQ ID NO: 7 - protein, 9 aa, synthetic construct
PFKIITVWH

SEQ ID NO: 8 - protein, 9 aa, synthetic construct
QYKIITWWS

SEQ ID NO: 9 - protein, 9 aa, synthetic construct
HYYVHYNDQ

SEQ ID NO: 10 - protein, 9 aa, synthetic construct
YYQIITWWQ

SEQ ID NO: 11 - protein, 9 aa, synthetic construct
VHGPHLEQA

SEQ ID NO: 12 - protein, 9 aa, synthetic construct
LQDQWPWRD

SEQ ID NO: 13 - skipped

SEQ ID NO: 14 - protein, 9 aa, synthetic construct
SWVKPDNEL

SEQ ID NO: 15 - protein, 9 aa, synthetic construct
NSLGVSEWV

SEQ ID NO: 16 - protein, 9 aa, synthetic construct
VYRIVSWWR

SEQ ID NO: 17 - protein, 9 aa, synthetic construct
YYRQLKFNT

SEQ ID NO: 18 - protein, 9 aa, synthetic construct
GWIKVDNVL

SEQ ID NO: 19 - protein, 9 aa, synthetic construct
TAYYIHVNA

SEQ ID NO: 20 - protein, 9 aa, synthetic construct
VRVEPTPND

SEQ ID NO: 21 - protein, 9 aa, synthetic construct
DLNSSVWYQ

SEQ ID NO: 22 - protein, 9 aa, synthetic construct
FTYQISYTV

SEQ ID NO: 23 - protein, 9 aa, synthetic construct
RENWYGEFA

SEQ ID NO: 24 - protein, 9 aa, synthetic construct
TALAWNTQA

SEQ ID NO: 25 - protein, 9 aa, synthetic construct
HIPALKVHA

SEQ ID NO: 26 - protein, 9 aa, synthetic construct
HHAYAAVYV

SEQ ID NO: 27 - protein, 9 aa, synthetic construct
YDELYHEKA

SEQ ID NO: 28 - protein, 9 aa, synthetic construct
SWYYPKNVL

SEQ ID NO: 29 - protein, 9 aa, synthetic construct
FLEWHFWEW

SEQ ID NO: 30 - protein, 9 aa, synthetic construct
SWVRGTNQL

SEQ ID NO: 31 - protein, 9 aa, synthetic construct
WRKLLSWWK

SEQ ID NO: 32 - protein, 9 aa, synthetic construct
PKSHQTNEQ

SEQ ID NO: 33 - protein, 9 aa, synthetic construct
NWNQIDNPL

SEQ ID NO: 34 - protein, 9 aa, synthetic construct
FWVGLHNQL

SEQ ID NO: 35 - protein, 9 aa, synthetic construct
FTQPFNEPI

SEQ ID NO: 36 - protein, 9 aa, synthetic construct
PEYQPQPKA

SEQ ID NO: 37 - protein, 9 aa, synthetic construct
NDPYFQSFS

SEQ ID NO: 38 - protein, 9 aa, synthetic construct
HQDDVAWQS

SEQ ID NO: 39 - protein, 9 aa, synthetic construct
KGYFHTVEA

SEQ ID NO: 40 - protein, 9 aa, synthetic construct
VDQVWSENA

SEQ ID NO: 41 - protein, 9 aa, synthetic construct
LSSIWQDVA

SEQ ID NO: 42 - protein, 9 aa, synthetic construct
TASLYWNYA

SEQ ID NO: 43 - protein, 9 aa, synthetic construct
SWKQWHNYL

SEQ ID NO: 44 - protein, 9 aa, synthetic construct
QVHLDNSPL

SEQ ID NO: 45 - protein, 9 aa, synthetic construct
QNNWYLSNY

SEQ ID NO: 46 - protein, 9 aa, synthetic construct
WEWALLLEY

SEQ ID NO: 47 - protein, 9 aa, synthetic construct
GDSYWEYVS

SEQ ID NO: 48 - protein, 9 aa, synthetic construct
GQKWWQHKI

SEQ ID NO: 49 - protein, 9 aa, synthetic construct
LDGLYHVNS

SEQ ID NO: 50 - protein, 9 aa, synthetic construct
HAPAPVQAI

SEQ ID NO: 51 - protein, 9 aa, synthetic construct
WEDHWNYQY

SEQ ID NO: 52 - protein, 9 aa, synthetic construct
SWRYKSNQL

SEQ ID NO: 53 - protein, 9 aa, synthetic construct
RVDWWLPEV

SEQ ID NO: 54 - protein, 9 aa, synthetic construct
SWIEISNQL

SEQ ID NO: 55 - protein, 9 aa, synthetic construct
SLGQAETWA

SEQ ID NO: 56 - protein, 9 aa, synthetic construct
KIGQLPSRV

SEQ ID NO: 57 - protein, 9 aa, synthetic construct
AWHTKNNSI

SEQ ID NO: 58 - protein, 9 aa, synthetic construct
QTKLVTWWW

SEQ ID NO: 59 - protein, 9 aa, synthetic construct
AVFDYADPS

SEQ ID NO: 60 - protein, 9 aa, synthetic construct
HVKIVTWWK

SEQ ID NO: 61 - protein, 9 aa, synthetic construct
HVLKVSSVS

SEQ ID NO: 62 - protein, 9 aa, synthetic construct
YQDYDSATH

SEQ ID NO: 63 - protein, 9 aa, synthetic construct
SWGKVSNEL

SEQ ID NO: 64 - protein, 9 aa, synthetic construct
LEDEWWSIL

SEQ ID NO: 65 - protein, 9 aa, synthetic construct
YYKLVTWWR

SEQ ID NO: 66 - protein, 9 aa, synthetic construct
AWVKEHNLI

SEQ ID NO: 67 - protein, 9 aa, synthetic construct
QEYLWKQYI

SEQ ID NO: 68 - protein, 9 aa, synthetic construct
FNKDLFLGW

SEQ ID NO: 69 - protein, 9 aa, synthetic construct
HFGIRYAHV

SEQ ID NO: 70 - protein, 9 aa, synthetic construct
WYAWQEGWQ

SEQ ID NO: 71 - protein, 9 aa, synthetic construct
GNAAWGSFA

SEQ ID NO: 72 - protein, 9 aa, synthetic construct
REDLDLFYT

SEQ ID NO: 73 - protein, 9 aa, synthetic construct
NENVYRVTQ

SEQ ID NO: 74 - protein, 9 aa, synthetic construct
WHKIVTQWP

SEQ ID NO: 75 - protein, 9 aa, synthetic construct
HYRIITHWE

SEQ ID NO: 76 - protein, 9 aa, synthetic construct
LVFDASTSA

SEQ ID NO: 77 - protein, 9 aa, synthetic construct
QYAEFLEHI

SEQ ID NO: 78 - protein, 9 aa, synthetic construct
GNVQEDWHI

SEQ ID NO: 79 - protein, 9 aa, synthetic construct
GWSASYPES

SEQ ID NO: 80 - protein, 9 aa, synthetic construct
YQFEHISYG

SEQ ID NO: 81 - protein, 9 aa, synthetic construct
AWITPVNVL

SEQ ID NO: 82 - protein, 9 aa, synthetic construct
ESYAAIFWG

SEQ ID NO: 83 - protein, 9 aa, synthetic construct
RVWPWTWDR

SEQ ID NO: 84 - protein, 10 aa, synthetic construct
QVKKWWEYFV

SEQ ID NO: 85 - protein, 9 aa, synthetic construct
IGQISEQQW

SEQ ID NO: 86 - protein, 9 aa, synthetic construct
AFHKLGWIL

SEQ ID NO: 87 - protein, 9 aa, synthetic construct
FKKWWWKYK

SEQ ID NO: 88 - protein, 9 aa, synthetic construct
ASQRLWIIY

SEQ ID NO: 89 - protein, 9 aa, synthetic construct
QRETHILWS

SEQ ID NO: 90 - protein, 9 aa, synthetic construct
GQYWFWAPG

SEQ ID NO: 91 - protein, 9 aa, synthetic construct
RDWAYYIYF

SEQ ID NO: 92 - protein, 9 aa, synthetic construct
IGYKLNYIW

SEQ ID NO: 93 - protein, 9 aa, synthetic construct
VSLTEQRWK

SEQ ID NO: 94 - protein, 9 aa, synthetic construct
WVSQQHYSH

SEQ ID NO: 95 - protein, 9 aa, synthetic construct
DDDNQHYGL

SEQ ID NO: 96 - protein, 9 aa, synthetic construct
PTRWISIYW

SEQ ID NO: 97 - protein, 9 aa, synthetic construct
TAFAPNKAS

SEQ ID NO: 98 - protein, 9 aa, synthetic construct
HYTDFKWHK

SEQ ID NO: 99 - protein, 9 aa, synthetic construct
NKFWSWAPG

SEQ ID NO: 100 - protein, 9 aa, synthetic construct
ELDEPAWDW

SEQ ID NO: 101 - protein, 9 aa, synthetic construct
HEKWGFWEL

SEQ ID NO: 102 - protein, 9 aa, synthetic construct
VATADLIKA

SEQ ID NO: 103 - protein, 9 aa, synthetic construct
YYKVISWWH

SEQ ID NO: 104 - protein, 9 aa, synthetic construct
HWHHYDNII

SEQ ID NO: 105 - protein, 9 aa, synthetic construct
VYKIITFWS

SEQ ID NO: 106 - protein, 9 aa, synthetic construct
GWIQRHNLI

SEQ ID NO: 107 - protein, 9 aa, synthetic construct
KTWEDTVVA

SEQ ID NO: 108 - protein, 9 aa, synthetic construct
LDQHHVYQV

SEQ ID NO: 109 - protein, 9 aa, synthetic construct
LEDVWGVWA

SEQ ID NO: 110 - protein, 9 aa, synthetic construct
WLPWTVELI

SEQ ID NO: 111 - protein, 9 aa, synthetic construct
QHFTWDWFW

SEQ ID NO: 112 - protein, 9 aa, synthetic construct
LFEVLQGSS

SEQ ID NO: 113 - protein, 9 aa, synthetic construct
PYSWQHEVV

SEQ ID NO: 114 - protein, 9 aa, synthetic construct
WWQENAEWI

SEQ ID NO: 115 - protein, 9 aa, synthetic construct
SYDPVYQDT

SEQ ID NO: 116 - protein, 9 aa, synthetic construct
RVFDTPLAA

SEQ ID NO: 117 - protein, 9 aa, synthetic construct
WWDIPWIWV

SEQ ID NO: 118 - protein, 9 aa, synthetic construct
YQKVITWWQ

SEQ ID NO: 119 - protein, 9 aa, synthetic construct
QWIKWGNIL

SEQ ID NO: 120 - protein, 9 aa, synthetic construct
ATPEKKYVV

SEQ ID NO: 121 - protein, 9 aa, synthetic construct
TSERLWILY

SEQ ID NO: 122 - protein, 9 aa, synthetic construct
EHHSQPLAH

SEQ ID NO: 123 - protein, 9 aa, synthetic construct
WTADWLWTV

SEQ ID NO: 124 - protein, 9 aa, synthetic construct
SFQREHYSW

SEQ ID NO: 125 - protein, 9 aa, synthetic construct
GKTWHKHNR

SEQ ID NO: 126 - protein, 9 aa, synthetic construct
KWWYRIGNP

SEQ ID NO: 127 - protein, 9 aa, synthetic construct
LERFNFHGE

SEQ ID NO: 128 - protein, 9 aa, synthetic construct
VYKNKRGGH

SEQ ID NO: 129 - protein, 9 aa, synthetic construct
WGENLFAKW

SEQ ID NO: 130 - protein, 9 aa, synthetic construct
GIRKKQQQR

SEQ ID NO: 131 - protein, 9 aa, synthetic construct
GHAFYFAFV

SEQ ID NO: 132 - protein, 9 aa, synthetic construct
TYGKKWYSW

SEQ ID NO: 133 - protein, 9 aa, synthetic construct
YFYRHWEDR

SEQ ID NO: 134 - protein, 9 aa, synthetic construct
WQVHDKQWL

SEQ ID NO: 135 - protein, 9 aa, synthetic construct
PYWYRKNNL

SEQ ID NO: 136 - protein, 9 aa, synthetic construct
ASDIYLRID

SEQ ID NO: 137 - protein, 9 aa, synthetic construct
FLHRKPQDR

SEQ ID NO: 138 - protein, 9 aa, synthetic construct
WQDHHNVKR

SEQ ID NO: 139 - protein, 9 aa, synthetic construct
PYGYRAKAP

SEQ ID NO: 140 - protein, 9 aa, synthetic construct
PLVQELWGF

SEQ ID NO: 141 - protein, 9 aa, synthetic construct
YWLLEIEHR

SEQ ID NO: 142 - protein, 9 aa, synthetic construct
RWRYPARKP

SEQ ID NO: 143 - protein, 9 aa, synthetic construct
LYIYRHPNY

SEQ ID NO: 144 - protein, 9 aa, synthetic construct
PWLYRQKSD

SEQ ID NO: 145 - protein, 9 aa, synthetic construct
PYWYRHDQE

SEQ ID NO: 146 - protein, 9 aa, synthetic construct
PYWYRKYPD

SEQ ID NO: 147 - protein, 8 aa, synthetic construct
EYKFRNLE

SEQ ID NO: 148 - protein, 9 aa, synthetic construct
EDFIAADDD

SEQ ID NO: 149 - protein, 9 aa, synthetic construct
DSQEFPSHL

SEQ ID NO: 150 - protein, 9 aa, synthetic construct
EPDWPDFLI

SEQ ID NO: 151 - protein, 9 aa, synthetic construct
ILNPKSWNF

SEQ ID NO: 152 - protein, 9 aa, synthetic construct
WNENHFSVW

SEQ ID NO: 153 - protein, 9 aa, synthetic construct
NKDWEDQII

SEQ ID NO: 154 - protein, 9 aa, synthetic construct
YYNGDNTQI

SEQ ID NO: 155 - protein, 9 aa, synthetic construct
YWDDKVGYY

SEQ ID NO: 156 - protein, 9 aa, synthetic construct
FHFRKPQDR

SEQ ID NO: 157 - protein, 9 aa, synthetic construct
PYWYREPEN

SEQ ID NO: 158 - protein, 9 aa, synthetic construct
EYGWYHLPY

SEQ ID NO: 159 - protein, 9 aa, synthetic construct
TYHFRTDLN

SEQ ID NO: 160 - protein, 9 aa, synthetic construct
PYLWRKHAV

SEQ ID NO: 161 - protein, 9 aa, synthetic construct
LYQFRKEPV

SEQ ID NO: 162 - protein, 9 aa, synthetic construct
FYYRGPQPV

SEQ ID NO: 163 - protein, 9 aa, synthetic construct
YPDGDLILY

SEQ ID NO: 164 - protein, 9 aa, synthetic construct
QFGEIIWWE

SEQ ID NO: 165 - protein, 9 aa, synthetic construct
VEERWGLWV

SEQ ID NO: 166 - protein, 9 aa, synthetic construct
HHPWQPAKQ

SEQ ID NO: 167 - protein, 9 aa, synthetic construct
PYRWRPDHA

SEQ ID NO: 168 - protein, 9 aa, synthetic construct
HSNAWNLQH

SEQ ID NO: 169 - protein, 9 aa, synthetic construct
FYYYRSEPT

SEQ ID NO: 170 - protein, 9 aa, synthetic construct
PYWYRPNRP

SEQ ID NO: 171 - protein, 9 aa, synthetic construct
ELYSNQIVW

SEQ ID NO: 172 - protein, 9 aa, synthetic construct
YKDWVVNPP

SEQ ID NO: 173 - protein, 9 aa, synthetic construct
PYQYRKDPK

SEQ ID NO: 174 - protein, 9 aa, synthetic construct
YYGNTSILI

SEQ ID NO: 175 - protein, 9 aa, synthetic construct
FKYRHQQDK

SEQ ID NO: 176 - protein, 9 aa, synthetic construct
YWYRNPADR

SEQ ID NO: 177 - protein, 9 aa, synthetic construct
ETKLAHEDI

SEQ ID NO: 178 - protein, 9 aa, synthetic construct
LGEQYIDVD

SEQ ID NO: 179 - protein, 9 aa, synthetic construct
PYRYRTHHK

SEQ ID NO: 180 - protein, 9 aa, synthetic construct
YNPKPGIDN

SEQ ID NO: 181 - protein, 9 aa, synthetic construct
FYYRKRYDR

SEQ ID NO: 182 - protein, 9 aa, synthetic construct
YQYRNPIDK

SEQ ID NO: 183 - protein, 9 aa, synthetic construct
IEYQSPALY

SEQ ID NO: 184 - protein, 9 aa, synthetic construct
PDAPAYSKS

SEQ ID NO: 185 - protein, 9 aa, synthetic construct
PDLNYEVTL

SEQ ID NO: 186 - protein, 9 aa, synthetic construct
YDQNQWSPH

SEQ ID NO: 187 - protein, 9 aa, synthetic construct
RWQYRHPQT

SEQ ID NO: 188 - protein, 9 aa, synthetic construct
RFSAGEEWE

SEQ ID NO: 189 - protein, 9 aa, synthetic construct
DYPRHHQDQ

SEQ ID NO: 190 - protein, 9 aa, synthetic construct
FRKKWAQLW

SEQ ID NO: 191 - protein, 9 aa, synthetic construct
PFFYRKDNP

SEQ ID NO: 192 - protein, 9 aa, synthetic construct
EFILQPWWY

SEQ ID NO: 193 - protein, 9 aa, synthetic construct
KYRHRLTYE

SEQ ID NO: 194 - protein, 9 aa, synthetic construct
YAQRGEVYF

SEQ ID NO: 195 - protein, 9 aa, synthetic construct
VRAFLYYKD

SEQ ID NO: 196 - protein, 9 aa, synthetic construct
PWEFRGDHK

SEQ ID NO: 197 - protein, 9 aa, synthetic construct
GWSHYFQYL

SEQ ID NO: 198 - protein, 9 aa, synthetic construct
FSEDLTLNG

SEQ ID NO: 199 - protein, 9 aa, synthetic construct
TQWEFIEWP

SEQ ID NO: 200 - protein, 9 aa, synthetic construct
FTYRLPQHR

SEQ ID NO: 201 - protein, 9 aa, synthetic construct
DEIFHGAYN

SEQ ID NO: 202 - protein, 9 aa, synthetic construct
WGEIQHWRQ

SEQ ID NO: 203 - protein, 9 aa, synthetic construct
EQWLPNPYF

SEQ ID NO: 204 - protein, 9 aa, synthetic construct
QLLSHQWTE

SEQ ID NO: 205 - protein, 9 aa, synthetic construct
HRYWTRDVT

SEQ ID NO: 206 - protein, 9 aa, synthetic construct
GDESHKWHK

SEQ ID NO: 207 - protein, 9 aa, synthetic construct
RVDEHYIQF

SEQ ID NO: 208 - protein, 9 aa, synthetic construct
SDWRHHGGW

SEQ ID NO: 209 - protein, 9 aa, synthetic construct
WFDNLHNNF

SEQ ID NO: 210 - protein, 9 aa, synthetic construct
PSSGGGPKW

SEQ ID NO: 211 - protein, 9 aa, synthetic construct
RSDTLHYWN

SEQ ID NO: 212 - protein, 9 aa, synthetic construct
IQYRWQDRV

SEQ ID NO: 213 - protein, 9 aa, synthetic construct
FQPIKLANA

SEQ ID NO: 214 - protein, 9 aa, synthetic construct
FAFRKYEDR

SEQ ID NO: 215 - protein, 9 aa, synthetic construct
LHNWWKGNW

SEQ ID NO: 216 - protein, 9 aa, synthetic construct
LNRYYHASI

SEQ ID NO: 217 - protein, 9 aa, synthetic construct
RYWAFRYLW

SEQ ID NO: 218 - protein, 9 aa, synthetic construct
PFWANEHGW

SEQ ID NO: 219 - protein, 9 aa, synthetic construct
ADSGFNATP

SEQ ID NO: 220 - protein, 9 aa, synthetic construct
RQHKKPHPN

SEQ ID NO: 221 - protein, 9 aa, synthetic construct
HSHRFHEAH

SEQ ID NO: 222 - protein, 9 aa, synthetic construct
PWHYPPNKW

SEQ ID NO: 223 - protein, 9 aa, synthetic construct
YKTPFPQYT

SEQ ID NO: 224 - protein, 9 aa, synthetic construct
YQLVDLDLD

SEQ ID NO: 225 - protein, 9 aa, synthetic construct
EYPPVQQVL

SEQ ID NO: 226 - protein, 9 aa, synthetic construct
YSEPPPVQN

SEQ ID NO: 227 - protein, 9 aa, synthetic construct
EYLYRPLNP

SEQ ID NO: 228 - protein, 9 aa, synthetic construct
ALLIHHDPW

SEQ ID NO: 229 - protein, 9 aa, synthetic construct
EFQHRTFNH

SEQ ID NO: 230 - protein, 9 aa, synthetic construct
GEIWSAKHK

SEQ ID NO: 231 - protein, 9 aa, synthetic construct
PEYKPVDHT

SEQ ID NO: 232 - protein, 9 aa, synthetic construct
GEIVWYQVE

SEQ ID NO: 233 - protein, 10 aa, synthetic construct
IVFYLQEYYE

SEQ ID NO: 234 - protein, 9 aa, synthetic construct
GYAVFHKYL

SEQ ID NO: 235 - protein, 9 aa, synthetic construct
FFHRTSVVY

SEQ ID NO: 236 - protein, 9 aa, synthetic construct
PWQYRNNPE

SEQ ID NO: 237 - protein, 9 aa, synthetic construct
QNDELYPYK

SEQ ID NO: 238 - protein, 9 aa, synthetic construct
YWENHRGHV

SEQ ID NO: 239 - protein, 9 aa, synthetic construct
YEAIERWHG

SEQ ID NO: 240 - protein, 9 aa, synthetic construct
WNANILRYW

SEQ ID NO: 241 - protein, 9 aa, synthetic construct
TPRYVPLNP

SEQ ID NO: 242 - protein, 9 aa, synthetic construct
PYHAFDWYW

SEQ ID NO: 243 - protein, 9 aa, synthetic construct
YDPSEWFPR

SEQ ID NO: 244 - protein, 9 aa, synthetic construct
FDIFYFNPI

SEQ ID NO: 245 - protein, 9 aa, synthetic construct
NLYFFFYQT

SEQ ID NO: 246 - protein, 111 aa, synthetic construct
MIPGGLSEAKPATPEIQEIVDKVKPQLEEKTGETYGKLEAVQYKTQVVHDVFYDQKAGTNYYIKVRAGDNKYMHLKVFKSLKWWYRIGNPEDLVLTGYQVDKNKDDELTGF

SEQ ID NO: 247 - protein, 111 aa, synthetic construct
MIPGGLSEAKPATPEIQEIVDKVKPQLEEKTGETYGKLEAVQYKTQVVPFKIITVWHGTNYYIKVRAGDNKYMHLKVFKSLLERFNFHGEEDLVLTGYQVDKNKDDELTGF

SEQ ID NO: 248 - protein, 111 aa, synthetic construct
MIPGGLSEAKPATPEIQEIVDKVKPQLEEKTGETYGKLEAVQYKTQVVQYKIITWWSGTNYYIKVRAGDNKYMHLKVFKSLVYKNKRGGHEDLVLTGYQVDKNKDDELTGF

SEQ ID NO: 249 - protein, 111 aa, synthetic construct
MIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGF

SEQ ID NO: 250 - protein, 111 aa, synthetic construct
MIPGGLSEAKPATPEIQEIVDKVKPQLEEKTGETYGKLEAVQYKTQVVYYQIITWWQGTNYYIKVRAGDNKYMHLKVFKSLGIRKKQQQREDLVLTGYQVDKNKDDELTGF

SEQ ID NO: 251 - protein, 111 aa, synthetic construct
MIPGGLSEAKPATPEIQEIVDKVKPQLEEKTGETYGKLEAVQYKTQVVVHGPHLEQAGTNYYIKVRAGDNKYMHLKVFKSLGHAFYFAFVEDLVLTGYQVDKNKDDELTGF

SEQ ID NO: 252 - protein, 111 aa, synthetic construct
MIPGGLSEAKPATPEIQEIVDKVKPQLEEKTGETYGKLEAVQYKTQVVLQDQWPWRDGTNYYIKVRAGDNKYMHLKVFKSLTYGKKWYSWEDLVLTGYQVDKNKDDELTGF

SEQ ID NO: 253 - protein, 103 aa, synthetic construct
MIPGGLSEAKPATPEIQEIVDKVKPQLEEKTGETYGKLEAVQYKTQVDAGTNYYIKVRAGDNKYMHLKVFKSLYFYRHWEDREDLVLTGYQVDKNKDDELTGF

SEQ ID NO: 254 - protein, 111 aa, synthetic construct
MIPGGLSEAKPATPEIQEIVDKVKPQLEEKTGETYGKLEAVQYKTQVVSWVKPDNELGTNYYIKVRAGDNKYMHLKVFKSLWQVHDKQWLEDLVLTGYQVDKNKDDELTGF

SEQ ID NO: 255 - protein, 111 aa, synthetic construct
MIPGGLSEAKPATPEIQEIVDKVKPQLEEKTGETYGKLEAVQYKTQVVNSLGVSEWVGTNYYIKVRAGDNKYMHLKVFKSLPYWYRKNNLEDLVLTGYQVDKNKDDELTGF

SEQ ID NO: 256 - protein, 111 aa, synthetic construct
MIPGGLSEAKPATPEIQEIVDKVKPQLEEKTGETYGKLEAVQYKTQVVVYRIVSWWRGTNYYIKVRAGDNKYMHLKVFKSLASDIYLRIDEDLVLTGYQVDKNKDDELTGF

SEQ ID NO: 257 - protein, 111 aa, synthetic construct
MIPGGLSEAKPATPEIQEIVDKVKPQLEEKTGETYGKLEAVQYKTQVVYYRQLKFNTGTNYYIKVRAGDNKYMHLKVFKSLFLHRKPQDREDLVLTGYQVDKNKDDELTGF

SEQ ID NO: 258 - protein, 111 aa, synthetic construct
MIPGGLSEAKPATPEIQEIVDKVKPQLEEKTGETYGKLEAVQYKTQVVGWIKVDNVLGTNYYIKVRAGDNKYMHLKVFKSLWQDHHNVKREDLVLTGYQVDKNKDDELTGF

SEQ ID NO: 259 - protein, 111 aa, synthetic construct
MIPGGLSEAKPATPEIQEIVDKVKPQLEEKTGETYGKLEAVQYKTQVVTAYYIHVNAGTNYYIKVRAGDNKYMHLKVFKSLPYGYRAKAPEDLVLTGYQVDKNKDDELTGF

SEQ ID NO: 260 - protein, 111 aa, synthetic construct
MIPGGLSEAKPATPEIQEIVDKVKPQLEEKTGETYGKLEAVQYKTQVVVRVEPTPNDGTNYYIKVRAGDNKYMHLKVFKSLPLVQELWGFEDLVLTGYQVDKNKDDELTGF

SEQ ID NO: 261 - protein, 111 aa, synthetic construct
MIPGGLSEAKPATPEIQEIVDKVKPQLEEKTGETYGKLEAVQYKTQVVDLNSSVWYQGTNYYIKVRAGDNKYMHLKVFKSLYWLLEIEHREDLVLTGYQVDKNKDDELTGF

SEQ ID NO: 262 - protein, 111 aa, synthetic construct
MIPGGLSEAKPATPEIQEIVDKVKPQLEEKTGETYGKLEAVQYKTQVVFTYQISYTVGTNYYIKVRAGDNKYMHLKVFKSLRWRYPARKPEDLVLTGYQVDKNKDDELTGF

SEQ ID NO: 263 - protein, 111 aa, synthetic construct
MIPGGLSEAKPATPEIQEIVDKVKPQLEEKTGETYGKLEAVQYKTQVVRENWYGEFAGTNYYIKVRAGDNKYMHLKVFKSLLYIYRHPNYEDLVLTGYQVDKNKDDELTGF

SEQ ID NO: 264 - protein, 111 aa, synthetic construct
MIPGGLSEAKPATPEIQEIVDKVKPQLEEKTGETYGKLEAVQYKTQVVTALAWNTQAGTNYYIKVRAGDNKYMHLKVFKSLPWLYRQKSDEDLVLTGYQVDKNKDDELTGF

SEQ ID NO: 265 - protein, 111 aa, synthetic construct
MIPGGLSEAKPATPEIQEIVDKVKPQLEEKTGETYGKLEAVQYKTQVVHIPALKVHAGTNYYIKVRAGDNKYMHLKVFKSLPYWYRHDQEEDLVLTGYQVDKNKDDELTGF

SEQ ID NO: 266 - protein, 111 aa, synthetic construct
MIPGGLSEAKPATPEIQEIVDKVKPQLEEKTGETYGKLEAVQYKTQVVHHAYAAVYVGTNYYIKVRAGDNKYMHLKVFKSLPYWYRKYPDEDLVLTGYQVDKNKDDELTGF

SEQ ID NO: 267 - protein, 110 aa, synthetic construct
MIPGGLSEAKPATPEIQEIVDKVKPQLEEKTGETYGKLEAVQYKTQVVYDELYHEKAGTNYYIKVRAGDNKYMHLKVFKSLEYKFRNLEEDLVLTGYQVDKNKDDELTGF

SEQ ID NO: 268 - protein, 111 aa, synthetic construct
MIPGGLSEAKPATPEIQEIVDKVKPQLEEKTGETYGKLEAVQYKTQVVSWYYPKNVLGTNYYIKVRAGDNKYMHLKVFKSLEDFIAADDDEDLVLTGYQVDKNKDDELTGF

SEQ ID NO: 269 - protein, 111 aa, synthetic construct
MIPGGLSEAKPATPEIQEIVDKVKPQLEEKTGETYGKLEAVQYKTQVVFLEWHFWEWGTNYYIKVRAGDNKYMHLKVFKSLDSQEFPSHLEDLVLTGYQVDKNKDDELTGF

SEQ ID NO: 270 - protein, 111 aa, synthetic construct
MIPGGLSEAKPATPEIQEIVDKVKPQLEEKTGETYGKLEAVQYKTQVVSWVRGTNQLGTNYYIKVRAGDNKYMHLKVFKSLEPDWPDFLIEDLVLTGYQVDKNKDDELTGF

SEQ ID NO: 271 - protein, 111 aa, synthetic construct
MIPGGLSEAKPATPEIQEIVDKVKPQLEEKTGETYGKLEAVQYKTQVVWRKLLSWWKGTNYYIKVRAGDNKYMHLKVFKSLILNPKSWNFEDLVLTGYQVDKNKDDELTGF

SEQ ID NO: 272 - protein, 111 aa, synthetic construct
MIPGGLSEAKPATPEIQEIVDKVKPQLEEKTGETYGKLEAVQYKTQVVPKSHQTNEQGTNYYIKVRAGDNKYMHLKVFKSLWNENHFSVWEDLVLTGYQVDKNKDDELTGF

SEQ ID NO: 273 - protein, 111 aa, synthetic construct
MIPGGLSEAKPATPEIQEIVDKVKPQLEEKTGETYGKLEAVQYKTQVVNWNQIDNPLGTNYYIKVRAGDNKYMHLKVFKSLNKDWEDQIIEDLVLTGYQVDKNKDDELTGF

SEQ ID NO: 274 - protein, 111 aa, synthetic construct
MIPGGLSEAKPATPEIQEIVDKVKPQLEEKTGETYGKLEAVQYKTQVVFWVGLHNQLGTNYYIKVRAGDNKYMHLKVFKSLYYNGDNTQIEDLVLTGYQVDKNKDDELTGF

SEQ ID NO: 275 - protein, 111 aa, synthetic construct
MIPGGLSEAKPATPEIQEIVDKVKPQLEEKTGETYGKLEAVQYKTQVVFTQPFNEPIGTNYYIKVRAGDNKYMHLKVFKSLYWDDKVGYYEDLVLTGYQVDKNKDDELTGF

SEQ ID NO: 276 - protein, 111 aa, synthetic construct
MIPGGLSEAKPATPEIQEIVDKVKPQLEEKTGETYGKLEAVQYKTQVVPEYQPQPKAGTNYYIKVRAGDNKYMHLKVFKSLFHFRKPQDREDLVLTGYQVDKNKDDELTGF

SEQ ID NO: 277 - protein, 111 aa, synthetic construct
MIPGGLSEAKPATPEIQEIVDKVKPQLEEKTGETYGKLEAVQYKTQVVNDPYFQSFSGTNYYIKVRAGDNKYMHLKVFKSLPYWYREPENEDLVLTGYQVDKNKDDELTGF

SEQ ID NO: 278 - protein, 111 aa, synthetic construct
MIPGGLSEAKPATPEIQEIVDKVKPQLEEKTGETYGKLEAVQYKTQVVHQDDVAWQSGTNYYIKVRAGDNKYMHLKVFKSLEYGWYHLPYEDLVLTGYQVDKNKDDELTGF

SEQ ID NO: 279 - protein, 111 aa, synthetic construct
MIPGGLSEAKPATPEIQEIVDKVKPQLEEKTGETYGKLEAVQYKTQVVKGYFHTVEAGTNYYIKVRAGDNKYMHLKVFKSLTYHFRTDLNEDLVLTGYQVDKNKDDELTGF

SEQ ID NO: 280 - protein, 111 aa, synthetic construct
MIPGGLSEAKPATPEIQEIVDKVKPQLEEKTGETYGKLEAVQYKTQVVVDQVWSENAGTNYYIKVRAGDNKYMHLKVFKSLPYLWRKHAVEDLVLTGYQVDKNKDDELTGF

SEQ ID NO: 281 - protein, 111 aa, synthetic construct
MIPGGLSEAKPATPEIQEIVDKVKPQLEEKTGETYGKLEAVQYKTQVVLSSIWQDVAGTNYYIKVRAGDNKYMHLKVFKSLLYQFRKEPVEDLVLTGYQVDKNKDDELTGF

SEQ ID NO: 282 - protein, 111 aa, synthetic construct
MIPGGLSEAKPATPEIQEIVDKVKPQLEEKTGETYGKLEAVQYKTQVVTASLYWNYAGTNYYIKVRAGDNKYMHLKVFKSLFYYRGPQPVEDLVLTGYQVDKNKDDELTGF

SEQ ID NO: 283 - protein, 111 aa, synthetic construct
MIPGGLSEAKPATPEIQEIVDKVKPQLEEKTGETYGKLEAVQYKTQVVSWKQWHNYLGTNYYIKVRAGDNKYMHLKVFKSLYPDGDLILYEDLVLTGYQVDKNKDDELTGF

SEQ ID NO: 284 - protein, 111 aa, synthetic construct
MIPGGLSEAKPATPEIQEIVDKVKPQLEEKTGETYGKLEAVQYKTQVVQVHLDNSPLGTNYYIKVRAGDNKYMHLKVFKSLQFGEIIWWEEDLVLTGYQVDKNKDDELTGF

SEQ ID NO: 285 - protein, 111 aa, synthetic construct
MIPGGLSEAKPATPEIQEIVDKVKPQLEEKTGETYGKLEAVQYKTQVVQNNWYLSNYGTNYYIKVRAGDNKYMHLKVFKSLVEERWGLWVEDLVLTGYQVDKNKDDELTGF

SEQ ID NO: 286 - protein, 111 aa, synthetic construct
MIPGGLSEAKPATPEIQEIVDKVKPQLEEKTGETYGKLEAVQYKTQVVWEWALLLEYGTNYYIKVRAGDNKYMHLKVFKSLHHPWQPAKQEDLVLTGYQVDKNKDDELTGF

SEQ ID NO: 287 - protein, 111 aa, synthetic construct
MIPGGLSEAKPATPEIQEIVDKVKPQLEEKTGETYGKLEAVQYKTQVVGDSYWEYVSGTNYYIKVRAGDNKYMHLKVFKSLPYRWRPDHAEDLVLTGYQVDKNKDDELTGF

SEQ ID NO: 288 - protein, 111 aa, synthetic construct
MIPGGLSEAKPATPEIQEIVDKVKPQLEEKTGETYGKLEAVQYKTQVVGQKWWQHKIGTNYYIKVRAGDNKYMHLKVFKSLHSNAWNLQHEDLVLTGYQVDKNKDDELTGF

SEQ ID NO: 289 - protein, 111 aa, synthetic construct
MIPGGLSEAKPATPEIQEIVDKVKPQLEEKTGETYGKLEAVQYKTQVVLDGLYHVNSGTNYYIKVRAGDNKYMHLKVFKSLFYYYRSEPTEDLVLTGYQVDKNKDDELTGF

SEQ ID NO: 290 - protein, 111 aa, synthetic construct
MIPGGLSEAKPATPEIQEIVDKVKPQLEEKTGETYGKLEAVQYKTQVVHAPAPVQAIGTNYYIKVRAGDNKYMHLKVFKSLPYWYRPNRPEDLVLTGYQVDKNKDDELTGF

SEQ ID NO: 291 - protein, 111 aa, synthetic construct
MIPGGLSEAKPATPEIQEIVDKVKPQLEEKTGETYGKLEAVQYKTQVVWEDHWNYQYGTNYYIKVRAGDNKYMHLKVFKSLELYSNQIVWEDLVLTGYQVDKNKDDELTGF

SEQ ID NO: 292 - protein, 111 aa, synthetic construct
MIPGGLSEAKPATPEIQEIVDKVKPQLEEKTGETYGKLEAVQYKTQVVSWRYKSNQLGTNYYIKVRAGDNKYMHLKVFKSLYKDWVVNPPEDLVLTGYQVDKNKDDELTGF

SEQ ID NO: 293 - protein, 111 aa, synthetic construct
MIPGGLSEAKPATPEIQEIVDKVKPQLEEKTGETYGKLEAVQYKTQVVRVDWWLPEVGTNYYIKVRAGDNKYMHLKVFKSLPYQYRKDPKEDLVLTGYQVDKNKDDELTGF

SEQ ID NO: 294 - protein, 111 aa, synthetic construct
MIPGGLSEAKPATPEIQEIVDKVKPQLEEKTGETYGKLEAVQYKTQVVSWIEISNQLGTNYYIKVRAGDNKYMHLKVFKSLYYGNTSILIEDLVLTGYQVDKNKDDELTGF

SEQ ID NO: 295 - protein, 111 aa, synthetic construct
MIPGGLSEAKPATPEIQEIVDKVKPQLEEKTGETYGKLEAVQYKTQVVSLGQAETWAGTNYYIKVRAGDNKYMHLKVFKSLFKYRHQQDKEDLVLTGYQVDKNKDDELTGF

SEQ ID NO: 296 - protein, 111 aa, synthetic construct
MIPGGLSEAKPATPEIQEIVDKVKPQLEEKTGETYGKLEAVQYKTQVVKIGQLPSRVGTNYYIKVRAGDNKYMHLKVFKSLYWYRNPADREDLVLTGYQVDKNKDDELTGF

SEQ ID NO: 297 - protein, 111 aa, synthetic construct
MIPGGLSEAKPATPEIQEIVDKVKPQLEEKTGETYGKLEAVQYKTQVVAWHTKNNSIGTNYYIKVRAGDNKYMHLKVFKSLETKLAHEDIEDLVLTGYQVDKNKDDELTGF

SEQ ID NO: 298 - protein, 111 aa, synthetic construct
MIPGGLSEAKPATPEIQEIVDKVKPQLEEKTGETYGKLEAVQYKTQVVQTKLVTWWWGTNYYIKVRAGDNKYMHLKVFKSLLGEQYIDVDEDLVLTGYQVDKNKDDELTGF

SEQ ID NO: 299 - protein, 111 aa, synthetic construct
MIPGGLSEAKPATPEIQEIVDKVKPQLEEKTGETYGKLEAVQYKTQVVAVFDYADPSGTNYYIKVRAGDNKYMHLKVFKSLPYRYRTHHKEDLVLTGYQVDKNKDDELTGF

SEQ ID NO: 300 - protein, 111 aa, synthetic construct
MIPGGLSEAKPATPEIQEIVDKVKPQLEEKTGETYGKLEAVQYKTQVVHVKIVTWWKGTNYYIKVRAGDNKYMHLKVFKSLYNPKPGIDNEDLVLTGYQVDKNKDDELTGF

SEQ ID NO: 301 - protein, 111 aa, synthetic construct
MIPGGLSEAKPATPEIQEIVDKVKPQLEEKTGETYGKLEAVQYKTQVVHVLKVSSVSGTNYYIKVRAGDNKYMHLKVFKSLFYYRKRYDREDLVLTGYQVDKNKDDELTGF

SEQ ID NO: 302 - protein, 111 aa, synthetic construct
MIPGGLSEAKPATPEIQEIVDKVKPQLEEKTGETYGKLEAVQYKTQVVYQDYDSATHGTNYYIKVRAGDNKYMHLKVFKSLYQYRNPIDKEDLVLTGYQVDKNKDDELTGF

SEQ ID NO: 303 - protein, 111 aa, synthetic construct
MIPGGLSEAKPATPEIQEIVDKVKPQLEEKTGETYGKLEAVQYKTQVVSWGKVSNELGTNYYIKVRAGDNKYMHLKVFKSLIEYQSPALYEDLVLTGYQVDKNKDDELTGF

SEQ ID NO: 304 - protein, 111 aa, synthetic construct
MIPGGLSEAKPATPEIQEIVDKVKPQLEEKTGETYGKLEAVQYKTQVVLEDEWWSILGTNYYIKVRAGDNKYMHLKVFKSLPDAPAYSKSEDLVLTGYQVDKNKDDELTGF

SEQ ID NO: 305 - protein, 111 aa, synthetic construct
MIPGGLSEAKPATPEIQEIVDKVKPQLEEKTGETYGKLEAVQYKTQVVYYKLVTWWRGTNYYIKVRAGDNKYMHLKVFKSLPDLNYEVTLEDLVLTGYQVDKNKDDELTGF

SEQ ID NO: 306 - protein, 111 aa, synthetic construct
MIPGGLSEAKPATPEIQEIVDKVKPQLEEKTGETYGKLEAVQYKTQVVAWVKEHNLIGTNYYIKVRAGDNKYMHLKVFKSLYDQNQWSPHEDLVLTGYQVDKNKDDELTGF

SEQ ID NO: 307 - protein, 111 aa, synthetic construct
MIPGGLSEAKPATPEIQEIVDKVKPQLEEKTGETYGKLEAVQYKTQVVQEYLWKQYIGTNYYIKVRAGDNKYMHLKVFKSLRWQYRHPQTEDLVLTGYQVDKNKDDELTGF

SEQ ID NO: 308 - protein, 111 aa, synthetic construct
MIPGGLSEAKPATPEIQEIVDKVKPQLEEKTGETYGKLEAVQYKTQVVFNKDLFLGWGTNYYIKVRAGDNKYMHLKVFKSLRFSAGEEWEEDLVLTGYQVDKNKDDELTGF

SEQ ID NO: 309 - protein, 111 aa, synthetic construct
MIPGGLSEAKPATPEIQEIVDKVKPQLEEKTGETYGKLEAVQYKTQVVHFGIRYAHVGTNYYIKVRAGDNKYMHLKVFKSLDYPRHHQDQEDLVLTGYQVDKNKDDELTGF

SEQ ID NO: 310 - protein, 111 aa, synthetic construct
MIPGGLSEAKPATPEIQEIVDKVKPQLEEKTGETYGKLEAVQYKTQVVWYAWQEGWQGTNYYIKVRAGDNKYMHLKVFKSLFRKKWAQLWEDLVLTGYQVDKNKDDELTGF

SEQ ID NO: 311 - protein, 111 aa, synthetic construct
MIPGGLSEAKPATPEIQEIVDKVKPQLEEKTGETYGKLEAVQYKTQVVGNAAWGSFAGTNYYIKVRAGDNKYMHLKVFKSLPFFYRKDNPEDLVLTGYQVDKNKDDELTGF

SEQ ID NO: 312 - protein, 111 aa, synthetic construct
MIPGGLSEAKPATPEIQEIVDKVKPQLEEKTGETYGKLEAVQYKTQVVREDLDLFYTGTNYYIKVRAGDNKYMHLKVFKSLEFILQPWWYEDLVLTGYQVDKNKDDELTGF

SEQ ID NO: 313 - protein, 111 aa, synthetic construct
MIPGGLSEAKPATPEIQEIVDKVKPQLEEKTGETYGKLEAVQYKTQVVNENVYRVTQGTNYYIKVRAGDNKYMHLKVFKSLKYRHRLTYEEDLVLTGYQVDKNKDDELTGF

SEQ ID NO: 314 - protein, 111 aa, synthetic construct
MIPGGLSEAKPATPEIQEIVDKVKPQLEEKTGETYGKLEAVQYKTQVVWHKIVTQWPGTNYYIKVRAGDNKYMHLKVFKSLYAQRGEVYFEDLVLTGYQVDKNKDDELTGF

SEQ ID NO: 315 - protein, 111 aa, synthetic construct
MIPGGLSEAKPATPEIQEIVDKVKPQLEEKTGETYGKLEAVQYKTQVVHYRIITHWEGTNYYIKVRAGDNKYMHLKVFKSLVRAFLYYKDEDLVLTGYQVDKNKDDELTGF

SEQ ID NO: 316 - protein, 111 aa, synthetic construct
MIPGGLSEAKPATPEIQEIVDKVKPQLEEKTGETYGKLEAVQYKTQVVLVFDASTSAGTNYYIKVRAGDNKYMHLKVFKSLPWEFRGDHKEDLVLTGYQVDKNKDDELTGF

SEQ ID NO: 317 - protein, 111 aa, synthetic construct
MIPGGLSEAKPATPEIQEIVDKVKPQLEEKTGETYGKLEAVQYKTQVVQYAEFLEHIGTNYYIKVRAGDNKYMHLKVFKSLGWSHYFQYLEDLVLTGYQVDKNKDDELTGF

SEQ ID NO: 318 - protein, 111 aa, synthetic construct
MIPGGLSEAKPATPEIQEIVDKVKPQLEEKTGETYGKLEAVQYKTQVVGNVQEDWHIGTNYYIKVRAGDNKYMHLKVFKSLFSEDLTLNGEDLVLTGYQVDKNKDDELTGF

SEQ ID NO: 319 - protein, 111 aa, synthetic construct
MIPGGLSEAKPATPEIQEIVDKVKPQLEEKTGETYGKLEAVQYKTQVVGWSASYPESGTNYYIKVRAGDNKYMHLKVFKSLTQWEFIEWPEDLVLTGYQVDKNKDDELTGF

SEQ ID NO: 320 - protein, 111 aa, synthetic construct
MIPGGLSEAKPATPEIQEIVDKVKPQLEEKTGETYGKLEAVQYKTQVVYQFEHISYGGTNYYIKVRAGDNKYMHLKVFKSLFTYRLPQHREDLVLTGYQVDKNKDDELTGF

SEQ ID NO: 321 - protein, 111 aa, synthetic construct
MIPGGLSEAKPATPEIQEIVDKVKPQLEEKTGETYGKLEAVQYKTQVVAWITPVNVLGTNYYIKVRAGDNKYMHLKVFKSLDEIFHGAYNEDLVLTGYQVDKNKDDELTGF

SEQ ID NO: 322 - protein, 111 aa, synthetic construct
MIPGGLSEAKPATPEIQEIVDKVKPQLEEKTGETYGKLEAVQYKTQVVESYAAIFWGGTNYYIKVRAGDNKYMHLKVFKSLWGEIQHWRQEDLVLTGYQVDKNKDDELTGF

SEQ ID NO: 323 - protein, 111 aa, synthetic construct
MIPGGLSEAKPATPEIQEIVDKVKPQLEEKTGETYGKLEAVQYKTQVVRVWPWTWDRGTNYYIKVRAGDNKYMHLKVFKSLEQWLPNPYFEDLVLTGYQVDKNKDDELTGF

SEQ ID NO: 324 - protein, 111 aa, synthetic construct
MIPGGLSEAKPATPEIQEIVDKVKPQLEEKTGETYGKLEAVQYKTQVVQVKKWWEYFVTNYYIKVRAGDNKYMHLKVFKSLQLLSHQWTEEDLVLTGYQVDKNKDDELTGF

SEQ ID NO: 325 - protein, 111 aa, synthetic construct
MIPGGLSEAKPATPEIQEIVDKVKPQLEEKTGETYGKLEAVQYKTQVVIGQISEQQWGTNYYIKVRAGDNKYMHLKVFKSLHRYWTRDVTEDLVLTGYQVDKNKDDELTGF

SEQ ID NO: 326 - protein, 111 aa, synthetic construct
MIPGGLSEAKPATPEIQEIVDKVKPQLEEKTGETYGKLEAVQYKTQVVAFHKLGWILGTNYYIKVRAGDNKYMHLKVFKSLGDESHKWHKEDLVLTGYQVDKNKDDELTGF

SEQ ID NO: 327 - protein, 111 aa, synthetic construct
MIPGGLSEAKPATPEIQEIVDKVKPQLEEKTGETYGKLEAVQYKTQVVFKKWWWKYKGTNYYIKVRAGDNKYMHLKVFKSLRVDEHYIQFEDLVLTGYQVDKNKDDELTGF

SEQ ID NO: 328 - protein, 111 aa, synthetic construct
MIPGGLSEAKPATPEIQEIVDKVKPQLEEKTGETYGKLEAVQYKTQVVASQRLWIIYGTNYYIKVRAGDNKYMHLKVFKSLSDWRHHGGWEDLVLTGYQVDKNKDDELTGF

SEQ ID NO: 329 - protein, 111 aa, synthetic construct
MIPGGLSEAKPATPEIQEIVDKVKPQLEEKTGETYGKLEAVQYKTQVVQRETHILWSGTNYYIKVRAGDNKYMHLKVFKSLWFDNLHNNFEDLVLTGYQVDKNKDDELTGF

SEQ ID NO: 330 - protein, 111 aa, synthetic construct
MIPGGLSEAKPATPEIQEIVDKVKPQLEEKTGETYGKLEAVQYKTQVVGQYWFWAPGGTNYYIKVRAGDNKYMHLKVFKSLPSSGGGPKWEDLVLTGYQVDKNKDDELTGF

SEQ ID NO: 331 - protein, 111 aa, synthetic construct
MIPGGLSEAKPATPEIQEIVDKVKPQLEEKTGETYGKLEAVQYKTQVVRDWAYYIYFGTNYYIKVRAGDNKYMHLKVFKSLRSDTLHYWNEDLVLTGYQVDKNKDDELTGF

SEQ ID NO: 332 - protein, 111 aa, synthetic construct
MIPGGLSEAKPATPEIQEIVDKVKPQLEEKTGETYGKLEAVQYKTQVVIGYKLNYIWGTNYYIKVRAGDNKYMHLKVFKSLIQYRWQDRVEDLVLTGYQVDKNKDDELTGF

SEQ ID NO: 333 - protein, 111 aa, synthetic construct
MIPGGLSEAKPATPEIQEIVDKVKPQLEEKTGETYGKLEAVQYKTQVVVSLTEQRWKGTNYYIKVRAGDNKYMHLKVFKSLFQPIKLANAEDLVLTGYQVDKNKDDELTGF

SEQ ID NO: 334 - protein, 111 aa, synthetic construct
MIPGGLSEAKPATPEIQEIVDKVKPQLEEKTGETYGKLEAVQYKTQVVWVSQQHYSHGTNYYIKVRAGDNKYMHLKVFKSLFAFRKYEDREDLVLTGYQVDKNKDDELTGF

SEQ ID NO: 335 - protein, 111 aa, synthetic construct
MIPGGLSEAKPATPEIQEIVDKVKPQLEEKTGETYGKLEAVQYKTQVVDDDNQHYGLGTNYYIKVRAGDNKYMHLKVFKSLLHNWWKGNWEDLVLTGYQVDKNKDDELTGF

SEQ ID NO: 336 - protein, 111 aa, synthetic construct
MIPGGLSEAKPATPEIQEIVDKVKPQLEEKTGETYGKLEAVQYKTQVVPTRWISIYWGTNYYIKVRAGDNKYMHLKVFKSLLNRYYHASIEDLVLTGYQVDKNKDDELTGF

SEQ ID NO: 337 - protein, 111 aa, synthetic construct
MIPGGLSEAKPATPEIQEIVDKVKPQLEEKTGETYGKLEAVQYKTQVVTAFAPNKASGTNYYIKVRAGDNKYMHLKVFKSLRYWAFRYLWEDLVLTGYQVDKNKDDELTGF

SEQ ID NO: 338 - protein, 111 aa, synthetic construct
MIPGGLSEAKPATPEIQEIVDKVKPQLEEKTGETYGKLEAVQYKTQVVHYTDFKWHKGTNYYIKVRAGDNKYMHLKVFKSLPFWANEHGWEDLVLTGYQVDKNKDDELTGF

SEQ ID NO: 339 - protein, 111 aa, synthetic construct
MIPGGLSEAKPATPEIQEIVDKVKPQLEEKTGETYGKLEAVQYKTQVVNKFWSWAPGGTNYYIKVRAGDNKYMHLKVFKSLADSGFNATPEDLVLTGYQVDKNKDDELTGF

SEQ ID NO: 340 - protein, 111 aa, synthetic construct
MIPGGLSEAKPATPEIQEIVDKVKPQLEEKTGETYGKLEAVQYKTQVVELDEPAWDWGTNYYIKVRAGDNKYMHLKVFKSLRQHKKPHPNEDLVLTGYQVDKNKDDELTGF

SEQ ID NO: 341 - protein, 111 aa, synthetic construct
MIPGGLSEAKPATPEIQEIVDKVKPQLEEKTGETYGKLEAVQYKTQVVHEKWGFWELGTNYYIKVRAGDNKYMHLKVFKSLHSHRFHEAHEDLVLTGYQVDKNKDDELTGF

SEQ ID NO: 342 - protein, 111 aa, synthetic construct
MIPGGLSEAKPATPEIQEIVDKVKPQLEEKTGETYGKLEAVQYKTQVVVATADLIKAGTNYYIKVRAGDNKYMHLKVFKSLPWHYPPNKWEDLVLTGYQVDKNKDDELTGF

SEQ ID NO: 343 - protein, 111 aa, synthetic construct
MIPGGLSEAKPATPEIQEIVDKVKPQLEEKTGETYGKLEAVQYKTQVVYYKVISWWHGTNYYIKVRAGDNKYMHLKVFKSLYKTPFPQYTEDLVLTGYQVDKNKDDELTGF

SEQ ID NO: 344 - protein, 111 aa, synthetic construct
MIPGGLSEAKPATPEIQEIVDKVKPQLEEKTGETYGKLEAVQYKTQVVHWHHYDNIIGTNYYIKVRAGDNKYMHLKVFKSLYQLVDLDLDEDLVLTGYQVDKNKDDELTGF

SEQ ID NO: 345 - protein, 111 aa, synthetic construct
MIPGGLSEAKPATPEIQEIVDKVKPQLEEKTGETYGKLEAVQYKTQVVVYKIITFWSGTNYYIKVRAGDNKYMHLKVFKSLEYPPVQQVLEDLVLTGYQVDKNKDDELTGF

SEQ ID NO: 346 - protein, 111 aa, synthetic construct
MIPGGLSEAKPATPEIQEIVDKVKPQLEEKTGETYGKLEAVQYKTQVVGWIQRHNLIGTNYYIKVRAGDNKYMHLKVFKSLYSEPPPVQNEDLVLTGYQVDKNKDDELTGF

SEQ ID NO: 347 - protein, 111 aa, synthetic construct
MIPGGLSEAKPATPEIQEIVDKVKPQLEEKTGETYGKLEAVQYKTQVVKTWEDTVVAGTNYYIKVRAGDNKYMHLKVFKSLEYLYRPLNPEDLVLTGYQVDKNKDDELTGF

SEQ ID NO: 348 - protein, 111 aa, synthetic construct
MIPGGLSEAKPATPEIQEIVDKVKPQLEEKTGETYGKLEAVQYKTQVVLDQHHVYQVGTNYYIKVRAGDNKYMHLKVFKSLALLIHHDPWEDLVLTGYQVDKNKDDELTGF

SEQ ID NO: 349 - protein, 111 aa, synthetic construct
MIPGGLSEAKPATPEIQEIVDKVKPQLEEKTGETYGKLEAVQYKTQVVLEDVWGVWAGTNYYIKVRAGDNKYMHLKVFKSLEFQHRTFNHEDLVLTGYQVDKNKDDELTGF

SEQ ID NO: 350 - protein, 111 aa, synthetic construct
MIPGGLSEAKPATPEIQEIVDKVKPQLEEKTGETYGKLEAVQYKTQVVWLPWTVELIGTNYYIKVRAGDNKYMHLKVFKSLGEIWSAKHKEDLVLTGYQVDKNKDDELTGF

SEQ ID NO: 351 - protein, 111 aa, synthetic construct
MIPGGLSEAKPATPEIQEIVDKVKPQLEEKTGETYGKLEAVKYKTQVVQHFTWDWFWGTNYYIKVRAGDNKYMHLKVFKSLPEYKPVDHTEDLVLTGYQVDKNKDDELTGF

SEQ ID NO: 352 - protein, 111 aa, synthetic construct
MIPGGLSEAKPATPEIQEIVDKVKPQLEEKTGETYGKLEAVQYKTQVVLFEVLQGSSGTNYYIKVRAGDNKYMHLKVFKSLGEIVWYQVEEDLVLTGYQVDKNKDDELTGF

SEQ ID NO: 353 - protein, 112 aa, synthetic construct
MIPGGLSEAKPATPEIQEIVDKVKPQLEEKTGETYGKLEAVQYKTQVVPYSWQHEVVGTNYYIKVRAGDNKYMHLKVFKSLIVFYLQEYYEEDLVLTGYQVDKNKDDELTGF

SEQ ID NO: 354 - protein, 111 aa, synthetic construct
MIPGGLSEAKPATPEIQEIVDKVKPQLEEKTGETYGKLEAVQYKTQVVWWQENAEWIGTNYYIKVRAGDNKYMHLKVFKSLGYAVFHKYLEDLVLTGYQVDKNKDDELTGF

SEQ ID NO: 355 - protein, 111 aa, synthetic construct
MIPGGLSEAKPATPEIQEIVDKVKPQLEEKTGETYGKLEAVQYKTQVVSYDPVYQDTGTNYYIKVRAGDNKYMHLKVFKSLFFHRTSVVYEDLVLTGYQVDKNKDDELTGF

SEQ ID NO: 356 - protein, 111 aa, synthetic construct
MIPGGLSEAKPATPEIQEIVDKVKPQLEEKTGETYGKLEAVQYKTQVVRVFDTPLAAGTNYYIKVRAGDNKYMHLKVFKSLPWQYRNNPEEDLVLTGYQVDKNKDDELTGF

SEQ ID NO: 357 - protein, 111 aa, synthetic construct
MIPGGLSEAKPATPEIQEIVDKVKPQLEEKTGETYGKLEAVQYKTQVVWWDIPWIWVGTNYYIKVRAGDNKYMHLKVFKSLQNDELYPYKEDLVLTGYQVDKNKDDELTGF

SEQ ID NO: 358 - protein, 111 aa, synthetic construct
MIPGGLSEAKPATPEIQEIVDKVKPQLEEKTGETYGKLEAVQYKTQVVYQKVITWWQGTNYYIKVRAGDNKYMHLKVFKSLYWENHRGHVEDLVLTGYQVDKNKDDELTGF

SEQ ID NO: 359 - protein, 111 aa, synthetic construct
MIPGGLSEAKPATPEIQEIVDKVKPQLEEKTGETYGKLEAVQYKTQVVQWIKWGNILGTNYYIKVRAGDNKYMHLKVFKSLYEAIERWHGEDLVLTGYQVDKNKDDELTGF

SEQ ID NO: 360 - protein, 111 aa, synthetic construct
MIPGGLSEAKPATPEIQEIVDKVKPQLEEKTGETYGKLEAVQYKTQVVATPEKKYVVGTNYYIKVRAGDNKYMHLKVFKSLWNANILRYWEDLVLTGYQVDKNKDDELTGF

SEQ ID NO: 361 - protein, 111 aa, synthetic construct
MIPGGLSEAKPATPEIQEIVDKVKPQLEEKTGETYGKLEAVQYKTQVVTSERLWILYGTNYYIKVRAGDNKYMHLKVFKSLTPRYVPLNPEDLVLTGYQVDKNKDDELTGF

SEQ ID NO: 362 - protein, 111 aa, synthetic construct
MIPGGLSEAKPATPEIQEIVDKVKPQLEEKTGETYGKLEAVQYKTQVVEHHSQPLAHGTNYYIKVRAGDNKYMHLKVFKSLPYHAFDWYWEDLVLTGYQVDKNKDDELTGF

SEQ ID NO: 363 - protein, 111 aa, synthetic construct
MIPGGLSEAKPATPEIQEIVDKVKPQLEEKTGETYGKLEAVQYKTQVVWTADWLWTVGTNYYIKVRAGDNKYMHLKVFKSLYDPSEWFPREDLVLTGYQVDKNKDDELTGF

SEQ ID NO: 364 - protein, 111 aa, synthetic construct
MIPGGLSEAKPATPEIQEIVDKVKPQLEEKTGETYGKLEAVQYKTQVVSFQREHYSWGTNYYIKVRAGDNKYMHLKVFKSLFDIFYFNPIEDLVLTGYQVDKNKDDELTGF

SEQ ID NO: 365 - protein, 111 aa, synthetic construct
MIPGGLSEAKPATPEIQEIVDKVKPQLEEKTGETYGKLEAVQYKTQVVGKTWHKHNRGTNYYIKVRAGDNKYMHLKVFKSLNLYFFFYQTEDLVLTGYQVDKNKDDELTGF

SEQ ID NO: 366 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccatgatgtgttttacgatcagaaagcaggtacgaactactacatcaaggttcgtgcgggtgacaacaagtatatgcacctgaaagtgtttaagagcctgaaatggtggtacagaatcggtaacccagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 367 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcccattcaaaatcatcaccgtttggcatggtacgaactactacatcaaggttcgtgcgggtgacaacaagtatatgcacctgaaagtgtttaagagcctgctggaaagattcaacttccatggtgaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 368 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccagtacaaaatcatcacctggtggtctggtacgaactactacatcaaggttcgtgcgggtgacaacaagtatatgcacctgaaagtgtttaagagcctggtttacaaaaacaaaagaggtggtcatgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 369 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccactactacgttcattacaacgatcagggtacgaactactacatcaaggttcgtgcgggtgacaacaagtatatgcacctgaaagtgtttaagagcctgtggggtgaaaacctgttcgcaaaatgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 370 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctactaccagatcatcacctggtggcagggtacgaactactacatcaaggttcgtgcgggtgacaacaagtatatgcacctgaaagtgtttaagagcctgggtatcagaaaaaaacagcagcagagagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 371 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgttcatggtccacatctggaacaggcaggtacgaactactacatcaaggttcgtgcgggtgacaacaagtatatgcacctgaaagtgtttaagagcctgggtcatgcattctacttcgcattcgttgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 372 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcctgcaggatcagtggccatggcgtgatggtacgaactactacatcaaggttcgtgcgggtgacaacaagtatatgcacctgaaagtgtttaagagcctgacctacggtaaaaaatggtactcttgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 373 - DNA, 378 bp, synthetic construct
  source 1..378: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgacgcgggtacgaactactacatcaaggttcgtgcgggtgacaacaagtatatgcacctgaaagtgtttaagagcctgtacttctacagacattgggaagatagagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 374 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctcttgggttaaaccagataacgaactgggtacgaactactacatcaaggttcgtgcgggtgacaacaagtatatgcacctgaaagtgtttaagagcctgtggcaggttcatgataaacagtggctggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 375 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcaactctctgggtgtttctgaatgggttggtacgaactactacatcaaggttcgtgcgggtgacaacaagtatatgcacctgaaagtgtttaagagcctgccatactggtacagaaaaaacaacctggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 376 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgtttaccgtatcgtttcttggtggcgtggtacgaactactacatcaaggttcgtgcgggtgacaacaagtatatgcacctgaaagtgtttaagagcctggcatctgatatctacctgagaatcgatgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 377 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctactaccgtcagctgaaattcaacaccggtacgaactactacatcaaggttcgtgcgggtgacaacaagtatatgcacctgaaagtgtttaagagcctgttcctgcatagaaaaccacaggatagagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 378 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcggttggatcaaagttgataacgttctgggtacgaactactacatcaaggttcgtgcgggtgacaacaagtatatgcacctgaaagtgtttaagagcctgtggcaggatcatcataacgttaaaagagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 379 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcaccgcatactacatccatgttaacgcaggtacgaactactacatcaaggttcgtgcgggtgacaacaagtatatgcacctgaaagtgtttaagagcctgccatacggttacagagcaaaagcaccagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 380 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgttcgtgttgaaccaaccccaaacgatggtacgaactactacatcaaggttcgtgcgggtgacaacaagtatatgcacctaaaagtgtttaagagcctgccactggttcaggaactgtggggtttcgaagatttggtgctgacgggctaccaagttgacaagaacaaagatgacgagctgacgggtttcgcggccgctgaacagaagctgatctccgaagaagatctggccgcgcatcaccatcaccaccactaatga

SEQ ID NO: 381 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgatctgaactcttctgtttggtaccagggtacgaactactacatcaaggttcgtgcgggtgacaacaagtatatgcacctgaaagtgtttaagagcctgtactggctgctggaaatcgaacatagagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 382 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcttcacctaccagatctcttacaccgttggtacgaactactacatcaaggttcgtgcgggtgacaacaagtatatgcacctgaaagtgtttaagagcctgagatggagatacccagcaagaaaaccagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 383 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccgtgaaaactggtacggtgaattcgcaggtacgaactactacatcaaggttcgtgcgggtgacaacaagtatatgcacctgaaagtgtttaagagcctgctgtacatctacagacatccaaactacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 384 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcaccgcactggcatggaacacccaggcaggtacgaactactacatcaaggttcgtgcgggtgacaacaagtatatgcacctgaaagtgtttaagagcctgccatggctgtacagacagaaatctgatgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 385 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccatatcccagcactgaaagttcatgcaggtacgaactactacatcaaggttcgtgcgggtgacaacaagtatatgcacctgaaagtgtttaagagcctgccatactggtacagacatgatcaggaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 386 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccatcatgcatacgcagcagtttacgttggtacgaactactacatcaaggttcgtgcgggtgacaacaagtatatgcacctgaaagtgtttaagagcctgccatactggtacagaaaatacccagatgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 387 - DNA, 399 bp, synthetic construct
  source 1..399: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctacgatgaactgtaccatgaaaaagcaggtacgaactactacatcaaggttcgtgcgggtgacaacaagtatatgcacctgaaagtgtttaagagcctggaatacaaattcagaaacctggaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 388 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctcttggtactacccaaaaaacgttctgggtacgaactactacatcaaggttcgtgcgggtgacaacaagtatatgcacctgaaagtgtttaagagcctggaagatttcatcgcagcagatgatgatgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 389 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcttcctggaatggcacttctgggaatggggtacgaactactacatcaaggttcgtgcgggtgacaacaagtatatgcacctgaaagtgtttaagagcctggattctcaggaattcccatctcatctggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 390 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctcttgggttcgtggtaccaaccagctgggtacgaactactacatcaaggttcgtgcgggtgacaacaagtatatgcacctgaaagtgtttaagagcctggaaccagattggccagatttcctgatcgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 391 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctggcgtaaactgctgtcttggtggaaaggtacgaactactacatcaaggttcgtgcgggtgacaacaagtatatgcacctgaaagtgtttaagagcctgatcctgaacccaaaatcttggaacttcgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 392 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcccaaaatctcatcagaccaacgaacagggtacgaactactacatcaaggttcgtgcgggtgacaacaagtatatgcacctgaaagtgtttaagagcctgtggaacgaaaaccatttctctgtttgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 393 - DNA, 414 bp, synthetic construct
  source 1..414: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcaactggaaccagatcgataacccactgggtacgaactactacatcaaggttcgtgcgggtgacaacaagtatatgcacctgaaagtgtttaagagcctgaacaaagattgggaagatcagatcatcgaagatttggtgctgacgggctaccaggttgacaagaacaaagatgacgagctgacgggtttcgcggccgcgggtggaagagcagaacagaagctgatctccgaagaagatctggccgcgcatcaccatcaccaccactaatga

SEQ ID NO: 394 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcttctgggttggtctgcataaccagctgggtacgaactactacatcaaggttcgtgcgggtgacaacaagtatatgcacctgaaagtgtttaagagcctgtactacaacggtgataacacccagatcgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 395 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcttcacccagccattcaacgaaccaatcggtacgaactactacatcaaggttcgtgcgggtgacaacaagtatatgcacctgaaagtgtttaagagcctgtattgggatgataaagttggttactacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 396 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcccagaataccagccacagccaaaagcaggtacgaactactacatcaaggttcgtgcgggtgacaacaagtatatgcacctgaaagtgtttaagagcctgttccatttcagaaaaccacaggatagagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 397 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcaacgatccatacttccagtctttctctggtacgaactactacatcaaggttcgtgcgggtgacaacaagtatatgcacctgaaagtgtttaagagcctgccatactggtacagagaaccagaaaacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 398 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccatcaggatgatgttgcatggcagtctggtacgaactactacatcaaggttcgtgcgggtgacaacaagtatatgcacctgaaagtgtttaagagcctggaatacggttggtaccatctgccatacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 399 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcaaaggttacttccataccgttgaagcaggtacgaactactacatcaaggttcgtgcgggtgacaacaagtatatgcacctgaaagtgtttaagagcctgacctaccatttcagaaccgatctgaacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 400 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgttgatcaggtttggtctgaaaacgcaggtacgaactactacatcaaggttcgtgcgggtgacaacaagtatatgcacctgaaagtgtttaagagcctgccatacctgtggagaaaacatgcagttgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 401 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcctgtcttctatctggcaggatgttgcaggtacgaactactacatcaaggttcgtgcgggtgacaacaagtatatgcacctgaaagtgtttaagagcctgctgtaccagttcagaaaagaaccagttgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 402 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcaccgcatctctgtactggaactacgcaggtacgaactactacatcaaggttcgtgcgggtgacaacaagtatatgcacctgaaagtgtttaagagcctgttctactacagaggtccacagccagttgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 403 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctcttggaaacagtggcataactacctgggtacgaactactacatcaaggttcgtgcgggtgacaacaagtatatgcacctgaaagtgtttaagagcctgtacccagatggtgatctgatcctgtacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 404 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccaggttcatctggataactctccactgggtacgaactactacatcaaggttcgtgcgggtgacaacaagtatatgcacctgaaagtgtttaagagcctgcagttcggtgaaatcatctggtgggaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 405 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccagaacaactggtacctgtctaactacggtacgaactactacatcaaggttcgtgcgggtgacaacaagtatatgcacctgaaagtgtttaagagcctggttgaagaaagatggggtctgtgggttgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 406 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctgggaatgggcactgctgctggaatacggtacgaactactacatcaaggttcgtgcgggtgacaacaagtatatgcacctgaaagtgtttaagagcctgcatcatccatggcagccagcaaaacaggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 407 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcggtgattcttactgggaatacgtttctggtacgaactactacatcaaggttcgtgcgggtgacaacaagtatatgcacctgaaagtgtttaagagcctgccatacagatggagaccagatcatgcagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 408 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcggtcagaaatggtggcagcataaaatcggtacgaactactacatcaaggttcgtgcgggtgacaacaagtatatgcacctgaaagtgtttaagagcctgcattctaacgcatggaacctgcagcatgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 409 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcctggatggtctgtaccatgttaactctggtacgaactactacatcaaggttcgtgcgggtgacaacaagtatatgcacctgaaagtgtttaagagcctgttctactactacagatctgaaccaaccgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 410 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccatgcaccagcaccagttcaggcaatcggtacgaactactacatcaaggttcgtgcgggtgacaacaagtatatgcacctgaaagtgtttaagagcctgccatactggtacagaccaaacagaccagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 411 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctgggaagatcattggaactaccagtacggtacgaactactacatcaaggttcgtgcgggtgacaacaagtatatgcacctgaaagtgtttaagagcctggaactgtactctaaccagatcgtttgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 412 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctcttggcgttacaaatctaaccagctgggtacgaactactacatcaaggttcgtgcgggtgacaacaagtatatgcacctgaaagtgtttaagagcctgtacaaagattgggttgttaacccaccagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 413 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccgtgttgattggtggctgccagaagttggtacgaactactacatcaaggttcgtgcgggtgacaacaagtatatgcacctgaaagtgtttaagagcctgccataccagtacagaaaagatccaaaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 414 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctcttggatcgaaatctctaaccagctgggtacgaactactacatcaaggttcgtgcgggtgacaacaagtatatgcacctgaaagtgtttaagagcctgtactacggtaacacctctatcctgatcgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 415 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctctctgggtcaggcagaaacctgggcaggtacgaactactacatcaaggttcgtgcgggtgacaacaagtatatgcacctgaaagtgtttaagagcctgttcaaatacagacatcagcaggataaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 416 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcaaaatcggtcagctgccatctcgtgttggtacgaactactacatcaaggttcgtgcgggtgacaacaagtatatgcacctgaaagtgtttaagagcctgtactggtacagaaacccagcagatagagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 417 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgcatggcataccaaaaacaactctatcggtacgaactactacatcaaggttcgtgcgggtgacaacaagtatatgcacctgaaagtgtttaagagcctggaaaccaaactggcacatgaagatatcgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 418 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccagaccaaactggttacctggtggtggggtacgaactactacatcaaggttcgtgcgggtgacaacaagtatatgcacctgaaagtgtttaagagcctgctgggtgaacagtacatcgatgttgatgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 419 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgcagttttcgattacgcagatccatctggtacgaactactacatcaaggttcgtgcgggtgacaacaagtatatgcacctgaaagtgtttaagagcctgccatacagatacagaacccatcataaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 420 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccatgttaaaatcgttacctggtggaaaggtacgaactactacatcaaggttcgtgcgggtgacaacaagtatatgcacctgaaagtgtttaagagcctgtacaacccaaaaccaggtatcgataacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 421 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccatgttctgaaagtttcttctgtttctggtacgaactactacatcaaggttcgtgcgggtgacaacaagtatatgcacctgaaagtgtttaagagcctgttctactacagaaaaagatacgatagagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 422 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctaccaggattacgattctgcaacccatggtacgaactactacatcaaggttcgtgcgggtgacaacaagtatatgcacctgaaagtgtttaagagcctgtaccagtacagaaacccaatcgataaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 423 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctcttggggtaaagtttctaacgaactgggtacgaactactacatcaaggttcgtgcgggtgacaacaagtatatgcacctgaaagtgtttaagagcctgatcgaataccagtctccagcactgtacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 424 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcctggaagatgaatggtggtctatcctgggtacgaactactacatcaaggttcgtgcgggtgacaacaagtatatgcacctgaaagtgtttaagagcctgccagatgcaccagcatactctaaatctgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 425 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctactacaaactggttacctggtggcgtggtacgaactactacatcaaggttcgtgcgggtgacaacaagtatatgcacctgaaagtgtttaagagcctgccagatctgaactacgaagttaccctggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 426 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgcatgggttaaagaacataacctgatcggtacgaactactacatcaaggttcgtgcgggtgacaacaagtatatgcacctgaaagtgtttaagagcctgtacgatcagaaccagtggtctccacatgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 427 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccaggaatacctgtggaaacagtacatcggtacgaactactacatcaaggttcgtgcgggtgacaacaagtatatgcacctgaaagtgtttaagagcctgagatggcagtacagacatccacagaccgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 428 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcttcaacaaagatctgttcctgggttggggtacgaactactacatcaaggttcgtgcgggtgacaacaagtatatgcacctgaaagtgtttaagagcctgagattctctgcaggtgaagaatgggaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 429 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccatttcggtatccgttacgcacatgttggtacgaactactacatcaaggttcgtgcgggtgacaacaagtatatgcacctgaaagtgtttaagagcctggattacccaagacatcatcaggatcaggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 430 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctggtacgcatggcaggaaggttggcagggtacgaactactacatcaaggttcgtgcgggtgacaacaagtatatgcacctgaaagtgtttaagagcctgttcagaaaaaaatgggcacagctgtgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 431 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcggtaacgcagcatggggttctttcgcaggtacgaactactacatcaaggttcgtgcgggtgacaacaagtatatgcacctgaaagtgtttaagagcctgccattcttctacagaaaagataacccagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 432 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccgtgaagatctggatctgttctacaccggtacgaactactacatcaaggttcgtgcgggtgacaacaagtatatgcacctgaaagtgtttaagagcctggaattcatcctgcagccatggtggtacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 433 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcaacgaaaacgtttaccgtgttacccagggtacgaactactacatcaaggttcgtgcgggtgacaacaagtatatgcacctgaaagtgtttaagagcctgaaatacagacatagactgacctacgaagaagatttggtgctgacgggctaccaagttgacaagaacaaagatgacgagctgacgggtttcgcggccgctgaacagaagctgatctccgaagaagatctggccgcgcatcaccatcaccaccactaatga

SEQ ID NO: 434 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctggcataaaatcgttacccagtggccaggtacgaactactacatcaaggttcgtgcgggtgacaacaagtatatgcacctgaaagtgtttaagagcctgtacgcacagagaggtgaagtttacttcgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 435 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccattaccgtatcatcacccattgggaaggtacgaactactacatcaaggttcgtgcgggtgacaacaagtatatgcacctgaaagtgtttaagagcctggttagagcattcctgtactacaaagatgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 436 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcctggttttcgatgcatctacctctgcaggtacgaactactacatcaaggttcgtgcgggtgacaacaagtatatgcacctgaaagtgtttaagagcctgccatgggaattcagaggtgatcataaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 437 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccagtacgcagaattcctggaacatatcggtacgaactactacatcaaggttcgtgcgggtgacaacaagtatatgcacctgaaagtgtttaagagcctgggttggtctcattacttccagtacctggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 438 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcggtaacgttcaggaagattggcatatcggtacgaactactacatcaaggttcgtgcgggtgacaacaagtatatgcacctgaaagtgtttaagagcctgttctctgaagatctgaccctgaacggtgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 439 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcggttggtctgcatcttacccagaatctggtacgaactactacatcaaggttcgtgcgggtgacaacaagtatatgcacctgaaagtgtttaagagcctgacccagtgggaattcatcgaatggccagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 440 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctaccagttcgaacatatctcctacggtggtacgaactactacatcaaggttcgtgcgggtgacaacaagtatatgcacctgaaagtgtttaagagcctgttcacctacagactgccacagcatagagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 441 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgcatggatcaccccagttaacgttctgggtacgaactactacatcaaggttcgtgcgggtgacaacaagtatatgcacctgaaagtgtttaagagcctggatgaaatcttccatggtgcatacaacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 442 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggaaaactggaagccgtccagtataagactcaagtcgtcgaatcttacgcagcaatcttctggggtggtacgaactactacatcaaggttcgtgcgggtgacaacaagtatatgcacctgaaagtgtttaagagcctgtggggtgaaatccagcattggagacaggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 443 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccgtgtttggccatggacctgggatcgtggtacgaactactacatcaaggttcgtgcgggtgacaacaagtatatgcacctgaaagtgtttaagagcctggagcagtggctgccaaacccatacttcgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 444 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccaggttaaaaaatggtgggaatacttcgttacgaactactacatcaaggttcgtgcgggtgacaacaagtatatgcacctgaaagtgtttaagagcctgcagctgctgtctcatcagtggaccgaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 445 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcatcggtcagatctctgaacagcagtggggtacgaactactacatcaaggttcgtgcgggtgacaacaagtatatgcacctgaaagtgtttaagagcctgcatagatactggaccagagatgttaccgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 446 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgcattccataaactgggttggatcctgggtacgaactactacatcaaggttcgtgcgggtgacaacaagtatatgcacctgaaagtgtttaagagcctgggtgatgaatctcataaatggcataaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 447 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcttcaaaaaatggtggtggaaatacaaaggtacgaactactacatcaaggttcgtgcgggtgacaacaagtatatgcacctgaaagtgtttaagagcctgagagttgatgaacattacatccagttcgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 448 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgcatctcagcgtctgtggatcatctacggtacgaactactacatcaaggttcgtgcgggtgacaacaagtatatgcacctgaaagtgtttaagagcctgtctgattggagacatcatggtggttgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 449 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccagcgtgaaacccatatcctgtggtctggtacgaactactacatcaaggttcgtgcgggtgacaacaagtatatgcacctgaaagtgtttaagagcctgtggttcgataacctgcataacaacttcgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 450 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcggtcagtactggttctgggcaccaggtggtacgaactactacatcaaggttcgtgcgggtgacaacaagtatatgcacctgaaagtgtttaagagcctgccatcttctggtggtggtccaaaatgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 451 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccgtgattgggcatactacatctacttcggtacgaactactacatcaaggttcgtgcgggtgacaacaagtatatgcacctgaaagtgtttaagagcctgagatctgataccctgcattactggaacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 452 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcatcggttacaaactgaactacatctggggtacgaactactacatcaaggttcgtgcgggtgacaacaagtatatgcacctgaaagtgtttaagagcctgatccagtacagatggcaggatagagttgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 453 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgtttctctgaccgaacagcgttggaaaggtacgaactactacatcaaggttcgtgcgggtgacaacaagtatatgcacctgaaagtgtttaagagcctgttccagccaatcaaactggcaaacgcagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 454 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctgggtttctcagcagcattactctcatggtacgaactactacatcaaggttcgtgcgggtgacaacaagtatatgcacctgaaagtgtttaagagcctgttcgcattcagaaaatacgaagatagagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 455 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgatgatgataaccagcattacggtctgggtacgaactactacatcaaggttcgtgcgggtgacaacaagtatatgcacctgaaagtgtttaagagcctgctgcataactggtggaaaggtaactgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 456 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcccaacccgttggatctctatctactggggtacgaactactacatcaaggttcgtgcgggtgacaacaagtatatgcacctgaaagtgtttaagagcctgctgaacagatactaccatgcatctatcgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 457 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcaccgcattcgcaccaaacaaagcatctggtacgaactactacatcaaggttcgtgcgggtgacaacaagtatatgcacctgaaagtgtttaagagcctgagatactgggcattcagatacctgtgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 458 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccattacaccgatttcaaatggcataaaggtacgaactactacatcaaggttcgtgcgggtgacaacaagtatatgcacctgaaagtgtttaagagcctgccattctgggcaaacgaacatggttgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 459 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcaacaaattctggtcttgggcaccaggtggtacgaactactacatcaaggttcgtgcgggtgacaacaagtatatgcacctgaaagtgtttaagagcctggcagattctggtttcaacgcaaccccagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 460 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgaactggatgaaccagcatgggattggggtacgaactactacatcaaggttcgtgcgggtgacaacaagtatatgcacctgaaagtgtttaagagcctgagacagcataaaaaaccacatccaaacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 461 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccatgaaaaatggggtttctgggaactgggtacgaactactacatcaaggttcgtgcgggtgacaacaagtatatgcacctgaaagtgtttaagagcctgcattctcatagattccatgaagcacatgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 462 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgttgcaaccgcagatctgatcaaagcaggtacgaactactacatcaaggttcgtgcgggtgacaacaagtatatgcacctgaaagtgtttaagagcctgccatggcattacccaccaaacaaatgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 463 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctactacaaagttatctcttggtggcatggtacgaactactacatcaaggttcgtgcgggtgacaacaagtatatgcacctgaaagtgtttaagagcctgtacaaaaccccattcccacagtacaccgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 464 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccattggcatcactacgataacatcatcggtacgaactactacatcaaggttcgtgcgggtgacaacaagtatatgcacctgaaagtgtttaagagcctgtaccagctggttgatctggatctggatgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 465 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgtttacaaaatcatcaccttctggtctggtacgaactactacatcaaggttcgtgcgggtgacaacaagtatatgcacctgaaagtgtttaagagcctggaatacccaccagttcagcaggttctggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 466 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcggttggatccagcgtcataacctgatcggtacgaactactacatcaaggttcgtgcgggtgacaacaagtatatgcacctgaaagtgtttaagagcctgtactctgaaccaccaccagttcagaacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 467 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcaaaacctgggaagataccgttgttgcaggtacgaactactacatcaaggttcgtgcgggtgacaacaagtatatgcacctgaaagtgtttaagagcctggaatacctgtacagaccactgaacccagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 468 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcctggatcagcatcatgtttaccaggttggtacgaactactacatcaaggttcgtgcgggtgacaacaagtatatgcacctgaaagtgtttaagagcctggcactgctgatccatcatgatccatgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 469 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcctggaagatgtttggggtgtttgggcaggtacgaactactacatcaaggttcgtgcgggtgacaacaagtatatgcacctgaaagtgtttaagagcctggaattccagcatagaaccttcaaccatgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 470 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctggctgccatggaccgttgaactgatcggtacgaactactacatcaaggttcgtgcgggtgacaacaagtatatgcacctgaaagtgtttaagagcctgggtgaaatctggtctgcaaaacataaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 471 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtcaagtataagactcaagtcgtccagcatttcacctgggattggttctggggtacgaactactacatcaaggttcgtgcgggtgacaacaagtatatgcacctgaaagtgtttaagagcctgccagaatacaaaccagttgatcataccgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 472 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcctgttcgaagttctgcaggggtcttctggtacgaactactacatcaaggttcgtgcgggtgacaacaagtatatgcacctgaaagtgtttaagagcctgggtgaaatcgtttggtaccaggttgaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 473 - DNA, 405 bp, synthetic construct
  source 1..405: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcccatactcttggcagcatgaagttgttggtacgaactactacatcaaggttcgtgcgggtgacaacaagtatatgcacctgaaagtgtttaagagcctgatcgttttctacctgcaggaatactacgaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 474 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctggtggcaggaaaacgcagaatggatcggtacgaactactacatcaaggttcgtgcgggtgacaacaagtatatgcacctgaaagtgtttaagagcctgggttacgcagttttccataaatacctggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 475 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctcttacgatccagtttaccaggataccggtacgaactactacatcaaggttcgtgcgggtgacaacaagtatatgcacctgaaagtgtttaagagcctgttcttccataggacctctgttgtttacgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 476 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccgtgttttcgataccccactggcagcaggtacgaactactacatcaaggttcgtgcgggtgacaacaagtatatgcacctgaaagtgtttaagagcctgccatggcagtacagaaacaacccagaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 477 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctggtgggatatcccatggatctgggttggtacgaactactacatcaaggttcgtgcgggtgacaacaagtatatgcacctgaaagtgtttaagagcctgcagaacgatgaactgtacccatacaaagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 478 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctaccagaaagttatcacctggtggcagggtacgaactactacatcaaggttcgtgcgggtgacaacaagtatatgcacctgaaagtgtttaagagcctgtactgggaaaaccatagaggtcatgttgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 479 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtccagtggatcaaatggggtaacatcctgggtacgaactactacatcaaggttcgtgcgggtgacaacaagtatatgcacctgaaagtgtttaagagcctgtacgaagcaatcgaaagatggcatggtgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 480 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgcaaccccagaaaaaaaatacgttgttggtacgaactactacatcaaggttcgtgcgggtgacaacaagtatatgcacctgaaagtgtttaagagcctgtggaacgcaaacatcctgagatactgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 481 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcacctctgaacgtctgtggatcctgtacggtacgaactactacatcaaggttcgtgcgggtgacaacaagtatatgcacctgaaagtgtttaagagcctgaccccaagatacgttccactgaacccagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 482 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcgaacatcattctcagccactggcacatggtacgaactactacatcaaggttcgtgcgggtgacaacaagtatatgcacctgaaagtgtttaagagcctgccataccatgcattcgattggtactgggaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 483 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctggaccgcagattggctgtggaccgttggtacgaactactacatcaaggttcgtgcgggtgacaacaagtatatgcacctgaaagtgtttaagagcctgtacgatccatctgaatggttcccaagagaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 484 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtctctttccagcgtgaacattactcttggggtacgaactactacatcaaggttcgtgcgggtgacaacaagtatatgcacctgaaagtgtttaagagcctgttcgatatcttctacttcaacccaatcgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 485 - DNA, 402 bp, synthetic construct
  source 1..402: mol_type = other DNA
atgattcccggcggactttcggaagccaagccagccactccggagattcaggaaatcgtggacaaggtcaaaccgcagctggaagagaaaaccggcgaaacctacggcaaactggaagccgtccagtataagactcaagtcgtcggtaaaacctggcataaacataaccgtggtacgaactactacatcaaggttcgtgcgggtgacaacaagtatatgcacctgaaagtgtttaagagcctgaacctgtacttcttcttctaccagaccgaagatttggtgctgacgggctaccaggttgacaagaacaaagatgacgagctgacgggtttcgcggccgctgaacagaagctgatctccgaagaagatctggccgcgcatcaccatcaccaccactaatga

SEQ ID NO: 486 - protein, 18 aa, unidentified
MKWVTFISLLFLFSSAYS

SEQ ID NO: 487 - protein, 22 aa, unidentified
MDMRAPAGIFGFLLVLFPGYRS

SEQ ID NO: 488 - protein, 19 aa, unidentified
MTRLTVLALLAGLLASSRA

SEQ ID NO: 489 - protein, 19 aa, unidentified
MELGLSWIFLLAILKGVQC

SEQ ID NO: 490 - protein, 19 aa, unidentified
MELGLRWVFLVAILEGVQC

SEQ ID NO: 491 - protein, 19 aa, unidentified
MKHLWFFLLLVAAPRWVLS

SEQ ID NO: 492 - protein, 19 aa, unidentified
MDWTWRILFLVAAATGAHS

SEQ ID NO: 493 - protein, 19 aa, unidentified
MDWTWRFLFVVAAATGVQS

SEQ ID NO: 494 - protein, 19 aa, unidentified
MEFGLSWLFLVAILKGVQC

SEQ ID NO: 495 - protein, 19 aa, unidentified
MEFGLSWVFLVALFRGVQC

SEQ ID NO: 496 - protein, 26 aa, unidentified
MDLLHKNMKHLWFFLLLVAAPRWVLS

SEQ ID NO: 497 - protein, 22 aa, unidentified
MDMRVPAQLLGLLLLWLSGARC

SEQ ID NO: 498 - protein, 22 aa, unidentified
MKYLLPTAAAGLLLLAAQPAMA

SEQ ID NO: 499 - protein, 17 aa, unidentified
MGVKVLFALICIAVAEA

SEQ ID NO: 500 - protein, 18 aa, unidentified
MAFLWLLSCWALLGTTFG

SEQ ID NO: 501 - protein, 14 aa, unidentified
MQLLSCIALILALV

SEQ ID NO: 502 - protein, 15 aa, unidentified
MNLLLILTFVAAAVA

SEQ ID NO: 503 - protein, 16 aa, unidentified
MPLLLLLPLLWAGALA

SEQ ID NO: 504 - protein, 30 aa, unidentified
MDSKGSSQKGSRLLLLLVVSNLLLCQGVVS

SEQ ID NO: 505 - protein, 23 aa, unidentified
MDAMKRGLCCVLLLCGAVFVSPS

SEQ ID NO: 506 - protein, 16 aa, synthetic construct
MLLLLLLLLLLALALA

SEQ ID NO: 507 - protein, 21 aa, synthetic construct
MWWRLWWLLLLLLLLWPMVWA

SEQ ID NO: 508 - protein, 5 aa, synthetic construct
  VARIANT 1..5: note = The entire sequence of amino acids 1-5 can be repeated one or more times
GGGGS

SEQ ID NO: 509 - protein, 5 aa, synthetic construct
  VARIANT 1..5: note = The entire sequence of amino acids 1-5 can be repeated one or more times
EAAAK

SEQ ID NO: 510 - protein, 8 aa, synthetic construct
GGGGGGGG

SEQ ID NO: 511 - protein, 6 aa, synthetic construct
GGGGGG

SEQ ID NO: 512 - protein, 18 aa, synthetic construct
KESGSVSSEQLAQFRSLD

SEQ ID NO: 513 - protein, 14 aa, synthetic construct
EGKSSGSGSESKST

SEQ ID NO: 514 - protein, 12 aa, synthetic construct
GSAGSAAGSGEF

SEQ ID NO: 515 - protein, 46 aa, synthetic construct
AEAAAKEAAAKEAAAKEAAAKALEAEAAAKEAAAKEAAAKEAAAKA

SEQ ID NO: 516 - protein, 5 aa, synthetic construct
PAPAP

SEQ ID NO: 517 - protein, 12 aa, synthetic construct
AEAAAKEAAAKA

SEQ ID NO: 518 - skipped

SEQ ID NO: 519 - protein, 4 aa, synthetic construct
GGSG

SEQ ID NO: 520 - protein, 4 aa, synthetic construct
GSGS

SEQ ID NO: 521 - protein, 4 aa, synthetic construct
GGGS

SEQ ID NO: 522 - protein, 4 aa, synthetic construct
  VARIANT 2..4: note = GGS residues can be repeated one or more times
SGGS

SEQ ID NO: 523 - protein, 7 aa, synthetic construct
GGGSGGG

SEQ ID NO: 524 - protein, 8 aa, synthetic construct
ESGGGGVT

SEQ ID NO: 525 - protein, 9 aa, synthetic construct
LESGGGGVT

SEQ ID NO: 526 - protein, 6 aa, synthetic construct
GRAQVT

SEQ ID NO: 527 - protein, 6 aa, synthetic construct
WRAQVT

SEQ ID NO: 528 - protein, 8 aa, synthetic construct
ARGRAQVT

SEQ ID NO: 529 - protein, 33 aa, synthetic construct
SGTTSGTTRLLSGHTCFTLTGLLGTLVTMGLLT

SEQ ID NO: 530 - protein, 26 aa, synthetic construct
SGTSPGLSAGATVGIMIGVLVGVALI

SEQ ID NO: 531 - protein, 24 aa, synthetic construct
SAPVLSAVATVGITIGVLARVALI

SEQ ID NO: 532 - protein, 24 aa, synthetic construct
SSPDLSAGTAVSIMIGVLAGMALI

SEQ ID NO: 533 - protein, 25 aa, synthetic construct
TLGGNSASYTFVSLLFSAVTLLLLC

SEQ ID NO: 534 - protein, 26 aa, synthetic construct
SGTSPGLSAGATVGIMIGVLVGVALI

SEQ ID NO: 535 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 536 - protein, 16 aa, Homo sapiens
DKTHTCPPCPAPELLG

SEQ ID NO: 537 - protein, 16 aa, synthetic construct
GSTHTCPPCPAPELLG

SEQ ID NO: 538 - protein, 21 aa, synthetic construct
EPKSCDKTHTCPPCPAPELLG

SEQ ID NO: 539 - protein, 24 aa, synthetic construct
EPKSCDKTHTCPPCPAPELLGGPS

SEQ ID NO: 540 - protein, 24 aa, synthetic construct
EPKSSDKTHTCPPCPAPELLGGPS

SEQ ID NO: 541 - protein, 24 aa, synthetic construct
EPKSSDKTHTCPPCPAPELLGGSS

SEQ ID NO: 542 - protein, 24 aa, synthetic construct
EPKSSGSTHTCPPCPAPELLGGSS

SEQ ID NO: 543 - protein, 19 aa, synthetic construct
DKTHTCPPCPAPELLGGPS

SEQ ID NO: 544 - protein, 19 aa, synthetic construct
DKTHTCPPCPAPELLGGSS

SEQ ID NO: 545 - protein, 227 aa, Homo sapiens; identical to SEQ ID NO: 535

SEQ ID NO: 546 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 547 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 548 - protein, 227 aa, Homo sapiens
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 549 - protein, 232 aa, Homo sapiens
EPKSSDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 550 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 551 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 552 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 553 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 554 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 555 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYHSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 556 - protein, 233 aa, Homo sapiens
DKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 557 - protein, 233 aa, Homo sapiens
DKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 558 - protein, 233 aa, Homo sapiens
SEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 559 - protein, 585 aa, Homo sapiens [sequence omitted]

SEQ ID NO: 560 - protein, 6 aa, Homo sapiens
RGVFRR

SEQ ID NO: 561 - protein, 9 aa, synthetic construct
  VARIANT 2: note = X can be Y, F or H
  VARIANT 3: note = X can be Y or W
  VARIANT 5: note = X can be A, S or R
  VARIANT 6: note = X can be K or Y
GXXAXXWPG

SEQ ID NO: 562 - protein, 9 aa, synthetic construct
WTQPKNEHH

SEQ ID NO: 563 - protein, 9 aa, synthetic construct
HLKHTDAQP

SEQ ID NO: 564 - protein, 9 aa, synthetic construct
HDQDVLHAW

SEQ ID NO: 565 - protein, 9 aa, synthetic construct
KFHRQEWAD

SEQ ID NO: 566 - protein, 9 aa, synthetic construct
PEDFWDPEH

SEQ ID NO: 567 - protein, 9 aa, synthetic construct
VVRTTGHVV

SEQ ID NO: 568 - protein, 9 aa, synthetic construct
YWWFCTGQS

SEQ ID NO: 569 - protein, 9 aa, synthetic construct
IHHRQARSL

SEQ ID NO: 570 - protein, 9 aa, synthetic construct
SHRRRAYIW

SEQ ID NO: 571 - protein, 9 aa, synthetic construct
WDSHHWRAP

SEQ ID NO: 572 - protein, 8 aa, synthetic construct
DKRVKYGQ

SEQ ID NO: 573 - protein, 9 aa, synthetic construct
SDWVYALQL

SEQ ID NO: 574 - protein, 6 aa, synthetic construct
FWWFWY

SEQ ID NO: 575 - protein, 9 aa, synthetic construct
VRDWPWNTF

SEQ ID NO: 576 - protein, 9 aa, synthetic construct
QKKRDEDYI

SEQ ID NO: 577 - protein, 9 aa, synthetic construct
GVHEEPRKL

SEQ ID NO: 578 - protein, 9 aa, synthetic construct
EWWQKHWPS

SEQ ID NO: 579 - protein, 9 aa, synthetic construct
NFFQRRWPG

SEQ ID NO: 580 - protein, 9 aa, synthetic construct
DWWQAKWPH

SEQ ID NO: 581 - protein, 9 aa, synthetic construct
GIWQSRWPG

SEQ ID NO: 582 - protein, 9 aa, synthetic construct
GYWAAKWPG

SEQ ID NO: 583 - protein, 9 aa, synthetic construct
GFYADHWPG

SEQ ID NO: 584 - protein, 9 aa, synthetic construct
NWYQQRWPG

SEQ ID NO: 585 - protein, 9 aa, synthetic construct
GFYARHWPG

SEQ ID NO: 586 - protein, 9 aa, synthetic construct
DFWKAHWPG

SEQ ID NO: 587 - protein, 9 aa, synthetic construct
DFYSVRWPG

SEQ ID NO: 588 - protein, 9 aa, synthetic construct
YWAANHASK

SEQ ID NO: 589 - protein, 9 aa, synthetic construct
IKRLEHWEY

SEQ ID NO: 590 - protein, 9 aa, synthetic construct
EWDSPWSEN

SEQ ID NO: 591 - protein, 9 aa, synthetic construct
KHKNLRWPF

SEQ ID NO: 592 - protein, 9 aa, synthetic construct
RHFPKQTNW

SEQ ID NO: 593 - protein, 9 aa, synthetic construct
VWGPEYQHQ

SEQ ID NO: 594 - protein, 9 aa, synthetic construct
TWKNNGQDV

SEQ ID NO: 595 - protein, 9 aa, synthetic construct
ATWLNYYLP

SEQ ID NO: 596 - protein, 9 aa, synthetic construct
DQESLFLNN

SEQ ID NO: 597 - protein, 9 aa, synthetic construct
GFYAQHWPD

SEQ ID NO: 598 - protein, 9 aa, synthetic construct
GHYARYWPG

SEQ ID NO: 599 - protein, 9 aa, synthetic construct
GFWASKWPG

SEQ ID NO: 600 - protein, 9 aa, synthetic construct
GFWQRKWPN

SEQ ID NO: 601 - protein, 9 aa, synthetic construct
VWPADNDLK

SEQ ID NO: 602 - protein, 9 aa, synthetic construct
HWAWTSPGY

SEQ ID NO: 603 - protein, 9 aa, synthetic construct
NFFQRRWPG

SEQ ID NO: 604 - protein, 9 aa, synthetic construct
HHSHRLKGQ

SEQ ID NO: 605 - protein, 9 aa, synthetic construct
YQNTIFLSI

SEQ ID NO: 606 - protein, 9 aa, synthetic construct
FQDQFTWSQ

SEQ ID NO: 607 - protein, 9 aa, synthetic construct
GEPHWPWQA

SEQ ID NO: 608 - protein, 9 aa, synthetic construct
ADPRHPWVE

SEQ ID NO: 609 - protein, 9 aa, synthetic construct
FHKRFQSQG

SEQ ID NO: 610 - protein, 9 aa, synthetic construct
EWWQNRWPN

SEQ ID NO: 611 - protein, 9 aa, synthetic construct
EWYQTRWPG

SEQ ID NO: 612 - protein, 9 aa, synthetic construct
EFWQRHWPG

SEQ ID NO: 613 - protein, 9 aa, synthetic construct
KFYERHWPG

SEQ ID NO: 614 - protein, 9 aa, synthetic construct
GWWQRRWPG

SEQ ID NO: 615 - protein, 9 aa, synthetic construct
RFKYFAHYQ

SEQ ID NO: 616 - protein, 9 aa, synthetic construct
FHDFWHRRW

SEQ ID NO: 617 - protein, 9 aa, synthetic construct
DWYHYWWEV

SEQ ID NO: 618 - protein, 9 aa, synthetic construct
STRSIHVTT

SEQ ID NO: 619 - protein, 9 aa, synthetic construct
KQHHHYLDK

SEQ ID NO: 620 - protein, 9 aa, synthetic construct
HSAQDREIP

SEQ ID NO: 621 - protein, 9 aa, synthetic construct
WVQSGYNSQ

SEQ ID NO: 622 - protein, 9 aa, synthetic construct
AVFWGKWSD

SEQ ID NO: 623 - protein, 9 aa, synthetic construct
QSFDKPWTT

SEQ ID NO: 624 - protein, 9 aa, synthetic construct
HYPLKYSFE

SEQ ID NO: 625 - protein, 9 aa, synthetic construct
WHHPWHRNR

SEQ ID NO: 626 - protein, 9 aa, synthetic construct
DPWWAWVVW

SEQ ID NO: 627 - protein, 9 aa, synthetic construct
FDNQDLIQY

SEQ ID NO: 628 - protein, 9 aa, synthetic construct
EKKNWYKWD

SEQ ID NO: 629 - protein, 9 aa, synthetic construct
DRHKSRWGI

SEQ ID NO: 630 - protein, 9 aa, synthetic construct
LNPFTPSVT

SEQ ID NO: 631 - protein, 9 aa, synthetic construct
YKGALLNHD

SEQ ID NO: 632 - protein, 9 aa, synthetic construct
WKFRNTERG

SEQ ID NO: 633 - protein, 9 aa, synthetic construct
YKVHQSSGG

SEQ ID NO: 634 - protein, 9 aa, synthetic construct
FHPIAGRPW

SEQ ID NO: 635 - protein, 9 aa, synthetic construct
FPNTSYDLQ

SEQ ID NO: 636 - protein, 9 aa, synthetic construct
FAHYNLKSG

SEQ ID NO: 637 - protein, 9 aa, synthetic construct
WHNYGESSG

SEQ ID NO: 638 - protein, 9 aa, synthetic construct
KFYYADHQW

SEQ ID NO: 639 - protein, 9 aa, synthetic construct
YTHADPHSQ

SEQ ID NO: 640 - protein, 9 aa, synthetic construct
FGVPQLGAG

SEQ ID NO: 641 - protein, 9 aa, synthetic construct
YSGFPFAGF

SEQ ID NO: 642 - protein, 9 aa, synthetic construct
WFSWPYTPL

SEQ ID NO: 643 - protein, 9 aa, synthetic construct
YYHPSIQST

SEQ ID NO: 644 - protein, 9 aa, synthetic construct
FLGWKDTVV

SEQ ID NO: 645 - protein, 9 aa, synthetic construct
DWWKWWWAK

SEQ ID NO: 646 - protein, 9 aa, synthetic construct
NAGWPLVPE

SEQ ID NO: 647 - protein, 9 aa, synthetic construct
YALDPFGGK

SEQ ID NO: 648 - protein, 9 aa, synthetic construct
GYKFWGVSD

SEQ ID NO: 649 - protein, 9 aa, synthetic construct
QGKQYILLR

SEQ ID NO: 650 - protein, 9 aa, synthetic construct
YKRHSAHDY

SEQ ID NO: 651 - protein, 9 aa, synthetic construct
WAQKSKVHQ

SEQ ID NO: 652 - protein, 9 aa, synthetic construct
FTAVSKKDA

SEQ ID NO: 653 - protein, 9 aa, synthetic construct
WGDKENIWF

SEQ ID NO: 654 - protein, 9 aa, synthetic construct
WSGHPWVQK

SEQ ID NO: 655 - protein, 9 aa, synthetic construct
YADYPLSPK

SEQ ID NO: 656 - protein, 9 aa, synthetic construct
WKFRNTDRG

SEQ ID NO: 657 - protein, 9 aa, synthetic construct
QTVATHYHY

SEQ ID NO: 658 - protein, 9 aa, synthetic construct
WHAKHLLSH

SEQ ID NO: 659 - protein, 9 aa, synthetic construct
SGIKKADSV

SEQ ID NO: 660 - protein, 9 aa, synthetic construct
KANLINVKS

SEQ ID NO: 661 - protein, 9 aa, synthetic construct
WKSHVEVRS

SEQ ID NO: 662 - protein, 9 aa, synthetic construct
WVTQKYIIQ

SEQ ID NO: 663 - protein, 9 aa, synthetic construct
WEHAKDWPT

SEQ ID NO: 664 - protein, 9 aa, synthetic construct
FHSKVLDKA

SEQ ID NO: 665 - protein, 9 aa, synthetic construct
YGAQKQAVW

SEQ ID NO: 666 - protein, 9 aa, synthetic construct
FSASHFTSQ

SEQ ID NO: 667 - protein, 9 aa, synthetic construct
  VARIANT 1..2: note = X can be any amino acid
  VARIANT 4: note = X can be any amino acid
  VARIANT 6: note = X can be any amino acid
  VARIANT 8: note = X can be any amino acid
XXAXKXDXQ

SEQ ID NO: 668 - protein, 112 aa, synthetic construct
MIPRGLSEAKPATPEIQEIVDKVKPQLEEKTNETYGKLEAVQYKTQVLADWWQAKWPHSTNYYIKVRAGDNKYMHLKVFNGPYKVHQSSGGADRVLTGYQVDKNKDDELTGF

SEQ ID NO: 669 - protein, 112 aa, synthetic construct
MIPRGLSEAKPATPEIQEIVDKVKPQLEEKTNETYGKLEAVQYKTQVLAGYWAAKWPGSTNYYIKVRAGDNKYMHLKVFNGPFPNTSYDLQADRVLTGYQVDKNKDDELTGF

SEQ ID NO: 670 - protein, 112 aa, synthetic construct
MIPRGLSEAKPATPEIQEIVDKVKPQLEEKTNETYGKLEAVQYKTQVLANWYQQRWPGSTNYYIKVRAGDNKYMHLKVFNGPWHNYGESSGADRVLTGYQVDKNKDDELTGF

SEQ ID NO: 671 - protein, 112 aa, synthetic construct
MIPRGLSEAKPATPEIQEIVDKVKPQLEEKTNETYGKLEAVQYKTQVLAVWGPEYQHQSTNYYIKVRAGDNKYMHLKVFNGPNAGWPLVPEADRVLTGYQVDKNKDDELTGF

SEQ ID NO: 672 - protein, 112 aa, synthetic construct
MIPRGLSEAKPATPEIQEIVDKVKPQLEEKTNETYGKLEAVQYKTQVLAGHYARYWPGSTNYYIKVRAGDNKYMHLKVFNGPWAQKSKVHQADRVLTGYQVDKNKDDELTGF

SEQ ID NO: 673 - protein, 112 aa, synthetic construct
MIPRGLSEAKPATPEIQEIVDKVKPQLEEKTNETYGKLEAVQYKTQVLAGFWASKWPGSTNYYIKVRAGDNKYMHLKVFNGPFTAVSKKDAADRVLTGYQVDKNKDDELTGF

SEQ ID NO: 674 - protein, 112 aa, synthetic construct
MIPRGLSEAKPATPEIQEIVDKVKPQLEEKTNETYGKLEAVQYKTQVLANFFQRRWPGSTNYYIKVRAGDNKYMHLKVFNGPWKFRNTDRGADRVLTGYQVDKNKDDELTGF

SEQ ID NO: 675 - DNA, 12 bp, synthetic construct
  source 1..12: mol_type = other DNA
accgttgccggt

SEQ ID NO: 676 - skipped

SEQ ID NO: 677 - protein, 251 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGF

SEQ ID NO: 678 - protein, 392 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGF

SEQ ID NO: 679 - protein, 251 aa, synthetic construct
IPGGLSEAKPATPEIQEIVDKVKPQLEEKTGETYGKLEAVQYKTQVVHHAYAAVYVGTNYYIKVRAGDNKYMHLKVFKSLPYWYRKYPDEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHHAYAAVYVGTNYYIKVRAGDNKYMHLKVFKSLPYWYRKYPDEDLVLTGYQVDKNKDDELTGF

SEQ ID NO: 680 - protein, 392 aa, synthetic construct
IPGGLSEAKPATPEIQEIVDKVKPQLEEKTGETYGKLEAVQYKTQVVHHAYAAVYVGTNYYIKVRAGDNKYMHLKVFKSLPYWYRKYPDEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHHAYAAVYVGTNYYIKVRAGDNKYMHLKVFKSLPYWYRKYPDEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHHAYAAVYVGTNYYIKVRAGDNKYMHLKVFKSLPYWYRKYPDEDLVLTGYQVDKNKDDELTGF

SEQ ID NO: 681 - protein, 394 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGF

SEQ ID NO: 682 - protein, 394 aa, synthetic construct
IPGGLSEAKPATPEIQEIVDKVKPQLEEKTGETYGKLEAVQYKTQVVFTYQISYTVGTNYYIKVRAGDNKYMHLKVFKSLRWRYPARKPEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVFTYQISYTVGTNYYIKVRAGDNKYMHLKVFKSLRWRYPARKPEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVFTYQISYTVGTNYYIKVRAGDNKYMHLKVFKSLRWRYPARKPEDLVLTGYQVDKNKDDELTGF

SEQ ID NO: 683 - protein, 394 aa, synthetic construct
IPGGLSEAKPATPEIQEIVDKVKPQLEEKTGETYGKLEAVQYKTQVVHIPALKVHAGTNYYIKVRAGDNKYMHLKVFKSLPYWYRHDQEEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHIPALKVHAGTNYYIKVRAGDNKYMHLKVFKSLPYWYRHDQEEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHIPALKVHAGTNYYIKVRAGDNKYMHLKVFKSLPYWYRHDQEEDLVLTGYQVDKNKDDELTGF

SEQ ID NO: 684 - protein, 394 aa, synthetic construct
IPGGLSEAKPATPEIQEIVDKVKPQLEEKTGETYGKLEAVQYKTQVVHHAYAAVYVGTNYYIKVRAGDNKYMHLKVFKSLPYWYRKYPDEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHHAYAAVYVGTNYYIKVRAGDNKYMHLKVFKSLPYWYRKYPDEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHHAYAAVYVGTNYYIKVRAGDNKYMHLKVFKSLPYWYRKYPDEDLVLTGYQVDKNKDDELTGF

SEQ ID NO: 685 - protein, 391 aa, synthetic construct
IPGGLSEAKPATPEIQEIVDKVKPQLEEKTGETYGKLEAVQYKTQVVYDELYHEKAGTNYYIKVRAGDNKYMHLKVFKSLEYKFRNLEEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVYDELYHEKAGTNYYIKVRAGDNKYMHLKVFKSLEYKFRNLEEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVYDELYHEKAGTNYYIKVRAGDNKYMHLKVFKSLEYKFRNLEEDLVLTGYQVDKNKDDELTGF

SEQ ID NO: 686 - protein, 394 aa, synthetic construct
IPGGLSEAKPATPEIQEIVDKVKPQLEEKTGETYGKLEAVQYKTQVVVDQVWSENAGTNYYIKVRAGDNKYMHLKVFKSLPYLWRKHAVEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVVDQVWSENAGTNYYIKVRAGDNKYMHLKVFKSLPYLWRKHAVEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVVDQVWSENAGTNYYIKVRAGDNKYMHLKVFKSLPYLWRKHAVEDLVLTGYQVDKNKDDELTGF

SEQ ID NO: 687 - protein, 394 aa, synthetic construct
IPGGLSEAKPATPEIQEIVDKVKPQLEEKTGETYGKLEAVQYKTQVVGDSYWEYVSGTNYYIKVRAGDNKYMHLKVFKSLPYRWRPDHAEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVGDSYWEYVSGTNYYIKVRAGDNKYMHLKVFKSLPYRWRPDHAEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVGDSYWEYVSGTNYYIKVRAGDNKYMHLKVFKSLPYRWRPDHAEDLVLTGYQVDKNKDDELTGF

SEQ ID NO: 688 - protein, 394 aa, synthetic construct
IPGGLSEAKPATPEIQEIVDKVKPQLEEKTGETYGKLEAVQYKTQVVLDGLYHVNSGTNYYIKVRAGDNKYMHLKVFKSLFYYYRSEPTEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVLDGLYHVNSGTNYYIKVRAGDNKYMHLKVFKSLFYYYRSEPTEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVLDGLYHVNSGTNYYIKVRAGDNKYMHLKVFKSLFYYYRSEPTEDLVLTGYQVDKNKDDELTGF

SEQ ID NO: 689 - protein, 394 aa, synthetic construct
IPGGLSEAKPATPEIQEIVDKVKPQLEEKTGETYGKLEAVQYKTQVVKTWEDTVVAGTNYYIKVRAGDNKYMHLKVFKSLEYLYRPLNPEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVKTWEDTVVAGTNYYIKVRAGDNKYMHLKVFKSLEYLYRPLNPEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVKTWEDTVVAGTNYYIKVRAGDNKYMHLKVFKSLEYLYRPLNPEDLVLTGYQVDKNKDDELTGF

SEQ ID NO: 690 - protein, 533 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGF

SEQ ID NO: 691 - protein, 536 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGF

SEQ ID NO: 692 - protein, 534 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGF

SEQ ID NO: 693 - protein, 675 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGF

SEQ ID NO: 694 - protein, 537 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGF

SEQ ID NO: 695 - protein, 679 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGF

SEQ ID NO: 696 - protein, 566 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSGGGGSGGGGSGGGGSGGGGSGGGGSGGGGSMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGF

SEQ ID NO: 697 - protein, 534 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSGGGGSGGGGSGGGGSGGGGSGGGGSGGGGSMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGF

SEQ ID NO: 698 - protein, 535 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKEAAAKEAAAKEAAAKEAAAKEAAAKEAAAKMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGF

SEQ ID NO: 699 - protein, 536 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFGGGGSGGGGSGGGGSGGGGSGGGGSGGGGSGGGGSGGGGSGGGGSGGGGSGGGGSGGGGSMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGF

SEQ ID NO: 700 - protein, 537 aa, synthetic construct
IPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKMIPGGLSEAKPATPEIQEIVDKVKPQLEEKTGETYGKLEAVQYKTQVVHYYVHYNDQGTNYYIKVRAGDNKYMHLKVFKSLWGENLFAKWEDLVLTGYQVDKNKDDELTGFAEAAAKEAAAKEAAAKEAAAKEAAAKEAAAKEAAAKEAAAKEAAAKEAAAKEAAAKEAAAKMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGF

SEQ ID NO: 701 - protein, 537 aa, synthetic construct
IPGGLSEAKPATPEIQEIVDKVKPQLEEKTGETYGKLEAVQYKTQVVGGGGGGGGGGTNYYIKVRAGDNKYMHLKVFKSLGGGGGGGGGEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVGGGGGGGGGGTNYYIKVRAGDNKYMHLKVFKSLGGGGGGGGGEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVGGGGGGGGGGTNYYIKVRAGDNKYMHLKVFKSLGGGGGGGGGEDLVLTGYQVDKNKDDELTGFAEAAAKEAAAKEAAAKEAAAKEAAAKEAAAKMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGF

SEQ ID NO: 702 - protein, 679 aa, synthetic construct
IPGGLSEAKPATPEIQEIVDKVKPQLEEKTGETYGKLEAVQYKTQVVGGGGGGGGGGTNYYIKVRAGDNKYMHLKVFKSLGGGGGGGGGEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVGGGGGGGGGGTNYYIKVRAGDNKYMHLKVFKSLGGGGGGGGGEDLVLTGYQVDKNKDDELTGFAEAAAKEAAAKEAAAKEAAAKEAAAKEAAAKMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVGGGGGGGGGGTNYYIKVRAGDNKYMHLKVFKSLGGGGGGGGGEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVGGGGGGGGGGTNYYIKVRAGDNKYMHLKVFKSLGGGGGGGGGEDLVLTGYQVDKNKDDELTGF

SEQ ID NO: 703 - DNA, 753 bp, synthetic construct
  source 1..753: mol_type = other DNA
atccctggcggactgtcggaagccaagccggccactccggaaatccaggagattgtggataaggtcaagcctcagctggaggaaaagacgggcgaaacttacgggaagctcgaggccgtgcaatacaagacccaggtcgtgcactattacgtgcattacaacgaccaggggaccaactactacattaaggtccgcgctggagacaacaagtatatgcacctgaaagtgttcaagtcgctgtggggggagaacctcttcgcgaagtgggaggaccttgtgctcaccgggtaccaagtggacaagaacaaggacgacgagctgacaggcttcggcggaggaggttccggaggaggcggatccggtggaggcggttcaggcggtggaggaagcggaggcggaggatccggaggcggtggtagcatgattcccggagggttgtccgaagcgaaaccagccacccccgagatccaggaaatcgtggacaaggtcaaaccgcagctggaagagaaaaccggcgaaacctacggaaagctggaagcggtgcagtacaagactcaagtggtgcactactacgtgcactacaacgatcagggcaccaattactacatcaaagtccgggccggcgataacaagtacatgcacctcaaggtctttaagtccctctggggcgaaaacctgttcgccaaatgggaggatctggtgctgactggctaccaagtcgacaagaataaggatgacgagttgaccgggttc

SEQ ID NO: 704 - DNA, 1176 bp, synthetic construct
  source 1..1176: mol_type = other DNA
attcccggcggactgtcagaagccaaaccggccactccggagatccaggagatcgtggacaaggtcaagccacagctggaggaaaagaccggcgaaacctacgggaagttggaggctgtgcagtacaagacccaggttgtgcactattacgtccactataacgaccaaggcaccaactactatatcaaggtccgagcgggcgataacaaatacatgcacctgaaggtctttaagtccctctggggagaaaacctttttgcgaagtgggaggacctcgtgctcaccggataccaagtggacaagaataaggacgacgaactgactggattcggagggggcggttcaggaggagggggatcagggggaggaggaagcggcggaggaggatccggaggaggcggaagcggtggtggcggatccatgattccgggaggcctttccgaggccaagcctgccacccccgaaatccaagagattgtggataaggtcaaacctcagctggaagaaaagacgggggagacttacggcaaactggaagccgtgcagtataagacccaagtggtgcactactacgtgcactacaacgatcaggggaccaattactacattaaggtccgcgctggagacaacaagtacatgcatctcaaggtattcaagtcgctgtggggcgaaaacctcttcgcgaaatgggaggacttggtgctgaccgggtaccaagtcgataagaacaaggacgatgaacttactgggttcggaggcggaggttcgggtggaggcggttccggtggaggagggtccggtggtggaggatcgggtggcggtggatcgggaggcggtggttccatgatccctggaggcctgtccgaagcaaagccggccaccccagaaatccaggaaatcgtggataaagtcaagccccagttggaggagaaaaccggagagacatacgggaagctggaggcggtgcaatacaagactcaagtggtccattattacgtgcattacaacgaccagggcactaactactacatcaaagtccgggccggggataacaagtatatgcacctcaaggtgttcaagagcctctggggggagaacctgttcgccaaatgggaagatctggtgttgaccggctaccaggtcgacaagaacaaagacgacgagctgacggggttc

SEQ ID NO: 705 - DNA, 753 bp, synthetic construct
  source 1..753: mol_type = other DNA
atccctggtggcctgtccgaagccaagcctgctacgcccgagatccaggagattgtggacaaagtcaagccgcagctcgaggaaaagaccggcgaaacctacggcaaactcgaagccgtgcagtacaagactcaagtggtgcatcacgcgtacgccgctgtgtacgtgggcacaaactactacatcaaagtccgggccggcgataacaaatacatgcatctgaaagtgttcaagtcgctgccgtactggtacagaaagtacccagacgaggacttggtgctgactgggtaccaggtcgacaagaacaaggatgacgagcttaccggcttcggtggtggaggatccggcggaggaggatcgggaggaggcggttccggaggaggaggcagcggtggaggaggaagcggagggggtggaagcatgattcctggggggctctcagaagcaaagcctgccaccccggaaattcaggagatcgtggataaggtcaagccccagctggaggagaaaactggcgaaacttacgggaagctggaagcggtgcaatacaagacccaggtcgtgcaccatgcctacgccgccgtctacgtggggaccaactattacatcaaggtccgcgcgggagataacaagtacatgcacctgaaggtctttaagtccctcccctactggtaccggaagtatcccgatgaggacctggtgctgaccggctatcaagtggacaagaataaggacgacgagctgaccggtttc

SEQ ID NO: 706 - DNA, 1176 bp, synthetic construct
  source 1..1176: mol_type = other DNA
attcccggaggactctccgaggccaaaccggccaccccggaaatccaggagatcgtggataaggtcaagccccagctggaggaaaagaccggagagacttacgggaagcttgaggccgtccagtacaagacccaggtggtgcaccatgcgtacgccgcggtctacgtcgggaccaactactacattaaggtccgcgctggcgataacaagtatatgcacttaaaggtgttcaaatccctgccatattggtacaggaagtaccccgacgaagatctggtgctcaccggctaccaggtcgataagaataaggacgacgaactgaccggcttcggaggaggtggaagcggcggtggaggatccggcggaggaggaagcggaggcggtggatccggtggaggagggtctggtggagggggctccatgattcctggaggcctgtccgaagccaagcccgccactcccgaaattcaagaaatcgtggacaaggtcaaaccgcagctggaagaaaagactggagaaacctacggaaagctcgaggcggtgcagtacaaaacccaagtggtgcatcatgcctacgccgccgtctacgtgggcacgaactactatatcaaagtcagagccggcgacaacaagtacatgcacctgaaagtgttcaagtcactgccgtactggtacagaaagtacccggacgaggacctggtgctgaccgggtaccaggtggacaaaaacaaggacgatgaactcaccgggtttggaggaggcggaagcggtggtggtggctccggaggaggaggctcaggaggcggaggatcgggagggggaggttccggagggggtggatcgatgatccctggcggtctgtccgaggctaagccagccacccctgagatccaggaaattgtggacaaagtcaagccgcagctcgaggagaaaaccggcgaaacttacggaaagttggaggccgtgcaatacaagactcaggtcgtgcaccacgcatacgcggctgtgtacgtggggactaactattacatcaaggttcgcgccggagacaacaaatacatgcatctgaaggtctttaagtccctcccctactggtaccggaagtaccctgacgaggacttggtgctgactgggtaccaagtggataagaacaaggatgacgagctgacagggttc

SEQ ID NO: 707 - DNA, 1182 bp, synthetic construct
  source 1..1182: mol_type = other DNA
atccctggagggctgtccgaggccaagcctgctacgccggaaattcaagaaatcgtcgataaggttaagccacagctcgaagagaaaactggggaaacatacggcaaacttgaggcggttcagtacaaaactcaggtcgtgcactactacgtgcactacaacgatcaagggaccaactactatatcaaggtccgcgcgggagacaacaaatacatgcatctcaaagtgttcaaatcgctgtggggcgagaaccttttcgcgaagtgggaggatttggtgctgactggatatcaggtcgataaaaacaaggatgacgagcttactggctttgccgaggccgctgctaaggaagcagccgcgaaagaagccgccgccaaggaagctgcagccaaggaggcggcagcaaaggaggccgcagccaagatgattcccggaggcctctcagaagccaaaccagccactcctgagattcaggaaattgtcgacaaggtcaagccgcagctggaagagaagactggcgaaacctacgggaagctagaggcggtccagtacaagacccaagtcgtccattactatgtacattacaacgaccagggaaccaattactacattaaagtgcgggccggagataataagtatatgcatcttaaggtgtttaagtcgttgtggggcgaaaacctgtttgccaagtgggaagatcttgtgctcaccggttaccaagtggacaagaacaaggacgacgagctgaccgggttcgctgaagctgccgcgaaggaagccgctgcgaaggaggctgccgcaaaggaagcggccgctaaggaggcagccgccaaagaggccgccgcgaagatgatacctggcggactgtcggaggcgaaaccggccacccctgaaatccaggagattgtggacaaggtgaaaccccaactcgaggaaaaaaccggcgaaacgtacggtaaactcgaagccgtgcagtataagacccaggtcgtacactactatgtgcactataacgatcagggaacgaactactacatcaaggtcagagctggagacaataagtacatgcacctcaaggttttcaagtcgttatggggagaaaacctcttcgcgaaatgggaggatctcgtgctgaccggctaccaggtggacaaaaataaggatgatgaactcaccggtttc

SEQ ID NO: 708 - DNA, 1182 bp, synthetic construct
  source 1..1182: mol_type = other DNA
atccctggcggactgagcgaggccaagcctgccactccggagatccaggaaatcgtggacaaagtcaagccccagctggaggaaaagactggcgaaacttacggaaagctggaagccgtgcaatacaagacccaagtggtgttcacctatcaaatttcgtacaccgtcgggaccaattactacatcaaagtgcgggccggggataacaaatacatgcacttgaaagtgtttaagtcgctgcggtggaggtaccctgcccgcaagcctgaagatctggtgctcaccggttaccaagtcgacaagaacaaggatgacgagctcaccggcttcgccgaagcagccgctaaagaggccgccgccaaagaagccgccgctaaggaagcagcagcgaaggaggctgccgcgaaagaggcggccgccaagatgattcccggcggattgtccgaagccaagccggctacccccgaaatccaggagatcgtcgacaaggtcaagccacagctcgaagagaaaacgggagagacttacgggaagctggaggcagtccagtataagactcaggtcgtgttcacgtaccaaatctcctacactgtggggactaactactatatcaaagtccgagccggcgacaacaagtacatgcacctcaaggtgttcaagagcttgcgctggcggtaccctgctcggaagccagaagatcttgtgctgactgggtaccaggtcgataagaacaaagacgacgagttgaccggtttcgccgaggcagctgcaaaggaagctgccgccaaggaggcagcggccaaggaagccgctgcgaaggaagcggcggcaaaggaggccgcggcaaagatgatcccgggaggcctgtcagaagccaaacccgcgacccctgagattcaggaaattgtggataaggtcaaaccgcagctggaagaaaagaccggcgaaacctacggaaagttggaggcggtgcagtacaagacacaggttgtgtttacctaccaaatttcctacaccgtgggaaccaactattacatcaaggtccgcgcgggagataacaagtatatgcaccttaaagtgttcaagtccctgagatggagatacccagccagaaagccggaggacctggtgctgaccggctaccaagtggacaaaaacaaggacgacgagctgactggtttc

SEQ ID NO: 709 - DNA, 1182 bp, synthetic construct
  source 1..1182: mol_type = other DNA
atcccgggaggactgtcggaagcgaagccggccactccagagatccaagaaattgtggacaaggtcaagccccagctcgaggaaaagactggggaaacctacggaaagctcgaggctgtgcagtataagacccaagtggtgcatattccggcgctgaaggtgcacgccggaaccaattactacatcaaagtccgggccggagacaataagtacatgcatctcaaagtgtttaagtccctgccatactggtaccggcacgaccaggaggaggatctcgtgctgaccggctatcaggtcgataagaacaaggacgacgaactgactggtttcgctgaggctgcggcaaaggaggccgccgctaaggaggcggcagcgaaggaagctgcggccaaggaggcagccgcgaaggaggctgccgcgaagatgattcccggtggactgtcagaagccaagccagccactcccgaaatccaggaaatcgtggataaggtcaaaccccagctggaagaaaagaccggagagacttacgggaagttggaggccgtgcagtacaagacccaggtcgtgcacattccggccctgaaagtgcacgcgggtaccaactactacattaaggttcgcgctggagacaacaagtatatgcatctgaaagtgttcaagtccctcccctactggtatcgccacgaccaagaggaagatctggtgctgactggctaccaagtggacaaaaacaaggatgatgaactgaccggcttcgccgaagccgcagcaaaggaagccgccgccaaagaggccgctgctaaggaagcggcggccaaagaagcggccgccaaggaagcagcggccaagatgatccccgggggattgagcgaggccaagcctgccacccctgagatccaggagatcgtggacaaagtcaagccgcagcttgaagagaaaaccggcgaaacatacgggaagctggaggccgtccagtacaaaacccaggtggtgcacatcccggcgctcaaggtccacgccggcactaactactatatcaaggtccgcgccggagataacaaatacatgcacctgaaggtgttcaagagcctcccgtactggtacagacacgatcaggaagaggacctcgtgcttaccgggtaccaggtcgacaagaacaaagacgacgagctgacgggcttc

SEQ ID NO: 710 - DNA, 1182 bp, synthetic construct
  source 1..1182: mol_type = other DNA
atccctggaggcctgagcgaagccaagcctgccactccggagattcaggagatcgtggacaaagtcaagccccagctggaggaaaagacaggagagacttatggaaagctggaagccgtgcagtataagacccaagtggtgcaccatgcctacgccgccgtctacgtgggaaccaactactacatcaaggttcgcgccggggataacaagtacatgcacctcaaggtgttcaagtccctgccatactggtacagaaagtaccctgatgaggacctggtgctgaccgggtaccaggtcgacaagaataaggacgatgagctgactggctttgccgaagcggcagccaaggaggctgcggcgaaggaagctgccgcgaaagaggcggccgctaaggaagcagctgcaaaggaagcggccgcaaagatgattcccggtggactctccgaagcgaagcctgctaccccggagatccaggagattgtggacaaggtcaaaccgcagctcgaagaaaagaccggggaaacttacgggaagctggaggccgtgcaatacaaaacccaggtggtacaccacgcgtacgccgctgtgtacgtcggcaccaactattacattaaggtccgcgcaggagacaacaagtatatgcacctgaaggtctttaagagcctgccgtactggtaccggaagtacccggacgaggacctcgtgctcaccggctaccaagtggataagaacaaagatgacgagctcacgggtttcgctgaagcagccgccaaggaagccgccgccaaagaggccgccgcaaaggaggcggcggccaaagaagccgcggctaaggaggccgcagccaagatgatccctggtggcctgtcggaggccaaacccgccactcccgaaatccaggaaatcgtggataaggtcaagccgcagctggaagagaaaaccggcgaaacctacggaaagctcgaagcggtgcagtacaagacccaggtcgtgcaccacgcctacgctgcggtgtacgtggggaccaattactatatcaaagtccgggccggcgataacaaatacatgcatctgaaagtgttcaaatcccttccatattggtacaggaagtaccccgacgaggacttggtgcttaccggctatcaagtggacaagaacaaggacgacgaactgactggattc

SEQ ID NO: 711 - DNA, 1173 bp, synthetic construct
  source 1..1173: mol_type = other DNA
atccccggtggactgtccgaagcaaagccagcgactcccgaaatccaggaaattgtggataaggttaagccccagctggaagaaaagactggagaaacctacggaaagctcgaggccgtgcagtacaaaacccaggtggtgtacgatgaactgtaccacgagaaagccggtactaattactacattaaggtccgcgcaggagacaacaagtatatgcacctcaaagtgtttaagagcctggagtataagttccgcaacctggaagaggacttggtgcttactggataccaagtcgacaaaaacaaggacgacgaactgaccgggttcgccgaagcagcggctaaggaggccgcagctaaggaagccgccgccaaagaagccgcggcgaaagaagcggccgcgaaggaagctgccgcgaagatgattccgggcggtctgtcagaagccaagccggctaccccggaaattcaggaaatcgtggacaaggtcaagccgcagctcgaggaaaagaccggagagacttacgggaagctggaggccgtccagtacaagacccaagtggtgtatgacgaactctaccacgaaaaggccgggacgaactactacatcaaagtcagggccggagataacaagtacatgcacctgaaggtgttcaagtccctggagtacaagttccggaaccttgaggaggacctggtgctgaccggctaccaagtggacaagaacaaggatgatgagctgacagggtttgccgaggctgctgcgaaggaggcggctgccaaggaagcggcggcaaaagaggccgccgcaaaggaggcagcggccaaagaggcagccgccaagatgatccccggcggattgtccgaggccaagccagccacccctgagatccaggagattgtggacaaagtcaagcctcagctcgaagagaaaaccggcgaaacttacggaaagttggaagctgtgcagtataagacccaggtcgtgtacgacgagctgtaccatgagaaggccggcaccaactactatatcaaagtccgggccggcgataacaaatacatgcatctgaaagtgttcaaatcgctcgaatacaagttcagaaaccttgaagaggaccttgtgctcaccgggtaccaggtcgacaagaataaggatgacgaattgactggattc

SEQ ID NO: 712 - DNA, 1182 bp, synthetic construct
  source 1..1182: mol_type = other DNA
attcctggaggcctgagcgaagccaagcccgccactcctgaaatccaagaaatcgtggacaaggtcaagccacagctggaagagaaaaccggggaaacttacgggaagctggaggccgtccagtacaagacacaagtcgtggtcgaccaagtctggtccgagaatgccggaaccaactactacatcaaggtccgggccggagataacaagtatatgcacctgaaagtgttcaagtcactcccgtacctttggaggaagcacgcggtcgaggacctggtgctgaccgggtaccaggtcgataagaacaaagacgacgaactcaccggattcgcggaagcagccgctaaggaagctgcagcgaaagaagcggccgcgaaggaagcagcggccaaagaggccgccgccaaagaagccgcggcgaagatgatcccgggaggactgtcggaagcgaagccagccacccccgaaatccaggagatcgtggataaggtcaaaccgcagctggaggagaaaactggcgaaacctacggaaagttggaggccgtgcagtataagacccaggtcgtggtggaccaagtgtggtccgaaaacgccggcactaactattacatcaaagtcagagccggcgacaacaagtacatgcatctgaaggtgttcaagagcctcccctacttgtggcgcaagcacgccgtagaggatctcgtgctgacgggctaccaagtggacaagaacaaggacgacgagcttaccggcttcgccgaagccgcagcaaaggaggcggccgcaaaggaagcggcagccaaggaggccgctgctaaggaggctgccgccaaggaagccgctgccaagatgattcccggtggactgtccgaggccaagcctgcgacccctgagattcaggaaatcgtcgacaaagtcaagccccagttggaagaaaagactggagagacttacggaaagctcgaggcggtgcagtacaaaacccaagtggtggtggatcaagtctggagcgagaacgcgggcaccaattactacattaaggtccgcgcaggagacaacaaatacatgcacctcaaggtctttaagtccctgccgtacctgtggcggaagcatgccgtggaggacctcgtgttgaccggttaccaggttgacaaaaacaaggatgatgagctgactggattc

SEQ ID NO: 713 - DNA, 1182 bp, synthetic construct
  source 1..1182: mol_type = other DNA
attccgggcggactctctgaggccaagcccgccactcctgagattcaggagattgtggataaggtcaaaccgcagctcgaagaaaaaaccggagagacttacggaaaactggaggcggtccagtacaaaacccaagtcgtgggcgactcctattgggaatatgtgtcggggaccaactactacatcaaagtccgcgccggagataacaaatacatgcacctcaaagtgttcaagagcttgccttaccgctggaggcctgaccacgcggaggatctggtgctcaccgggtaccaggtcgacaaaaacaaggacgacgagctgactggctttgccgaagctgccgcaaaagaggctgccgctaaggaggcggccgcgaaagaggcagcagccaaggaggccgctgccaaagaggccgcagccaagatgatcccgggtggactgtccgaggccaaacccgcgacccctgagatccaggaaatcgtggacaaggtcaagcctcagctggaagaaaagactggcgaaacctacggaaagctggaagctgtgcagtacaagacccaggtcgtgggagactcatactgggaatacgtgtccggtaccaactattacattaaggtccgcgcgggagataataagtacatgcatctgaaggtgttcaagtccctgccgtaccggtggaggccggatcatgccgaggacctggtcctcaccggataccaagtggataagaacaaggatgacgaactgaccggcttcgccgaagcagccgcaaaggaagcagcggctaaggaagctgcggccaaagaagccgccgcgaaggaagcggccgccaaggaagccgcggcgaagatgattcctggcggtctgtccgaagctaagcccgctaccccggaaatccaagagatcgtggataaagtcaagccccaactcgaggaaaagaccggagaaacttacgggaagctcgaagccgtgcagtataagactcaagtggtcggggactcgtactgggagtacgtcagcggcactaactactatatcaaagttcgggccggcgacaacaagtatatgcaccttaaagtgtttaagagcctgccctaccgatggcggcccgatcatgcggaggacttggtgctgacgggataccaggtggacaagaataaggacgatgagctgaccggattc

SEQ ID NO: 714 - DNA, 1182 bp, synthetic construct
  source 1..1182: mol_type = other DNA
atccctggtggcctgtccgaagccaagcctgcgacccctgagatccaagagattgtggacaaagtcaagccgcagctggaagaaaagaccggggaaacctacggcaaactggaggccgtacagtacaagacccaggtcgtgcttgacggcctgtaccatgtcaactccggtacaaactactacatcaaggtccgagccggagacaacaaatacatgcatctcaaagtgttcaagagcctgttctactattaccggtccgaaccgactgaggacctggtgctcacgggttaccaggtcgacaaaaacaaggacgacgaactgaccggttttgccgaagccgcagccaaggaagctgcggccaaagaagccgccgcaaaggaagcagccgccaaggaggctgccgcgaaggaagccgcggcgaagatgatcccgggaggactctcagaggccaaacccgccaccccggaaatccaggagatcgtggacaaggtcaagccacagctggaggaaaagactggagagacttacggaaagctcgaggccgtgcagtacaaaacccaagtggtgctggacggcctttatcacgtgaacagcggcaccaactactatatcaaggttcgcgcgggagacaataagtacatgcacctgaaggtgttcaagtccctgttctattactaccgctcggagccaaccgaggatttggtgctgaccgggtatcaagtggataagaacaaggatgacgagctgaccggcttcgctgaggccgcagcaaaggaggccgccgccaaagaggccgcggctaaggaggcagccgctaaggaagcggccgcgaaagaagcggcggccaagatgattcccggcggactgtcagaagctaagccggccacccctgaaattcaggaaatcgtggataaggtcaaaccccagttggaggagaaaaccggcgaaacttacgggaagctcgaagccgtgcaatacaagactcaggtcgtcctggacgggctgtaccacgtgaactccggcaccaattactacattaaggtccgcgccggagataacaagtatatgcatctgaaagtgtttaagtccctcttctactactaccggtcggagcctactgaggatctggtgctgactgggtaccaagtggacaagaacaaagacgacgagctcaccggattc

SEQ ID NO: 715 - DNA, 1182 bp, synthetic construct
  source 1..1182: mol_type = other DNA
attcccggaggcctctcagaggccaagcctgccacccccgaaattcaggaaatcgtggataaggtcaagccgcagctggaggagaaaaccggggaaacctacggaaagctggaagcggtgcaatacaaaacccaggtcgtcaagacttgggaggatactgtggtcgccggtaccaattactatatcaaggtccgcgctggcgataacaaatatatgcacctgaaggtgttcaagtccctcgagtacctgtatcggcctctgaaccccgaggacctggtgctgactggataccaggtcgacaagaacaaggacgatgaactgacggggttcgctgaagctgccgccaaggaggctgcggcaaaggaagcagccgcgaaggaggcagcggccaaggaagctgcggccaaagaggccgcggcgaagatgatcccgggtggactctccgaagccaagccagccaccccggaaatccaagagatcgtggacaaggtcaaaccgcagcttgaggaaaagaccggcgaaacttacgggaagttggaagccgtgcagtacaagacccaagtcgtgaaaacctgggaagatacagtggtggcaggcaccaactactacatcaaagtccgcgccggagacaacaaatacatgcatctgaaagtgttcaagagcctggaatacctttaccggccgctgaatcccgaggatttggtgctcactggctaccaagtggacaagaataaggacgacgaactcaccggcttcgccgaagccgctgctaaggaagcggctgcgaaggaagccgccgcaaaggaggcggccgctaaggaggccgccgccaaagaagcggccgccaagatgattcctggtggcctgtcggaggcgaaaccggccacccctgagattcaggagatcgtcgacaaagtgaagccacagctggaagaaaagactggcgaaacctatgggaagcttgaggcagtgcagtataagactcaagtggtcaagacctgggaggacactgtggtggccggaactaattactacattaaggtccgggccggagataacaagtacatgcacctcaaagtgtttaagtccctggaatacttgtacagaccactgaaccctgaggacctcgtgctgaccgggtaccaggttgataagaacaaagacgacgagcttactggattc

SEQ ID NO: 716 - DNA, 1599 bp, synthetic construct
  source 1..1599: mol_type = other DNA
atccctgggggactcagcgaggctaagcctgcgactcccgaaatccaagagatcgtggacaaagtgaagccgcaactggaagaaaagaccggagaaacttacgggaaactggaggcagtgcaatacaagacacaagtggtccactattacgtccactacaatgatcagggcaccaattattacattaaggtgcgagccggcgataacaaatatatgcacctgaaggtctttaagtccctgtggggagagaatctgttcgcaaagtgggaggacctcgtgcttactggttaccaggtagacaagaataaggacgatgagctgactggtttcggaggcggtggtagcggagggggtggaagcggaggagggggaagtggtggcggtggatcaggcggagggggatcagggggaggagggtccatgatccctggagggctgtccgaggccaagcctgctacgccggaaattcaagaaatcgtcgataaggttaagccacagctcgaagagaaaactggggaaacatacggcaaacttgaggcggttcagtacaaaactcaggtcgtgcactactacgtgcactacaacgatcaagggaccaactactatatcaaggtccgcgcgggagacaacaaatacatgcatctcaaagtgttcaaatcgctgtggggcgagaaccttttcgcgaagtgggaggatttggtgctgactggatatcaggtcgataaaaacaaggatgacgagcttactggctttggcggaggcggatcaggaggaggtggcagcggtggaggaggttccggtggaggcggttccggaggaggaggctcaggagggggaggctccatgattcccggaggcctctcagaagccaaaccagccactcctgagattcaggaaattgtcgacaaggtcaagccgcagctggaagagaagactggcgaaacctacgggaagctagaggcggtccagtacaagacccaagtcgtccattactatgtacattacaacgaccagggaaccaattactacattaaagtgcgggccggagataataagtatatgcatcttaaggtgtttaagtcgttgtggggcgaaaacctgtttgccaagtgggaagatcttgtgctcaccggttaccaagtggacaagaacaaggacgacgagctgaccgggttcggaggaggcggaagcggtggtggaggatcgggaggcggaggatccggtggtggcggatctggcggtggaggttctggggggggaggatccatgatacctggcggactgtcggaggcgaaaccggccacccctgaaatccaggagattgtggacaaggtgaaaccccaactcgaggaaaaaaccggcgaaacgtacggtaaactcgaagccgtgcagtataagacccaggtcgtacactactatgtgcactataacgatcagggaacgaactactacatcaaggtcagagctggagacaataagtacatgcacctcaaggttttcaagtcgttatggggagaaaacctcttcgcgaaatgggaggatctcgtgctgaccggctaccaggtggacaaaaataaggatgatgaactcaccggtttc

SEQ ID NO: 717 - DNA, 1608 bp, synthetic construct
  source 1..1608: mol_type = other DNA
atccctgggggactcagcgaggctaagcctgcgactcccgaaatccaagagatcgtggacaaagtgaagccgcaactggaagaaaagaccggagaaacttacgggaaactggaggcagtgcaatacaagacacaagtggtccactattacgtccactacaatgatcagggcaccaattattacattaaggtgcgagccggcgataacaaatatatgcacctgaaggtctttaagtccctgtggggagagaatctgttcgcaaagtgggaggacctcgtgcttactggttaccaggtagacaagaataaggacgatgagctgactggtttcgccgaagccgcagcaaaagaggccgccgcgaaagaagccgctgctaaggaggcggccgcaaaggaggccgcggcaaaagaagcggcggccaaaatgatccctggagggctgtccgaggccaagcctgctacgccggaaattcaagaaatcgtcgataaggttaagccacagctcgaagagaaaactggggaaacatacggcaaacttgaggcggttcagtacaaaactcaggtcgtgcactactacgtgcactacaacgatcaagggaccaactactatatcaaggtccgcgcgggagacaacaaatacatgcatctcaaagtgttcaaatcgctgtggggcgagaaccttttcgcgaagtgggaggatttggtgctgactggatatcaggtcgataaaaacaaggatgacgagcttactggctttgctgaggctgccgccaaggaggctgcggcgaaggaagcagcagcgaaagaggcagccgccaaagaagctgcagcgaaggaggcagcagccaagatgattcccggaggcctctcagaagccaaaccagccactcctgagattcaggaaattgtcgacaaggtcaagccgcagctggaagagaagactggcgaaacctacgggaagctagaggcggtccagtacaagacccaagtcgtccattactatgtacattacaacgaccagggaaccaattactacattaaagtgcgggccggagataataagtatatgcatcttaaggtgtttaagtcgttgtggggcgaaaacctgtttgccaagtgggaagatcttgtgctcaccggttaccaagtggacaagaacaaggacgacgagctgaccgggttcgctgaagctgcggccaaagaggcggcggcaaaggaagcggccgctaaagaagcagcggccaaggaagctgccgctaaggaagccgcggcgaagatgatacctggcggactgtcggaggcgaaaccggccacccctgaaatccaggagattgtggacaaggtgaaaccccaactcgaggaaaaaaccggcgaaacgtacggtaaactcgaagccgtgcagtataagacccaggtcgtacactactatgtgcactataacgatcagggaacgaactactacatcaaggtcagagctggagacaataagtacatgcacctcaaggttttcaagtcgttatggggagaaaacctcttcgcgaaatgggaggatctcgtgctgaccggctaccaggtggacaaaaataaggatgatgaactcaccggtttc

SEQ ID NO: 718 - DNA, 1602 bp, synthetic construct
  source 1..1602: mol_type = other DNA
atccctggagggctgtccgaggccaagcctgctacgccggaaattcaagaaatcgtcgataaggttaagccacagctcgaagagaaaactggggaaacatacggcaaacttgaggcggttcagtacaaaactcaggtcgtgcactactacgtgcactacaacgatcaagggaccaactactatatcaaggtccgcgcgggagacaacaaatacatgcatctcaaagtgttcaaatcgctgtggggcgagaaccttttcgcgaagtgggaggatttggtgctgactggatatcaggtcgataaaaacaaggatgacgagcttactggctttggaggaggagggtccggtggaggaggctctggtggaggcggaagcggcggaggaggatcgggtggcggaggaagcggtggtggaggctccatgattcccggaggcctctcagaagccaaaccagccactcctgagattcaggaaattgtcgacaaggtcaagccgcagctggaagagaagactggcgaaacctacgggaagctagaggcggtccagtacaagacccaagtcgtccattactatgtacattacaacgaccagggaaccaattactacattaaagtgcgggccggagataataagtatatgcatcttaaggtgtttaagtcgttgtggggcgaaaacctgtttgccaagtgggaagatcttgtgctcaccggttaccaagtggacaagaacaaggacgacgagctgaccgggttcggaggaggcggatcgggaggtggaggatccggtggcggtggaagcggaggtggcggatctgggggagggggatcaggaggcggcggatccatgatacctggcggactgtcggaggcgaaaccggccacccctgaaatccaggagattgtggacaaggtgaaaccccaactcgaggaaaaaaccggcgaaacgtacggtaaactcgaagccgtgcagtataagacccaggtcgtacactactatgtgcactataacgatcagggaacgaactactacatcaaggtcagagctggagacaataagtacatgcacctcaaggttttcaagtcgttatggggagaaaacctcttcgcgaaatgggaggatctcgtgctgaccggctaccaggtggacaaaaataaggatgatgaactcaccggtttcggaggcggaggatcaggcggagggggttcaggagggggtggctcaggaggagggggtagtggaggcggtggatccggaggaggtggcagcatgatcccacggggtctgtccgaagccaagccagccaccccagagatccaagaaatagtggataaggtgaagccccagcttgaggaaaagactggagagacttacggaaagctcgaggctgtccaatacaaaacacaggtgctggcgaacttcttccaaaggagatggccaggcagtactaactattacatcaaagtgcgcgcaggagataacaaatatatgcacttgaaggtgttcaatggaccgtggaagttccggaacaccgacagaggcgccgacagggtcttgaccgggtaccaagtcgataagaacaaagacgacgaactgaccggtttc

SEQ ID NO: 719 - DNA, 2025 bp, synthetic construct
  source 1..2025: mol_type = other DNA
atccctgggggactcagcgaggctaagcctgcgactcccgaaatccaagagatcgtggacaaagtgaagccgcaactggaagaaaagaccggagaaacttacgggaaactggaggcagtgcaatacaagacacaagtggtccactattacgtccactacaatgatcagggcaccaattattacattaaggtgcgagccggcgataacaagtatatgcacctgaaggtctttaagtccctgtggggagagaatctgttcgcaaagtgggaggacctcgtgcttactggttaccaggtagacaagaataaggacgatgagctgactggtttcgggggcggaggatctgggggtggaggaagcggtggtggaggctcgggtggaggagggtctgggggaggaggttcaggcggcggaggttccatgatccctggagggctgtccgaggccaagcctgctacgccggaaattcaagaaatcgtcgataaggttaagccacagctcgaagagaaaactggggaaacatacggcaaacttgaggcggttcagtacaaaactcaggtcgtgcactactacgtgcactacaacgatcaagggaccaactactatatcaaggtccgcgcgggagacaacaaatacatgcatctcaaagtgttcaaatcgctgtggggcgagaaccttttcgcgaagtgggaggatttggtgctgactggatatcaggtcgataaaaacaaggatgacgagcttactggctttggaggcggtggatcgggagggggtggttcgggaggaggaggctcaggcggaggaggatcaggaggcggcggaagtggaggcggaggctccatgattcccggaggcctctcagaagccaaaccagccactcctgagattcaggaaattgtcgacaaggtcaagccgcagctggaagagaagactggcgaaacctacgggaagctagaggcggtccagtacaagacccaagtcgtccattactatgtacattacaacgaccagggaaccaattactacattaaagtgcgggccggagataataagtatatgcatcttaaggtgtttaagtcgttgtggggcgaaaacctgtttgccaagtgggaagatcttgtgctcaccggttaccaagtggacaagaacaaggacgacgagctgaccgggttcggaggaggcggatcagggggtggtggttccggcggaggtggatccggaggaggtggaagcgggggaggtggttcaggagggggcggatccatgatacctggcggactgtcggaggcgaaaccggccacccctgaaatccaggagattgtggacaaggtgaaaccccaactcgaggaaaaaaccggcgaaacgtacggtaaactcgaagccgtgcagtataagacccaggtcgtacactactatgtgcactataacgatcagggaacgaactactacatcaaggtcagagctggagacaataagtacatgcacctcaaggttttcaagtcgttatggggagaaaacctcttcgcgaaatgggaggatctcgtgctgaccggctaccaggtggacaaaaataaggatgatgaactcaccggtttcggcggtggaggatcgggcggtggtggtagcggagggggaggatccggtggagggggtagcggtggaggcggttccggtggtggcggaagcatgatcccacggggtctgtccgaagccaagccagccaccccagagatccaagaaatagtggataaggtgaagccccagcttgaggaaaagactggagagacttacggaaagctcgaggctgtccaatacaaaacacaggtgctggcgaacttcttccaaaggagatggccaggcagtactaactattacatcaaagtgcgcgcaggagataacaaatatatgcacttgaaggtgttcaatggaccgtggaagttccggaacaccgacagaggcgccgacagggtcttgaccgggtaccaagtcgataagaacaaagacgacgaactgaccggtttc

SEQ ID NO: 720 - DNA, 1611 bp, synthetic construct
  source 1..1611: mol_type = other DNA
atccctggagggctgtccgaggccaagcctgctacgccggaaattcaagaaatcgtcgataaggttaagccacagctcgaagagaaaactggggaaacatacggcaaacttgaggcggttcagtacaaaactcaggtcgtgcactactacgtgcactacaacgatcaagggaccaactactatatcaaggtccgcgcgggagacaacaaatacatgcatctcaaagtgttcaaatcgctgtggggcgagaaccttttcgcgaagtgggaggatttggtgctgactggatatcaggtcgataaaaacaaggatgacgagcttactggctttgccgaggctgcagccaaagaagccgcggcgaaggaagcggcggcaaaggaggcagccgcaaaggaagctgccgctaaagaggccgccgcaaagatgattcccggaggcctctcagaagccaaaccagccactcctgagattcaggaaattgtcgacaaggtcaagccgcagctggaagagaagactggcgaaacctacgggaagctagaggcggtccagtacaagacccaagtcgtccattactatgtacattacaacgaccagggaaccaattactacattaaagtgcgggccggagataataagtatatgcatcttaaggtgtttaagtcgttgtggggcgaaaacctgtttgccaagtgggaagatcttgtgctcaccggttaccaagtggacaagaacaaggacgacgagctgaccgggttcgctgaggctgccgccaaggaagccgcagcgaaggaggccgctgcgaaagaagcggcagccaaggaggcggctgccaaagaggcggccgcgaagatgatacctggcggactgtcggaggcgaaaccggccacccctgaaatccaggagattgtggacaaggtgaaaccccaactcgaggaaaaaaccggcgaaacgtacggtaaactcgaagccgtgcagtataagacccaggtcgtacactactatgtgcactataacgatcagggaacgaactactacatcaaggtcagagctggagacaataagtacatgcacctcaaggttttcaagtcgttatggggagaaaacctcttcgcgaaatgggaggatctcgtgctgaccggctaccaggtggacaaaaataaggatgatgaactcaccggtttcgctgaagcagcggcgaaagaggcagcggccaaggaagccgctgccaaggaagccgccgctaaggaggctgcggctaaggaagcagccgccaagatgatcccacggggtctgtccgaagccaagccagccaccccagagatccaagaaatagtggataaggtgaagccccagcttgaggaaaagactggagagacttacggaaagctcgaggctgtccaatacaaaacacaggtgctggcgaacttcttccaaaggagatggccaggcagtactaactattacatcaaagtgcgcgcaggagataacaaatatatgcacttgaaggtgttcaatggaccgtggaagttccggaacaccgacagaggcgccgacagggtcttgaccgggtaccaagtcgataagaacaaagacgacgaactgaccggattc

SEQ ID NO: 721 - DNA, 2037 bp, synthetic construct
  source 1..2037: mol_type = other DNA
atccctgggggactcagcgaggctaagcctgcgactcccgaaatccaagagatcgtggacaaagtgaagccgcaactggaagaaaagaccggagaaacttacgggaaactggaggcagtgcaatacaagacacaagtggtccactattacgtccactacaatgatcagggcaccaattattacattaaggtgcgagccggcgataacaagtatatgcacctgaaggtctttaagtccctgtggggagagaatctgttcgcaaagtgggaggacctcgtgcttactggttaccaggtagacaagaataaggacgatgagctgactggtttcgccgaagctgcagcgaaagaggcagctgcgaaagaagcggcagccaaagaagccgctgccaaggaggctgccgccaaagaggcggccgccaaaatgatccctggagggctgtccgaggccaagcctgctacgccggaaattcaagaaatcgtcgataaggttaagccacagctcgaagagaaaactggggaaacatacggcaaacttgaggcggttcagtacaaaactcaggtcgtgcactactacgtgcactacaacgatcaagggaccaactactatatcaaggtccgcgcgggagacaacaaatacatgcatctcaaagtgttcaaatcgctgtggggcgagaaccttttcgcgaagtgggaggatttggtgctgactggatatcaggtcgataaaaacaaggatgacgagcttactggctttgccgaagccgcagcgaaggaagcagccgcgaaggaggcagcggcaaaagaggccgcggctaaagaagctgccgcaaaggaggccgccgcaaagatgattcccggaggcctctcagaagccaaaccagccactcctgagattcaggaaattgtcgacaaggtcaagccgcagctggaagagaagactggcgaaacctacgggaagctagaggcggtccagtacaagacccaagtcgtccattactatgtacattacaacgaccagggaaccaattactacattaaagtgcgggccggagataataagtatatgcatcttaaggtgtttaagtcgttgtggggcgaaaacctgtttgccaagtgggaagatcttgtgctcaccggttaccaagtggacaagaacaaggacgacgagctgaccgggttcgctgaggcagccgcaaaagaagcagcagcaaaggaggccgccgccaaggaagcggcggcaaaggaagctgctgctaaggaggcggctgccaagatgatacctggcggactgtcggaggcgaaaccggccacccctgaaatccaggagattgtggacaaggtgaaaccccaactcgaggaaaaaaccggcgaaacgtacggtaaactcgaagccgtgcagtataagacccaggtcgtacactactatgtgcactataacgatcagggaacgaactactacatcaaggtcagagctggagacaataagtacatgcacctcaaggttttcaagtcgttatggggagaaaacctcttcgcgaaatgggaggatctcgtgctgaccggctaccaggtggacaaaaataaggatgatgaactcaccggtttcgctgaagcggccgctaaagaggctgcggctaaggaagccgcggcgaaggaagcagcggccaaggaggctgctgcaaaggaggccgccgctaagatgatcccacggggtctgtccgaagccaagccagccaccccagagatccaagaaatagtggataaggtgaagccccagcttgaggaaaagactggagagacttacggaaagctcgaggctgtccaatacaaaacacaggtgctggcgaacttcttccaaaggagatggccaggcagtactaactattacatcaaagtgcgcgcaggagataacaaatatatgcacttgaaggtgttcaatggaccgtggaagttccggaacaccgacagaggcgccgacagggtcttgaccgggtaccaagtcgataagaacaaagacgacgaactgaccggattc

SEQ ID NO: 722 - DNA, 1698 bp, synthetic construct
  source 1..1698: mol_type = other DNA
atacctggcggtctgagcgaagcaaagccggccactcctgaaattcaggaaatcgtggacaaggttaagccccagctcgaggaaaagaccggcgaaacctatggcaaactggaagcggtgcagtacaagactcaggtcgtgcactattacgtgcactataacgaccaaggcactaactattacatcaaggtccgagcgggagacaacaagtacatgcacctgaaagtgtttaagagcctgtggggggaaaacctcttcgccaaatgggaagatctggtgctcaccgggtaccaggtcgacaagaacaaagacgacgagctgaccgggttcgctgaggcagcggcaaaagaagccgcggctaaagaggctgccgccaaggaagccgccgcaaaggaggccgctgccaaagaggccgccgccaagatgattcccgggggactttcggaagccaagcctgccacaccggagatccaagagattgtggacaaagtgaagcctcagctcgaagaaaagactggagagacttatggaaagctggaggccgtccagtacaaaacccaagtcgtgcattactacgtgcattacaacgatcaggggactaactactatatcaaagtgcgcgccggggataacaaatatatgcatcttaaggtgtttaagtccctctggggcgaaaacttgttcgcgaaatgggaggacttggtgcttactggataccaagtcgacaaaaacaaggatgatgaactgacgggattcgccgaagccgctgctaaggaggcagccgcgaaagaagcggccgctaaggaagcggcggccaaagaagctgcggcgaaggaagctgccgcgaagatgatcccgggaggactgagcgaggccaagccagcaaccccggagattcaagaaatcgtcgacaaggtcaagccccagttggaagagaaaaccggggaaacctacggcaaactcgaagccgtgcagtataagactcaagtggtgcactactacgtccactacaacgaccagggcaccaactactacattaaggtccgcgctggtgacaacaaatacatgcatctgaaggtctttaagtcgctgtggggagagaacctgttcgcgaagtgggaggacctggtgctgaccggctaccaagtggacaagaataaggatgacgagctcaccggcttcggaggaggagggtccggtggtggaggttcgggagggggtggttccggagggggaggttccggtggaggcggaagtggcggaggaggatccggcggtggtggatccggtggcggtggatcaggaggcggaggctcaggaggtggcggaagcggaggagggggttcaggaggaggcggctccatgattccgaggggtctgtcagaggctaagcccgcgacccctgaaatccaggagatcgtggataaggtcaaaccgcagctggaggagaaaactggggagacttacgggaagctcgaggccgtgcaatacaagacccaagtgctcgccaacttcttccagcggagatggcccggaagcacgaattactacatcaaagtccgggccggagataataagtatatgcacttgaaggtgttcaacgggccctggaagttccggaacaccgatagaggcgcagatcgcgtgctgactggctaccaggtggacaaaaataaggacgacgaactgaccggtttc

SEQ ID NO: 723 - DNA, 1602 bp, synthetic construct
  source 1..1602: mol_type = other DNA
atccctggaggcctgagcgaggccaagccagctaccccggagattcaggaaatcgtggacaaggtcaaaccgcagctcgaggaaaagactggagaaacctatggcaaactcgaagcggtccagtacaagactcaagtggtgcactactacgtgcattacaatgaccagggcacgaactactacatcaaagtccgggctggcgacaacaagtatatgcatctaaaggtctttaagtccctctggggcgaaaacctgttcgccaaatgggaagatctggtgctcactgggtaccaagtcgataagaacaaagacgacgaactcaccgggttcggaggagggggttccggtggaggtggcagcggaggtggtggctcaatgattccgggaggactgtcggaagctaagccggccactcccgaaattcaagagatcgtggataaggttaaaccccagctcgaagaaaagaccggggaaacttatggaaagctcgaagccgtgcagtacaaaacccaagtcgtgcattactacgtccactataacgatcaggggaccaactactatatcaaggtccgcgccggagataataagtacatgcatctgaaggtgttcaagtcgctctggggggaaaaccttttcgcgaaatgggaggatctcgtgctgaccgggtaccaggtcgacaagaacaaggatgacgagctgaccggcttcggtggaggaggaagcggcggaggtggaagcggtggtggcggatccatgatccccggaggcctttccgaagcgaagcctgccacccctgagatccaagagattgtggacaaagtcaagccacagctggaggagaaaaccggagaaacttacggaaagctggaggccgtgcaatataagacccaggtcgtgcactattacgtgcactacaacgaccagggaactaactattacatcaaggtcagagcgggagataacaagtacatgcacctgaaagtgtttaagtcgctgtggggagagaatctgttcgctaagtgggaggacctggtgctgactggctaccaggtggacaagaataaggacgatgagcttaccggattcggagggggaggctctggaggaggcggctcaggaggtggaggatcggggggtggaggttccggcggaggaggatcagggggcggaggaagtggtggaggcggttcgggaggaggtggatctggcggaggcggatcaggaggaggagggagtggcggtggtggatcaggcggtggagggtccatgattcctcggggactgagcgaagccaagcccgcgactccggaaatccaggagatcgtcgataaggtcaagccccagcttgaagaaaaaaccggcgaaacctacgggaagctcgaggccgtccaatacaagacacaagtgctggccaacttcttccaacggcgctggccaggatccacgaattactacattaaggtccgagccggcgataacaaatacatgcacttgaaagtgttcaacggaccttggaagttcaggaacactgaccggggcgcagacagagtgcttaccggctaccaagtggacaaaaacaaggacgacgagttgactggattc

SEQ ID NO: 724 - DNA, 1605 bp, synthetic construct
  source 1..1605: mol_type = other DNA
attccgggaggccttagcgaagcgaagcctgcgacgccagagatccaggaaattgtggacaaagtgaagccccaactggaggagaaaaccggagaaacctatggaaagctggaagcggtgcagtacaagacccaagtggtgcattactacgtgcactataacgatcaggggactaattactacattaaggtccgggctggggataacaagtatatgcaccttaaggtattcaagtcactgtggggggagaacctgtttgctaagtgggaggacctggtgctgaccggttaccaggtggacaaaaataaggacgacgaactgactgggttcggtggcggaggttcgggagggggaggaagcggaggcggaggatccatgattccgggtggactctccgaggccaagcctgccactcccgaaatccaggagatcgtggacaaggtcaagcctcagctggaggaaaagactggagaaacatacgggaaactggaagccgtgcagtataagacccaggtggtgcactattacgtacactacaacgaccagggcaccaactactacatcaaagtccgggccggcgataataagtacatgcacctgaaggtgttcaagtccctctggggcgaaaacctgttcgcgaagtgggaagatctcgtgctcaccgggtaccaagtggacaagaacaaggatgacgagctgaccggctttggcggaggaggctcaggagggggtggtagcggaggagggggttccatgatccccggaggtctgagcgaagccaagcccgcaacaccggagatccaagagatcgtcgacaaggttaagccgcagctggaagaaaagaccggcgaaacctacggcaaactggaggccgtgcaatacaaaacccaggtcgtgcactactacgtccactacaatgaccaaggcactaattattacatcaaggtccgcgctggcgataacaaatatatgcatctgaaagtgtttaagtcgctgtggggagagaacctcttcgccaaatgggaggacttggtgttgaccggctaccaggtcgacaaaaacaaggacgatgagcttaccggctttgccgaagccgcggcaaaggaggccgccgcgaaagaagcggcggccaaagaagccgccgctaaggaggcggccgctaaagaggcggcagccaaagaggctgccgccaaggaagctgcggctaaggaagcagcggcgaaggaggctgctgccaaggaggcagccgcgaaggaagcggccgccaagatgatccctaggggcctgtccgaagctaagccagccacccctgagattcaggaaatcgtggataaagtcaagccacaactggaagagaaaactggggaaacttacgggaagctcgaagccgtccagtacaaaactcaagtgctggccaacttcttccaacggagatggccgggatccaccaactattacataaaagtccgcgccggagacaacaaatacatgcatctcaaggtctttaacgggccgtggaagttccgcaacactgaccggggagcagacagagtgctgactggttaccaagtcgacaagaataaggatgatgagctgacgggattc

SEQ ID NO: 725 - DNA, 1608 bp, synthetic construct
  source 1..1608: mol_type = other DNA
atccctggagggctcagcgaggccaaacccgccactcctgagattcaggagatcgtggataaggtcaaaccgcagctcgaggagaaaacgggcgaaacttacgggaagctggaagccgtgcagtataagacccaagtggtgcattactacgtccattacaacgatcagggcaccaattactacattaaggtccgggctggtgacaacaaatacatgcatctgaaggtctttaagtcgctctggggggaaaacctcttcgcgaagtgggaagatctggtccttaccggataccaagtggacaagaacaaagatgacgagttgaccgggtttgccgaggcggcagctaaggaggcagccgcgaaggaagcggccgcgaagatgatccctggcggactgtccgaagccaagcctgcgacccctgaaattcaggaaatcgtggacaaagtcaagccccagttggaagaaaagaccggcgaaacatacggaaagctagaggcagtgcagtacaagacccaggtcgtgcactactatgtgcactacaacgaccagggtactaactactatatcaaggttcgcgcgggagacaacaagtatatgcatctcaaagtgttcaagagcctgtggggcgaaaacctgttcgccaaatgggaggacctggtgctgaccggctaccaggtggataagaacaaggacgacgagctgaccgggttcgctgaagccgcagccaaggaggctgccgccaaggaagccgccgcaaagatgattccgggaggattgtccgaagcgaagccggccactccagagatccaagaaattgtggacaaggttaaaccgcaactggaggaaaagactggcgaaacctatggaaagctcgaggccgtccagtacaaaactcaggtcgtacactactacgtgcactataacgaccaagggaccaattattacatcaaggtccgcgccggagataacaaatatatgcacctcaaggtgttcaagtcgctgtggggggagaacctgtttgccaagtgggaggatcttgtgctgacggggtaccaggttgacaagaataaggacgatgaacttaccggctttggagggggtggtagcggaggcggtggttccggtggtggcggaagcggaggaggaggctccggtggaggaggatcaggaggcggaggatcgggtggcggaggttctggcggaggaggttccggagggggaggaagtggcggtggaggttcgggcggtggtggatcagggggaggcggatccatgatcccccgaggactgagcgaggctaagccagcgaccccggaaatccaagagattgtcgacaaggtcaagccgcagctggaagagaaaaccggggaaacctacggcaaactggaagcggtgcaatacaagactcaggtgctcgcaaacttcttccaacggcgctggcctggatccaccaactactacatcaaagtccgggccggggataacaagtacatgcacctgaaagtgtttaacggcccgtggaagttccggaacaccgacagaggagccgacagagtgctgactggataccaggtcgacaaaaacaaagacgacgaactgactggcttc

SEQ ID NO: 726 - DNA, 1611 bp, synthetic construct
  source 1..1611: mol_type = other DNA
attccgggtggactgtccgaggccaagccagccacccccgaaatccaggaaattgtggacaaggtcaagccacagctggaggaaaagaccggcgaaacctatggaaagctcgaggcagtgcagtataagacccaggtggtccactactatgtgcactacaatgaccaagggaccaactattacatcaaagtccgcgccggagacaacaagtacatgcatctgaaggttttcaagtccctttggggcgaaaacctgtttgccaaatgggaggacttggtgctgacggggtaccaagtggataagaacaaagatgacgaactcactgggttcgccgaagcagccgctaaggaggcggccgccaaagaggcggctgccaagatgatcccggggggtctttcggaagcgaagcctgcaacccccgagatccaagagatcgtggacaaagtcaagccccaactggaagagaaaactggagagacttacgggaagctcgaagccgtgcagtacaagactcaggtcgtgcactactacgtccactacaacgatcagggcaccaattactacatcaaggtccgggccggagataataagtatatgcatctcaaggtgttcaagtcgttgtggggagagaacctcttcgcgaagtgggaggatctcgtgctgactggttatcaagtggacaagaacaaggatgacgagctgaccggattcgctgaagcggcagccaaagaagccgcggcgaaggaagccgccgcgaagatgattcccggaggcctgtcagaggctaagcctgccactccggaaattcaggagatcgtcgacaaggttaagccgcagctggaagaaaaaaccggggaaacctacggaaagctggaagcggtgcaatacaagacccaagtggtgcattactacgtgcactataacgaccagggtacaaactactatatcaaagtcagagcgggagataacaaatacatgcaccttaaggtctttaagtcactgtggggggagaacctgttcgctaagtgggaagatctggtgctcaccgggtaccaggtcgacaaaaacaaggacgacgagctcaccggctttgccgaagccgctgcaaaggaagccgcagccaaggaggctgcggcaaaagaagcagcggcgaaagaggctgccgcaaaagaggccgcggcaaaggaggcagcggctaaagaagctgccgccaaggaagcagcagctaaggaagctgcagcgaaggaggccgctgcgaaagaagcggccgcgaaaatgattccgagaggcctgagcgaagccaagccggccactcctgagattcaggaaatcgtggataaagtgaagcctcagttggaagaaaagactggcgaaacttacggaaagttggaagccgtacagtacaaaacccaggtcctggccaacttctttcaacgccgctggcctggatccaccaactactacataaaagtgcgggccggcgataacaagtatatgcacctgaaagtgttcaacggaccgtggaagttccggaacactgacaggggtgccgacagagtcctgaccggataccaggtggataaaaacaaagacgacgaactgactggattc

SEQ ID NO: 727 - DNA, 1611 bp, synthetic construct
  source 1..1611: mol_type = other DNA
atccctgggggactctccgaagcgaagcctgccacccccgaaatccaggaaatcgtggacaaagtcaagccacagttggaggagaaaactggagagacttacggcaaactggaagccgtgcagtataagacccaggtcgtgggtggagggggaggtggaggagggggtggaaccaattactacatcaaagtgcgggccggcgataacaagtacatgcatttgaaggtgttcaagtcactaggcggaggcggaggaggaggtggtggagaagatctggtgctcacgggataccaggtggacaagaataaggacgacgagcttactggattcgcggaagctgctgcaaaggaagctgcagcgaaggaagccgccgcgaaggaggctgctgccaaagaggctgccgctaaggaggcagcggccaagatgatccctggaggcctctccgaggctaagccggctactccagagattcaagagatcgtcgacaaagtgaagccccagctggaagaaaagactggggaaacttacgggaagctggaggccgtgcaatacaaaactcaagtcgttggggggggaggaggtggtggtgggggagggaccaactactacataaaggtccgcgcaggagataataagtacatgcacctcaaagtgtttaagtcactgggtggcggtggtggaggaggcggtggagaggacctcgtgctgacgggctaccaggtcgataagaacaaagacgacgaactgactggcttcgccgaagctgcggcaaaggaggccgcagccaaggaggcggcagctaaagaggccgctgcaaaagaagccgcggccaaagaagcggcggcgaagatgattcctggggggctctcagaggccaagccggcaacccctgagattcaggaaattgtggataaggtcaaaccgcagctggaggaaaagaccggggaaacctacgggaagttggaggcagtgcagtacaaaacacaagtggtcggtggaggtgggggtggaggcgggggaggtactaattactatatcaaggtccgagctggcgacaacaagtatatgcacctgaaggtctttaagtccttgggcggtggaggaggtggtggcggaggagaggacttggtgttgactgggtaccaagtggataaaaacaaggatgacgagctgactggtttcgctgaggcagccgcaaaggaagctgccgcaaaagaggcggctgccaaggaagcggccgcgaaagaagcagcggctaaggaagcagccgccaaaatgatcccgagagggctgtccgaagccaagcccgcgactcctgagatccaggagattgtggacaaggtcaagcctcagcttgaagagaaaaccggggagacttatgggaagctcgaggccgtccagtacaagacccaagtgctcgccaacttcttccaacggagatggcccggctcgaccaactattacattaaggtcagagccggagacaacaaatacatgcatctgaaagtgttcaacggcccgtggaagttccggaacaccgaccgcggagcagacagggtcctgaccggatatcaggtcgacaagaacaaggacgatgagctcaccggattc

SEQ ID NO: 728 - DNA, 2037 bp, synthetic construct
  source 1..2037: mol_type = other DNA
atccctggaggcctctccgaggctaagccggccactccagagattcaagagatcgtcgacaaagtcaagccccagctggaagaaaagactggggaaacttacgggaagctggaggcggtgcaatacaaaactcaggtcgtcggaggcggaggaggaggtggtggcggagggaccaactactatataaaggtccgcgcgggagataataagtacatgcacctcaaagtgtttaagtcactgggtggcggtggtggaggaggcggtggagaggacctcgtgctgacgggataccaggtcgataagaacaaagatgacgaactgactggattcgccgaagctgcggcaaaggaggccgcagccaaggaggcagcggctaaggaagcagctgcaaaggaggccgcagccaaggaagcggccgcgaagatgattcctggggggctctcagaggcaaagccggcgacccctgaaattcaggaaattgtggataaggtcaaacctcagctggaggaaaaaaccggggaaacctatgggaagttggaagccgtgcagtacaaaacacaggtggtcgggggaggtggaggtggaggcggcggtggaactaattactatatcaaggtccgagctggcgacaacaagtatatgcacctgaaggttttcaagagcttgggcggtggaggaggtggtggcggaggagaggacttagtgctgactgggtaccaagtcgataaaaacaaggatgacgagctgactggtttcgctgaggcagccgcaaaggaagctgccgcgaaagaggcggctgccaaggaagctgccgcaaaagaagcagcggccaaggaagcagccgctaagatgatacccagaggcctgtccgaagccaagcccgccactcctgagatccaggagattgtggacaaggtcaagcctcaacttgaagagaaaaccggagagacttatgggaagctcgaggccgtccagtacaagacacaagtcctggccaacttcttccaacggagatggcccggatcgaccaattactacatcaaagtccgcgccggagacaacaaatacatgcatctgaaagtgttcaacggcccgtggaagttccgcaacaccgaccggggagcagacagggtgctcactggctaccaagtggacaagaataaggatgatgaactgaccggctttgctgaggctgcggcgaaggaagctgccgctaaagaagccgccgccaaggaagccgctgcgaaggaggctgccgccaaagaagcggcggccaagatgatccctggcggactgtccgaggccaagcctgcaacccccgagattcaggagatcgtggacaaagtgaagccgcagctcgaggaaaagaccggagaaacttatggaaagctggaagccgtacagtataagacccaggtcgtgggtggcggcggaggtggaggagggggtggcaccaactattacatcaaggttcgcgccggcgataacaaatatatgcatttgaaggtctttaagtcgttgggaggtggcgggggtggaggaggaggggaggacctggtgctgaccggctaccaggtggacaaaaacaaagacgacgaactcaccgggttcgctgaagcggctgcaaaagaggccgcggctaaagaggcagctgccaaagaggctgcagcgaaggaggctgctgctaaggaggcggccgccaaaatgatcccgggaggcctgtcagaagcgaagccagccaccccggaaattcaagaaatcgtggataaagttaagcctcagctcgaagaaaaaacgggcgaaacctacggcaaacttgaagccgtccaatacaagacccaagtggtcggaggtgggggtggtggtggtgggggaggaacgaactactacattaaggtccgggccggagataacaagtacatgcacttgaaggtgttcaagtcccttggtggagggggaggaggaggaggcggagaagatctcgtcctgaccggatatcaggtcgacaagaacaaggacgatgagctgaccggattc

SEQ ID NO: 729 - DNA, 8566 bp, synthetic construct
  source 1..8566: mol_type = other DNA
aatattttgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggccctcctatagtgagtcgtattatactatgccgatatactatgccgatgattaattgtcaacacgtgctgcagactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagagctagcctagcggatccgaattctcgatcacaagtttgtacaaaaaagcaggctttaaaggaaccaattcagtcgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaattagcgctactagtgatatcaagatgatctagacccagctttcttgtacaaagtggtatcgagatatctgcatttaattaatctagagtttaaacgcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggttattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 730 - DNA, 9590 bp, synthetic construct
  source 1..9590: mol_type = other DNA
aatattttgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcctagcggatccgaattctcgatcacaagtttgtacaaaaaagcaggcttcgctagccgccaccatgcccctgctcctgctgctgcctctgctgtgggccggtgcattggccatccctggagggctgtccgaggccaagcctgctacgccggaaattcaagaaatcgtcgataaggttaagccacagctcgaagagaaaactggggaaacatacggcaaacttgaggcggttcagtacaaaactcaggtcgtgcactactacgtgcactacaacgatcaagggaccaactactatatcaaggtccgcgcgggagacaacaaatacatgcatctcaaagtgttcaaatcgctgtggggcgagaaccttttcgcgaagtgggaggatttggtgctgactggatatcaggtcgataaaaacaaggatgacgagcttactggctttgccgaggctgcagccaaagaagccgcggcgaaggaagcggcggcaaaggaggcagccgcaaaggaagctgccgctaaagaggccgccgcaaagatgattcccggaggcctctcagaagccaaaccagccactcctgagattcaggaaattgtcgacaaggtcaagccgcagctggaagagaagactggcgaaacctacgggaagctagaggcggtccagtacaagacccaagtcgtccattactatgtacattacaacgaccagggaaccaattactacattaaagtgcgggccggagataataagtatatgcatcttaaggtgtttaagtcgttgtggggcgaaaacctgtttgccaagtgggaagatcttgtgctcaccggttaccaagtggacaagaacaaggacgacgagctgaccgggttcgctgaggctgccgccaaggaagccgcagcgaaggaggccgctgcgaaagaagcggcagccaaggaggcggctgccaaagaggcggccgcgaagatgatacctggcggactgtcggaggcgaaaccggccacccctgaaatccaggagattgtggacaaggtgaaaccccaactcgaggaaaaaaccggcgaaacgtacggtaaactcgaagccgtgcagtataagacccaggtcgtacactactatgtgcactataacgatcagggaacgaactactacatcaaggtcagagctggagacaataagtacatgcacctcaaggttttcaagtcgttatggggagaaaacctcttcgcgaaatgggaggatctcgtgctgaccggctaccaggtggacaaaaataaggatgatgaactcaccggtttcgctgaagcagcggcgaaagaggcagcggccaaggaagccgctgccaaggaagccgccgctaaggaggctgcggctaaggaagcagccgccaagatgatcccacggggtctgtccgaagccaagccagccaccccagagatccaagaaatagtggataaggtgaagccccagcttgaggaaaagactggagagacttacggaaagctcgaggctgtccaatacaaaacacaggtgctggcgaacttcttccaaaggagatggccaggcagtactaactattacatcaaagtgcgcgcaggagataacaaatatatgcacttgaaggtgttcaatggaccgtggaagttccggaacaccgacagaggcgccgacagggtcttgaccgggtaccaagtcgataagaacaaagacgacgaactgaccggattctgaatctagagacccagctttcttgtacaaagtggtatcgagatatctgcatttaattaatctagagtttaaacgcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggttattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 731 - DNA, 9895 bp, synthetic construct
  source 1..9895: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcattaaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacaactttattatacatagttgatcaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 732 - DNA, 9361 bp, synthetic construct
  source 1..9361: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcatggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 733 - DNA, 9951 bp, synthetic construct
  source 1..9951: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcatggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 734 - DNA, 9004 bp, synthetic construct
  source 1..9004: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggcaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcatggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 735 - DNA, 9272 bp, synthetic construct
  source 1..9272: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggcaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcatggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 736 - DNA, 9287 bp, synthetic construct
  source 1..9287: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgtgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcattcaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcatggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 737 - DNA, 9570 bp, synthetic construct
  source 1..9570: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgcgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcaagtttgtacaaaaaagcaggctgccaccatgccattgcttcttctgctgccactcctttgggctggtgcgctggctattccggggggtttgtcagaagcaaagcccgcgaccccagaaattcaggagatcgtggataaagtaaaacctcagcttgaagaaaaaaccggggagacatacggcaagctggaagcagtacaatataaaacgcaggtagtacattactacgtccattataacgatcaaggtacaaattattatataaaagtacgggcaggggacaacaaatacatgcatctgaaagtatttaagagtctgtggggtgagaacttgtttgcaaagtgggaagatttggtgcttaccggctaccaggtcgataagaataaggatgacgagcttacaggttttgcagaagctgcagcgaaagaggctgccgcaaaagaagcagcggctaaggaagcggccgctaaagaagcggccgcgaaggaagcggccgctaagatgatcccgggtggtctgtccgaagccaaaccggcgacgccggaaatccaggagatagtcgataaggtcaagccacaactcgaagagaagactggagaaacatatggaaaacttgaggccgttcaatacaaaacgcaggtggtccactactacgttcactataacgatcaaggaacaaactattacattaaagtcagggcgggagacaacaagtacatgcatcttaaagtgtttaaatctctttggggggagaatcttttcgccaagtgggaagacttggtcctcaccggataccaggtcgacaaaaacaaagacgatgaactcaccggctttgccgaagcggctgcgaaagaagcggctgccaaagaggctgcagccaaagaagctgcggcaaaggaggccgccgccaaagaagcggcggccaaaatgatccccggaggacttagcgaggctaaacccgcgacacctgaaatacaggagatcgtggataaggttaaaccacaattggaggagaaaactggtgagacctatggcaagctggaagcggttcagtataaaacgcaagttgtacactactatgtacattacaacgatcaggggacgaactactatattaaggtcagggcaggagataataaatatatgcacttgaaagttttcaaaagcctgtggggggaaaacttgttcgccaagtgggaagatctcgtcttgactgggtaccaggttgataagaacaaagacgatgaactgacaggcttcgctgaagcggctgcaaaggaggcggcagcgaaggaggctgccgcgaaagaagcggcggctaaagaagcggccgcaaaggaagcagcagcgaagatgataccgaggggtttgagcgaagcaaagcccgcaacgcctgagatacaagaaatcgttgacaaagtaaaaccacaattggaagagaagacgggagaaacgtatggaaagcttgaggccgtacaatacaagacacaggtgttggcaaattttttccaaaggcgatggcccggatcaacgaattactatatcaaggtacgcgcgggggataacaagtacatgcatctcaaggttttcaacggtccgtggaaattccgaaatacagaccgcggagccgacagggtcctcaccggctatcaggtcgacaagaataaggatgatgagttgacaggtttcgccgccgccggtgagcagaaattgatctccgaagaggatctcggtgccgcgcaccaccaccaccatcatggaagcggagagggcaggggaagtcttctaacatgcggggacgtggaggaaaatcccggccccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 738 - DNA, 9581 bp, synthetic construct
  source 1..9581: mol_type = other DNA
aatattttgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgacgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcctagcggatccgaattctcgatcacaagtttgtacaaaaaagcaggcttcgctagccgccaccatgccgctcctgctgctgctgcccctgctgtgggcaggagctctggccatccctggagggctgtccgaggccaagcctgctacgccggaaattcaagaaatcgtcgataaggttaagccacagctcgaagagaaaactggggaaacatacggcaaacttgaggcggttcagtacaaaactcaggtcgtgcactactacgtgcactacaacgatcaagggaccaactactatatcaaggtccgcgcgggagacaacaaatacatgcatctcaaagtgttcaaatcgctgtggggcgagaaccttttcgcgaagtgggaggatttggtgctgactggatatcaggtcgataaaaacaaggatgacgagcttactggctttggaggaggagggtccggtggaggaggctctggtggaggcggaagcggcggaggaggatcgggtggcggaggaagcggtggtggaggctccatgattcccggaggcctctcagaagccaaaccagccactcctgagattcaggaaattgtcgacaaggtcaagccgcagctggaagagaagactggcgaaacctacgggaagctagaggcggtccagtacaagacccaagtcgtccattactatgtacattacaacgaccagggaaccaattactacattaaagtgcgggccggagataataagtatatgcatcttaaggtgtttaagtcgttgtggggcgaaaacctgtttgccaagtgggaagatcttgtgctcaccggttaccaagtggacaagaacaaggacgacgagctgaccgggttcggaggaggcggatcgggaggtggaggatccggtggcggtggaagcggaggtggcggatctgggggagggggatcaggaggcggcggatccatgatacctggcggactgtcggaggcgaaaccggccacccctgaaatccaggagattgtggacaaggtgaaaccccaactcgaggaaaaaaccggcgaaacgtacggtaaactcgaagccgtgcagtataagacccaggtcgtacactactatgtgcactataacgatcagggaacgaactactacatcaaggtcagagctggagacaataagtacatgcacctcaaggttttcaagtcgttatggggagaaaacctcttcgcgaaatgggaggatctcgtgctgaccggctaccaggtggacaaaaataaggatgatgaactcaccggtttcggaggcggaggatcaggcggagggggttcaggagggggtggctcaggaggagggggtagtggaggcggtggatccggaggaggtggcagcatgatcccacggggtctgtccgaagccaagccagccaccccagagatccaagaaatagtggataaggtgaagccccagcttgaggaaaagactggagagacttacggaaagctcgaggctgtccaatacaaaacacaggtgctggcgaacttcttccaaaggagatggccaggcagtactaactattacatcaaagtgcgcgcaggagataacaaatatatgcacttgaaggtgttcaatggaccgtggaagttccggaacaccgacagaggcgccgacagggtcttgaccgggtaccaagtcgataagaacaaagacgacgaactgaccggtttctgaatctagagacccagctttcttgtacaaagtggtatcgagatatctgcatttaattaatctagagtttaaacgcggccgcaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggttattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc

SEQ ID NO: 739 - protein, 554 aa, synthetic construct
IPGGLSEAKPATPEIQEIVDKVKPQLEEKTGETYGKLEAVQYKTQVVFQYVFSDEDGTNYYIKVRAGDNKYMHLKVFKSLFRWQRGNPEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVFQYVFSDEDGTNYYIKVRAGDNKYMHLKVFKSLFRWQRGNPEDLVLTGYQVDKNKDDELTGFGGGGSGGGGSGGGGSGGGGSGGGGSGGGGSMIPGGLSEAKPATPEIQEIVDKVKPQLEEKTGETYGKLEAVQYKTQVVFQYVFSDEDGTNYYIKVRAGDNKYMHLKVFKSLFRWQRGNPEDLVLTGYQVDKNKDDELTGFGGGGSGGGGSGGGGSGGGGSGGGGSGGGGSMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGFAAAGEQKLISEEDLGAAHHHHHH

SEQ ID NO: 740 - protein, 557 aa, synthetic construct
IPGGLSEAKPATPEIQEIVDKVKPQLEEKTGETYGKLEAVQYKTQVVFQYVFSDEDGTNYYIKVRAGDNKYMHLKVFKSLFRWQRGNPEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVFQYVFSDEDGTNYYIKVRAGDNKYMHLKVFKSLFRWQRGNPEDLVLTGYQVDKNKDDELTGFAEAAAKEAAAKEAAAKEAAAKEAAAKEAAAKMIPGGLSEAKPATPEIQEIVDKVKPQLEEKTGETYGKLEAVQYKTQVVFQYVFSDEDGTNYYIKVRAGDNKYMHLKVFKSLFRWQRGNPEDLVLTGYQVDKNKDDELTGFAEAAAKEAAAKEAAAKEAAAKEAAAKEAAAKMIPRGLSEAKPATPEIQEIVDKVKPQLEEKTGETYGKLEAVQYKTQVLANFFQRRWPGSTNYYIKVRAGDNKYMHLKVFNGPWKFRNTDRGADRVLTGYQVDKNKDDELTGFAAAGEQKLISEEDLGAAHHHHHH

SEQ ID NO: 741 - protein, 49 aa, synthetic construct
MIPGGLSEAKPATPEIQEIVDKVKPQLEEKTNETYGKLEAVQYKTQVLA

SEQ ID NO: 742 - protein, 95 aa, synthetic construct
  VARIANT 49: note = X residue can be repeated one or more times
  VARIANT 74: note = X residue can be repeated one or more times
  VARIANT 49: note = X can be any amino acid
  VARIANT 74: note = X can be any amino acid
MIPGGLSEAKPATPEIQEIVDKVKPQLEEKTGETYGKLEAVQYKTQVVXGTNYYIKVRAGDNKYMHLKVFKSLXEDLVLTGYQVDKNKDDELTGF

SEQ ID NO: 743 - protein, 96 aa, synthetic construct
  VARIANT 50: note = X residue can be repeated one or more times
  VARIANT 75: note = X residue can be repeated one or more times
  VARIANT 50: note = X can be any amino acid
  VARIANT 75: note = X can be any amino acid
MIPGGLSEAKPATPEIQEIVDKVKPQLEEKTGETYGKLEAVQYKTQVLAXGTNYYIKVRAGDNKYMHLKVFKSLXEDLVLTGYQVDKNKDDELTGF

(19) United States
(12) Patent Application Publication
(10) Pub. No.: US 2026/0022160 A1
(43) Pub. Date: Jan. 22, 2026

(54) GENETICALLY MODIFIED CELLS COMPRISING A NUCLEIC ACID ENCODING A CD40L BINDING AGENT AND USES THEREOF

(71) Applicant: AffyXell Therapeutics Co., Ltd., Gimhae-si (KR)

(72) Inventors: Kyong-Hoon Anh, Seoul (KR); Sung Hyun Choi, Anyang-Si Gyeonggi-Do (KR); Kinam Kim, Anyang-Si Gyeonggi-Do (KR); Jong Sang Ryu, Anyang-Si Gyeonggi-Do (KR); Sengho Jeon, Anyang-Si Gyeonggi-Do (KR)

(21) Appl. No.: 19/143,788

(22) PCT Filed: Dec. 29, 2023
(86) PCT No.: PCT/IB23/00797

§ 371 Date: Jun. 26, 2025

(30) Foreign Application Priority Data
Dec. 29, 2022 (KR) 10-2022-0188961

(51) Int. Cl. C07K 14/81; A61K 35/28; A61P 37/00; C12N 5/0775; C12N 15/86

(52) U.S. Cl. CPC C07K 14/8139; A61K 35/28; A61P 37/00; C12N 5/0662; C12N 15/86; C12N 2510/00; C12N 2740/15043

(57) ABSTRACT

Disclosed are a genetically modified cell in which a nucleic acid encoding a CD40L binding agent (e.g., a stefin A protein variant specifically binding to CD40L or a fusion protein including the same) is introduced into a host cell and uses thereof. Provided are genetically modified cells expressing a CD40L binding agent enabling secretion thereof, expression thereof on a cell membrane, and/or intracellular localization thereof, and are thereby capable of reducing or inhibiting the activity of CD40L. Provided genetically modified cells may inhibit T-cell activity and B-cell activity and exhibit an immunosuppressive effect, and may be thus useful for the prevention or treatment of immune diseases such as autoimmune diseases or inflammatory diseases.

### CROSS-REFERENCE TO RELATED APPLICATIONS

This application claims the benefit of priority to Korean Patent Application serial number 10-2022-0188961, filed Dec. 29, 2022; the contents of which is incorporated herein by reference.

### FIELD OF THE INVENTION

The present invention relates to a genetically modified cell in which a nucleic acid encoding a CD40L-binding protein (e.g., a stefin A protein variant that specifically binds to CD40L) or a fusion protein including the same is introduced into a cell (e.g., a stem cell, e.g., a human mesenchymal stromal cell), and uses thereof.

### BACKGROUND

The immune system is a biological network composed of various cells and organs built to protect an organism from invasion from the outside. The immune system operates based on the specific functions of the organs and cells that form the same and the signaling and interaction between cells. The immune system maintains immune homeostasis by balancing between immune tolerance that regulates and modulates immunity and immune response that enhances immunity. Imbalance of immune tolerance and immune response may be caused by various causes, and this imbalance in immunity leads to the development of various diseases. For example, when the immune tolerance mechanism is strengthened, occurrence of cancer or invasion of external infectious agents is facilitated, thereby causing cancer, infectious diseases, and the like. Conversely, when the immune response mechanism is strengthened, inflammatory diseases such as autoimmune diseases and allergies may occur.

Recently, thorough research into immune synapses, which are signaling systems between immune cells constituting the immune system or between target cells and immune cells, is ongoing. Immune synapses are composed of various cytokines and signal transmitters secreted from cells, and various costimulatory molecules and receptors expressed on the cell surface. As various factors involved in maintaining homeostasis of the immune system have been reported, interest in immunotherapeutic agents for regulating immune responses by targeting such factors is increasing. Most immunotherapeutic agents developed to date are antibody drugs against cytokines or cell surface molecules, but the use thereof is very limited due to insufficient efficacy or side effects. Recently, in order to more effectively treat immune diseases, cell therapeutic agents using differentiated immune cells such as T cells, NK cells, etc. or stem cells capable of differentiating into various immune cells are being developed. Cell therapeutic agents based on differentiated immune cells such as CAR-T or CAR-NK are expensive due to the use of autologous cells and have limited targets, and mesenchymal stem cells have a low therapeutic effect relative to the cost of treatment and have limitations in that it is difficult to explain a clear mechanism of action (Blood Cancer J. 11, 69 (2021); World J. Stem Cells. 2019; 11(4):212-221).

Meanwhile, a CD40 ligand (also called CD40L, or CD154) is a protein that binds to CD40 of antigen-presenting cells, and exhibits various effects depending on target cells (The Journal of Experimental Medicine. 175 (4): 1091-101). CD40L typically has three binding partners: CD40, α5β1 integrin, and αIIbβ3. It is reported that CD40L acts as a costimulatory molecule and is particularly important for a subset of T cells called T follicular helper cells (TFH cells) (Journal of Immunology. 149 (12): 3817-26). CD40L is mainly expressed in activated CD4+ T cells, but is also found in a solvable form, and is reported to be expressed not only in T cells, but also in platelets, mast cells, macrophages, basophils, NK cells, B cells, and non-hematopoietic cells such as endothelial cells and epithelial cells (Cellular and Molecular Life Sciences. 58 (1): 4-43). CD40L is classified as a member of the tumor necrosis factor (TNF) superfamily, and signaling of CD40/CD40L as a costimulatory factor plays an important role in T-cell activation and T-cell-mediated B-cell differentiation and activation. Moreover, stimulation of CD40/CD40L signaling plays an important role in regulating the expression and signaling mechanism of OX40/OX40L, which is a costimulatory factor of the same type, and is thus involved in the survival of T cells and the development of memory T cells.

In various immune homeostasis-related diseases, it is reported that CD40L plays an important role in the interaction between antigen-presenting cells (APCs) and T cells. In particular, the CD40L/CD40 interaction acts as a pathogenic factor in autoimmune diseases or inflammatory diseases in which the activation of T cells and B cells has a major influence on pathology. Specifically, it is a pathogenic factor of various diseases such as type 1 diabetes, thyroiditis, psoriasis, lupus (systemic lupus erythematosus; SLE), rheumatoid arthritis (RA), multiple sclerosis (MS), and the like. Various compounds or antibodies targeting CD40L have been developed for the treatment of these diseases (Semin Immunol. 2009; 21(5):293-300; Advanced Drug Delivery Reviews Volume 141, 15 Feb. 2019, Pages 92-103).

Against this background, the present inventors have made great efforts to develop new types of cell therapeutic agents that express a CD40L binding agent.

### SUMMARY

The present disclosure provides, at least in part, genetically modified cells that express a CD40L binding agent. In some embodiments, provided cells exhibit superior immunomodulatory effects and therapeutic effects on immune-related diseases.

In some embodiments, provided are populations of genetically modified cells, wherein the cells comprise an exogenous nucleic acid comprising a coding sequence that encodes a CD40L binding agent, wherein the CD40L binding agent comprises one or more binding domains from an antibody or antibody mimetic.

In some embodiments, provided genetically modified cells and populations thereof are eukaryotic cells. In some embodiments, provided genetically modified cells and populations thereof are mammalian cells. In some embodiments, provided genetically modified cells and populations thereof are human cells.

In some embodiments, provided are populations of genetically modified immune cells, wherein the immune cells comprise an exogenous nucleic acid comprising a coding sequence that encodes a CD40L binding agent, wherein the CD40L binding agent comprises one or more binding domains from an antibody or antibody mimetic. In some embodiments, the immune cells are selected from T cells, B cells, NK cells, monocytes, macrophages, Treg cells, and antigen presenting cells (e.g., dendritic cells).

In some embodiments, provided are populations of genetically modified stem cells, wherein the stem cells comprise an exogenous nucleic acid comprising a coding sequence that encodes a CD40L binding agent, wherein the CD40L binding agent comprises one or more binding domains from an antibody or antibody mimetic.

In some embodiments, provided genetically modified cells and populations thereof are mammalian stem cells. In some embodiments, provided genetically modified cells and populations thereof are human stem cells. In some embodiments, the human stem cells are pluripotent stem cells. In some embodiments, the human stem cells are multipotent stem cells.

In some embodiments, provided are genetically modified mesenchymal stromal cells (MSCs) comprising a nucleic acid encoding a CD40L binding agent. In some embodiments, a CD40L binding agent is CD40L binding protein. In some embodiments, a CD40L binding protein is an antibody, antibody fragment, stefin A protein variant, or a fusion of any thereof.

In some embodiments, provided are populations of genetically modified MSCs, wherein the MSCs comprise an exogenous nucleic acid comprising a coding sequence that encodes a CD40L binding agent, wherein the CD40L binding agent comprises one or more binding domains from an antibody or antibody mimetic.

In some embodiments, provided are methods of producing a population of genetically modified MSCs, wherein the MSCs comprise an exogenous nucleic acid comprising a coding sequence that encodes a CD40L binding agent, wherein the CD40L binding agent comprises one or more binding domains from an antibody or antibody mimetic. In some embodiments, provided methods comprise contacting a population of MSCs with a lentiviral vector comprising an exogenous nucleic acid comprising a coding sequence that encodes a CD40L binding agent, and culturing the population of MSCs. In some embodiments, the population of MSCs are unattached when the contacting is initiated. In some embodiments, provided methods comprise a step of selecting those MSCs that express a selection gene.

In some embodiments, the CD40L binding agent is or comprises a stefin A protein variant, Fab, Fab′, F(ab′)2, Fv, Fd, scFv, sdFv), VL, VH, Camel Ig, V-NAR, VHH, trispecific (Fab3), bispecific (Fab2), diabody ((VL-VH)2 or (VH-VL)2), triabody (trivalent), tetrabody (tetravalent), minibody ((scFv-CH3)2), bispecific single-chain Fv (Bis-scFv), a shark heavy-chain-only antibody (VNAR), a microprotein (cysteine knot protein, knottin), affibody, aptamer, avimer, nanobody, unibody, a single domain antibody, affilin, affitin, adnectin, atrimer, evasin, DARPin, anticalin, avimer, fynomer, versabody, repebody or a duocalin.

In some embodiments, the CD40L binding agent is a CD40L binding protein. In some embodiments, the CD40L binding protein is or comprises a stefin A protein variant, Fab, Fab′, F(ab′)2, Fv, Fd, scFv, sdFv), VL, VH, Camel Ig, V-NAR, VHH, trispecific (Fab3), bispecific (Fab2), diabody ((VL-VH)2 or (VH-VL)2), triabody (trivalent), tetrabody (tetravalent), minibody ((scFv-CH3)2), bispecific single-chain Fv (Bis-scFv), a shark heavy-chain-only antibody (VNAR), a microprotein (cysteine knot protein, knottin), affibody, avimer, nanobody, unibody, a single domain antibody, affilin, affitin, adnectin, atrimer, evasin, DARPin, anticalin, avimer, fynomer, versabody, repebody or a duocalin.

In some embodiments, a CD40L binding agent is based on stefin A protein variant technology. The present disclosure describes, among other things, development of genetically modified cells that secrete the anti-CD40L stefin A protein variant or express the same on cell membranes by introducing a gene encoding an anti-CD40L stefin A protein variant into immune cells, stem cells, or somatic cells, and ascertained that the genetically modified cells may not only exhibit highly enhanced immunomodulatory activity compared to host cells, but also lower the immune rejection response due to allogeneic cell transplantation, and also that the genetically modified cells may exhibit excellent therapeutic effects on immune-related diseases such as inflammatory diseases and autoimmune diseases, thus culminating in the present invention. In some embodiments, the CD40L binding agent is or comprises a stefin A protein variant.

In some embodiments, an exogenous nucleic acid comprises a transcriptional regulatory sequence that is operably linked to the coding sequence. In some embodiments, a transcriptional regulatory sequence is a promoter selected from a CMV promoter, a EFS promoter, a CBh promoter, a MSCV promoter, a SFFV promoter, and a E1FA promoter. In some embodiments, an exogenous nucleic acid comprises an IRES or 2A sequence. In some embodiments, an exogenous nucleic acid comprises a selection gene. In some embodiments, an exogenous nucleic acid comprises, in order, a promoter, a coding sequence that encodes a CD40L binding agent (e.g., CD40L binding protein, e.g., CD40L antibody, CD40L antibody fragment, CD40L binding stefin A protein variant, or fusion of any thereof), a IRES or 2A sequence, and an antibiotic selection gene.

In some embodiments, the MSCs are derived from pluripotent stem cells. In some embodiments, the MSCs are derived or differentiated from induced pluripotent stem cells. In some embodiments, the MSCs are derived or differentiated from embryonic stem cells.

In some embodiments, the MSCs express at least one cell surface marker selected from CD29, CD44, CD73, CD90, and CD105. In some embodiments, the MSCs express at least two cell surface markers selected from CD29, CD44, CD73, CD90, and CD105. In some embodiments, the MSCs express at least three cell surface markers selected from CD29, CD44, CD73, CD90, and CD105. In some embodiments, the MSCs express all of the following cell surface markers: CD29, CD44, CD73, CD90, and CD105.

In some embodiments, at least 85%, at least 90%, at least 91%, at least 92%, at least 93%, at least 94%, at least 95%, at least 96%, at least 97%, or at least 98, of the MSCs express at least one cell surface marker selected from CD29, CD44, CD73, CD90, and CD105. In some embodiments, at least 85%, at least 90%, at least 91%, at least 92%, at least 93%, at least 94%, at least 95%, at least 96%, at least 97%, or at least 98% of the MSCs express at least two cell surface markers selected from CD29, CD44, CD73, CD90, and CD105. In some embodiments, at least 85%, at least 90%, at least 91%, at least 92%, at least 93%, at least 94%, at least 95%, at least 96%, at least 97%, or at least 98% of the MSCs express at least three cell surface markers selected from CD29, CD44, CD73, CD90, and CD105. In some embodiments, at least 85%, at least 90%, at least 91%, at least 92%, at least 93%, at least 94%, at least 95%, at least 96%, at least 97%, or at least 98% of the MSCs express all of the following cell surface markers: CD29, CD44, CD73, CD90, and CD105.

In some embodiments, at least 85%, at least 90′, at least 91%, at least 92%, at least 93%, at least 94%, at least 95%, at least 96%, at least 97%, or at least 98% of the MSCs express CD90.

In some embodiments, at least 85%, at least 90%, at least 91%, at least 92%, at least 93%, at least 94%, or at least 95% of expression of the cell surface marker is maintained in the population of MSCs after at least 15 passages.

In some embodiments, the MSCs do not express or less than 1% of the MSCs of the population express at least one cell surface marker selected from among CD11b, CD14, CD34, CD45, CD79, HLA-DR, TRA-1-60, and TRA-1-81. In some embodiments, the MSCs do not express or less than 1% of the MSCs of the population express at least two cell surface markers selected from among CD11b, CD14, CD34, CD45, CD79, HLA-DR, TRA-1-60, and TRA-1-81. In some embodiments, the MSCs do not express or less than 1% of the MSCs of the population express at least three cell surface markers selected from among CD11b, CD14, CD34, CD45, CD79, HLA-DR, TRA-1-60, and TRA-1-81. In some embodiments, the MSCs do not express or less than 1% of the MSCs of the population express at least four cell surface markers selected from among CD11b, CD14, CD34, CD45, CD79, HLA-DR, TRA-1-60, and TRA-1-81. In some embodiments, the MSCs do not express or less than 1% of the MSCs of the population express any of the following cell surface markers: CD34, CD45, HLA-DR, TRA-1-60, and TRA-1-81.

In some embodiments, at least 95° of the MSCs are CD73+ and CD105+, and less than 1% express CD45, SSEA-3, TRA-1-60, TRA-1-81, and HLA-DR.

In some embodiments, a CD40L binding agent exhibits a Kd value of 1×10⁻⁶ M, 1×10⁻⁷ M, 1×10⁻⁸ M, 1×10⁻⁹ M or less for CD40L.

In some embodiments, a CD40L binding agent is a stefin A protein variant that exhibits a Kd value of 1×10⁻⁶ M, 1×10⁻⁷ M, 1×10⁻⁸ M, 1×10⁻⁹ M or less for CD40L.

In some embodiments, a CD40L binding agent is a stefin A protein variant, wherein the stefin A protein variant comprises an amino acid sequence represented below:

(SEQ ID NO: 742)
MIPGGLSEAKPATPEIQEIVDKVKPQLEEKTGETYGKLEAVQYKTQVV-
(Xaa)n-GTNYYIKVRAGDNKYMHLKVFKSL-(Xaa)m-EDLVLTGYQVDKNKDDELTGF;

wherein Xaa is an amino acid residue, and n and m are each independently an integer from 3 to 20. In some embodiments, (Xaa)n comprises an amino acid sequence selected from the group consisting of SEQ ID NOs: 6 to 125. In some embodiments, (Xaa)m comprises an amino acid sequence selected from the group consisting of SEQ ID NOs: 126 to 245.

In some embodiments, a CD40L binding agent is a stefin A protein variant, wherein the stefin A protein variant comprises or consists of an amino acid sequence that is at least 95%, at least 96%, at least 97%, or at least 98%, at least 99% identical to a sequence selected from the group consisting of SEQ ID NOs: 246 to 365. In some embodiments, a CD40L binding agent is a stefin A protein variant, wherein the stefin A protein variant comprises or consists of an amino acid sequence of any one of SEQ ID NOs: 246 to 365.

In some embodiments, a CD40L binding agent is a stefin A protein variant, wherein the stefin A protein variant further comprises a signal peptide.

In some embodiments, a CD40L binding agent comprises a trimer or tetramer of stefin A protein variants.

In some embodiments, at least 85%, at least 90%, at least 91%, at least 92%, at least 93%, at least 94%, at least 95%, at least 96%, at least 97%, or at least 98% of the MSCs of the population comprise the exogenous nucleic acid.

In some embodiments, the CD40L binding agent is expressed on the surface of the MSCs. In some embodiments, the CD40L binding agent is secreted extracellularly. In some embodiments, the CD40L binding agent is expressed intracellularly.

In some embodiments, where the CD40L binding agent is secreted extracellularly, the population of cells express and secrete CD40L binding agent at an average level of 200 fg/cell/day or greater. In some embodiments, where the CD40L binding agent is secreted extracellularly, the population of cells express and secrete CD40L binding agent at an average level of 300 fg/cell/day or greater. In some embodiments, where the CD40L binding agent is secreted extracellularly, the population of cells express and secrete CD40L binding agent at an average level of 400 fg/cell/day or greater.

In some embodiments, where the CD40L binding agent is secreted extracellularly, the population of cells express and secrete CD40L binding agent at an average level of 200 to 1500 fg/cell/day. In some embodiments, where the CD40L binding agent is secreted extracellularly, the population of cells express and secrete CD40L binding agent at an average level of 300 to 1000 fg/cell/day. In some embodiments, where the CD40L binding agent is secreted extracellularly, the population of cells express and secrete CD40L binding agent at an average level of 400 to 800 fg/cell/day.

In some embodiments, provided are uses of genetically modified MSCs as described herein, for preventing or treating an immune disease.

In some embodiments, provided are pharmaceutical compositions comprising genetically modified MSCs as described herein. In some embodiments, provided pharmaceutical compositions are useful for preventing or treating an immune disease.

In some embodiments, providing are methods of treating an immune disease using genetically modified MSCs as described herein and/or pharmaceutical compositions comprising the same.

In some embodiments, an immune disease is selected from the group consisting of lupus (SLE), lupus nephritis (e.g. drug-induced lupus nephritis), immune thrombocytopenia (ITP), rheumatoid arthritis (RA), multiple sclerosis (MS), inflammatory bowel disease (IBD) (e.g. Crohn's disease and colitis/ulcerative colitis), graft-versus-host disease (GVHD) or allograft rejection, transplantation/solid organ transplantation (SOT), primary biliary cholangitis (PBC), psoriasis, psoriatic arthritis, collagen-induced arthritis, oophoritis, allergic rhinitis, asthma, Sjogren's syndrome, atopic eczema, myasthenia gravis, Graves' disease, and glomerulosclerosis.

In some embodiments, provided are lentiviral vectors suitable for use in preparing and/or transfecting MSCs of the present disclosure. In some embodiments, provided are lentiviral vectors comprising a nucleic acid comprising a transcriptional regulatory sequence operably linked to a sequence that encodes a CD40L binding agent as described herein, an IRES or 2A sequence and a selection gene. In some embodiments, a transcriptional regulatory sequence is a promoter selected from a CMV promoter, a EFS promoter, a CBh promoter, a MSCV promoter, a SFFV promoter, and a E1FA promoter. In some embodiments, a nucleic acid comprises, in order, a promoter, the sequence that encodes a CD40L binding agent (e.g., CD40L binding protein, e.g., CD40L antibody, CD40L antibody fragment, CD40L binding stefin A protein variant, or fusion of any thereof), a IRES or 2A sequence, and an antibiotic selection gene.

In some embodiments, provided are methods of genetically modifying a cell to comprise a nucleic acid encoding a CD40L binding agent (e.g., a stefin A protein variant specifically binding to CD40L and/or a fusion protein including the same).

In some embodiments, provided is a cell therapeutic agent including a genetically modified cell comprising a nucleic acid encoding CD40L binding agent and/or the culture fluid thereof. In some embodiments, the CD40L binding agent is an anti-CD40L antibody, antibody fragment, stefin A protein variant, or a fusion of any thereof. In some embodiments, the genetically modified cell is a MSC (e.g., a human MSC).

In some embodiments, the disclosure provides the use of the genetically modified cells (e.g., genetically modified MSCs) and/or the culture fluid thereof for the manufacture of a cell therapeutic agent.

In some embodiments, the disclosure provides a pharmaceutical composition for preventing or treating an immune disease including the genetically modified cells (e.g., genetically modified MSCs) and/or the culture fluid thereof.

In some embodiments, the disclosure provides the use of the genetically modified cells (e.g., genetically modified MSCs) and/or the culture fluid thereof for the prevention or treatment of an immune disease.

In some embodiments, the disclosure provides the use of the genetically modified cells (e.g., genetically modified MSCs) and/or the culture fluid thereof for the manufacture of a pharmaceutical composition for the prevention or treatment of an immune disease.

In some embodiments, the present disclosure provides a method of preventing or treating an immune disease including administering the genetically modified cells (e.g., genetically modified MSCs) and/or the culture fluid thereof to a subject.

In some embodiments, the present disclosure provides a composition for drug delivery including the genetically modified cells (e.g., genetically modified MSCs) and/or the culture fluid thereof.

In some embodiments, the present disclosure provides the use of the genetically modified cells (e.g., genetically modified MSCs) and/or the culture fluid thereof for drug delivery.

In some embodiments, the present disclosure provides a drug delivery method using the genetically modified cells (e.g., genetically modified MSCs) and/or the culture fluid thereof.

### BRIEF DESCRIPTION OF THE DRAWINGS

The above and other embodiments, features, and other advantages of the present invention will be more clearly understood from the following detailed description taken in conjunction with the accompanying drawings. The drawings included herein are for illustration purposes only and not for limitation.

FIG. 1 shows the results of a direct binding ELISA for monomeric clones to huCD40L.

FIG. 2 shows a validation of hCD40L expression on HEK293 cells following monomeric clone binding determined by flow cytometry.

FIG. 3 is a validation of hCD40L expression of hCD40L-HEK293 cells (top) compared to control HEK 293 cells (bottom).

FIG. 4 shows the binding of clone 230 (SEQ ID NO: 249) to hCD40L-HEK293 cells at different concentrations.

FIG. 5 shows the dose effect of the Stefin A protein variant in blockading the binding of hCD40-L to CD40 in a CD40L HEK-Blue reporter cell assay.

FIG. 6 shows various in line fusion (ILF) formats used to increase avidity to CD40L. 1 μg (left) and 5 μg (right) concentrations were tested for each format. Formats are depicted above the gels (e.g., monomer, dimer, or trimer).

FIG. 7 shows the binding of different clones, including ILF dimeric and trimeric structures, to hCD40L using a BIACORE™ assay.

FIG. 8 shows the binding of different clones, including ILF dimeric and trimeric structures, to hCD40L using flow cytometry.

FIG. 9 shows the dose effect of the Stefin A protein variant having different formats (monomeric, dimeric, trimeric) in blockading the binding of hCD40L to CD40 in a CD40L HEK-Blue reporter cell assay.

FIG. 10 shows the results of a competitive ELISA for the binding of clones having different formats (DT, trimer with a rigid linker; XT75, trimer with a rigid linker and an HSA binding stefin A protein variant; DS, tetramer with a rigid linker; XT76, tetramer with a rigid linker and an HSA binding stefin A protein variant) to hCD40L.

FIG. 11 shows the dose effect of Stefin A protein variant having different formats (DT, trimer with a rigid linker; XT75, trimer with a rigid linker and an HSA binding stefin A protein variant; DS, tetramer with a rigid linker; XT76, tetramer with a rigid linker and an HSA binding stefin A protein variant) in blockading the binding of hCD40L to CD40 in a CD40L HEK-Blue reporter cell assay.

FIGS. 12A-12C show the results of a hCD40L/HSA bridging ELISAs using clone-230 XT75 (an ILF protein comprising three clone 230 monomers and an HSA binding stefin A protein variant) and clone-230 XT76 (an ILF protein comprising four clone 230 monomers and an HSA binding stefin A protein variant). 3t0 Gly XT58 is an ILF protein comprising a trimer of non-hCD40L-targeting protein and an HSA binding stefin A protein variant, joined together with a rigid linker, and 3t0 Gly XT59 is an ILF protein comprising two non-hCD40L-targeting proteins, an HSA binding stefin A protein variant, and two non-hCD40L-targeting proteins joined together with a rigid linker, FIG. 12A shows stefin A protein variant binding to bound hCD40L (measured with an anti-cystatin antibody). FIG. 12B shows stefin A protein variant binding to bound hCD40L (measured with an anti-cystatin antibody) in the presence of HSA. FIG. 12C shows stefin A protein variant binding to bound hCD40L and to HSA (measured by an anti-HSA antibody).

FIG. 13 shows the results of comparing the gene introduction efficiency of transduction enhancers upon transduction of mesenchymal stromal cells using a lentiviral vector (SEQ ID NO: 729) including an eGFP gene, in which eGFP gene introduction efficiency was measured based on the fluorescence wavelength of GFP after treatment with polybrene (2, 4, and 8 μg/mL), protamine sulfate (5, 10, and 20 μg/mL), and LentiBOOST (1:500, 1:100, and 1:20).

FIG. 14 shows the results confirming the gene introduction efficiency based on the fluorescence wavelength of GFP when using a process of treating attached mesenchymal stromal cells with the lentiviral vector including the eGFP gene and a process of treating non-attached mesenchymal stromal cells with the lentiviral vector (reverse transduction).

FIG. 15A shows the results of evaluating the concentration conditions of G418 for selecting gene-introduced cells, in which Naïve MSC was treated with 500, 250, 125, 62.5, 31.25, 15.625, and 7.8125 μg/mL of G418, and cell death was observed at intervals of 1, 3, 5, and 7 days using CCK-8 assay.

FIG. 15B shows the results of selecting cells expressing the eGFP gene (Vector sequence=SEQ ID NO: 729) using G418 and of analyzing the cells by FACS before (left) and after (right) selection.

FIG. 16A shows the results of comparing the gene introduction efficiency using a fluorescence microscope after transduction of mesenchymal stromal cells using a Lentivirus Promoter Blast™ kit (Applied Biological Materials Inc.) in order to evaluate the expression efficiency of the GFP gene depending on the type of promoter.

FIG. 16B shows the results of comparing the gene introduction efficiency for each promoter using the GFP gene by FACS analysis (CMV, Vector sequence=SEQ ID NO: 729).

FIG. 17A shows the results of comparing the expression of the anti-CD40L stefin A protein variant by evaluating the combination of the promoter and the gene-linked peptide, in which a lentiviral vector expressing the anti-CD40L stefin A protein variant was synthesized using CMV, EFS, CBh, MSCV, SFFV, and EF1A promoters and was then introduced into mesenchymal stromal cells to construct a cell line, the anti-CD40L stefin A protein variant secreted by the cell line thus constructed was quantified using an ELISA kit, and a difference in expression was represented in multiples.

FIG. 17B shows the results of analysis of the amount of the anti-CD40L stefin A protein variant that is secreted by constructing a cell line in which the expression of the anti-CD40L stefin A protein variant was regulated by an EF1A promoter (Vector sequence=SEQ ID NO: 733) or a CBh promoter (Vector sequence=SEQ ID NO: 737) and then subjecting the cells to continuous subculture.

FIGS. 18A, 18B, and 18C show results confirming the passage stability of a cell line in which the expression of the anti-CD40L stefin A protein variant was regulated by the EF1A promoter (Vector sequence=SEQ ID NO: 733) or the CBh promoter (Vector sequence=SEQ ID NO: 737), FIG. 18A showing results of comparative analysis of the cell size through continuous subculture from PN9 to PN19, FIG. 18B showing results of cell proliferation time (PDT), and FIG. 18C showing results of comparative analysis of cell proliferation rate (PDL).

FIG. 19 shows the results of analyzing the ability of the anti-CD40L stefin A protein variant secreted out of the cells for each promoter to bind to CD40L through binding ELISA, *5C8: anti-CD40L monoclonal antibody, *eMSC (XT75, w/EF1A), eMSC (XT75, w/CBh).

FIG. 20 shows the results of analyzing the ability of the anti-CD40L stefin A protein variant secreted out of the cells for each promoter to inhibit the binding between CD40L and CD40 through cell-based assay (HEK-Blue Assay), *5C8: anti-CD40L monoclonal antibody, *eMSC (XT75, w/EF1A), eMSC (XT75, w/CBh).

FIG. 21 shows the results confirming whether cells secreting the anti-CD40L stefin A protein variant are effective at inhibiting activation of PBMC (PBMC clustering assay), in which each of Naïve MSC, XT73 gene (SEQ ID NO: 692)-introduced cell line, and XT75 gene (SEQ ID NO: 694)-introduced cell line was co-cultured with PBMC at different ratios (1:20 to 1:1), and the effect of inhibiting clustering of activated PBMC was confirmed, *eMSC (XT73, SEQ ID NO: 692), eMSC (XT75, SEQ ID NO: 694).

FIG. 22 shows the results of comparing whether cells secreting the anti-CD40L stefin A protein variant inhibit an increase in CD3+ T-cell activity through FACS analysis, *eMSC (XT73, SEQ ID NO: 692), eMSC (XT75, SEQ ID NO: 694).

FIG. 23 shows the results of analyzing whether a protein secreted by cells into which the anti-CD40L stefin A protein variant gene (SEQ ID NO: 694) was introduced is effective at inhibiting clustering of B cells.

FIG. 24 shows the results of analyzing whether the protein secreted by the cells into which the anti-CD40L stefin A protein variant gene (SEQ ID NO: 694) was introduced inhibits the activation of B cells, using a CD86 surface marker.

FIG. 25 shows the results of comparing the expression of immunomodulatory factors in Naïve MSC and MSC into which the anti-CD40L stefin A protein variant gene (SEQ ID NO: 694) was introduced through Western blot.

FIG. 26 shows the results of analyzing the purity of the cells into which the anti-CD40L stefin A protein variant gene (SEQ ID NO: 694) was introduced by FACS.

FIG. 27 shows a schematic view (left) and an experimental group design (right) of a xenograft GVHD animal model.

FIG. 28 shows clinical scores representing the efficacy of cells into which the anti-CD40L stefin A protein variant gene (SEQ ID NO: 694) was introduced using the xenograft GVHD mouse model, *G1: normal control, *G2: CS10, *G3: 5c8, *G4: Naïve MSC, *G5: XT75 (eMSC).

FIG. 29 schematically shows a haploidentical GVHD mouse model.

FIG. 30 shows the administration frequency and administration interval of the anti-CD40L stefin A protein variant (XT54 SEQ ID NO: 739, XT55 SEQ ID NO: 740) in the haploidentical GVHD mouse model.

FIG. 31 shows the efficacy of the anti-CD40L stefin A protein variant (XT54 SEQ ID NO: 739, XT55 SEQ ID NO: 740) in the haploidentical GVHD mouse model depending on body weight change (top) and GVHD clinical score (bottom).

### DETAILED DESCRIPTION

Unless otherwise defined, all technical and scientific terms used herein have the same meanings as those typically understood by those skilled in the art to which the present invention belongs. In general, the nomenclature used herein is well known in the art and is typical.

For the most part, the amino acids and amino acid sequences used in the application are those naturally occurring amino acids found in proteins, or the naturally occurring anabolic or catabolic products of such amino acids which contain amino and carboxyl groups, and isomer thereof (e.g. D- or L-stereoisomers).

Amino acid residues further include analogs, derivatives and congeners of any specific amino acid referred to herein, as for instance, the subject AFFIMERC polypeptide (particularly if generated by chemical synthesis) can include an amino acid analog such as, for example, cyanoalanine, canavanine, djenkolic acid, norleucine, 3-phosphoserine, homoserine, dihydroxy-phenylalanine, 5-hydroxytryptophan, 1-methylhistidine, 3-methylhistidine, diaminiopimelic acid, ornithine, or diaminobutyric acid.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity.

The protein or polypeptide described herein, for example, binding protein, e.g., a stefin A protein variant, and/or a fusion protein, may include not only the amino acid sequence described in regard thereto, but also a protein or polypeptide in which a portion of the amino acid sequence is substituted through conservative substitution.

As used herein, “conservative substitution” refers to a modification of a polypeptide comprising substituting one or more amino acids with amino acids having similar biochemical properties that do not cause loss of biological or biochemical functions of the polypeptide.

A conservative amino acid substitution is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, proteins of the present invention do not cause functional loss, for example, a stefin A variant that specifically binds to CD40L does not abrogate its binding to CD40L by its conservative substitution. Methods of identifying amino acid conservative substitutions which do not eliminate binding are well-known in the art. Avacta has applied for a patent using the Affimer-platform technology to select Stefin A protein variants (anti-CD40L Stefin A protein variants or anti-CD40L AFFIMER® proteins) that specifically bind to CD40L. (U.S. Patent Application No. 63/308,629, Avacta Life Sciences Limited, unpublished). In the above patent application, it was confirmed that the anti-CD40L binding Stepin A protein variant can bind to CD40L with excellent affinity and specificity, and that it can be used as an antagonist by inhibiting the activity of CD40L.

Accordingly, the following inventions are claimed in U.S. Patent Application No. 63/308,629 filed by Avacta Life Sciences Limited or the priority application filed therewith and are excluded from the scope of the present invention:
(i) Genetically engineered cells for use in the manufacturing of a Stepin A protein variant that specifically binds to CD40L or a fusion protein containing the same;
(ii) Use only for manufacturing a Stepin A protein variant that specifically binds to CD40L of the genetically engineered cells, or a fusion protein or conjugate containing the same; and
(iii) A method of manufacturing a Stepin A protein variant that specifically binds to CD40L, or a fusion protein or conjugate containing the same, using the genetically engineered cells.

In this context, the term “manufacturing” refers to “production for manufacturing” the Stepin A protein variant that specifically binds to CD40L or a fusion protein containing the same.

In some embodiments, the present disclosure describes, development of cells having excellent immunomodulatory activity through genetic modification. In some embodiments, provided is a genetically modified cell that stably secretes a stefin A protein variant specifically binding to CD40L, expresses the same on a cell membrane, and/or intracellularly expresses the same. In some embodiments, provided cells may be produced by introducing a gene encoding a stefin A protein variant specifically binding to CD40L into the cells.

In examples of the present disclosure, it has been confirmed that the genetically modified cell is capable of specifically binding to a target cell through expression of a stefin A protein variant specifically binding to CD40L or a fusion protein including the same on the cell membrane, and also that passage stability and immunomodulatory effect of the host cell are maintained despite the gene introduction and the genetically modified cell exhibits a vastly superior immunomodulatory effect compared to the host cell.

Accordingly, an aspect of the present invention pertains to a genetically modified cell in which a nucleic acid encoding a stefin A protein variant specifically binding to CD40L or a fusion protein including the same is introduced into a host cell.

### CD40L

As used herein, the term “CD40L” or “CD40 ligand” refers to a protein that binds to CD40, its receptor, and is also called “CD154”. Aliases for CD40L include TNF-Related Activation Protein, TRAP, Tumor Necrosis Factor (Ligand) Superfamily Member, T-B Cell-Activating Molecule, CD40 Antigen Ligand, T-Cell Antigen Gp39, TNFSF5, HCD40L, CD154, Gp39, Tumor Necrosis Factor (Ligand) Superfamily, Member 5 (Hyper-IgM Syndrome), Tumor Necrosis Factor Ligand Superfamily Member, Hyper-IgM Syndrome, CD154 Antigen, CD40LG, HIGM1, T-BAM, IMD3, IGM, and CD40-L.

Cluster of Differentiation 40 ligand (CD40L) is a protein that, in the case of humans, is encoded by the CD40L gene. CD40L is a protein that acts as a ligand to CD40/TNFRSF5 and costimulates T-cell proliferation and cytokine production. Its cross-linking on T-cells generates a costimulatory signal which enhances the production of IL4 and IL10 in conjunction with the TCR/CD3 ligation and CD28 costimulation. CD40L induces the activation of NF-kappa-B, induces the activation of kinases MAPK8 and PAK2 in T-cells, and induces tyrosine phosphorylation of isoform 3 of CD28. It also mediates B-cell proliferation in the absence of co-stimulus as well as IgE production in the presence of IL4. CD40 L is also involved in immunoglobulin class switching. The human amino acid and nucleic acid sequences can be found in a public database, such as GenBank, UniProt and Swiss-Prot. For example, the amino acid sequence of human CD40L can be found as UniProt/Swiss-Prot. Accession No. P29965 and the nucleotide sequence encoding of the human CD40L can be found at Accession No. NM_000074.2.

The CD40L includes any native, mature CD40L which results from processing of a CD40L precursor protein in a cell. The term encompasses CD40L from any vertebrate source, including mammals such as primates (e.g., humans and cynomolgus monkeys) and rodents (e.g., mice and rats), unless otherwise indicated but is not limited to.

The term also includes any CD40L proteins comprising mutations, e.g., point mutations, fragments, insertions, deletions, and splice variants of full length wild-type CD40L, but is not limited to. The CD40L includes any CD40L proteins comprising mutations, e.g., point mutations, fragments, insertions, deletions, and splice variants of full length wild-type CD40L.

### CD40L Binding Agents

The present disclosure provides, among other things, cells that comprise an exogenous nucleic acid encoding a CD40L binding agent.

In some embodiments, a CD40L binding agent is a recombinant protein comprising one or more binding domains from an antibody or antibody mimetic which bind to CD40L. In some embodiments, a CD40L binding agent is a stefin A protein variant, Fab, Fab′, F(ab′)2, Fv, Fd, scFv, sdFv), VL, VH, Camel Ig, V-NAR, VHH, trispecific (Fab3), bispecific (Fab2), diabody ((VL-VH)2 or (VH-VL)2), triabody (trivalent), tetrabody (tetravalent), minibody ((scFv-CH3)2), bispecific single-chain Fv (Bis-scFv), a shark heavy-chain-only antibody (VNAR), a microprotein (cysteine knot protein, knottin), affibody, aptamer, avimer, nanobody, unibody, a single domain antibody, affilin, affitin, adnectin, atrimer, evasin, DARPin, anticalin, avimer, fynomer, versabody, repebody or a duocalin.

In some embodiments, a CD40L binding domain may be selected from the group consisting of, for example, a stefin A protein variant, an antibody or fragment thereof, an antibody-like material, an antigen-binding peptide, a ligand-binding site of a receptor (e.g. a receptor trap polypeptide), a receptor-binding ligand (e.g. a cytokine or a growth factor), an engineered T-cell receptor, and an enzyme or a catalytic fragment thereof, but is not limited thereto.

In some embodiments, a CD40L binding agent is an anti-CD40L antibody. As used herein, the term “antibody” includes not only a complete antibody form that specifically binds to a target (antigen), but also an antigen-binding fragment of the antibody molecule. The complete antibody has a structure having two full-length light chains and two full-length heavy chains, and each light chain is connected to the heavy chain by a disulfide bond. As used herein, the term “heavy chain” refers to a full-length heavy chain including a variable region domain VH including an amino acid sequence having a sufficient variable region sequence to confer specificity to an antigen and three constant region domains CH1, CH2, and CH3, and fragments thereof. In addition, as used herein, the term “light chain” refers to a full-length light chain including a variable region domain VL including an amino acid sequence having a sufficient variable region sequence to confer specificity to an antigen and a constant region domain CL, and fragments thereof. The whole antibody includes subtypes of IgA, IgD, IgE, IgM, and IgG, and in particular, IgG includes IgG1, IgG2, IgG3, and IgG4. The heavy-chain constant region has gamma (γ), mu (μ), alpha (α), delta (δ), and epsilon (ε) types, and subclasses such as gamma 1 (γ1), gamma 2 (γ2), gamma 3 (γ3), gamma 4 (γ4), alpha 1 (α1), and alpha 2 (α2). The constant region of the light chain has kappa (κ) and lambda (λ) types.

In some embodiments, a CD40L binding agent is an anti-CD40L antigen-binding antibody fragment. The antigen-binding fragment of an antibody or antibody fragment refers to a fragment having an antigen-binding function, and includes Fab, F(ab′), F(ab′)2, and Fv. Among the antibody fragments, Fab has a structure having variable regions of light and heavy chains, a constant region of a light chain, and a first constant region (CH1) of a heavy chain, and has one antigen-binding site. Fab′ differs from Fab in that Fab′ has a hinge region including at least one cysteine residue at the C-terminus of the heavy-chain CH1 domain. F(ab′)2 is formed by a disulfide bond between cysteine residues in the hinge region of Fab′.

Fv is a minimal antibody fragment having only a heavy-chain variable region and a light-chain variable region. A two-chain Fv is a fragment in which a heavy-chain variable region and a light-chain variable region are linked by a non-covalent bond, and a single-chain Fv (scFv) is a fragment in which a heavy-chain variable region and a light-chain variable region are generally linked by a covalent bond via a peptide linker therebetween, or are directly linked at the C-terminus, forming a dimeric structure, like the two-chain Fv. Such antibody fragments may be obtained using proteases (for example, Fab may be obtained by restriction-cleaving a whole antibody with papain, and the F(ab′)2 fragment may be obtained by restriction-cleaving a whole antibody with pepsin), or may be constructed through genetic recombination technology.

An “Fv” fragment is an antibody fragment that contains a complete antibody recognition and binding site. This region is a dimer in which one heavy-chain variable domain and one light-chain variable domain are joined.

A “Fab” fragment includes variable and constant domains of a light chain and variable and first constant domains (CH1) of a heavy chain. An F(ab′)2 antibody fragment generally includes a pair of Fab′ fragments covalently linked by cysteines in the hinge region present at the C-terminus of the Fab′ fragment.

A “single-chain Fv (scFv)” antibody fragment is a construct composed of a single polypeptide chain including the VH and VL domains of an antibody. scFv may further include a polypeptide linker between the VH domain and the VL domain so as form the desired structure for antigen binding.

Examples of the antibody of the present invention may include, but are not limited to, monoclonal antibodies, multispecific antibodies, human antibodies, humanized antibodies, chimeric antibodies, scFv, Fab fragments, F(ab′)2 fragments, disulfide-linked Fvs (sdFv), and anti-idiotype (anti-Id) antibodies, epitope-binding fragments of such antibodies, and the like.

The heavy-chain constant region may be selected from among isotypes such as gamma (γ), mu (μ), alpha (α), delta (δ), and epsilon (ε). For example, the constant region is gamma 1 (IgG1), gamma 2 (IgG2), gamma 3 (IgG3), or gamma 4 (IgG4). The light-chain constant region may be a kappa or lambda type.

A monoclonal antibody is an antibody obtained from a population of substantially homogeneous antibodies, in which the individual antibodies that make up the population are identical, except for possible naturally-occurring mutations that may be present in small amounts. A monoclonal antibody is highly specific and is induced against a single epitope on the antigen. In contrast to typical (polyclonal) antibodies, which typically include different antibodies directed against different determinants (epitopes), each monoclonal antibody is directed against a single determinant on the antigen.

Examples of CD40L antibodies and antigen-binding antibody fragments are described in the art. In humans, at least two different anti-CD40L mAb clones have been used in clinical trials for treatment of different autoimmune diseases. Maribel et al., Mol. Immunol., 45:937-944 (2008). Example anti-CD40L antibodies are described in, e.g., WO 2006/030220A1 (which describes antibody polypeptides that monovalently bind CD40L; incorporated by reference), WO 2013/056068A1 (which describes antibody polypeptides that specifically bind human CD40L; incorporated by reference), and WO 2016/040571A1 (which describes methods of treatment using anti-CD40L antibodies; incorporated by reference).

In certain embodiments, a CD40L binding agent comprises a ligand binding domain of a receptor that acts as an inhibitor/antagonist of the ligand (i.e., target) to which it binds, e.g., an “inhibitory receptor trap” or “decoy receptor”. Inhibitory receptor traps binds to and/or sequesters its target, which in essence inhibits the target from carrying out its function which may contribute to a disease and/or disorder to be treated.

In some embodiments, a CD40L binding agent is or comprises a binding domain of a CD40 receptor. In some embodiments, a CD40L binding agent is or comprises a fusion protein comprising a binding domain of a CD40 receptor.

### Stefin A Protein Variants Specifically Binding to CD40L

As used herein, the term “Stefin A protein variant (Stefin A protein variant)” a scaffold based on a Stefin A polypeptide, meaning that it has a sequence which is derived from a Stefin A polypeptide, for example, a mammalian Stefin A polypeptide, for example, a human Stefin A polypeptide.

In the present invention, the “Stefin A protein Variant” may be used interchangeably in substantially the same sense as “Stefin A protein variant” or “AFFIMER® protein”. In the present invention, the “Stefin A protein Variant” may be used interchangeably in substantially the same sense as “Stefin A polypeptide variant” or “AFFIMER® protein”.

Affimer®, developed by Avacta, is a small stable protein molecule engineered based on a stefin A protein, which is an in-vivo protein. Affimer® includes two short peptide sequences having a random sequence and an N-terminal sequence, and is able to bind to a target material with high affinity and specificity in a manner similar to a monoclonal antibody. Affimer® shows remarkably improved binding affinity and specificity compared to the free peptide library, and has a very small size and high stability compared to antibodies, and is therefore receiving great attention as a next-generation alternative pharmaceutical platform to replace antibodies (U.S. Pat. Nos. 9,447,170, 8,853,131; incorporated by reference).

Avacta has previously applied for a patent on Affimer® platform technology by selecting an anti-CD40L stefin A protein variant (anti-CD40L AFFIMER® protein) (U.S. Patent Application No. 63/308,629, unpublished). In this patent application, it was confirmed that the anti-CD40L stefin A protein variant is able to bind to CD40L with excellent affinity and specificity, and also that it is usable as an antagonist by inhibiting the activity of CD40L.

In some embodiments, the stefin A protein variants of the present disclosure may display two peptide loops and an N-terminal sequence that can all be randomized to bind to desired target proteins with high affinity and specificity, in a similar manner to antibodies. A target protein-specific binding platform using such a stefin A protein variant is disclosed in detail in U.S. Pat. Nos. 9,447,170 and 8,853,131.

In some embodiments, the stefin A protein variant is featured in that it can bind to CD40L, a target protein, with high affinity and specificity through engineering of the stefin A protein.

In some embodiments, the stefin A protein variant specifically binding to CD40L may bind to CD40L, thereby reducing or inhibiting the activity of CD40L.

In some embodiments, the stefin A protein variant may comprise at least one of the solvent accessible loops from the wild-type Stefin A protein having the ability to bind CD40L.

In some embodiments, the stefin A protein variant may bind to CD40L with Kd of 10⁻⁶ M or less.

In some embodiments, the stefin A protein variant may be variant derived from is derived from the Stefin A polypeptide having a backbone sequence and in which one or both of loop 2 [designated (Xaa)n] and loop 4 [designated (Xaa)m] are replaced with alternative loop sequences (Xaa)n and (Xaa)m.

In some embodiments, the stefin A protein variant may comprise amino acid sequence represented by Formula I

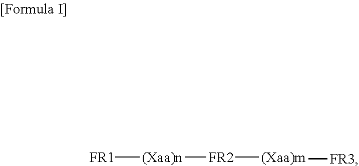

Wherein, FR1 is a polypeptide sequence comprising the amino acid sequence of MIPGGLSEAK PATPEIQEIV DKVKPQLEEK TGETYGKLEA VQYKTQVX (SEQ ID NO: 1), MIPGGLSEAK PATPEIQEIV DKVKPQLEEK TNETYGKLEA VQYKTQVLA (SEQ ID NO: 741) or a polypeptide sequence having at least 70% (e.g., at least 75%, at least 80%, at least 85%, at least 90%, at least 95%, or 100%) identity to the amino acid sequence of SEQ ID NO: 1, wherein X is V or D;
FR2 is a polypeptide sequence comprising the amino acid sequence of GTNYYIKVRA GDNKYMHLKV FKSL (SEQ ID NO: 2) or a polypeptide sequence having at least 70% (e.g., at least 75%, at least 80%, at least 85%, at least 90%, at least 95%, or 100%) identity to the amino acid sequence of SEQ ID NO: 2;
FR3 is a polypeptide sequence comprising the amino acid sequence of EDLVLTGYQV DKNKDDELTG F (SEQ ID NO: 3) or a polypeptide sequence having at least 70% (e.g., at least 75%, at least 80%, at least 85%, at least 90%, at least 95%, or 100%) identity to the amino acid sequence of SEQ ID NO: 3; and
Xaa, individually for each occurrence, is an amino acid residue; and n and m are each, independently, an integer from 3 to 20.

In some embodiments, (Xaa)n and (Xaa)m, individually for each occurrence, is a sequence of 3 to 20 arbitrary amino acids.

In some embodiments, FR1 may have a polypeptide sequence having at least 80%, 85%, 90%, 95% or even 98% homology with SEQ ID NO: 1. In some embodiments, FR1 may have a polypeptide sequence having at least 80%, 85%, 90%, 95% or even 98% identity with SEQ ID NO: 1.

In some embodiments, FR2 may have a polypeptide sequence having at least 80%, 85%, 90%, 95% or even 98% homology with SEQ ID NO: 2. In some embodiments, FR2 may have a polypeptide sequence having at least 80%, 85%, 90%, 95% or even 98% identity with SEQ ID NO: 2.

In some embodiments, FR3 may have a polypeptide sequence having at least 80%, 85%, 90%, 95% or even 98% homology with SEQ ID NO: 3. In some embodiments, FR3 may have a polypeptide sequence having at least 80%, 85%, 90%, 95% or even 98% identity with SEQ ID NO: 3.

In some embodiments, the stefin A protein variant comprises an amino acid sequence represented in the general Formula II:

[Formula II]
(SEQ ID NO: 4)
MIP-Xaa1-GLSEAKPATPEIQEIVDKVKPQLEEKTGETYGKLEAVQYKTQV-
Xaa2-(Xaa)n-Xaa3-TNYYIKVRAGDNKYMHLKVF-Xaa4-Xaa5-Xaa6-(Xaa)m-
Xaa7-D-Xaa8-VLTGYQVDKNKDDELTGF,

wherein
Xaa, individually for each occurrence, is any number of an amino acid residue, more suitably three or fewer (preferably, one or two) independently selected amino acids; and n and m are each, independently, an integer from 3-20.

In some embodiments, the stefin A protein variant may comprises an amino acid sequence having at least 90% (e.g., at least 95%, at least 96%, at least 97%, at least 98%, at least 99%, or 100%) identity to the amino acid sequence represented below:

(SEQ ID NO: 742)
MIPGGLSEAKPATPEIQEIVDKVKPQLEEKTGETYGKLEAVQYKTQVV-
(Xaa)n-GTNYYIKVRAGDNKYMHLKVFKSL-(Xaa)m-
EDLVLTGYQVDKNKDDELTGF;
(SEQ ID NO: 5)
MIPGGLSEAKPATPEIQEIVDKVKPQLEEKTGETYGKLEAVQYKTQVD-
(Xaa)n-GTNYYIKVRAGDNKYMHLKVFKSL-(Xaa)m-
EDLVLTGYQVDKNKDDELTGF;
or
(SEQ ID NO: 743)
MIPGGLSEAKPATPEIQEIVDKVKPQLEEKTGETYGKLEAVQYKTQVLA-
(Xaa)n-GTNYYIKVRAGDNKYMHLKVFKSL-(Xaa)m-
EDLVLTGYQVDKNKDDELTGF

Wherein, Xaa, individually for each occurrence, is an amino acid residue; and n and m are each, independently, an integer from 3-20.

In some embodiments, Xaa1 may be Gly, Ala, Val, Arg, Lys, Asp, or Glu, more preferably Gly, Ala, Arg or Lys, and more even more preferably Gly or Arg;
In some embodiments, Xaa2 is Val, Asp or ‘Leu-Ala’;
In some embodiments, Xaa3 may be Gly, Ala, Val, Ser or Thr, more preferably Gly or Ser

In some embodiments, Xaa4 may be Arg, Lys, Asn, Gln, Ser, Thr, more preferably Arg, Lys, Asn or Gln, and even more preferably Lys or Asn.

In some embodiments, Xaa5 may be Gly, Ala, Val, Ser or Thr, more preferably Gly or Ser.

In some embodiments, Xaa6 may be Ala, Val, Ile, Leu, Gly or Pro, more preferably Ile, Leu or Pro, and even more preferably Leu or Pro.

In some embodiments, Xaa7 may be Gly, Ala, Val, Asp or Glu, more preferably Ala, Val, Asp or Glu, and even more preferably Ala or Glu.

In some embodiments, Xaa8 may be Ala, Val, Ile, Leu, Arg or Lys, more preferably Ile, Leu or Arg, and even more preferably Leu or Arg.

In some embodiments, n may be 3 to 15, 3 to 12, 3 to 9, 3 to 7, 5 to 7, 5 to 9, 5 to 12, 5 to 15, 7 to 12 or 7 to 9.

In some embodiments, m may be 3 to 15, 3 to 12, 3 to 9, 3 to 7, 5 to 7, 5 to 9, 5 to 12, 5 to 15, 7 to 12 or 7 to 9.

In some embodiments, Xaa, independently for each occurrence, may be an amino acid that can be added to a polypeptide by recombinant expression in a prokaryotic or eukaryotic cell, and even more preferably one of the 20 naturally occurring amino acids.

In the above sequences and formulas, (Xaa)n may be an amino acid sequence selected from SEQ ID NOs: 6 to 125, or an amino acid sequence having at least 80%, 85%, 90%, 95% or even 98% homology with a sequence selected from SEQ ID NOs: 6 to 125. In some embodiments, (Xaa)n is an amino acid sequence having at least 80%, 85%, 90%, 95% or even 98% identity with a sequence selected from SEQ ID NOs: 6 to 125.

In some embodiments of the above sequences and formulas, (Xaa)n may be an amino acid sequence selected from SEQ ID NOs: 6 to 125, or an amino acid sequence having at least 80%, 85%, 90%, 95% or even 98% homology with a sequence selected from SEQ ID NOs: 6 to 125. In some embodiments, (Xaa)n may be an amino acid sequence having at least 80%, 85%, 90%, 95% or even 98% identity with a sequence selected from SEQ ID NOs: 6 to 125.

TABLE 1
Loop 2 Sequence

| Clone # | Loop 2 | SEQ ID NO: |
|---|---|---|
| 227 | [symbol] | 6 |
| 228 | [symbol] | 7 |
| 229 | [symbol] | 8 |
| 230 | [symbol] | 9 |
| 231 | [symbol] | 10 |
| 232 | [symbol] | 11 |
| 233 | [symbol] | 12 |
| 234 | [symbol] | 13 |
| 235 | [symbol] | 14 |
| 236 | [symbol] | 15 |
| 237 | [symbol] | 16 |
| 238 | [symbol] | 17 |
| 239 | [symbol] | 18 |
| 240 | [symbol] | 19 |
| 241 | [symbol] | 20 |
| 242 | [symbol] | 21 |
| 243 | [symbol] | 22 |
| 244 | [symbol] | 23 |
| 245 | [symbol] | 24 |
| 246 | [symbol] | 25 |
| 247 | [symbol] | 26 |
| 248 | [symbol] | 27 |
| 249 | [symbol] | 28 |
| 250 | [symbol] | 29 |
| 251 | [symbol] | 30 |
| 252 | [symbol] | 31 |
| 253 | [symbol] | 32 |
| 254 | [symbol] | 33 |
| 255 | [symbol] | 34 |
| 256 | [symbol] | 35 |
| 257 | [symbol] | 36 |
| 258 | [symbol] | 37 |
| 259 | [symbol] | 38 |
| 260 | [symbol] | 39 |
| 261 | [symbol] | 40 |
| 262 | [symbol] | 41 |
| 263 | [symbol] | 42 |
| 264 | [symbol] | 43 |
| 265 | [symbol] | 44 |
| 266 | [symbol] | 45 |
| 267 | [symbol] | 46 |
| 268 | [symbol] | 47 |
| 269 | [symbol] | 48 |
| 270 | [symbol] | 49 |
| 271 | [symbol] | 50 |
| 272 | [symbol] | 51 |
| 273 | [symbol] | 52 |
| 274 | [symbol] | 53 |
| 275 | [symbol] | 54 |
| 276 | [symbol] | 55 |
| 278 | [symbol] | 56 |
| 279 | [symbol] | 57 |
| 280 | [symbol] | 58 |
| 281 | [symbol] | 59 |
| 282 | [symbol] | 60 |
| 283 | [symbol] | 61 |
| 284 | [symbol] | 62 |
| 286 | [symbol] | 63 |
| 287 | [symbol] | 64 |
| 288 | [symbol] | 65 |
| 289 | [symbol] | 66 |
| 290 | [symbol] | 67 |
| 291 | [symbol] | 68 |
| 292 | [symbol] | 69 |
| 293 | [symbol] | 70 |
| 294 | [symbol] | 71 |
| 295 | [symbol] | 72 |
| 296 | [symbol] | 73 |
| 297 | [symbol] | 74 |
| 298 | [symbol] | 75 |
| 299 | [symbol] | 76 |
| 300 | [symbol] | 77 |
| 301 | [symbol] | 78 |
| 302 | [symbol] | 79 |
| 303 | [symbol] | 80 |
| 304 | [symbol] | 81 |
| 305 | [symbol] | 82 |
| 306 | [symbol] | 83 |
| 307 | [symbol] | 84 |
| 308 | [symbol] | 85 |
| 309 | [symbol] | 86 |
| 310 | [symbol] | 87 |
| 311 | [symbol] | 88 |
| 312 | [symbol] | 89 |
| 313 | [symbol] | 90 |
| 314 | [symbol] | 91 |
| 315 | [symbol] | 92 |
| 316 | [symbol] | 93 |
| 317 | [symbol] | 94 |
| 318 | [symbol] | 95 |
| 319 | [symbol] | 96 |
| 320 | [symbol] | 97 |
| 321 | [symbol] | 98 |
| 322 | [symbol] | 99 |
| 323 | [symbol] | 100 |
| 324 | [symbol] | 101 |
| 328 | [symbol] | 102 |
| 326 | [symbol] | 103 |
| 327 | [symbol] | 104 |
| 328 | [symbol] | 105 |
| 329 | [symbol] | 106 |
| 330 | [symbol] | 107 |
| 331 | [symbol] | 108 |
| 332 | [symbol] | 109 |
| 333 | [symbol] | 110 |
| 334 | [symbol] | 111 |
| 335 | [symbol] | 112 |
| 336 | [symbol] | 113 |
| 337 | [symbol] | 114 |
| 338 | [symbol] | 115 |
| 339 | [symbol] | 116 |
| 340 | [symbol] | 117 |
| 341 | [symbol] | 118 |
| 342 | [symbol] | 119 |
| 343 | [symbol] | 120 |
| 344 | [symbol] | 121 |
| 345 | [symbol] | 122 |
| 346 | [symbol] | 123 |
| 347 | [symbol] | 124 |
| 348 | [symbol] | 125 |

[symbol]

indicates data missing or illegible when filed

In some embodiments of the above sequences and formulas, (Xaa)m may be an amino acid sequence selected from SEQ ID NOs: 126 to 245, or an amino acid sequence having at least 80%, 85%, 90%, 95% or even 98% homology with a sequence selected from SEQ ID NOs: 126 to 245. In some embodiments, (Xaa)m may be an amino acid sequence having at least 80%, 85%, 90%, 95% or even 98% identity with a sequence selected from SEQ ID NOs: 126 to 245.

TABLE 2
Loop 4 Sequence

| Clone # | Loop 4 | SEQ ID NO: |
|---|---|---|
| 227 | [symbol] | 126 |
| 228 | [symbol] | 127 |
| 229 | [symbol] | 128 |
| 230 | [symbol] | 129 |
| 231 | [symbol] | 130 |
| 232 | [symbol] | 131 |
| 233 | [symbol] | 132 |
| 234 | [symbol] | 133 |
| 235 | [symbol] | 135 |
| 236 | [symbol] | 138 |
| 237 | [symbol] | 136 |
| 238 | [symbol] | 137 |
| 239 | [symbol] | 138 |
| 240 | [symbol] | 139 |
| 241 | [symbol] | 140 |
| 242 | [symbol] | 141 |
| 243 | [symbol] | 142 |
| 244 | [symbol] | 143 |
| 245 | [symbol] | 144 |
| 246 | [symbol] | 145 |
| 247 | [symbol] | 146 |
| 248 | [symbol] | 147 |
| 249 | [symbol] | 148 |
| 250 | [symbol] | 149 |
| 251 | [symbol] | 150 |
| 252 | [symbol] | 151 |
| 253 | [symbol] | 152 |
| 254 | [symbol] | 153 |
| 255 | [symbol] | 154 |
| 256 | [symbol] | 155 |
| 257 | [symbol] | 156 |
| 258 | [symbol] | 157 |
| 259 | [symbol] | 158 |
| 260 | [symbol] | 159 |
| 261 | [symbol] | 160 |
| 262 | [symbol] | 161 |
| 263 | [symbol] | 162 |
| 264 | [symbol] | 163 |
| 265 | [symbol] | 164 |
| 266 | [symbol] | 165 |
| 267 | [symbol] | 166 |
| 268 | [symbol] | 167 |
| 269 | [symbol] | 168 |
| 270 | [symbol] | 169 |
| 271 | [symbol] | 170 |
| 272 | [symbol] | 171 |
| 273 | [symbol] | 172 |
| 274 | [symbol] | 173 |
| 275 | [symbol] | 174 |
| 276 | [symbol] | 175 |
| 278 | [symbol] | 176 |
| 279 | [symbol] | 177 |
| 280 | [symbol] | 178 |
| 281 | [symbol] | 179 |
| 282 | [symbol] | 180 |
| 283 | [symbol] | 181 |
| 284 | [symbol] | 182 |
| 286 | [symbol] | 183 |
| 287 | [symbol] | 184 |
| 288 | [symbol] | 185 |
| 289 | [symbol] | 186 |
| 290 | [symbol] | 187 |
| 291 | [symbol] | 188 |
| 292 | [symbol] | 189 |
| 293 | [symbol] | 190 |
| 294 | [symbol] | 191 |
| 295 | [symbol] | 192 |
| 296 | [symbol] | 193 |
| 297 | [symbol] | 194 |
| 298 | [symbol] | 195 |
| 299 | [symbol] | 196 |
| 300 | [symbol] | 197 |
| 301 | [symbol] | 198 |
| 302 | [symbol] | 199 |
| 303 | [symbol] | 200 |
| 304 | [symbol] | 201 |
| 305 | [symbol] | 202 |
| 306 | [symbol] | 203 |
| 307 | [symbol] | 204 |
| 308 | [symbol] | 205 |
| 309 | [symbol] | 206 |
| 310 | [symbol] | 207 |
| 311 | [symbol] | 208 |
| 312 | [symbol] | 209 |
| 313 | [symbol] | 210 |
| 314 | [symbol] | 211 |
| 315 | [symbol] | 212 |
| 316 | [symbol] | 213 |
| 317 | [symbol] | 214 |
| 318 | [symbol] | 215 |
| 319 | [symbol] | 216 |
| 320 | [symbol] | 217 |
| 321 | [symbol] | 218 |
| 322 | [symbol] | 219 |
| 323 | [symbol] | 220 |
| 324 | [symbol] | 222 |
| 325 | [symbol] | 223 |
| 326 | [symbol] | 224 |
| 327 | [symbol] | 225 |
| 328 | [symbol] | 226 |
| 330 | [symbol] | 227 |
| 331 | [symbol] | 228 |
| 332 | [symbol] | 229 |
| 333 | [symbol] | 230 |
| 334 | [symbol] | 231 |
| 335 | [symbol] | 232 |
| 336 | [symbol] | 233 |
| 337 | [symbol] | 234 |
| 338 | [symbol] | 235 |
| 339 | [symbol] | 236 |
| 340 | [symbol] | 237 |
| 341 | [symbol] | 238 |
| 342 | [symbol] | 239 |
| 343 | [symbol] | 240 |
| 344 | [symbol] | 241 |
| 345 | [symbol] | 242 |
| 346 | [symbol] | 243 |
| 347 | [symbol] | 244 |
| 348 | [symbol] | 245 |

[symbol]

indicates data missing or illegible when filed

In some embodiments, the stefin A protein variant comprises an amino acid sequence selected from SEQ ID NOs: 246 to 365. In some embodiments, the stefin A protein variant comprises an amino acid sequence having at least 70%, 75% 80%, 85%, 900, 95% or even 98% identity with a sequence selected from SEQ ID NOs: 246 to 365.

TABLE 3
Exemplary stefin A protein variant specifically binding to
CD40L

| Clone# | Amino Acid Sequence | SEQ ID NO: |
|---|---|---|
| 227 | [symbol] | 246 |
| | [symbol] | |
| | [symbol] | |
| 228 | [symbol] | 247 |
| | [symbol] | |
| | [symbol] | |
| 229 | [symbol] | 248 |
| | [symbol] | |
| | [symbol] | |
| 230 | [symbol] | 249 |
| | [symbol] | |
| | [symbol] | |
| 231 | [symbol] | 250 |
| | [symbol] | |
| | [symbol] | |
| 232 | [symbol] | 251 |
| | [symbol] | |
| | [symbol] | |
| 233 | [symbol] | 252 |
| | [symbol] | |
| | [symbol] | |
| 234 | [symbol] | 253 |
| | [symbol] | |
| | [symbol] | |
| 235 | [symbol] | 254 |
| | [symbol] | |
| | [symbol] | |
| 236 | [symbol] | 255 |
| | [symbol] | |
| | [symbol] | |
| 237 | [symbol] | 256 |
| | [symbol] | |
| | [symbol] | |
| 238 | [symbol] | 257 |
| | [symbol] | |
| | [symbol] | |
| 239 | [symbol] | 258 |
| | [symbol] | |
| | [symbol] | |
| 240 | [symbol] | 259 |
| | [symbol] | |
| | [symbol] | |
| 241 | [symbol] | 260 |
| | [symbol] | |
| | [symbol] | |
| 242 | [symbol] | 261 |
| | [symbol] | |
| | [symbol] | |
| 243 | [symbol] | 262 |
| | [symbol] | |
| | [symbol] | |
| 244 | [symbol] | 263 |
| | [symbol] | |
| | [symbol] | |
| 245 | [symbol] | 264 |
| | [symbol] | |
| | [symbol] | |
| 246 | [symbol] | 265 |
| | [symbol] | |
| | [symbol] | |
| 247 | [symbol] | 266 |
| | [symbol] | |
| | [symbol] | |
| 248 | [symbol] | 267 |
| | [symbol] | |
| | [symbol] | |
| 249 | [symbol] | 268 |
| | [symbol] | |
| | [symbol] | |
| 250 | [symbol] | 269 |
| | [symbol] | |
| | [symbol] | |
| 251 | [symbol] | 270 |
| | [symbol] | |
| | [symbol] | |
| 252 | [symbol] | 271 |
| | [symbol] | |
| | [symbol] | |
| 253 | [symbol] | 272 |
| | [symbol] | |
| | [symbol] | |
| 254 | [symbol] | 273 |
| | [symbol] | |
| | [symbol] | |
| 255 | [symbol] | 274 |
| | [symbol] | |
| | [symbol] | |
| 256 | [symbol] | 275 |
| | [symbol] | |
| | [symbol] | |
| 257 | [symbol] | 276 |
| | [symbol] | |
| | [symbol] | |
| 258 | [symbol] | 277 |
| | [symbol] | |
| | [symbol] | |
| 259 | [symbol] | 278 |
| | [symbol] | |
| | [symbol] | |
| 260 | [symbol] | 289 |
| | [symbol] | |
| | [symbol] | |
| 261 | [symbol] | 280 |
| | [symbol] | |
| | [symbol] | |
| 262 | [symbol] | 281 |
| | [symbol] | |
| | [symbol] | |
| 263 | [symbol] | 282 |
| | [symbol] | |
| | [symbol] | |
| 264 | [symbol] | 283 |
| | [symbol] | |
| | [symbol] | |
| 265 | [symbol] | 284 |
| | [symbol] | |
| | [symbol] | |
| 266 | [symbol] | 285 |
| | [symbol] | |
| | [symbol] | |
| 267 | [symbol] | 286 |
| | [symbol] | |
| | [symbol] | |
| 268 | [symbol] | 287 |
| | [symbol] | |
| | [symbol] | |
| 269 | [symbol] | 288 |
| | [symbol] | |
| | [symbol] | |
| 270 | [symbol] | 289 |
| | [symbol] | |
| | [symbol] | |
| 271 | [symbol] | 290 |
| | [symbol] | |
| | [symbol] | |
| 272 | [symbol] | 291 |
| | [symbol] | |
| | [symbol] | |
| | [symbol] | |
| 273 | [symbol] | 292 |
| | [symbol] | |
| | [symbol] | |
| 274 | [symbol] | 293 |
| | [symbol] | |
| | [symbol] | |
| 275 | [symbol] | 294 |
| | [symbol] | |
| | [symbol] | |
| 276 | [symbol] | 295 |
| | [symbol] | |
| | [symbol] | |
| 278 | [symbol] | 296 |
| | [symbol] | |
| | [symbol] | |
| 279 | [symbol] | 297 |
| | [symbol] | |
| | [symbol] | |
| 280 | [symbol] | 298 |
| | [symbol] | |
| | [symbol] | |
| 281 | [symbol] | 299 |
| | [symbol] | |
| | [symbol] | |
| 282 | [symbol] | 300 |
| | [symbol] | |
| | [symbol] | |
| 283 | [symbol] | 301 |
| | [symbol] | |
| | [symbol] | |
| 284 | [symbol] | 302 |
| | [symbol] | |
| | [symbol] | |
| 286 | [symbol] | 303 |
| | [symbol] | |
| | [symbol] | |
| 287 | [symbol] | 304 |
| | [symbol] | |
| | [symbol] | |
| 288 | [symbol] | 305 |
| | [symbol] | |
| | [symbol] | |
| 289 | [symbol] | 306 |
| | [symbol] | |
| | [symbol] | |
| 290 | [symbol] | 307 |
| | [symbol] | |
| | [symbol] | |
| 291 | [symbol] | 308 |
| | [symbol] | |
| | [symbol] | |
| 292 | [symbol] | 309 |
| | [symbol] | |
| | [symbol] | |
| 293 | [symbol] | 310 |
| | [symbol] | |
| | [symbol] | |
| 294 | [symbol] | 311 |
| | [symbol] | |
| | [symbol] | |
| 295 | [symbol] | 312 |
| | [symbol] | |
| | [symbol] | |
| 296 | [symbol] | 313 |
| | [symbol] | |
| | [symbol] | |
| 297 | [symbol] | 314 |
| | [symbol] | |
| | [symbol] | |
| 298 | [symbol] | 315 |
| | [symbol] | |
| | [symbol] | |
| 299 | [symbol] | 316 |
| | [symbol] | |
| | [symbol] | |
| 300 | [symbol] | 317 |
| | [symbol] | |
| | [symbol] | |
| 301 | [symbol] | 318 |
| | [symbol] | |
| | [symbol] | |
| 302 | [symbol] | 319 |
| | [symbol] | |
| | [symbol] | |
| 303 | [symbol] | 320 |
| | [symbol] | |
| | [symbol] | |
| 304 | [symbol] | 321 |
| | [symbol] | |
| | [symbol] | |
| 305 | [symbol] | 322 |
| | [symbol] | |
| | [symbol] | |
| 306 | [symbol] | 323 |
| | [symbol] | |
| | [symbol] | |
| 307 | [symbol] | 324 |
| | [symbol] | |
| | [symbol] | |
| 308 | [symbol] | 325 |
| | [symbol] | |
| | [symbol] | |
| 309 | [symbol] | 326 |
| | [symbol] | |
| | [symbol] | |
| 310 | [symbol] | 327 |
| | [symbol] | |
| | [symbol] | |
| 311 | [symbol] | 328 |
| | [symbol] | |
| | [symbol] | |
| 312 | [symbol] | 329 |
| | [symbol] | |
| | [symbol] | |
| 313 | [symbol] | 330 |
| | [symbol] | |
| | [symbol] | |
| 314 | [symbol] | 331 |
| | [symbol] | |
| | [symbol] | |
| 315 | [symbol] | 332 |
| | [symbol] | |
| | [symbol] | |
| 316 | [symbol] | 333 |
| | [symbol] | |
| | [symbol] | |
| 317 | [symbol] | 334 |
| | [symbol] | |
| | [symbol] | |
| 318 | [symbol] | 335 |
| | [symbol] | |
| | [symbol] | |
| 319 | [symbol] | 336 |
| | [symbol] | |
| | [symbol] | |
| 320 | [symbol] | 337 |
| | [symbol] | |
| | [symbol] | |
| 321 | [symbol] | 338 |
| | [symbol] | |
| | [symbol] | |
| 322 | [symbol] | 339 |
| | [symbol] | |
| | [symbol] | |
| 323 | [symbol] | 340 |
| | [symbol] | |
| | [symbol] | |
| 324 | [symbol] | 341 |
| | [symbol] | |
| | [symbol] | |
| 325 | [symbol] | 342 |
| | [symbol] | |
| | [symbol] | |
| 326 | [symbol] | 343 |
| | [symbol] | |
| | [symbol] | |
| 327 | [symbol] | 344 |
| | [symbol] | |
| | [symbol] | |
| 328 | [symbol] | 345 |
| | [symbol] | |
| | [symbol] | |
| 329 | [symbol] | 346 |
| | [symbol] | |
| | [symbol] | |
| 330 | [symbol] | 347 |
| | [symbol] | |
| | [symbol] | |
| 331 | [symbol] | 348 |
| | [symbol] | |
| | [symbol] | |
| 332 | [symbol] | 349 |
| | [symbol] | |
| | [symbol] | |
| 333 | [symbol] | 350 |
| | [symbol] | |
| | [symbol] | |
| 334 | [symbol] | 351 |
| | [symbol] | |
| | [symbol] | |
| 335 | [symbol] | 352 |
| | [symbol] | |
| | [symbol] | |
| 336 | [symbol] | 353 |
| | [symbol] | |
| | [symbol] | |
| 337 | [symbol] | 354 |
| | [symbol] | |
| | [symbol] | |
| 338 | [symbol] | 355 |
| | [symbol] | |
| | [symbol] | |
| 339 | [symbol] | 356 |
| | [symbol] | |
| | [symbol] | |
| 340 | [symbol] | 357 |
| | [symbol] | |
| | [symbol] | |
| 341 | [symbol] | 358 |
| | [symbol] | |
| | [symbol] | |
| 342 | [symbol] | 359 |
| | [symbol] | |
| | [symbol] | |
| 343 | [symbol] | 360 |
| | [symbol] | |
| | [symbol] | |
| 344 | [symbol] | 361 |
| | [symbol] | |
| | [symbol] | |
| 345 | [symbol] | 362 |
| | [symbol] | |
| | [symbol] | |
| 346 | [symbol] | 363 |
| | [symbol] | |
| | [symbol] | |
| 347 | [symbol] | 364 |
| | [symbol] | |
| | [symbol] | |
| 348 | [symbol] | 365 |
| | [symbol] | |
| | [symbol] | |

[symbol]

indicates data missing or illegible when filed

In some embodiments, the stefin A protein variant may comprise small deletions or additions—beyond the loop 2 and loop 4 inserts described above—to the Stefin A or Stefin. A derived sequences disclosed herein, such as addition or deletion of up to 10 amino acids relative to Stefin A protein or the Stefin A protein variant.

### Nucleic Acids Encoding CD40L-Binding Proteins

As used herein, “Nucleic acid” is a polynucleotide of any length and may comprise DNA, RNA or a combination of DNA and RNA. The nucleotides can be deoxyribonucleotides, ribonucleotides, modified nucleotides or bases, and/or their analogs, or any substrate that can be incorporated into a polymer by DNA or RNA polymerase.

As used herein, “nucleic acid encoding” refers to a nucleic acid sequence encoding a specific protein or polypeptide. In the art, when the sequence of a specific protein or polypeptide is known, methods for designing or deriving a nucleic acid encoding the same are well known.

Therefore, nucleic acids encoding a CD40L binding agent (e.g., a stefin A protein variant specifically binding to CD40L) can be easily understood from the description above (for example, under the heading “Stefin A protein variants specifically binding to CD40L”.

In some embodiments, a stefin A protein variant specifically binding to CD40L has an amino acid sequence that is encoded by a nucleic acid having a coding sequence at least 70%, 75% 80%, 85%, 90%, 95% or even 98% identical with a sequence selected from SEQ ID NOs: 366 to 485. In some embodiments, the stefin A protein variant specifically binding to CD40L has an amino acid sequence that is encoded by a nucleic acid that having a coding sequence that hybridizes to a sequence selected from SEQ ID NOs: 366 to 485 under stringent conditions (such as in the presence of 6× sodium chloride/sodium citrate (SSC) at 45° C. followed by a wash in 0.2×SSC at 65° C.

TABLE 4
Exemplary A nucleic acid encoding the stefin A protein
variant specifically binding to CD40L

| | | SEQ ID |
|---|---|---|
| Clone # | Nucleic Acid Sequence | NO: |
| 227 | ATGATTCCCGGCGGACTTTCGGAAGCCAAGCCAGCCACTCCGGAGATTCAGG | 366 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCATGATGTGTT | |
| | TTACGATCAGAAAGCAGGTACGAACTACTACATCAAGGTTCGTGCGGGTGAC | |
| | AACAAGTATATGCACCTGAAAGTGTTTAAGAGCCTGAAATGGTGGTACAGA | |
| | ATCGGTAACCCAGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 228 | ATGATTCCCGGCGGACTTTCGGAAGCCAAGCCAGCCACTCCGGAGATTCAGG | 367 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCCATTCAAAAT | |
| | CATCACCGTTTGGCATGGTACGAACTACTACATCAAGGTTCGTGCGGGTGAC | |
| | AACAAGTATATGCACCTGAAAGTGTTTAAGAGCCTGCTGGAAAGATTCAACT | |
| | TCCATGGTGAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 229 | ATGATTCCCGGCGGACTTTCGGAAGCCAAGCCAGCCACTCCGGAGATTCAGG | 368 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCAGTACAAAA | |
| | TCATCACCTGGTGGTCTGGTACGAACTACTACATCAAGGTTCGTGCGGGTGA | |
| | CAACAAGTATATGCACCTGAAAGTGTTTAAGAGCCTGGTTTACAAAAACAAA | |
| | AGAGGTGGTCATGAAGATTTGGTGCTGACGGGCTACCAGGTTGACAAGAAC | |
| | AAAGATGACGAGCTGAGGGTTTCGCGGCCGCTGAACAGAAGCTGATCTCC | |
| | GAAGAAGATCTGGCCGCGCATCACCATCACCACCACTAATGA | |
| 230 | ATGATTCCCGGCGGACTTTCGGAAGCCAAGCCAGCCACTCCGGAGATTCAGG | 369 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCACTACTACGT | |
| | TCATTACAACGATCAGGGTACGAACTACTACATCAAGGTTCGTGCGGGTGAC | |
| | AACAAGTATATGCACCTGAAAGTGTTTAAGAGCCTGTGGGGTGAAAACCTGT | |
| | TCGCAAAATGGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 231 | ATGATTCCCGGCGGACTTTCGGAAGCCAAGCCAGCCACTCCGGAGATTCAGG | 370 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTACTACCAGAT | |
| | CATCACCTGGTGGCAGGGTACGAACTACTACATCAAGGTTCGTGCGGGTGAC | |
| | AACAAGTATATGCACCTGAAAGTGTTTAAGAGCCTGGGTATCAGAAAAAAA | |
| | CAGCAGCAGAGAGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 232 | ATGATTCCCGGCGGACTTTCGGAAGCCAAGCCAGCCACTCCGGAGATTCAGG | 371 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGTTCATGGTCC | |
| | ACATCTGGAACAGGCAGGTACGAACTACTACATCAAGGTTCGTGCGGGTGAC | |
| | AACAAGTATATGCACCTGAAAGTGTTTAAGAGCCTGGGTCATGCATTCTACT | |
| | TCGCATTCGTTGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 227 | ATGATTCCCGGCGGACTTTCGGAAGCCAAGCCAGCCACTCCGGAGATTCAGG | 366 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCATGATGTGTT | |
| | TTACGATCAGAAAGCAGGTACGAACTACTACATCAAGGTTCGTGCGGGTGAC | |
| | AACAAGTATATGCACCTGAAAGTGTTTAAGAGCCTGAAATGGTGGTACAGA | |
| | ATCGGTAACCCAGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 228 | ATGATTCCCGGCGGACTTTCGGAAGCCAAGCCAGCCACTCCGGAGATTCAGG | 367 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCCATTCAAAAT | |
| | CATCACCGTTTGGCATGGTACGAACTACTACATCAAGGTTCGTGCGGGTGAC | |
| | AACAAGTATATGCACCTGAAAGTGTTTAAGAGCCTGCTGGAAAGATTCAACT | |
| | TCCATGGTGAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATACCACTAATGA | |
| 229 | ATGATTCCCGGCGGACTTTCGGAAGCCAAGCCAGCCACTCCGGAGATTCAGG | 368 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCAGTACAAAA | |
| | TCATCACCTGGTGGTCTGGTACGAACTACTACATCAAGGTTCGTGCGGGTGA | |
| | CAACAAGTATATGCACCTGAAAGTGTTTAAGAGCCTGGTTTACAAAAACAAA | |
| | AGAGGTGGTCATGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 230 | ATGATTCCCGGCGGACTTTCGGAAGCCAAGCCAGCCACTCCGGAGATTCAGG | 369 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCACTACTACGT | |
| | TCATTACAACGATCAGGGTACGAACTACTACATCAAGGTTCGTGCGGGTGAC | |
| | AACAAGTATATGCACCTGAAAGTGTTTAAGAGCCTGTGGGGTGAAAACCTGT | |
| | TCGCAAAATGGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 231 | ATGATTCCCGGCGGACTTTCGGAAGCCAAGCCAGCCACTCCGGAGATTCAGG | 370 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTACTACCAGAT | |
| | CATCACCTGGTGGCAGGGTACGAACTACTACATCAAGGTTCGTGCGGGTGAC | |
| | AACAAGTATATGCACCTGAAAGTGTTTAAGAGCCTGGGTATCAGAAAAAAA | |
| | CAGCAGCAGAGAGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 232 | ATGATTCCCGGCGGACTTTCGGAAGCCAAGCCAGCCACTCCGGAGATTCAGG | 371 |
| | AAATCGTGGACAAGGTCAAACCGCAGCTGGAAGAGAAAACCGGCGAAACCT | |
| | ACGGCAAACTGGAAGCCGTCCAGTATAAGAACTCAAGTCGTCGTTCATGGTCC | |
| | ACATCTGGAACAGGCAGGTACGAACTACTACATCAAGGTTCGTGCGGGTGAC | |
| | AACAAGTATATGCACCTGAAAGTGTTTAAGAGCCTGGGTCATGCATTCTACT | |
| | TCGCATTCGTTGAAGATTTGGTGCTGACGGGCTACCAGGTTGACAAGAACAA | |
| | AGATGACGAGCTGACGGGTTTCGCCGGCCGCTGAACAGAAGCTGATCTCCGA | |
| | AGAAGATCTGGCCGCGCATCACCATCACCACCACTAATGA | |
| 233 | ATGATTCCCGGCGGACTTTCGGAAGCCAAGCCAGCCACTCCGGAGATTCAGG | 372 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCTGCAGGATCA | |
| | GTGGCCATGGCGTGATGGTACGAACTACTACATCAAGGTTCGTGCGGGTGAC | |
| | AACAAGTATATGCACCTGAAAGTGTTTAAGAGCCTGACCTACGGTAAAAAAT | |
| | GGTACTCTTGGGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 234 | ATGATTCCCGGCGGACTTTCGGAAGCCAAGCCAGCCACTCCGGAGATTCAGG | 373 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGACGCGGGTACGA | |
| | ACTACTACATCAAGGTTCGTGCGGGTGACAACAAGTATATGCACCTGAAAGT | |
| | GTTTAAGAGCCTGTACTTCTACAGACATTGGGAAGATAGAGAAGATTTGGTG | |
| | CTGACGGGCTACCAGGTTGACAAGAACAAAGATGACGAGCTGACGGGTTTC | |
| | GCGGCCGCTGAACAGAAGCTGATCTCCGAAGAAGATCTGGCCGCGCATCAC | |
| | CATCACCACCACTAATGA | |
| 235 | ATGATTCCCGGCGGACTTTCGGAAGCCAAGCCAGCCACTCCGGAGATTCAGG | 374 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTCTTGGGTTAA | |
| | ACCAGATAACGAACTGGGTACGAACTACTACATCAAGGTTCGTGCGGGTGAC | |
| | AACAAGTATATGCACCTGAAAGTGTTTAAGAGCCTGTGGCAGGTTCATGATA | |
| | AACAGTGGCTGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 236 | ATGATTCCCGGCGGACTTTCGGAAGCCAAGCCAGCCACTCCGGAGATTCAGG | 375 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCAACTCTCTGGG | |
| | TGTTTCTGAATGGGTTGGTACGAACTACTACATCAAGGTTCGTGCGGGTGAC | |
| | AACAAGTATATGCACCTGAAAGTGTTTAAGAGCCTGCCATACTGGTACAGAA | |
| | AAAACAACCTGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 237 | ATGATTCCCGGCGGACTTTCGGAAGCCAAGCCAGCCACTCCGGAGATTCAGG | 376 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGTTTACCGTAT | |
| | CGTTTCTTGGTGGCGTGGTACGAACTACTACATCAAGGTTCGTGCGGGTGAC | |
| | AACAAGTATATGCACCTGAAAGTGTTTAAGAGCCTGGCATCTGATATCTACC | |
| | TGAGAATCGATGAAGATTTGGTGCTGACGGGCTACCAGGTTGACAAGAACA | |
| | AAGATGACGAGCTGACGGGTTTCCCGGCCGCTGAACAGAAGCTGATCTCCG | |
| | AAGAAGATCTGGCCGCGCATCACCATCACCACCACTAATGA | |
| 238 | ATGATTCCCGGCGGACTTTCGGAAGCCAAGCCAGCCACTCCGGAGATTCAGG | 377 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTACTACCGTCA | |
| | GCTGAAATTCAACACCGGTACGAACTACTACATCAAGGTTCGTGCGGGTGAC | |
| | AACAAGTATATGCACCTGAAAGTGTTTAAGAGCCTGTTCCTGCATAGAAAAC | |
| | CACAGGATAGAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 239 | ATGATTCCCGGCGGACTTTCGGAAGCCAAGCCAGCCACTCCGGAGATTCAGG | 378 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGGTTGGATCAA | |
| | AGTTGATAACGTTCTGGGTACGAACTACTACATCAAGGTTCGTGCGGGTGAC | |
| | AACAAGTATATGCACCTGAAAGTGTTTAAGAGCCTGTGGCAGGATCATCATA | |
| | ACGTTAAAAGAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 240 | ATGATTCCCGGCCGACTTTCGGAAGCCAAGCCAGCCACTCCGGAGATTCAGG | 379 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCACCGCATACTA | |
| | CATCCATGTTAACGCAGGTACGAACTACTACATCAAGGTTCGTGCGGGTGAC | |
| | AACAAGTATATGCACCTGAAAGTGTTTAAGAGCCTGCCATACGGTTACAGAG | |
| | CAAAAGCACCAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 241 | ATGATTCCCGGCGGACTTTCGGAAGCCAAGCCAGCCACTCCGGAGATTCAGG | 380 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGTTCGTGTTGA | |
| | ACCAACCCCAAACGATGGTACGAACTACTACATCAAGGTTCGTGCGGGTGAC | |
| | AACAAGTATATGCACCTAAAAGTGTTTAAGAGCCTGCCACTGGTTCAGGAAC | |
| | TGTGGGGTTTCGAAGATTTGGTGCTGACGGGCTACCAAGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 242 | ATGATTCCCGGCGGACTTTCGGAAGCCAAGCCAGCCACTCCGGAGATTCAGG | 381 |
| | AAATCGTGGACAAGGTCAAACCGCAGCTGGAAGAGAAAACCGGCGAAACCT | |
| | ACGGCAAACTGGAACCCGTCCAGTATAAGACTCAAGTCGTCGATCTGAACTC | |
| | TTCTGTTTGGTACCAGGGTACGAACTACTACATCAAGGTTCGTGCGGGTGAC | |
| | AACAAGTATATGCACCTGAAAGTGTTTAAGAGCCTGTACTGGCTGCTGGAAA | |
| | TCGAACATAGAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 243 | ATGATTCCCGGCGGACTTTCGGAAGCCAAGCCAGCCACTCCGGAGATTCAGG | 382 |
| | AAATCGTGGACAAGGTCAAACCGCAGCTGGAAGAGAAAACCCGCGAAACCT | |
| | ACGGCAAACTGGAAGCCGTCCAGTATAAGACTCAAGTCGTCTTCACCTACCA | |
| | GATCTCTTACACCGTTGGTACGAACTACTACATCAAGGTTCGTGCGGGTGAC | |
| | AACAAGTATATGCACCTGAAAGTGTTTAAGAGCCTGAGATGGAGATACCCA | |
| | GCAAGAAAACCAGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 244 | ATGATTCCCGGCGGACTTTCGGAAGCCAAGCCAGCCACTCCGGAGATTCAGG | 838 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCGTGAAAACT | |
| | GGTACGGTGAATTCGCAGGTACGAACTACTACATCAAGGTTCGTGCGGGTGA | |
| | CAACAAGTATATGCACCTGAAAGTGTTTAAGAGCCTGCTGTACATCTACAGA | |
| | CATCCAAACTACGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 245 | ATGATTCCCGGCGGACTTTCGGAAGCCAAGCCAGCCACTCCGGAGATTCAGG | 384 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCACCGCACTGGC | |
| | ATGGAACACCCAGGCAGGTACGAACTACTACATCAAGGTTCGTGCGGGTGA | |
| | CAACAAGTATATGCACCTGAAAGTGTTTAAGAGCCTGCCATGGCTGTACAGA | |
| | CAGAAATCTGATGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 246 | ATGATTCCCGGCGGACTTTCGGAAGCCAAGCCAGCCACTCCGGAGATTCAGG | 385 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCATATCCCAGC | |
| | ACTGAAAGTTCATGCAGGTACGAACTACTACATCAAGGTTCGTGCGGGTGAC | |
| | AACAAGTATATGCACCTGAAAGTGTTTAAGAGCCTGCCATACTGGTACAGAC | |
| | ATGATCAGGAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 247 | ATGATTCCCGGCGGACTTTCGGAAGCCAAGCCAGCCACTCCGGAGATTCAGG | 386 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCATCATGCATA | |
| | CGCAGCAGTTTACGTTGGTACGAACTACTACATCAAGGTTCGTGCGGGTGAC | |
| | AACAAGTATATGCACCTGAAAGTGTTTAAGAGCCTGCCATACTGGTACAGAA | |
| | AATACCCAGATGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 248 | ATGATTCCCGGCGGACTTTCGGAAGCCAAGCCAGCCACTCCGGAGATTCAGG | 387 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTACGATGAACT | |
| | GTACCATGAAAAAGCAGGTACGAACTACTACATCAAGGTTCGTGCGGGTGA | |
| | CAACAAGTATATGCACCTGAAAGTGTTTAAGAGCCTGGAATACAAATTCAGA | |
| | AACCTGGAAGAAGATTTGGTGCTGACGGGCTACCAGGTTGACAAGAACAAA | |
| | GATGACGAGCTGACGGGTTTCGCGGCCGCTGAACAGAAGCTGATCTCCGAA | |
| | GAAGATCTGGCCGCGCATCACCATCACCACCACTAATGA | |
| 249 | ATGATTCCCGGCGGACTTTCGGAAGCCAAGCCAGCCACTCCGGAGATTCAGG | 388 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTCTTGGTACTA | |
| | CCCAAAAAACGTTCTGGGTACGAACTACTACATCAACGTTCGTGCGGGTGAC | |
| | AACAAGTATATGCACCTGAAAGTGTTTAAGAGCCTGGAAGATTTCATCGCAG | |
| | CAGATGATGATGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 250 | ATGATTCCCGGCGGACTTTCGGAAGCCAAGCCAGCCACTCCGGAGATTCAGG | 389 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTTCCTGGAATG | |
| | GCACTTCTGGGAATGGGGTACGAACTACTACATCAAGGTTCGTGCGGGTGAC | |
| | AACAAGTATATGCACCTGAAAGTGTTTAAGAGCCTGGATTCTCAGGAATTCC | |
| | CATCTCATCTGGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 251 | ATGATTCCCGGCGGACTTTCGGAAGCCAAGCCAGCCACTCCGGAGATTCAGG | 390 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTCTTGGGTTCG | |
| | TGGTACCAACCAGCTGGGTACGAACTACTACATCAAGGTTCGTGCGGGTGAC | |
| | AACAAGTATATGCACCTGAAAGTGTTTAAGAGCCTGGAACCAGATTGGCCAG | |
| | ATTTCCTGATCGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 252 | ATGATTCCCGGCGGACTTTCGGAAGCCAAGCCAGCCACTCCGGAGATTCAGG | 391 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTGGCGTAAACT | |
| | GCTGTCTTGGTGGAAAGGTACGAACTACTACATCAAGGTTCGTGCGGGTGAC | |
| | AACAAGTATATGCACCTGAAAGTGTTTAAGAGCCTGATCCTGAACCCAAAAT | |
| | CTTGGAACTTCGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 253 | ATGATTCCCGGCGGACTTTCGGAAGCCAAGCCAGCCACTCCGGAGATTCAGG | 392 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCCAAAATCTCA | |
| | TCAGACCAACGAACAGGGTACGAACTACTACATCAAGGTTCGTGCGGGTGA | |
| | CAACAAGTATATGCACCTGAAAGTGTTTAAGAGCCTGTGGAACGAAAACCAT | |
| | TTCTCTGTTTGGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 254 | ATGATTCCCGGCGGACTTTCGGAAGCCAAGCCAGCCACTCCGGAGATTCAGG | 393 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCAACTGGAACC | |
| | AGATCGATAACCCACTGGGTACGAACTACTACATCAAGGTTCGTGCGGGTGA | |
| | CAACAAGTATATGCACCTGAAAGTGTTTAAGAGCCTGAACAAAGATTGGGA | |
| | AGATCAGATCATCGAAGATTTGGTGCTGACGGGCTACCAGGTGACAAGAAC | |
| | AAAGATGACGAGCTGACGGGTTTCGCGGCCGCGGGTGGAAGAGCAGAACAG | |
| | AAGCTGATCTCCGAAGAAGATCTGGCCGCGCATCACCATCACCACCACTAAT | |
| | GA | |
| 255 | ATGATTCCCGGCGGACTTTCGGAAGCCAAGCCAGCCACTCCGGAGATTCAGG | 394 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTTCTGGGTTGG | |
| | TCTGCATAACCAGCTGGGTACGAACTACTACATCAAGGTTCGTGCGGGTGAC | |
| | AACAAGTATATGCACCTGAAAGTGTTTAAGAGCCTGTACTACAACGGTGATA | |
| | ACACCCAGATCGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 256 | ATGATTCCCGGCGGACTTTCGGAAGCCAAGCCAGCCACTCCGGAGATTCAGG | 395 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTTCACCCAGCC | |
| | ACGGCAAACTGGAAGCCGTCCAGTATAAGACTCAAGTCGTCTTCACCCAGCC | |
| | ATTCAACGAACCAATCGGTACGAACTACTACATCAAGGTTCGTGCGGGTGAC | |
| | AACAAGTATATGCACCTGAAAGTGTTTAAGAGCCTGTATTGGGATGATAAG | |
| | TTGGTTACTACGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 257 | ATGATTCCCGGCGGACTTTCGGAAGCCAAGCCAGCCACTCCGGAGATTCAGG | 396 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCCAGAATACC | |
| | AGCCACAGCCAAAAGCAGGTACGAACTACTACATCAAGGTTCGTGCGGGTG | |
| | ACAACAAGTATATGCACCTGAAAGTGTTTAAGAGCCTGTTCCATTTCAGAAA | |
| | ACCACAGGATAGAGAAGATTTGGTGCTGACGGGCTACCAGGTTGACAAGAA | |
| | CAAAGATGACGAGCTGACGGGTTTCGCGGCCGCTGAACAGAAGCTGATCTCC | |
| | GAAGAAGATCTGGCCGCGCATCACCATCACCACCACTAATGA | |
| 258 | ATGATTCCCGGCGGACTTTCGGAAGCCAAGCCAGCCACTCCGGAGATTCAGG | 397 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCAACGATCCATA | |
| | CTTCCAGTCTTTCTCTGGTACGAACTACTACATCAAGGTTCGTGCGGGTGACA | |
| | ACAAGTATATGCACCTGAAAGTGTTTAAGAGCCTGCCATACTGGTACAGAGA | |
| | ACCAGAAAACGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 259 | ATGATTCCCGGCGGACTTTCGGAAGCCAAGCCAGCCACTCCGGAGATTCAGG | 398 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCATCAGGATG | |
| | ATGTTGCATGGCAGTCTGGTACGAACTACTACATCAAGGTTCGTGCGGGTGA | |
| | CAACAAGTATATGCACCTGAAAGTGTTTAAGAGCCTGGAATACGGTTGGTAC | |
| | CATCTGCCATACGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 260 | ATGATTCCCGGCGGACTTTCGGAAGCCAAGCCAGCCACTCCGGAGATTCAGG | 399 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCAAAGGTTACTT | |
| | CCATACCGTTGAAGCAGGTACGAACTACTACATCAAGGTTCGTGCGGGTGAC | |
| | AACAAGTATATGCACCTGAAAGTGTTTAAGAGCCTGACCTACCATTTCAGAA | |
| | CCGATCTGAACGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 261 | ATGATTCCCGGCGGACTTTCGGAAGCCAAGCCAGCCACTCCGGAGATTCAGG | 400 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGTTGATCAGGT | |
| | TTGGTCTGAAAACGCAGGTACGAACTACTACATCAAGGTTCGTGCGGGTGAC | |
| | AACAAGTATATGCACCTGAAAGTGTTTAAGAGCCTGCCATACCTGTGGAGAA | |
| | AACATGCAGTTGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 262 | ATGATTCCCGGCGGACTTTCGGAAGCCAAGCCAGCCACTCCGGAGATTCAGG | 401 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCTGTCTTCTAT | |
| | CTGGCAGGATGTTGCAGGTACGAACTACTACATCAAGGTTCGTGCGGGTGAC | |
| | AACAAGTATATGCACCTGAAAGTGTTTAAGAGCCTGCTGTACCAGTTCAGAA | |
| | AAGAACCAGTTGAAGATTTGGTGCTGACGGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 263 | ATGATTCCCGGCGGACTTTCGGAAGCCAAGCCAGCCACTCCGGAGATTCAGG | 402 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCACCGCATCTCT | |
| | GTACTGGAACTACGCAGGTACGAACTACTACATCAAGGTTCGTGCGGGTGAC | |
| | AACAAGTATATGCACCTGAAAGTGTTTAAGAGCCTGTTCTACTACAGAGGTC | |
| | CACAGCCAGTTGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 264 | ATGATTCCCGGCGGACTTTCGGAAGCCAAGCCAGCCACTCCGGAGATTCAGG | 403 |
| | AAATCGTGGACAAGGTCAAACCGCAGCTGGAAGAGAAAACCGGCGAAACCT | |
| | ACGGCAAACTGGAACCCGTCCAGTATAAGACTCAAGTCGTCTCTTGGAAACA | |
| | GTGGCATAACTACCTGGGTACGAACTACTACATCAAGGTTCGTGCGGGTGAC | |
| | AACAAGTATATGCACCTGAAAGTGTTTAAGAGCCTGTACCCAGATGGTGATC | |
| | TGATCCTGTACGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 265 | ATGATTCCCGGCGGACTTTCGGAAGCCAAGCCAGCCACTCCGGAGATTCAGG | 404 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCAGGTTCATCT | |
| | GGATAACTCTCCACTGGGTACGAACTACTACATCAAGGTTCGTGCGGGTGAC | |
| | AACAAGTATATGCACCTGAAAGTGTTTAAGAGCCTGCAGTTCGGTGAAATCA | |
| | TCTGGTGGGAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 266 | ATGATTCCCGGCGGACTTTCGGAAGCCAAGCCAGCCACTCCGGAGATTCAGG | 405 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCAGAACAACT | |
| | GGTACCTGTCTAACTACGGTACGAACTACTACATCAAGGTTCGTGCGGGTGA | |
| | CAACAAGTATATGCACCTGAAAGTGTTTAAGAGCCTGGTTGAAGAAAGATG | |
| | GGGTCTGTGGGTTGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 267 | ATGATTCCCGGCGGACTTTCGGAAGCCAAGCCAGCCACTCCGGAGATTCAGG | 406 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTGGGAATGGG | |
| | CACTGCTGCTGGAATACGGTACGAACTACTACATCAAGGTTCGTGCGGGTGA | |
| | CAACAAGTATATGCACCTGAAAGTGTTTAAGAGCCTGCATCATCCATGGCAG | |
| | CCAGAAAACAGGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 268 | ATGATTCCCGGCGGACTTTCGGAAGCCAAGCCAGCCACTCCGGAGATTCAGG | 407 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGGTGATTCTTA | |
| | CTGGGAATACGTTTCTGGTACGAACTACTACATCAAGGTTCGTGCGGGTGAC | |
| | AACAAGTATATGCACCTGAAAGTGTTTAAGAGCCTGCCATACAGATGGAGAC | |
| | CAGATCATGCAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 269 | ATGATTCCCGGCGGACTTTCGGAAGCCAAGCCAGCCACTCCGGAGATTCAGG | 408 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGGTCAGAAAT | |
| | GGTGGCAGCATAAAATCGGTACGAACTACTACATCAAGGTTCGTGCCCGTGA | |
| | CAACAAGTATATGCACCTGAAAGTGTTTAAGAGCCTGCATTCTAACGCATGG | |
| | AACCTGCAGCATGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 270 | ATGATTCCCGGCGGACTTTCGGAAGCCAAGCCAGCCACTCCGGAGATTCAGG | 409 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCTGGATGGTCT | |
| | GTACCATGTTAACTCTGGTACGAACTACTACATCAAGGTTCGTGCGGGTGAC | |
| | AACAAGTATATGCACCTGAAAGTGTTTAAGAGCCTGTTCTACTACTACAGAT | |
| | CTGAACCAACCGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 271 | ATGATTCCCGGCGGACTTTCGGAAGCCAAGCCAGCCACTCCGGAGATTCAGG | 410 |
| | AAATCGTGGACAAGGTCAAACCGCAGCTGGAAGAGAAAACCGGCGAAACCT | |
| | ACGGCAAACTGGAAGCCGTCCAGTATAGACTCAAGTCGTCCATGCACCAGC | |
| | ACCAGTTCAGGCAATCGGTACGAACTACTACATCAAGGTTCGTGCGGGTGAC | |
| | AACAAGTATATGCACCTGAAAGTGTTTAAGAGCCTGCCATACTGGTACAGAC | |
| | CAAACAGACCAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 272 | ATGATTCCCGGCGGACTTTCGGAAGCCAAGCCAGCCACTCCGGAGATTCAGG | 411 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTGGGAAGATC | |
| | ATTGGAACTACCAGTACGGTACGAACTACTACATCAAGGTTCGTGCGGGTGA | |
| | CAACAAGTATATGCACCTGAAAGTGTTTAAGAGCCTGGAACTGTACTCTAAC | |
| | CAGATCGTTTGGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 273 | ATGATTCCCGGCGGACTTTCGGAAGCCAAGCCAGCCACTCCGGAGATTCAGG | 412 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTCTTGGCGTTA | |
| | CAAATCTAACCAGCTGGGTACGAACTACTACATCAAGGTTCGTGCGGGTGAC | |
| | AACAAGTATATGCACCTGAAAGTGTTTAAGAGCCTGTACAAAGATTGGGTTG | |
| | TTAACCCACCAGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 274 | ATGATTCCCGGCGGACTTTCGGAAGCCAAGCCAGCCACTCCGGAGATTCAGG | 413 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCGTGTTGATTG | |
| | GTGGCTGCCAGAAGTTGGTACGAACTACTACATCAAGGTTCGTGCGGGTGAC | |
| | AACAAGTATATGCACCTGAAAGTGTTTAAGAGCCTGCCATACCAGTACAGAA | |
| | AAGATCCAAAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 275 | ATGATTCCCGGCGGACTTTCGGAAGCCAAGCCAGCCACTCCGGAGATTCAGG | 414 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTCTTGGATCGA | |
| | AATCTCTAACCAGCTGGGTACGAACTACTACATCAAGGTTCGTGCGGGTGAC | |
| | AACAAGTATATGCACCTGAAAGTGTTTAAGAGCCTGTACTACGGTAACACCT | |
| | CTATCCTGATCGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 276 | ATGATTCCCGGCGGACTTTCGGAAGCCAAGCCAGCCACTCCGGAGATTCAGG | 415 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTCTCTGGGTCA | |
| | GGCAGAAACCTGGGCAGGTACGAACTACTACATCAAGGTTCGTGCGGGTGA | |
| | CAACAAGTATATGCACCTGAAAGTGTTTAAGAGCCTGTTCAAATACAGACAT | |
| | CAGCAGGATAAAGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 278 | ATGATTCCCGGCGGACTTTCGGAAGCCAAGCCAGCCACTCCGGAGATTCAGG | 416 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCAAAATCGGTC | |
| | AGCTGCCATCTCGTGTTGGTACGAACTACTACATCAAGGTTCGTGCGGGTGA | |
| | CAACAAGTATATGCACCTGAAAGTGTTTAAGAGCCTGTACTGGTACAGAAAC | |
| | CCAGCAGATAGAGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 279 | ATGATTCCCGGCGGACTTTCGGAAGCCAAGCCAGCCACTCCGGAGATTCAGG | 417 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGCATGGCATAC | |
| | CAAAAACAACTCTATCGGTACGAACTACTACATCAAGGTTCGTGCGGGTGAC | |
| | AACAAGTATATGCACCTGAAAGTGTTTAAGAGCCTGGAAACCAAACTGGCA | |
| | CATGAAGATATCGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 280 | ATGATTCCCGGCGGACTTTCGGAAGCCAAGCCAGCCACTCCGGAGATTCAGG | 418 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCAGACCAAAC | |
| | TGGTTACCTGGTGGTGGGGTACGAACTACTACATCAAGGTTCGTGCGGGTGA | |
| | CAACAAGTATATGCACCTGAAAGTGTTTAAGAGCCTGCTGGGTGAACAGTAC | |
| | ATCGATGTTGATGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 281 | ATGATTCCCGGCGGACTTTCGGAAGCCAAGCCAGCCACTCCGGAGATTCAGG | 419 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGCAGTTTTCGA | |
| | TTACGCAGATCCATCTGGTACGAACTACTACATCAAGGTTCGTGCGGGTGAC | |
| | AACAAGTATATGCACCTGAAAGTGTTTAAGAGCCTGCCATACAGATACAGAA | |
| | CCCATCATAAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 282 | ATGATTCCCGGCGGACTTTCGGAAGCCAAGCCAGCCACTCCGGAGATTCAGG | 420 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCATGTTAAAAT | |
| | CGTTACCTGGTGGAAAGGTACGAACTACTACATCAAGGTTCGTGCGGGTGAC | |
| | AACAAGTATATGCACCTGAAAGTGTTTAAGAGCCTGTACAACCCAAAACCAG | |
| | GTATCGATAACGAAGATTTGGTGCTGACGGGCTACCAGGTTGACAAGAACA | |
| | AAGATGACGAGCTGACGGGTTCGCGGCCGCTGAACAGAAGCTGATCTCCG | |
| | AAGAAGATCTGGCCGCGCATCACCATCACCACCACTAATGA | |
| 283 | ATGATTCCCGGCGGACTTTCGGAAGCCAAGCCAGCCACTCCGGAGATTCAGG | 421 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCATGTTCTGAA | |
| | AGTTTCTTCTGTTTCTGGTACGAACTACTACATCAAGGTTCGTGCGGGTGACA | |
| | ACAAGTATATGCACCTGAAAGTGTTTAAGAGCCTGTTCTACTACAGAAAAAG | |
| | ATACGATAGAGAAGATTTGGTGCTGACGGGCTACCACGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 284 | ATGATTCCCGGCGGACTTTCGGAAGCCAAGCCAGCCACTCCGGAGATTCAGG | 422 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTACCAGGATTA | |
| | CGATTCTGCAACCCATGGTACGAACTACTACATCAAGGTTCGTGCGGGTGAC | |
| | AACAAGTATATGCACCTGAAAGTGTTTAAGAGCCTGTACCAGTACAGAAACC | |
| | CAATCGATAAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 286 | ATGATTCCCGGCGGACTTTCGGAAGCCAAGCCAGCCACTCCGGAGATTCAGG | 423 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTCTTGGGTAA | |
| | AGTTTCTAACGAACTGGGTACGAACTACTACATCAAGGTTCGTGCGGGTGAC | |
| | AACAAGTATATGCACCTGAAAGTGTTTAAGAGCCTGATCGAATACCAGTCTC | |
| | CAGCACTGTACGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 287 | ATGATTCCCGGCGGACTTTCGGAAGCCAAGCCAGCCACTCCGGAGATTCAGG | 424 |
| | AAATCGTGGGACAAGGTCAAACCGCAGCTGGAAGAGAAAACCGGCGAAACCT | |
| | ACGGCAAACTGGAAGCCGTCCAGTATAAGACTCAAGTCGTCCTGGAAGATG | |
| | AATGGTGGTCTATCCTGGGTACGAACTACTACATCAAGGTTCGTGCGGGTGA | |
| | CAACAAGTATATGCACCTGAAAGTGTTTAAGAGCCTGCCAGATGCACCAGCA | |
| | TACTCTAAATCTGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCTCACCACCACTAATGA | |
| 288 | ATGATTCCCGGCGGACTTTCGGAAGCCAAGCCAGCCACTCCGGAGATTCAGG | 425 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTACTACAAACT | |
| | GGTTACCTGGTGGCGTGGTACGAACTACTACATCAAGGTTCGTGCGGGTGAC | |
| | AACAAGTATATGCACCTGAAAGTGTTTAAGAGCCTGCCAGATCTGAACTACG | |
| | AAGTTACCCTGGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 289 | ATGATTCCCGGCGGACTTTCGGAAGCCAAGCCAGCCACTCCGGAGATTCAGG | 426 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGCATGGGTTAA | |
| | AGAACATAACCTGATCGGTACGAACTACTACATCAAGGTTCGTGCGGGTGAC | |
| | AACAAGTATATGCACCTGAAAGTGTTTAAGAGCCTGTACGATCAGAACCAGT | |
| | GGTCTCCACATGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 290 | ATGATTCCCGGCGGACTTTCGGAAGCCAAGCCAGCCACTCCGGAGATTCAGG | 427 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCAGGAATACCT | |
| | GTGGAAACAGTACATCGGTACGAACTACTACATCAAGGTTCGTGCGGGTGAC | |
| | AACAAGTATATGCACCTGAAAGTGTTTAAGAGCCTGAGATGGCAGTACAGA | |
| | CATCCACAGACCGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 291 | ATGATTCCCGGCGGACTTTCGGAAGCCAAGCCAGCCACTCCGGAGATTCAGG | 428 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTTCAACAAAG | |
| | ATCTGTTCCTGGGTTGGGGTACGAACTACTACATCAAGGTTCGTGCGGGTGA | |
| | CAACAAGTATATGCACCTGAAAGTGTTTAAGAGCCTGAGATTCTCTGCAGGT | |
| | GAAGAATGGGAAGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 292 | ATGATTCCCGGCGGACTTTCGGAAGCCAAGCCAGCCACTCCGGAGATTCAGG | 429 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCATTTCGGTAT | |
| | CCGTTACGCACATGTTGGTACGAACTACTACATCAAGGTTCGTGCGGGTGAC | |
| | AACAAGTATATGCACCTGAAAGTGTTTAAGAGCCTGGATTACCCAAGACATC | |
| | ATCGGATCAGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 293 | ATGATTCCCGGCGGACTTTCGGAAGCCAAGCCAGCCACTCCGGAGATTCAGG | 430 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTGGTACGCATG | |
| | GCAGGAAGGTTGGCAGGGTACGAACTACTACATCAAGGTTCGTGCGGGTGA | |
| | CAACAAGTATATGCACCTGAAAGTGTTTAAGAGCCTGTTCAGAAAAAAATGG | |
| | GCACAGCTGTGGGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 294 | ATGATTCCCGGCGGACTTTCGGAAGCCAAGCCAGCCACTCCGGAGATTCAGG | 431 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGGTAACGCAG | |
| | CATGGGGTTCTTTCGCAGGTACGAACTACTACATCAAGGTTCGTGCGGGTGA | |
| | CAACAAGTATATGCACCTGAAAAGTGTTTAAGAGCCTGCCATTCTTCTACAGA | |
| | AAAGATAACCCAGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 295 | ATGATTCCCGGCCGGACTTTCGGAAGCCAAGCCAGCCACTCCGGAGATTCAGG | 432 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCGTGAAGATCT | |
| | GGATCTGTTCTACACCGGTACGAACTACTACATCAAGGTTCGTGCGGGTGAC | |
| | AACAAGTATATGCACCTGAAAGTGTTTAAGAGCCTGGAATTCATCCTGCAGC | |
| | CATGGTGGTACGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 296 | ATGATTCCCGGCGGACTTTCGGAAGCCAAGCCAGCCACTCCGGAGATTCAGG | 433 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCAACGAAAACG | |
| | TTTACCGTGTTACCCAGGGTACGAACTACTACATCAAGGTTCGTGCGGGTGA | |
| | CAACAAGTATATGCACCTGAAAGTGTTTAAGAGCCTGAAATACAGACATAG | |
| | ACTGACCTACGAAGAAGATTTGGTGCTGACGGGCTACCCAAGTTGACAAGAA | |
| | CAAAGATGACGAGCTGACGGGTTTCGCGGCCGCTGAACAGAAGCTGATCTCC | |
| | GAAGAAGATCTGGCCGCGCATCACCATCACCACCACTAATGA | |
| 297 | ATGATTCCCGGCGGACTTTCGGAAGCCAAGCCAGCCACTCCGGAGATTCAGG | 434 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTGGCATAAAAT | |
| | CGTTACCCAGTGGCCAGGTACGAACTACTACATCAAGGTTCGTGCGGGTGAC | |
| | AACAAGTATATGCACCTGAAAGTGTTTAAGAGCCTGTACGCACAGAGAGGT | |
| | GAAGTTTACTTCGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 298 | ATGATTCCCGGCGGACTTTCGGAAGCCAAGCCAGCCACTCCGGAGATTCAGG | 435 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCATTACCGTAT | |
| | CATCACCCATTGGGAAGGTACGAACTACTACATCAAGGTTCGTGCGGGTGAC | |
| | AACAAGTATATGCACCTGAAAGTGTTTAAGAGCCTGGTTAGAGCATTCCTGT | |
| | ACTACAAAGATGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 299 | ATGATTCCCGGCGGACTTTCGGAAGCCAAGCCAGCCACTCCGGAGATTCAGG | 436 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCTGGTTTTCGA | |
| | TGCATCTACCTCTGCAGGTACGAACTACTACATCAAGGTTCGTGCGGGTGAC | |
| | AACAAGTATATGCACCTGAAAGTGTTTAAGAGCCTGCCATGGGAATTCAGAG | |
| | GTGATCATAAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 300 | ATGATTCCCGGCGGACTTTCGGAAGCCAAGCCAGCCACTCCGGAGATTCAGG | 437 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCAGTACGCAG | |
| | AATTCCTGGAACATATCGGTACGAACTACTACATCAAGGTTCGTGCGGGTGA | |
| | CAACAAGTATATGCACCTGAAAGTGTTTAAGAGCCTGGGTTGGTCTCATTAC | |
| | TTCCAGTACCTGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 301 | ATGATTCCCGGCGGACTTTCGGAAGCCAAGCCAGCCACTCCGGAGATTCAGG | 438 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGGTAACGTTCA | |
| | GGAAGATTGGCATATCGGTACGAACTACTACATCAAGGTTCGTGCGGGTGAC | |
| | AACAAGTATATGCACCTGAAAGTGTTTAAGAGCCTGTTCTCTGAAGATCTGA | |
| | CCCTGAACGGTGAAGATTTGGTGCTGACGGGCTACCAGGTTGACAAGAACA | |
| | AAGATGACGAGTGACGGGTTTCGCGGCCGCTGAACAGAAGCTGATCTCCG | |
| | AAGAAGATCTGGCCGCGCATCACCATCACCACCACTAATGA | |
| 302 | ATGATTCCCGGCGGACTTTCGGAAGCCAAGCCAGCCACTCCGGAGATTCAGG | 439 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGGTTGGTCTGC | |
| | ATCTTACCCAGAATCTGGTACGAACTACTACATCAAGGTTCGTGCGGGTGAC | |
| | AACAAGTATATGCACCTGAAAGTGTTTAAGAGCCTGACCCAGTGGGAATTCA | |
| | TCGAATGGCCAGAAGATTTGGTGCTGACGGGCTACCAGGTTGACAAGAACA | |
| | AAGAAGATCTGGCCGCGCATCACCCATCACCACCACTAATGA | |
| 303 | ATGATTCCCGGCGGACTTTCGGAAGCCAAGCCAGCCACTCCGGAGATTCAGG | 440 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTACCAGTTCGA | |
| | ACATATCTCCTACGGTGGTACGAACTACTACATCAAGGTTCGTGCGGGTGAC | |
| | AACAAGTATATGCACCTGAAAGTGTTTAAGAGCCTGTTCACCTACAGACTGC | |
| | CACAGCATAGAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 304 | ATGATTCCCGGCGGACTTTCGGAAGCCAAGCCAGCCACTCCGGAGATTCAGG | 441 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGCATGGATCAC | |
| | CCCAGTTAACGTTCTGGGTACGAACTACTACATCAAGGTTCGTGCGGGTGAC | |
| | AACAAGTATATGCACCTGAAAGTGTTTAAGAGCCTGGATGAAATCTTCCATG | |
| | GTGCATACAACGAAGATTTGGTGCTGAGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 305 | ATGATTCCCGGCGGACTTTCGGAAGCCAAGCCAGCCACTCCGGAGATTCAGG | 442 |
| | AAATCGTGGACAAGGTCAAACCGCAGCTGGAAGAGAAAACCGGCGAAACCT | |
| | ACGGAAAACTGGAAGCCGTCCAGTATAAGACTCAAGTCGTCGAATCTTACGC | |
| | AGCAATCTTCTGGGGTGGTACGAACTACTACATCAAGGTTCGTGCGGGTGAC | |
| | AACAAGTATATGCACCTGAAAGTGTTTAAGAGCCTGTGGGGTGAAATCCAGC | |
| | ATTGGAGACAGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 306 | ATGATTCCCGGCGGACTTTCGGAAGCCAAGCCAGCCACTCCGGAGATTCAGG | 443 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCGTGTTTGGCC | |
| | ATGGACCTGGGATCGTGGTACGAACTACTACATCAAGGTTCGTGCGGGTGAC | |
| | AACAAGTATATGCACCTGAAAGTGTTTAAGAGCCTGGAGCAGTGGCTGCCAA | |
| | ACCCATACTTCGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 307 | ATGATTCCCGGCGGACTTTCGGAAGCCAAGCCAGCCACTCCGGAGATTCAGG | 444 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCAGGTTAAAA | |
| | AATGGTGGGAATACTTCGTTACGAACTACTACATCAAGGTTCGTGCGGGTGA | |
| | CAACAAGTATATGCACCTGAAAGTGTTTAAGAGCCTGCAGCTGCTGTCTCAT | |
| | CAGTGGACCGAAGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCCATCACCACCACTAATGA | |
| 308 | ATGATTCCCGGCGGACTTTCGGAAGCCAAGCCAGCCACTCCGGAGATTCAGG | 445 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCATCGGTCAGAT | |
| | CTCGAACAGCAGTGGGGTACGAACTACTACATCAAGGTTCGTGCGGGTGAC | |
| | AACAAGTATATGCACCTGAAAGTGTTTAAGAGCCTGCATAGATACTGGACCA | |
| | GAGATGTTACCGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 309 | ATGATTCCCGGCGGACTTTCGGAAGCCAAGCCAGCCACTCCGGAGATTCAGG | 446 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGCATTCCATAA | |
| | ACTGGGTTGGATCCTGGGTACGAACTACTACATCAAGGTTCGTGCGGGTGAC | |
| | AACAAGTATATGCACCTGAAAGTGTTTAAGAGCCTGGGTGATGAATCTCATA | |
| | AATGGCATAAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 310 | ATGATTCCCGGCGGACTTTCGGAAGCCAAGCCAGCCACTCCGGAGATTCAGG | 447 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTTCAAAAAATG | |
| | GTGGTGGAAATACAAAGGTACGAACTACTACATCAAGGTTCGTGCGGGTGA | |
| | CAACAAGTATATGCACCTGAAAGTGTTTAAGAGCCTGAGAGTTGATGAACAT | |
| | TACATCCAGTTCGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 311 | ATGATTCCCGGCGGACTTTCGGAAGCCAAGCCAGCCACTCCGGAGATTCAGG | 448 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGCATCTCAGCG | |
| | TCTGTGGATCATCTACGGTACGAACTACTACATCAAGGTTCGTGCGGGTGAC | |
| | AACAAGTATATGCACCTGAAAGTGTTTAAGAGCCTGTCTGATTGGAGACATC | |
| | ATGGTGGTTGGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 312 | ATGATTCCCGGCGGACTTTCGGAAGCCAAGCCAGCCACTCCGGAGATTCAGG | 449 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCAGCGTGAAA | |
| | CCCATATCCTGTGGTCTGGTACGAACTACTACATCAAGGTTCGTGCGGGTGA | |
| | CAACAAGTATATGCACCTGAAAGTGTTTAAGAGCCTGTGGTTCGATAACCTG | |
| | CATAACAACTTCGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 313 | ATGATTCCCGGCGGACTTTCGGAAGCCAAGCCAGCCACTCCGGAGATTCAGG | 450 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGGTCAGTACTG | |
| | GTTCTGGGCACCAGGTGGTACGAACTACTACATCAAGGTTCGTGCGGGTGAC | |
| | AACAAGTATATGCACCTGAAAGTGTTTAAGAGCCTGCCATCTTCTGGTGGTG | |
| | GTCCAAAATGGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 314 | ATGATTCCCGGCGGACTTTCGGAAGCCAAGCCAGCCACTCCGGAGATTCAGG | 451 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCGTGATTGGGC | |
| | ATACTACATCTACTTCGGTACGAACTACTACATCAAGGTTCGTGCGGGTGAC | |
| | AACAAGTATATGCACCTGAAAGTGTTTAAGAGCCTGAGATCTGATACCCTGC | |
| | ATTACTGGAACGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATG | |
| 315 | ATGATTCCCGGCGGACTTTCGGAAGCCAAGCCAGCCACTCCGGAGATTCAGG | 452 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCATCGGTTACAA | |
| | ACTGAACTACATCTGGGGTACGAACTACTACATCAAGGTTCGTGCGGGTGAC | |
| | AACAAGTATATGCACCTGAAAGTGTTTAAGAGCCTGATCCAGTACAGATGGC | |
| | AGGATAGAGTTGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 316 | ATGATTCCCGGCGGACTTTCGGAAGCCAAGCCAGCCACTCCGGAGATTCAGG | 453 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGTTTCTCTGAC | |
| | CGAACAGCGTTGGAAAGGTACGAACTACTACATCAAGGTTCGTGCCGGTGA | |
| | CAACAAGTATATGCACCTGAAAGTGTTTAAGAGCCTGTTCCAGCCAATCAAA | |
| | CTGGCAAACGCAGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 317 | ATGATTCCCGGCGGACTTTCGGAAGCCAAGCCAGCCACTCCGGAGATTCAGG | 454 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTGGGTTTCTCA | |
| | GCAGCATTACTCTCATGGTACGAACTACTACATCAAGGTTCGTGCGGGTGAC | |
| | AACAAGTATATGCACCTGAAAGTGTTTAAGAGCCTGTTCGCATTCAGAAAAT | |
| | ACGAAGATAGAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACAACTAATGA | |
| 318 | ATGATTCCCGGCGGACTTTCGGAAGCCAAGCCAGCCACTCCGGAGATTCAGG | 455 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGATGATGATA | |
| | ACCAGCATTACGGTCTGGGTACGAACTACTACATCAAGGTTCGTGCGGGTGA | |
| | CAACAAGTATATGCACCTGAAAGTGTTTAAGAGCCTGCTGCATAACTGGTGG | |
| | AAAGGTAACTGGGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 319 | ATGATTCCCGGCGGACTTTCGGAAGCCAAGCCAGCCACTCCGGAGATTCAGG | 456 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCCAACCCGTTG | |
| | GATCTCTATCTACTGGGGTACGAACTACTACATCAAGGTTCGTGCGGGTGAC | |
| | AACAAGTATATGCACCTGAAAGTGTTTAAGAGCCTGCTGAACAGATACTACC | |
| | ATGCATCTATCGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 320 | ATGATTCCCGGCGGACTTTCGGAAGCCAAGCCAGCCACTCCGGAGATTCAGG | 457 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCACCGCATTCGC | |
| | ACCAAACAAAGCATCTGGTACGAACTACTACATCAAGGTTCGTGCGGGTGAC | |
| | AACAAGTATATGCACCTGAAAGTGTTTAAGAGCCTGAGATACTGGGCATTCA | |
| | GATACCTGTGGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 321 | ATGATTCCCGGCGGACTTTCGGAAGCCAAGCCAGCCACTCCGGAGATTCAGG | 458 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCATTACACCGA | |
| | TTTCAAATGGCATAAAGGTACGAACTACTACATCAAGGTTCGTGCGGGTGAC | |
| | AACAAGTATATGCACCTGAAAGTGTTTAAGAGCCTGCCATTCTGGGCAAACG | |
| | AACATGGTTGGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 322 | ATGATTCCCGGCGGACTTTCGGAAGCCAAGCCAGCCACTCCGGAGATTCAGG | 459 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCAACAAATTCTG | |
| | GTCTTGGGCACCAGGTGGTACGAACTACTACATCAAGGTTCGTGCGGGTGAC | |
| | AACAAGTATATGCACCTGAAAGTGTTTAAGAGCCTGGCAGATTCTGGTTTCA | |
| | ACGCAACCCCAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 323 | ATGATTCCCGGCGGACTTTCGGAAGCCAAGCCAGCCACTCCGGAGATTCAGG | 460 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGAACTGGATG | |
| | AACCAGCATGGGATTGGGGTACGAACTACTACATCAAGGTTCGTGCGGGTGA | |
| | CAACAAGTATATGCACCTGAAAGTGTTTAAGAGCCTGAGACAGCATAAAAAA | |
| | ACCACATCCAAACGAAGATTTGGTGCTGACGGGCTACCAGGTTGACAAGAA | |
| | CAAAGATGACGAGCTGACGGGTTTCGCGGCCGCTGAACAGAAGCTGATCTCC | |
| | GAAGAAGATCTGGCCGCGCATCACCATCACCACCACTAATGA | |
| 324 | ATGATTCCCGGCGGACTTTCGGAAGCCAAGCCAGCCACTCCGGAGATTCAGG | 461 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCATGAAAAAT | |
| | GGGGTTTCTGGGAACTGGGTACGAACTACTACATCAAGGTTCGTGCGGGTGA | |
| | CAACAAGTATATGCACCTGAAAGTGTTTAAGAGCCTGCATTCTCATAGATTC | |
| | CATGAAGCACATGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 325 | ATGATTCCCGGCGGACTTTCGGAAGCCAAGCCAGCCACTCCGGAGATTCAGG | 462 |
| | AAATCGTGGACAACGTCAAACCGCAGCTGGAAGAGAAAACCGGCGAAACCT | |
| | ACGGCAAACTGGAAGCCGTCCAGTATAAGACTCAAGTCGTCGTTGCAACCGC | |
| | AGATCTGATCAAAGCAGGTACGAACTACTACATCAAGGTTCGTGCGGGTGAC | |
| | AACAAGTATATGCACCTGAAAGTGTTTAAGAGCCTGCCATGGCATTACCCAC | |
| | CAAACAAATGGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 326 | ATGATTCCCGGCGGACTTTCGGAAGCCAAGCCAGCCACTCCGGAGATTCAGG | 463 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTACTACAAAGT | |
| | TATCTCTTGGTGGCATGGTACGAACTACTACATCAAGGTTCGTGCGGGTGAC | |
| | AACAAGTATATGCACCTGAAAGTGTTTAAGAGCCTGTACAAAACCCCATTCC | |
| | CACAGTACACCGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 327 | ATGATTCCCGGCGGACTTTCGGAAGCCAAGCCAGCCACTCCGGAGATTCAGG | 464 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCATTGGCATCA | |
| | CTACGATAACATCATCGGTACGAACTACTACATCAAGGTTCGTGCGGGTGAC | |
| | AACAAGTATATGCACCTGAAAGTGTTTAAGAGCCTGTACCAGCTGGTTGATC | |
| | TGGATCTGGATGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 328 | ATGATTCCCGGCGGACTTTCGGAAGCCAAGCCAGCCACTCCGGAGATTCAGG | 465 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGTTTACAAAAT | |
| | CATCACCTTCTGGTCTGGTACGAACTACTACATCAAGGTTCGTGCGGGTGAC | |
| | AACAAGTATATGCACCTGAAAGTGTTTAAGAGCCTGGAATACCCACCAGTTC | |
| | AGCAGGTTCTGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 329 | ATGATTCCCGGCGGACTTTCGGAAGCCAAGCCAGCCACTCCGGAGATTCAGG | 466 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGGTTGGATCCA | |
| | GCGTCATAACCTGATCGGTACGAACTACTACATCAAGGTTCGTGCGGGTGAC | |
| | AACAAGTATATGCACCTGAAAGTGTTTAAGAGCCTGTACTCTGAACCACCAC | |
| | CAGTTCAGAACGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 330 | ATGATTCCCGGCGGACTTTCGGAAGCCAAGCCAGCCACTCCGGAGATTCAGG | 467 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCAAAACCTGGG | |
| | AAGATACCGTTGTTGCAGGTACGAACTACTACATCAAGGTTCGTGCGGGTGA | |
| | CAACAAGTATATGCACCTGAAAGTGTTTAAGAGCCTGGAATACCTGTACAGA | |
| | CCACTGAACCCAGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 331 | ATGATTCCCGGCGGACTTTCGGAAGCCAAGCCAGCCACTCCGGAGATTCAGG | 468 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCTGGATCAGCA | |
| | TCATGTTTACCAGGTTGGTACGAACTACTACATCAAGGTTCGTGCGGGTGAC | |
| | AACAAGTATATGCACCTGAAAGTGTTTAAGAGCCTGGCACTGCTGATCCATC | |
| | ATGATCCATGGGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 332 | ATGATTCCCGGCGGACTTTCGGAAGCCAAGCCAGCCACTCCGGAGATTCAGG | 469 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCTGGAAGATGT | |
| | TTGGGGTGTTTGGGCAGGTACGAACTACTACATCAAGGTTCGTGCGGGTGAC | |
| | AACAAGTATATGCACCTGAAAGTGTTTAAGAGCCTGGAATTCCAGCATAGAA | |
| | CCTTCAACCATGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCCATCACCACCACTAATGA | |
| 333 | ATGATTCCCGGCGGACTTTCGGAAGCCAAGCCAGCCACTCCGGAGATTCAGG | 470 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTGGCTGCCATG | |
| | GACCGTTGAACTGATCGGTACGAACTACTACATCAAGGTTCGTGCGGGTGAC | |
| | AACAAGTATATGCACCTGAAAGTGTTTAAGAGCCTGGGTGAAATCTGGTCTG | |
| | CAAAACATAAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 334 | ATGATTCCCGGCGGACTTTCGGAAGCCAAGCCAGCCACTCCGGAGATTCAGG | 471 |
| | AAATCGTGGACAAGGTCAAACCGCAGCTGGAAGAGAAAACCGGCGAAACCT | |
| | ACGGCAAACTGGAAGCCGTCAAGTATAAGACTCAAGTCGTCCAGCATTTCAC | |
| | CTGGGATTGGTTCTGGGGTACGAACTACTACATCAAGGTTCGTGCGGGTGAC | |
| | AACAAGTATATGCACCTGAAAGTGTTTAAGAGCCTGCCAGAATACAAACCA | |
| | GTTGATCATACCGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 335 | ATGATTCCCGGCGGACTTTCGGAAGCCAAGCCAGCCACTCCGGAGATTCAGG | 472 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCTGTTCGAAGT | |
| | TCTGCAGGGGTCTTCTGGTACGAACTACTACATCAAGGTTCGTGCGGGTGAC | |
| | AACAAGTATATGCACCTGAAAGTGTTTAAGAGCCTGGGTGAAATCGTTTGGT | |
| | ACCAGGTTGAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 336 | ATGATTCCCGGCGGACTTTCGGAAGCCAAGCCAGCCACTCCGGAGATTCAGG | 473 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCCATACTCTTG | |
| | GCAGCATGAAGTTGTTGGTACGAACTACTACATCAAGGTTCGTGCGGGTGAC | |
| | AACAAGTATATGCACCTGAAAGTGTTTAAGAGCCTGATCGTTTTCTACCTGC | |
| | AGGAATACTACGAAGAAGATTTGGTGCTGACGGGCTACCAGGTTGACAAGA | |
| | ACAAAGATGACGAGCTGACGGGTTTCGCGGCCGCTGAACAGAAGCTGATCT | |
| | CCGAAGAAGATCTGGCCGCGCATCACCATCACCACCACTAATGA | |
| 337 | ATGATTCCCGGCGGACTTTCGGAAGCCAAGCCAGCCACTCCGGAGATTCAGG | 474 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTGGTGGCAGG | |
| | AAAACGCAGAATGGATCGGTACGAACTACTACATCAAGGTTCGTGCGGGTG | |
| | ACAACAAGTATATGCACCTGAAAGTGTTTAAGAGCCTGGGTTACGCAGTTTT | |
| | CCATAAATACCTGGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 338 | ATGATTCCCGGCGGACTTTCGGAAGCCAAGCCAGCCACTCCGGAGATTCAGG | 475 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTCTTACGATCC | |
| | AGTTTACCAGGATACCGGTACGAACTACTACATCAAGGTTCGTGCGGGTGAC | |
| | AACAAGTATATGCACCTGAAAGTGTTTAAGAGCCTGTTCTTCCATAGGACCT | |
| | CTGTTGTTTACGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 339 | ATGATTCCCGGCGGACTTTCGGAAGCCAAGCCAGCCACTCCGGAGATTCAGG | 476 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCGTGTTTTCGA | |
| | TACCCCACTGGCAGCAGGTACGAACTACTACATCAAGGTTCGTGCGGGTGAC | |
| | AACAAGTATATGCACCTGAAAGTGTTTAAGAGCCTGCCATGGCAGTACAGAA | |
| | ACAACCCAGAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 340 | ATGATTCCCGGCGGACTTTCGGAAGCCAAGCCAGCCACTCCGGAGATTCAGG | 477 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTGGTGGGATAT | |
| | CCCATGGATCTGGGTTGGTACGAACTACTACATCAAGGTTCGTGCGGGTGAC | |
| | AACAAGTATATGCACCTGAAAGTGTTTAAGAGCCTGCAGAACGATGAACTGT | |
| | ACCCATACAAAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 341 | ATGATTCCCGGCGGACTTTCGGAAGCCAAGCCAGCCACTCCGGAGATTCAGG | 478 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTACCAGAAAG | |
| | TTATCACCTGGTGGCAGGGTACGAACTACTACATCAAGGTTCGTGCGGGTGA | |
| | CAACAAGTATATGCACCTGAAAGTGTTTAAGAGCCTGTACTGGGAAAACCAT | |
| | AGAGGTCATGTTGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 342 | ATGATTCCCGGCGGACTTTCGGAAGCCAAGCCAGCCACTCCGGAGATTCAGG | 479 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCCAGTGGATCA | |
| | AATGGGGTAACATCCTGGGTACGAACTACTACATCAAGGTTCGTGCGGGTGA | |
| | CAACAAGTATATGCACCTGAAAGTGTTTAAGAGCCTGTACGAAGCAATCGAA | |
| | AGATGGCATGGTGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 343 | ATGATTCCCGGCGGACTTTCGGAAGCCAAGCCAGCCACTCCGGAGATTCAGG | 480 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGCAACCCCAG | |
| | AAAAAAAATACGTTGTTGGTACGAACTACTACATCAAGGTTCGTGCGGGTGA | |
| | CAACAAGTATATGCACCTGAAAGTGTTTAAGAGCCTGTGGAACGCAAACATC | |
| | CTGAGATACTGGGAAGATTTGGTGCTGACGGGCTACCAGGTTGACAAGAAC | |
| | AAAGATGACGAGCTGACGGGTTTCGCGGCCGCTGAACAGAAGCTGATCTCC | |
| | GAAGAAGATCTGGCCGCGCATCACCATCACCACCACTAATGA | |
| 344 | ATGATTCCCGGCGGACTTTCGGAAGCCAAGCCAGCCACTCCGGAGATTCAGG | 481 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCACCTCTGAACG | |
| | TCTGTGGATCCTGTACGGTACGAACTACTACATCAAGGTTCGTGCGGGTGAC | |
| | AACAAGTATATGCACCTGAAAGTGTTTAAGAGCCTGACCCCAAGATACGTTC | |
| | CACTGAACCCAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 345 | ATGATTCCCGGCGGACTTTCGGAAGCCAAGCCAGCCACTCCGGAGATTCAGG | 482 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGAACATCATTC | |
| | TCAGCCACTGGCACATGGTACGAACTACTACATCAAGGTTCGTGCGGGTGAC | |
| | AACAAGTATATGCACCTGAAAGTGTTTAAGAGCCTGCCATACCATGCATTCG | |
| | ATTGGTACTGGGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 346 | ATGATTCCCGGCGGACTTTCGGAAGCCAAGCCAGCCACTCCGGAGATTCAGG | 483 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTGGACCGCAG | |
| | ATTGGCTGTGGACCGTTGGTACGAACTACTACATCAAGGTTCGTGCGGGTGA | |
| | CAACAAGTATATGCACCTGAAAGTGTTTAAGAGCCTGTACGATCCATCTGAA | |
| | TGGTTCCCAAGAGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |
| 347 | ATGATTCCCGGCGGACTTTCGGAAGCCAAGCCAGCCACTCCGGAGATTCAGG | 484 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCTCTTTCCAGCG | |
| | TGAACATTACTCTTGGGGTACGAACTACTACATCAAGGTTCGTGCGGGTGAC | |
| | AACAAGTATATGCACCTGAAAGTGTTTAAGAGCCTGTTCGATATCTTCTACTT | |
| | CAACCCAATCGAAGATTTGGTGCTGACGGGCTACCAGGTTGACAAGAACAA | |
| | AGATGACGAGCTGACGGGTTTCGCGGCCGCTGAACAGAAGCTGATCTCCGA | |
| | AGAAGATCTGGCCGCGCATCACCATCACCACCACTAATGA | |
| 348 | ATGATTCCCGGCGGACTTTCGGAAGCCAAGCCAGCCACTCCGGAGATTCAGG | 485 |
| | AAATCGTGGACAAGGTCAAACCGCAGCTGGAAGAGAAAACCGGCGAAACCT | |
| | ACGGCAAACTGGAAGCCGTCCAGTATAAGACTCAAGTCGTCGGTAAAACCT | |
| | GGCATAAACATAACCGTGGTACGAACTACTACATCAAGGTTCGTGCGGGTGA | |
| | CAACAAGTATATGCACCTGAAAGTGTTTAAGAGCCTGAACCTGTACTTCTTC | |
| | TTCTACCAGACCGAAGATTTGGTGCTGACGGGCTACCAGGTTGACAAGAACA | |
| | AAGATGACGAGCTGACGGGTTTCGCGGCCGCTGAACAGAAGCTGATCTCCG | |
| | AAGAAGATCTGGCCGCGCATCACCATCACCACCACTAATGA | |

### Characteristics of CD40L-Binding Proteins

In some embodiments, a CD40L binding agent (e.g., a stefin A protein variant) can bind human CD40L as a monomer with a dissociation constant (KD) of about 1 μM or less, about 100 nM or less, about 40 nM or less, about 20 nM or less, about 10 nM or less, about 1 nM or less, or about 0.1 nM or less.

In some embodiments, a CD40L binding agent (e.g., a stefin A protein variant) can bind human CD40L with an off-rate constant (Koff), such as measured by BIACORE™ assay, of about 10⁻³ s⁻¹ (e.g., unit of 1/second) or slower; of about 10⁻⁴ s⁻¹ or slower or even of about 10⁻⁵ s⁻¹ or slower.

In some embodiments, a CD40L binding agent (e.g., a stefin A protein variant) can bind human CD40L with an association constant (Kon), such as measured by BIACORE™ assay, of at least about 10³ M⁻¹s⁻¹ or faster; at least about 10⁴ M⁻¹s⁻¹ or faster; at least about 10⁵ M⁻¹s⁻¹ or faster; or even at least about 10⁶ M⁻¹s⁻¹ or faster.

In some embodiments, a CD40L binding agent (e.g., a stefin A protein variant) can bind human CD40L with an IC50 in a competitive binding assay with human CD40L of 1 μM or less, about 100 nM or less, about 40 nM or less, about 20 nM or less, about 10 nM or less, about 1 nM or less, or about 0.1 nM or less.

In some embodiments, a CD40L binding agent (e.g., a stefin A protein variant) may have a melting temperature (Tm, e.g., temperature at which both the folded and unfolded states are equally populated) of 65° C. or higher, and preferably at least 70° C., 75° C., 80° C. or even 85° C. or higher. Melting temperature is a particularly useful indicator of protein stability. The relative proportions of folded and unfolded proteins can be determined by many techniques known to the skilled person, (Pace et al. (1997) “Measuring the conformational stability of a protein” in Protein structure: A practical approach 2: 299-321).

In some embodiments, a CD40L binding agent (e.g., a stefin A protein variant specifically binding to CD40L) is expressed in the genetically modified cell, and may be secreted and/or anchored to the membrane and presented on the cell surface.

In some embodiments, a CD40L binding agent (e.g., a stefin A protein variant specifically binding to CD40L) is expressed in the genetically modified cell, and may be localized to a specific organ or location within the cell.

In some embodiments, when a CD40L binding agent (e.g., a stefin A protein variant) expressed by the genetically modified cell is secreted, a signal sequence may be included for transport and secretion of the stefin A protein variant.

In some embodiments, the signal sequences (also referred to as signal peptides or leader sequences) are located at the N-terminus of the CD40L binding agent (e.g., stefin A protein variant). The signal sequences target the CD40L binding agent (e.g., stefin A protein variant) to the endoplasmic reticulum, and secrete the CD40L binding agent (e.g., stefin A protein variant). Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence, but is not limited to.

In some embodiments, the signal peptide is about 5 to about 40 amino acids in length (such as about 5 to about 7, about 7 to about 10, about 10 to about 15, about 15 to about 20, about 20 to about 25, or about 25 to about 30, about 30 to about 35, or about 35 to about 40 amino acids in length).

In some embodiments the signal peptide is a native signal peptide from a human protein. In other embodiments, the signal peptide is a non-native signal peptide. For example, in some embodiments, the non-native signal peptide is a mutant native signal peptide from the corresponding native secreted human protein, and can include at least one substitution, insertions and/or deletions.

In some embodiments, the signal peptide is a signal peptide or mutant thereof from a non-IgSF protein family, such as a signal peptide from an immunoglobulin (such as IgG heavy chain or IgG-kappa light chain), a cytokine (such as interleukin-2 (IL-2), or CD33), a serum albumin protein (e.g. HSA or albumin), a human azurocidin preprotein signal sequence, a luciferase, a trypsinogen (e.g. chymotrypsinogen or trypsinogen) or other signal peptide able to efficiently secrete a protein from a cell, but is not limited to.

Table 5 below lists examples of signal peptides that can be used for secretion of CD40L binding agents (e.g., stefin A protein variants) or fusion proteins thereof of the present disclosure, but is not limited thereto.

TABLE 5
Exemplary Signal Sequences

| | | SEQ |
|---|---|---|
| Native Protein | Signal Sequence | ID NO: |
| Human Serum Albumin | MKWVTFISLLFLFSSAYS | 486 |
| (HSA) | | |
| Ig kappa light chain | MDMRAPAGIFGFLLVLFP | 487 |
| | GYRS | |
| Human azurecidin | MTRLTVLALLAGLLSSRA | 488 |
| preprotein | | |
| IgG heavy chain | MELGLSWIFLLAILKGVQC | 489 |
| IgG heavy chain | MELGLRWVFLVAILEGVQC | 490 |
| IgG heavy chain | MKHLWFFLLLVAAPRWVLS | 491 |
| IgG heavy chain | MDWTWRILFLVAAATGAHS | 492 |
| IgG heavy chain | MDWTWRPLFVVAAATGVQS | 493 |
| IgG heavy chain | MEFGLSWLFLVAILKGVQC | 494 |
| IgG heavy chain | MEFGLSWVFLVALFRGVQC | 495 |
| IgG heavy chain | MDLLHKNMKHLWFFLLLVA | 496 |
| | APRWVLS | |
| IgG heavy light | MDMRVPAQLLGLLLLWLSG | 497 |
| | ARC | |
| IgG heavy light | MKYLLPTAAAGLLLLAAQP | 498 |
| | AMA | |
| Gaussia luciferase | MGVKVLFALICIAVAEA | 499 |
| Human chymotrypsinogen | MAFLWLLSCWALLGTTFG | 500 |
| sHuman interleukin-2 | MQLLSCIALILALV | 501 |
| Human trypsinogen-2 | MNLLLILTFVAAAVA | 502 |
| Human CD33 | MPLLLLLPLLWAGALA | 503 |
| Prolactin | MDSKGSSQKGSRLLLLLVV | 504 |
| | SNLLLCQGVVS | |
| Human tPA | MDAMKRGLCCVLLLCGAVF | 505 |
| | VSPS | |
| Synthetic/Consensus | MLLLLLLLLLLALALA | 506 |
| Synthetic/Consensus | MWWRLWWLLLLLLLLWPMV | 507 |
| | WA | |

In some embodiments, when the CD40L binding agent (e.g., stefin A protein variant) is anchored to a membrane or expressed on a cell surface, the CD40L binding agent (e.g., stefin A protein variant) may be directly/indirectly connected to a transmembrane domain, and may be expressed in the form of a fusion protein including a transmembrane domain. In the case of membrane-anchored or cell-surface-expressed CD40L binding agent (e.g., stefin A protein variant), the fusion protein will be described in detail in the section on fusion proteins below.

In some embodiments, the CD40L binding agent can form a multimer. In some embodiments, the CD40L binding agent can form a multimer. In some embodiments, the CD40L binding agent is a multimeric CD40L binding agent. In some embodiments, a multimeric CD40L protein is a dimer, trimer, tetramer, pentamer or higher multimer. In some embodiments, the CD40L binding agent is a multimeric stefin A protein variant.

In some embodiments, the stefin A protein variant can form a multimer.

In some embodiments, the stefin A protein variant can form a dimer, trimer, tetramer, pentamer or higher multimer.

In some embodiments, the multimer may be formed by covalently or non-covalently linked by an interaction between amino acid residues of the stefin A protein variant.

In some embodiments, the multimer may be a fusion protein formed through a fusion domain fused with a stefin A protein variant.

In some embodiments, the multimer may be formed by in-line fusion of a stefin A protein variant, and the in-line fusion protein in the form of such a multimer will be described in detail in the section on fusion proteins below.

### Fusion Protein

In some embodiments, the genetically modified cell may be introduced with a nucleic acid encoding a fusion protein comprising the CD40L binding agent (e.g., Stefin A protein variant specifically binding to CD40L). In some embodiments, the fusion protein further comprise an additional insertion, substitution and/or deletion that modulates biological activity of the CD40L binding agent (e.g., stefin A protein variant) or that gives additional biological functions. For example, the CD40L binding agent (e.g., stefin A protein variant) can fused with other polypeptide for binding to and inhibiting CD40L, modulate the circulating half-life, modulate the therapeutic half-life, modulate the stability of the AFFIMER® polypeptide, modulate cleavage by proteases, modulate dose, modulate release or bioavailability, facilitate purification, decrease deamidation, improve shelf-life, or improve or alter a particular route of administration.

For Example, the fusion protein may further comprise protease cleavage sequences, reactive groups, antibody binding domains, fusion domains for protein expression and purification (e.g. FLAG, poly-His, GST, c-myc etc.), linked molecules (e.g. biotin etc.), or other therapeutic peptide or protein, but is not limited thereto.

In some embodiments, the fusion protein may be one in which an additional peptide sequence (a fusion domain) is fused to one end and/or the other end of the CD40L binding agent (e.g., stefin A protein variant).

In some embodiments, the fusion domain, for example, may be fused to confer expression properties such as secretion from the cell, or anchoring to the cell surface, or intracellular localization; to serve as substrate or other recognition sequences for post-translational modifications; to create multimeric structures aggregating through protein-protein interactions; to alter (often to extend) serum half-life; or to alter tissue localization or tissue exclusion and other ADME properties; to add other functional proteins or peptides.

In some embodiments, the fusion domain may further comprise therapeutic peptide or protein.

In another embodiments, example, the fusion domain may be fused to for isolation and/or purification of the fusion proteins. Well known examples of such fusion domains that facilitate expression or purification include, merely to illustrate, affinity tags such as polyhistidine (e.g., a His6 tag), Strep II tag, streptavidin-binding peptide (SBP) tag, calmodulin-binding peptide (CBP), glutathione S-transferase (GST), maltose-binding protein (MBP), S-tag, HA tag, c-Myc tag, thioredoxin, protein A and protein G.

In some embodiments, the fusion protein may also comprise at least one linker separating the stefin A protein variant and or the fusion domains. In some embodiments, the “Linker” may inserted between a first polypeptide (e.g., the stefin A protein variant specifically binding to CD40L) and a second polypeptide (e.g., other stefin A protein variant or the fusion domain).

In some embodiments, the linker may be generally classified into 3 categories according to their structures: flexible linkers, rigid linkers, and in vivo cleavable linkers.

In some embodiments, besides the basic role in linking the fusion domains together, the linker may offer many other advantages for the production of fusion proteins, such as improving biological activity, increasing expression yield, and achieving desirable pharmacokinetic profiles.

In some embodiments, the linkers, preferably, should not adversely affect the expression, secretion, or activity of each domain. In some embodiments, the linkers, preferably, should not be antigenic and should not elicit an immune response.

In some embodiments, the linker is a GS linker (glycine-serin linker) including glycine and serine residues, but is not limited thereto. In some embodiments, the linker may include threonine and alanine, but is not limited to.

In some embodiments, the linker can range in length, for example from 1-50 amino acids in length, 1-22 amino acids in length, 1-10 amino acids in length, 1-5 amino acids in length, or 1-3 amino acids in length.

In some embodiments, the linker may comprise a cleavage site.

In some embodiments, the linker can be characterized as flexible. Flexible linkers are usually applied when the fusion domains require a certain degree of movement or interaction, but not limited to.

Examples of the flexible linker are described in Argos P. (1990) “An investigation of oligopeptides linking domains in protein tertiary structures [symbol] possible candidates for general gene fusion” J Mol Biol. 211:943-958, but are not limited thereto. In some embodiments, the flexible linker can provide flexibility and allows for mobility of the connecting fusion domains. The incorporation of Ser or Thr can maintain the stability of the linker in aqueous solutions by forming hydrogen bonds with the water molecules, and therefore reduces the unfavorable interaction between the linker and the protein moieties. The most commonly used flexible linkers have sequences consisting primarily of stretches of Gly and Ser residues (“GS” linker). An example of the most widely used flexible linker has the sequence of (Gly-Gly-Gly-Gly-Ser)n (SEQ ID NO: 508), but is not limited thereto. In SEQ ID NO: 508, n is an integer greater than or equal to 1, and the length of this GS linker can be optimized to achieve appropriate separation of the fusion domains by adjusting the copy number “n”. the flexible linker may further include amino acids such as Thr, Ala, Lys, and Glu in addition to Gly and Ser.

In some embodiments, the linker can be characterized as rigid. While flexible linkers have the advantage to connect the fusion domains passively and permitting certain degree of movements, the lack of rigidity of these linkers can be a limitation in certain fusion protein embodiments, such as in expression yield or biological activity. Under these situations, rigid linkers have been successfully applied to keep a fixed distance between the domains and to maintain their independent functions.

Many natural linkers exhibited α-helical structures. The α-helical structure was rigid and stable, with intra-segment hydrogen bonds and a closely packed backbone. Therefore, the stiff α-helical linkers can act as rigid spacers between protein domains (George et al. (2002) “An analysis of protein domain linkers: their classification [symbol] role in protein folding” Protein Eng. 15(11):871-9).

In general, the rigid linkers exhibit relatively stiff structures by adopting α-helical structures or by containing multiple Pro residues. Under many circumstances, they separate the functional domains more efficiently than the flexible linkers. The length of the linkers can be easily adjusted by changing the copy number to achieve an optimal distance between domains. As a result, rigid linkers are chosen when the spatial separation of the domains is critical to preserve the stability or bioactivity of the fusion proteins. In this regard, alpha helix-forming linkers with the sequence of (EAAAK)n (SEQ ID NO: 509) have been applied to the construction of many recombinant fusion proteins. Another type of rigid linkers has a Pro-rich sequence, (XP)n, with X designating any amino acid, preferably Ala, Lys, or Glu, but is not limited thereto.

In some embodiments, Table 6 lists examples of linkers that can be used for the secretion of the CD40L binding agent (e.g., stefin A protein variant) or fusion protein thereof of the present disclosure, but is not limited thereto.

TABLE 6
Exemplary Linkers

| Type | Sequence | SEQ ID NO: |
|---|---|---|
| Flexible | (GGGGS)ₙ (ex., n = 1-6) | 508 |
| Flexible | (Gly) [symbol] | 510 |
| Flexible | (Gly) [symbol] | 511 |
| Flexible | KESGSVSSEQLAQFRSLD | 512 |
| Flexible | EGKSSGSGSESKST | $13 |
| Flexible | GSAGSAAGSGEF | $14 |
| Rigid | (EAAAK)ₙ (e.g., n = 1-6) | 509 |
| Rigid | A(EAAAK)₄ALEA(EAAAK)₄A | $15 |
| Rigid | PAPAP | 516 |
| Rigid | AEAAAKEAAAKA | 517 |
| Rigid | (Ala-Pro)n(10 to 34 aa) | 518 |

[symbol]

indicates data missing or illegible when filed

Other linkers that may be used in the fusion proteins include but are not limited to:

SerGly, GGSG (SEQ ID NO: 519), GSGS (SEQ ID NO: 520), GGGS (SEQ ID NO: 521), S (GGS) n (SEQ ID NO: 522) where n is 1-7, GRA, poly(Gly), poly(Ala), GGGSGGG (SEQ ID NO: 523), ESGGGGVT (SEQ ID NO: 524), LESGGGGVT (SEQ ID NO: 525), GRAQVT (SEQ ID NO: 526), WRAQVT (SEQ ID NO: 527), and ARGRAQVT (SEQ ID NO: 528). In some embodiments, when the Fc domain is fused to the stefin A protein variant, hinge regions may also be considered linkers.

In some embodiments, the fusion protein may comprise one or more linker domains selected from below:

```
  (SEQ ID NO: 529)
SGTTSGTTRLLSGHTCFTLTGLLGTLVTMGLLT
  (SEQ ID NO: 530)
SGTSPGLSAGATVGIMIGVLVGVALI
  (SEQ ID NO: 531)
SAPVLSAVATVGITIGVLARVALI
  (SEQ ID NO: 532)
SSPDLSAGTAVSIMIGVLAGMALI
  (SEQ ID NO: 533)
TLGGNSASYTFVSLLESAVTLLLLC
  (SEQ ID NO: 534)
SGTSPGLSAGATVGIMIGVLVGVALI
```

Still other modifications that can be made to the polypeptide sequence of the CD40L binding agent (e.g., AFFIMER® polypeptide sequence) or to a flanking polypeptide moiety provided as part of a fusion protein is at least one sequence that is a site for post-translational modification by an enzyme. These can include, but are not limited to, glycosylation, acetylation, acylation, lipid-modification, palmitoylation, palmitate addition, phosphorylation, glycolipid-linkage modification, and the like.

In some embodiments, a nucleic acid encoding the fusion protein is introduced into a genetically modified cell. The genetically modified cell may be may express a fusion protein and secrete extracellularly, and/or may be anchored on a cell membrane and presented on the cell surface.

In some embodiments, the fusion protein is expressed in a genetically modified cell, and may be localized to a specific organ or location within the cell.

In some embodiments, the fusion protein is a secretory fusion protein and/or a membrane-anchored fusion protein.

In some embodiments, when the fusion protein is a secretory fusion protein, it may include a signal sequence.

In some embodiments, the signal sequence may have the same characteristics as those described in the stefin A protein variant, unless otherwise described.

In some embodiments, the fusion protein may further include at least one fusion domain.

As used herein, the term “fusion domain” refers to an additional domain or moiety capable of fusion with the CD40L binding agent (e.g., stefin A protein variant that specifically binds to CD40L) of the present disclosure.

In some embodiments, the fusion protein may include a fusion domain selected from the group consisting of an antigen-binding protein (domain), a cytokine, a half-life extension domain, a growth factor, an enzyme, and a cell-penetrating domain, but is not limited thereto.

In some embodiments, the fusion protein may further include a therapeutic peptide or protein.

In some embodiments, the “therapeutic peptide or protein” refers to any peptide or protein having a preventive or therapeutic effect on a specific disease. In some embodiments, the therapeutic peptide or protein may be a stefin A protein variant (which may be the same as or different from the stefin A protein variant specifically binding to CD40L of the present invention). It includes, without limitation, peptides and proteins reported to have preventive or therapeutic effects on a specific disease in the art.

In some embodiments, the fusion protein may include a binding domain.

In some embodiments, when the fusion protein includes a binding domain, it may have multispecificity capable of binding to at least one target molecule in addition to CD40L.

In some embodiments, the binding domain may be selected from the group consisting of, for example, a stefin A protein variant (which may be the same as or different from the stefin A protein variant specifically binding to CD40L of the present invention), an antibody or fragment thereof, an antibody-like material, an antigen-binding peptide, a ligand-binding site of a receptor (e.g. a receptor trap polypeptide), a receptor-binding ligand (e.g. a cytokine or a growth factor), an engineered T-cell receptor, and an enzyme or a catalytic fragment thereof, but is not limited thereto.

In some embodiments, examples of the binding domain may include, but are not limited to, adnectins/monobodies, affilins, affibodies, affitins, anticalin, atrimers, avimers, bicyclic peptides, C7 peptide, centyrin, carbohydrate-binding module (CBM), cys-knots, darpin, el-tandem, fynomers, knottin, Kunitz domains, O bodies, pronectin, scFv, Sac7d, Sso7d, Tn3, and the like.

In some embodiments, the fusion domain fused to the stefin A protein variant may be the same or different stefin A protein variant specifically binding to CD40L, and/or a stefin A protein variant specifically binding to another target.

In some embodiments, when the fusion domain is a stefin A protein variant specifically binding to CD40L, the two stefin A protein variants included in the fusion protein of the present invention may bind to the same or different sites of CD40L. In some embodiments, the fusion protein may bind to two sites (biparatopic) or two or more sites (multiparatopic) of CD40L.

In some embodiments, the fusion protein may include an Fc portion of an immunoglobulin. For example, when the fusion protein includes an Fc portion, it may bind to an Fc receptor to activate Fc receptor-positive cells, thereby initiating or increasing the expression of cytokines and/or costimulatory antigens, and inducing antibody-dependent cytotoxicity (ADCC).

In some embodiments, the fusion domain may be an immune checkpoint protein, an immune costimulatory receptor, a receptor (or receptor agonist), a cytokine, a growth factor, or a tumor-associated antigen, but is not limited thereto.

In some embodiments, the cytokine is used as a generic term for secretory proteins that play an important role in signaling between cells. Examples of the cytokines may include, but are not limited to, chemokines, interferons, lymphokines, interleukins, tumor necrosis factors, and the like.

In some embodiments, the growth factor refers to a naturally-occurring material or a variant thereof that may stimulate cell proliferation, wound healing, and/or cell differentiation. Examples of the growth factors may include, but are not limited to, GH, EGF, VEGF, FGF, bFGF, HGF, BMPs, M-CSF, G-CSF, GM-CSF, EPO, GDNF, IGF, KGF, BDNF, NGF, PDGF, TPO, TGF, and the like.

In some embodiments, the enzyme is used as a generic term for proteins that catalyze a biological reaction.

Examples of the enzyme may include, but are not limited to, α-chymotrypsin, lysozyme, urate oxidase, acetylcholinesterase, Thermomyces lanuginosus lipase, glucose oxidase, superoxie dismutase, caspase, β-glucosidase, Trametes versicolor laccase, alcohol oxidase, Cas9, Cas12, Cas13, Cas14, zinc-finger nuclease, TALLEN, dimethyl sulfoxide, uricase, agalsidase beta, agalsidase alfa, imiglucerase, taliglucerase alfa, velaglucerase alfa, alglucerase, sebelipase alpha, laronidase, idursulfase, elosulfase alpha, galsulfase, alglucosidase alpha, and the like.

In some embodiments, the cell-penetrating peptide is a short peptide that promotes cellular uptake and absorption of various molecules. In some embodiments, when the fusion protein includes a cell-penetrating peptide, the stefin A protein variant may be absorbed into the cell. Examples of the cell-penetrating peptide may include, but are not limited thereto, Tat, penetratin, transporant, Peptl, Pept 2, pVEC, DPV3, DPV6, R8, R9, MPG, MAP, Bip4, C105Y, melittin, and the like.

In some embodiments, the fusion protein may be fixed to the cell membrane of the genetically modified cell or may be expressed on the cell surface.

In some embodiments, when the fusion protein is fixed to a cell membrane or is expressed on a cell surface, it may further include a transmembrane domain. As used herein, the term “transmembrane domain” refers to a protein domain that spans the width of a cell membrane. In some embodiments, the transmembrane domain preferably has an alpha-helical structure, but is not limited thereto.

In some embodiments, the transmembrane domain may be a transmembrane domain derived from, for example, CD3, CD4, CD5, CD8, CD28, CD99, PDGFR, PTGFRN, etc. or a variant thereof, but is not limited thereto.

In some embodiments, the fusion protein may further include a hinge domain in addition to the transmembrane domain. As used herein, the term “hinge domain” refers to a series of amino acid sequences that exist between the extracellular domain and the transmembrane domain of a membrane-anchoring protein. In some embodiments, the hinge domain may be located between the CD40L binding agent (e.g., stefin A protein variant that specifically binds to CD40L) and the transmembrane domain.

In some embodiments, the hinge domain may be a hinge domain derived from, for example, CD3, CD4, CD5, CD8, CD28, CD99, immunoglobulin (e.g. IgG1, IgG4, IgD, etc.), PDGFR, PTGFRN, etc., or a variant thereof, but is not limited thereto. In some embodiments, the CD40L binding fusion protein comprises a hinge domain derived from an immunoglobulin (e.g., IgG1, IgG4, IgD, etc.).

In some embodiments, the fusion protein may further include a coiled coil domain. As used herein, the term “coiled coil domain” refers to a structural motif of a protein in which two to seven alpha helices are wound like a rope strand. Preferably, the coiled coil domain is configured such that two or three alpha helices are wound.

In some embodiments, the coiled coil domain may be a coiled coil domain derived from a leucine zipper, foldon, cardiac phospholamban, a water-soluble analogue of a membrane phospholamban, COMP (cartilage oligomeric matrix protein), thrombospondin 3, thrombospondin 4, or VASP (vasodilator-stimulated phosphoprotein), or a variant thereof, but is not limited thereto.

In some embodiments, the coiled coil domain may be located between the stefin A protein variant specifically binding to CD40L and the transmembrane domain.

In some embodiments, the fusion protein may further include a virus-derived peptide or protein. In some embodiments, examples of the virus-derived peptide or protein may include, but are not limited to, syncytin-1, syncytin-2, VSVG (vesicular stomatitis virus glycoprotein), F and G proteins of Nipah virus, F and H proteins of measles virus, F and H proteins of Tupaia paramnyxovirus, F and G proteins, F and H proteins, or F and HN proteins of paramyxovirus, F and G proteins of Hendra virus, F and G proteins of Henipavirus, F and H proteins of Morbillivirus, F and HN proteins of respirovirus, F and HN proteins of Sendai virus, F and HN proteins of rubulavirus, F and HN proteins of avulavirus, variants thereof, and combinations thereof.

In some embodiments, the fusion protein may further include an immunomodulatory domain or an intracellular signaling domain.

In some embodiments, the immunomodulatory domain or intracellular signaling domain is a domain located in the cytoplasmic direction of a membrane-anchoring protein, and indicates a site that activates or inhibits an immune response when a target antigen is bound to the extracellular domain.

In some embodiments, the immunomodulatory domain or intracellular signaling domain may be an immunomodulatory domain derived from CD3, CD28, CD40L, ICOS, OX40, 4-1BB, TNFR2, etc., but is not limited thereto.

In some embodiments, the fusion protein may be a chimeric antigen receptor (CAR). In some embodiments, when the fusion protein is a chimeric antigen receptor, the CD40L binding agent (e.g., stefin A protein variant that specifically binds to CD40L) may function as an extracellular binding domain.

In some embodiments, when the fusion protein is a chimeric antigen receptor, it may further include the above-described transmembrane domain, hinge domain, and intracellular signaling domain, but the present invention is not limited thereto. The fusion protein may be easily designed and prepared by changing the extracellular antigen-binding domain of various chimeric antigen receptors known in the art or analogues thereof to the CD40L binding agent (e.g., stefin A protein variant that specifically binds to CD40L) of the present disclosure.

In some embodiments, the fusion protein may further include a localization domain. In some embodiments, when the fusion protein is expressed intracellularly, it is preferable to further include a localization domain. As used herein, the term “localization domain” refers to a peptide or protein sequence that functions to localize a protein to a specific organ within a cell or a specific location within a cell. In some embodiments, the localization domain may be an organ-specific localization domain or an intracellular protein localization domain.

In some embodiments, the localization domain may be a nucleus-specific localization domain derived from VACM-1/CUL5, CXCR4, VP1, 53BP1, ING4, IER5, ERK5, Hrp1, UL79, EWS, PTHrP, Pho4, and rpL23a, a mitochondria-specific localization domain derived from ATP synthase F1b, cytochrome c oxidase polypeptide VIII, SOD2, citrate synthase, Tu translation elongation factor, etc., or a peroxisome localization domain derived from PTS1, PTS2, etc., but is not limited thereto.

In some embodiments, the fusion protein may further include a half-life extension domain. In some embodiments, the half-life extension domain is a domain or moiety that is fused to extend the half-life of the stefin A protein variant of the present invention, and examples of the half-life extension domain may include, but are not limited to, an Fc domain, an Fc-binding protein or peptide, albumin (e.g. HSA), an albumin-binding protein or peptide, transferrin, transferrin-binding protein or peptide, etc.

### Pharmacokinetic and ADME (Absorption, Distribution, Metabolism, Excretion) Properties Engineered Fusion Proteins

As used herein, a “half-life” is the amount of time it takes for a substance, such as the stefin A protein variant or the fusion protein of the present invention, to lose half of its pharmacologic or physiologic activity or concentration. Biological half-life can be affected by elimination, excretion, degradation (e.g., enzymatic) of the substance, or absorption and concentration in certain organs or tissues of the body.

In some embodiments, biological half-life can be assessed by determining the time it takes for the blood plasma concentration of the substance to reach half its steady state level (“plasma half-life”).

In some embodiments, the CD40L binding agent (e.g., stefin A protein variant) or the fusion protein comprising thereof, may not have an adequate half-life and/or pharmacokinetic profile (PK profile). Accordingly, the fusion protein may further comprise a half-life extending domain (or half-life extending moiety) to further improve the half-life or PK profile.

The term “half-life extending domain” refers to a pharmaceutically acceptable moiety, domain, or molecule directly fused or indirectly conjugated or fused through a linker or etc., to the stefin A protein variant of the present invention.

In some embodiments, the half-life extending domain may prevent or mitigate in vivo proteolytic degradation or other activity-diminishing modification of the CD40L binding agent (e.g., stefin A protein variant), increases half-life, and/or improves or alters other pharmacokinetic or biophysical properties including but not limited to increasing the rate of absorption, reducing toxicity, improving solubility, reducing protein aggregation, increasing biological activity and/or target selectivity of the CD40L binding agent (e.g., modified AFFIMER® polypeptide), increasing manufacturability, and/or reducing immunogenicity.

In some embodiments, the half-life extending domain may comprise non-proteinaceous, half-life extending moieties, such as a water soluble polymer such as polyethylene glycol (PEG) or discrete PEG, hydroxyethyl starch (HES), a lipid, a branched or unbranched acyl group, a branched or unbranched C8-C30 acyl group, a branched or unbranched alkyl group, and a branched or unbranched C8-C30 alkyl group; and proteinaceous half-life extending moieties, such as serum albumin, transferrin, adnectins (e.g., albumin-binding or pharmacokinetics extending (PKE) adnectins), Fc domain, and unstructured polypeptide, such as XTEN and PAS polypeptide (e.g. conformationally disordered polypeptide sequences composed of the amino acids Pro, Ala, and/or Ser), and a fragment of any of the foregoing, but are not limited thereto. In some embodiments, the half-life extending domain may extend the half-life of the stefin A protein variant of the present invention circulating in the serum of the subject, compared to the half-life of the fusion protein not comprising it.

In some embodiments, the half-life is extended by greater than or greater than about 1.2-fold, 1.5-fold, 2.0-fold, 3.0-fold, 4.0-fold, 5.0-fold, or 6.0-fold, but is not limited thereto. In some embodiments, the half-life is extended by more than 6 hours, more than 12 hours, more than 24 hours, more than 48 hours, more than 72 hours, more than 96 hours or more than 1 week after in vivo administration compared to the protein without the half-life extending moiety, but is not limited thereto.

In some embodiments, examples of preparation of the fusion protein further comprising the half-life extension domain are as follows, but are not limited thereto:
Genetic fusion of the CD40L binding agent (e.g., stefin A protein variant) sequence to a naturally long-half-life protein or protein domain (e.g., Fc fusion, transferrin [Tf] fusion, or albumin fusion. See, for example, Beck et al. (2011) “Therapeutic Fc-fusion proteins and peptides as successful alternatives to antibodies. MAbs. 3:1-2; Czajkowsky et al. (2012) “Fc-fusion proteins: new developments and future perspectives. EMBO Mol Med. 4:1015-28; Huang et al. (2009) “Receptor-Fc fusion therapeutics, traps, and Mimetibody technology” Curr Opin Biotechnol. 2009; 20:692-9; Keefe et al. (2013) “Transferrin fusion protein therapies: acetylcholine receptor-transferrin fusion protein as a model. In: Schmidt S, editor. Fusion protein technologies for biopharmaceuticals: applications and challenges. Hoboken: Wiley; p. 345-56; Weimer et al. (2013) “Recombinant albumin fusion proteins. In: Schmidt S, editor. Fusion protein technologies for biopharmaceuticals: applications and challenges. Hoboken: Wiley; 2013. p. 297-323; Walker et al. (2013) “Albumin-binding fusion proteins in the development of novel long-acting therapeutics. In: Schmidt S, editor. Fusion protein technologies for biopharmaceuticals: applications and challenges. Hoboken: Wiley; 2013. p. 325-43.
Genetic fusion of the CD40L binding agent (e.g., stefin A protein variant) to an inert polypeptide, e.g., XTEN (also known as recombinant PEG or “rPEG”), a homoamino acid polymer (HAP; HAPylation), a proline-alanine-serine polymer (PAS; PASylation), or an elastin-like peptide (ELP; ELPylation). See, for example, Schellenberger et al. (2009) “A recombinant polypeptide extends the in vivo half-life of peptides and proteins in a tunable manner. Nat Biotechnol. 2009; 27:1186-90; Schlapschy et al. Fusion of a recombinant antibody fragment with a homo-amino-acid polymer: effects on biophysical properties and prolonged plasma half-life. Protein Eng Des Sel. 2007; 20:273-84; Schlapschy (2013) PASylation: a biological alternative to PEGylation for extending the plasma halflife of pharmaceutically active proteins. Protein Eng Des Sel. 26:489-501. Floss et al. (2012) “Elastin-like polypeptides revolutionize recombinant protein expression and their biomedical application. Trends Biotechnol. 28:37-45. Floss et al. “ELP-fusion technology for biopharmaceuticals. In: Schmidt S, editor. Fusion protein technologies for biopharmaceuticals: application and challenges. Hoboken: Wiley; 2013. p. 372-98.
Increasing the hydrodynamic radius by chemical conjugation of the pharmacologically active peptide or protein to repeat chemical moieties, e.g., to PEG (PEGylation) or hyaluronic acid. See, for example, Caliceti et al. (2003) “Pharmacokinetic and biodistribution properties of poly(ethylene glycol)-protein conjugates” Adv Drug Delivery Rev. 55:1261-77; Jevsevar et al. (2010) PEGylation of therapeutic proteins. Biotechnol J 5:113-28; Kontermann (2009) “Strategies to extend plasma half-lives of recombinant antibodies” BioDrugs. 23:93-109; Kang et al. (2009) “Emerging PEGylated drugs” Expert Opin Emerg Drugs. 14:363-80; and Mero et al. (2013) “Conjugation of hyaluronan to proteins” Carb Polymers. 92:2163-70.
Significantly increasing the negative charge of fusing the pharmacologically active peptide or protein by polysialylation; or, alternatively, (b) fusing a negatively charged, highly sialylated peptide (e.g., carboxy-terminal peptide [CTP; of chorionic gonadotropin (CG) b-chain]), known to extend the half-life of natural proteins such as human CG b-subunit, to the biological drug candidate. See, for example, Gregoriadis et al. (2005) “Improving the therapeutic efficacy of peptides and proteins: a role for polysialic acids” Int J Pharm. 2005; 300:125-30; Duijkers et al. “Single dose pharmacokinetics and effects on follicular growth and serum hormones of a long-acting recombinant FSH preparation (FSHCTP) in healthy pituitary-suppressed females” (2002) Hum Reprod. 17:1987-93; and Fares et al. “Design of a long acting follitropin agonist by fusing the C-terminal sequence of the chorionic gonadotropin beta subunit to the follitropin beta subunit” (1992) Proc Natl Acad Sci USA. 89:4304-8. 35; and Fares “Half-life extension through O-glycosylation.
Binding non-covalently, via attachment of a peptide or protein-binding domain to the bioactive protein, to normally long-half-life proteins such as HSA, human IgG, transferrin or fibronectin. See, for example, Andersen et al. (2011) “Extending half-life by indirect targeting of the neonatal Fc receptor (FcRn) using a minimal albumin binding domain” J Biol Chem. 286:5234-41; O'Connor-Semmes et al. (2014) “GSK2374697, a novel albumin-binding domain antibody (albudAb), extends systemic exposure of extendin-4: first study in humans-PK/PD and safety” Clin Pharmacol Ther. 2014; 96:704-12. Sockolosky et al. (2014) “Fusion of a short peptide that binds immunoglobulin G to a recombinant protein substantially increases its plasma half-life in mice” PLoS One. 2014; 9:e102566.

Classical genetic fusions to long-lived serum proteins offer an alternative method of half-life extension distinct from chemical conjugation to PEG or lipids. Two major proteins have traditionally been used as fusion partners: antibody Fc domains and human serum albumin (HSA). Fc fusions involve the fusion of peptides, proteins or receptor exodomains to the Fc portion of an antibody. Both Fc and albumin fusions achieve extended half-lives not only by increasing the size of the peptide drug, but both also take advantage of the body's natural recycling mechanism: the neonatal Fc receptor, FcRn. The pH-dependent binding of these proteins to FcRn prevents degradation of the fusion protein in the endosome. Fusions based on these proteins can have half-lives in the range of 3-16 days, much longer than typical PEGylated or lipidated peptides. Fusion to antibody Fc domains can improve the solubility and stability of the peptide or protein drug. An example of a peptide Fc fusion is dulaglutide, a GLP-1 receptor agonist currently in late-stage clinical trials. Human serum albumin, the same protein exploited by the fatty acylated peptides is the other popular fusion partner. Albiglutide is a GLP-1 receptor agonist based on this platform. A major difference between Fc and albumin is the dimeric nature of Fc versus the monomeric structure of HSA leading to presentation of a fused peptide as a dimer or a monomer depending on the choice of fusion partner. The dimeric nature of an antibody and/or AFFIMER®-Fc fusion can produce an avidity effect if the antibody and/or AFFIMER® targets are spaced closely enough together or are themselves dimers. This may be desirable or not depending on the target.

Classical genetic fusions to long-lived serum proteins offer an alternative method of half-life extension distinct from chemical conjugation to PEG or lipids. Two major proteins have traditionally been used as fusion partners: antibody Fc domains and human serum albumin (HSA). Fc fusions involve the fusion of peptides, proteins or receptor exodomains to the Fc portion of an antibody. Both Fc and albumin fusions achieve extended half-lives not only by increasing the size of the peptide drug, but both also take advantage of the body's natural recycling mechanism: the neonatal Fc receptor, FcRn. The pH-dependent binding of these proteins to FcRn prevents degradation of the fusion protein in the endosome. Fusions based on these proteins can have half-lives in the range of 3-16 days, much longer than typical PEGylated or lipidated peptides. Fusion to antibody Fc domains can improve the solubility and stability of the peptide or protein drug. An example of a peptide Fc fusion is dulaglutide, a GLP-1 receptor agonist currently in late-stage clinical trials. Human serum albumin, the same protein exploited by the fatty acylated peptides is the other popular fusion domain.

The main difference between the Fc domain and albumin is that Fc is generally used in dimerization, and HSA is used in monomer structure. The fusion protein of the stefin A protein variant and the Fc domain of the present invention may be used as a dimer, but is not limited thereto.

### Fc Fusions

In some embodiments, the fusion protein may include an immunoglobulin Fc domain (an Fc domain) or a fragment or variant thereof, for example, a functional Fc region. In some embodiments, the Fc region is a FcγR null-binding Fc region. In some embodiments, the fusion protein may comprise at least one stefin A protein variant specifically binding to CD40L covalently linked through a peptide backbone (directly or indirectly) to an Fc region of an immunoglobulin. In some embodiments, the fusion protein may comprise the Fc region of an antibody (which facilitates effector functions and pharmacokinetics) and the CD40L binding agent (e.g., stefin A protein variant that specifically binds to CD40L) as part of the same polypeptide. An immunoglobulin Fc region may also be linked indirectly to at least one stefin A protein variant specifically binding to CD40L. Various linkers are known in the art for use in the fusion proteins of the invention. In some embodiments, the fusion protein comprising Fc domain may may be used as dimer, and may be used as a homodimer or a heterodimer.

In some embodiments, when the fusion protein includes an Fc domain, stability may be improved, and antibody-like properties conferred by the Fc region may be used. In some embodiments, when the fusion protein includes an Fc domain, The Fc domain is the salvage neonatal FcRn receptor pathway involving FcRn-mediated recycling of the fusion protein to the cell surface post endocytosis, avoiding lysosomal degradation and resulting in release back into the bloodstream, thus contributing to an extended serum half-life. The fusion of the Fc domain may be usefully used not only to extend the half-life, but also to isolate and purify the fusion protein of the present invention.

Fc domain will include the constant region of an antibody excluding the first constant region immunoglobulin domain. Thus, Fc domain refers to the last two constant region immunoglobulin domains of IgA, IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ2. Although the boundaries of the Fc domain may vary, the human IgG heavy chain Fc region is usually defined to comprise residues C226 or P230 to its carboxyl-terminus. Fc may refer to this region in isolation, or this region in the context of a whole antibody, antibody fragment, or Fc fusion protein. Polymorphisms have been observed at a number of different Fc positions and are also included as Fc domains as used herein. Polymorphisms have been observed at a number of different Fc positions and are also included as fusion domains as used herein.

In some embodiments, the Fc domain is a functional Fc region, the “functional Fc region” refers to an Fc domain or fragment thereof which retains the ability to bind FcRn. A functional Fc region binds to FcRn but does not possess effector function. The ability of the Fc region or fragment thereof to bind to FcRn can be determined by standard binding assays known in the art. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions can be assessed using various assays known in the art for evaluating such antibody effector functions.

In some embodiments, the Fc domain may be derived from, for example, IgG1, IgG2, IgG3 or IgG4.

In some embodiments, the Fc domain is derived from an IgG1 subclass, for example the Fc domain may comprise the following sequence:

(SEQ ID NO: 535)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK.

In some embodiments, the Fc region used in the fusion protein may comprise the hinge region. An exemplary hinge region comprises the core hinge residues spanning positions 1-16 (e.g., DKTHTCPPCPAPELLG ((SEQ ID NO: 536)) of the exemplary human IgG1 immunoglobulin Fc domain sequence provided above. In some embodiments, the fusion protein may adopt a multimeric structure (e.g., dimer) owing, in part, to the cysteine residues at positions 6 and 9 within the hinge region of the exemplary human IgG1 immunoglobulin Fc domain sequence provided above. In other embodiments, the hinge region as used herein, may further include residues derived from the CH1 and CH2 regions that flank the core hinge sequence of the exemplary human IgG1 immunoglobulin Fc domain sequence provided above. In yet other embodiments, the hinge sequence may comprise or consist of GSTHTCPPCPAPELLG (SEQ ID NO: 537) or EPKSCDKTHTCPPCPAPELLG (SEQ ID NO: 538).

In some embodiments, the hinge sequence may include at least one substitution that confer desirable pharmacokinetic, biophysical, and/or biological properties. Some exemplary hinge sequences include or is composed of:

```
(SEQ ID NO: 539)
EPKSCDKTHTCPPCPAPELLGGPS;
(SEQ ID NO: 540)
EPKSSDKTHTCPPCPAPELLGGPS;
(SEQ ID NO: 541)
EPKSSDKTHTCPPCPAPELLGGSS;
(SEQ ID NO: 542)
EPKSSGSTHTCPPCPAPELLGGSS;
  (SEQ ID NO: 543)
DKTHTCPPCPAPELLGGPS;
and
  (SEQ ID NO: 544)
DKTHTCPPCPAPELLGGSS.
```

In some embodiments, the residue P at position 18 of the human IgG1 immunoglobulin Fc domain sequence provided above may be replaced with S to ablate Fc effector function; this replacement is exemplified in hinges having the sequences:

(SEQ ID NO: 541)
EPKSSDKTHTCPPCPAPELLGGSS;
(SEQ ID NO: 542)
EPKSSGSTHTCPPCPAPELLGGSS;
and
(SEQ ID NO: 544)
DKTHTCPPCPAPELLGGSS

In another embodiment, the residues DK at positions 1-2 of the exemplary human IgG1 immunoglobulin Fc domain sequence provided above may be replaced with GS to remove a potential clip site; this replacement is exemplified in the sequence:

(SEQ ID NO: 542)
EPKSSGSTHTCPPCPAPELLGGSS.

In another embodiment, the C at the position 103 of the heavy chain constant region of human IgG1 (e.g., domains CH1-CH3), may be replaced with S to prevent improper cysteine bond formation in the absence of a light chain; this replacement is exemplified by:

(SEQ ID NO: 540)
EPKSSDKTHTCPPCPAPELLGGPS,
(SEQ ID NO: 541)
EPKSSDKTHTCPPCPAPELLGGSS,
and
(SEQ ID NO: 542)
EPKSSGSTHTCPPCPAPELLGGSS.

In some embodiments, the Fc domain may be a mammal-derived Fc domain, preferably a human Fc domain. In some embodiments, the Fc domain may be an Fc domain derived from IgG1, IgG2, IgG3 or IgG4. In some embodiments, the Fc region may possess at least about 80%, 85%, 90%, 95%, 96%, 97%, 98%, or 99% sequence identity with a native Fc region and/or with an Fc region of a parent polypeptide. In some embodiments, the Fc region may have at least about 90% sequence identity with a native Fc region and/or with an Fc region of a parent polypeptide.

In some embodiments, the Fc domain comprises an amino acid sequence selected from SEQ ID NOs: 545-558 or an Fc sequence from the examples provided by SEQ ID NOs: 545-558. It should be understood that the C-terminal lysine of an Fc domain is an optional component of a fusion protein comprising an Fc domain. In some embodiments, the Fc domain comprises an amino acid sequence selected from SEQ ID NOs: 545-558, except that the C-terminal lysine thereof is omitted. In some embodiments, the Fc domain comprises the amino acid sequence selected from SEQ ID NOs: 545-558. In some embodiments, the Fc domain comprises the amino acid sequence selected from SEQ ID NOs: 545-558 except the C-terminal lysine thereof is omitted.

TABLE 7
Exemplary Immunoglobulin Sequence

| | | SEQ ID |
|---|---|---|
| Name | Sequence | NO: |
| h1gG1a_191 | DKTHTCPPCPAPELLGGPSVPLFPPKPKDTLMISRTPEV | 545 |
| [A subtype] | TCVVVDVSHEDPEVKPNWYVDGVEVHNAKTKPREEQYNS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGBVFSCSVMHEALHNHYTQKSLSLSPGK | |
| hIgG1a_189 | DKTHTCPPCPAPELLGGPSVPLFPPKPKDTLMISRTPEV | 546 |
| [hIgG1a_191 sans | TCVVVDVSHEDPEVKPNWYVDGVEVHNAKTKPREEQYNS | |
| “GK” on C term; | TYRVVSVLTVLHQDWLNGKEYKCKVSNKALPAPIEKTIS | |
| A subtype] | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGBVFSCSVMHEALHNHYTQKSLSLSP | |
| hIgG1a_191b | DKTHTCPPCPAPELLGGPSVPLFPPKPKDTLMISRTPEV | 547 |
| [A/F subtype] | TCVVVDVSHEDPEVKPNWYVDGVEVHNAKTKPREEQYNS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGBVFSCSVMHEALHNHYTQKSLSLSPK | |
| hIgG1f_1.1_191 | DKTHTCPPCPAPELLGGPSVPLFPPKPKDTLMISRTPEV | 548 |
| [Contains five | TCVVVDVSHEDPEVKPNWYVDGVEVHNAKTKPREEQYNS | |
| point-mutations | TYRVVSVLTVLHQDWLNGKEYKCKVSNKALPAPIEKTIS | |
| to alter ADCC | KAKGQPREPQVYTLPPSRDELTKNQVSLTCLVKGFYPSD | |
| function, F | IAVEWESNGQPENNYKTTPPVLDSDGSFFLYSKLTVDKS | |
| subtype] | RWQQGBVFSCSVMHEALHNHYTQKSLSLSPGK | |
| hIgG1f_1.1_186 | EPKSSDKTHTCPPCPAPELLGGPSVPLFPPKPKDTLMIS | 549 |
| [Contains five | RTPEVTCVVVDVSHEDPEVKPNWYVDGVEVHNAKTKPRE | |
| point-mutations | EQYNSTYRVVSVLTVLHQDWLNGKEYKCKVSNKALPAPI | |
| to alter ADCC | EKTISKAKGQPREPQVYTLPPSRDELTKNQVSLTCLVKG | |
| function and | FYPSDIAVEWESNGQPENNYKTTPPVLDSDGSFFLYSKL | |
| C225S (Edlemen | TVDKSRWQQGBVFSCSVMHEALHNHYTQKSLSLSP | |
| numbering); F | | |
| subtype | | |
| hIgG1a_{N297G)_ | DKTHTCPPCPAPELLGGPSVPLFPPKPKDTLMISRTPEV | 550 |
| 191 [A subtype] | TCVVVDVSHEDPEVKPNWYVDGVEVHNAKTKPREEQYNS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGBVFSCSVMHEALHNHYTQKSLSLSPGK | |
| hIgG1a_190 | DKTHTCPPCPAPELLGGPSVPLFPPKPKDTLMISRTPEV | 551 |
| [hIgG1a_190 | TCVVVDVSHEDPEVKPNWYVDGVEVHNAKTKPREEQYNS | |
| sans “K” on C | TYRVVSVLTVLHQDWLNGKEYKCKVSNKALPAPIEKTIS | |
| term; A subtype] | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGBVFSCSVMHEALHNHYTQKSLSLSPG | |
| hIgG1a_{N297Q)_ | DKTHTCPPCPAPELLGGPSVPLFPPKPKDTLMISRTPEV | 552 |
| 191 [A subtype] | TCVVVDVSHEDPEVKPNWYVDGVEVHNAKTKPREEQYNS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGBVFSCSVMHEALHNHYTQKSLSLSPGK | |
| hIgG1a_{N297S)_ | DKTHTCPPCPAPELLGGPSVPLFPPKPKDTLMISRTPEV | 553 |
| 191 [A subtype] | TCVVVDVSHEDPEVKPNWYVDGVEVHNAKTKPREEQYNS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGBVFSCSVMHEALHNHYTQKSLSLSPGK | |
| hIgG1a_{N297A)_ | DKTHTCPPCPAPELLGGPSVPLFPPKPKDTLMISRTPEV | 554 |
| 191 [A subtype] | TCVVVDVSHEDPEVKPNWYVDGVEVHNAKTKPREEQYNS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGBVFSCSVMHEALHNHYTQKSLSLSPGK | |
| hIgG1a_{N297H)_ | DKTHTCPPCPAPELLGGPSVPLFPPKPKDTLMISRTPEV | 555 |
| 191 [A subtype] | TCVVVDVSHEDPEVKPNWYVDGVEVHNAKTKPREEQYNS | |
| | TYRVVSVLTVLHQDWLNGKEYKCKVSNKALPAPIEKTIS | |
| | KAKGQPREPQVYTLPPSRDELTKNQVSLTCLVKGFYPSD | |
| | IAVEWESNGQPENNYKTTPPVLDSDGSFFLYSKLTVDKS | |
| | RWQQGBVFSCSVMHEALHNHYTQKSLSLSPGK | |
| hIgG4 | DKRVESKYGPPCPSCPAPEFLGGPSVFLFPPKPKDTLMI | 556 |
| | SRTPEVTCVVVDVSQEDPEVKPNWYVDGVEVHNAKTKPR | |
| | EEQYNSTYRVVSVLTVLHQDWLNGKEYKCKVSNKALPAP | |
| | IEKTISKAKGQPREPQVYTLPPSRDELTKNQVSLTCLVK | |
| | GFYPSDIAVEWESNGQPENNYKTTPPVLDSDGSFFLYSK | |
| | LTVDKSRWQQGBVFSCSVMHEALHNHYTQKSLSLSPGK | |
| hIgG4_(S241P) | DKRVESKYGPPCPSCPAPEFLGGPSVFLFPPKPKDTLMI | 557 |
| | SRTPEVTCVVVDVSQEDPEVKPNWYVDGVEVHNAKTKPR | |
| | EEQYNSTYRVVSVLTVLHQDWLNGKEYKCKVSNKALPAP | |
| | IEKTISKAKGQPREPQVYTLPPSRDELTKNQVSLTCLVK | |
| | GFYPSDIAVEWESNGQPENNYKTTPPVLDSDGSFFLYSK | |
| | LTVDKSRWQQGBVFSCSVMHEALHNHYTQKSLSLSPGK | |
| hIgG1 (Contain | SEPKSCDKTHTCPPCPAPEAAGGPSVFLFPPKPKDTLMI | 558 |
| two point- | SRTPEVTCVVVDVSQEDPEVKPNWYVDGVEVHNAKTKPR | |
| mutations to | EEQYNSTYRVVSVLTVLHQDWLNGKEYKCKVSNKALPAP | |
| alter ADCC | IEKTISKAKGQPREPQVYTLPPSRDELTKNQVSLTCLVK | |
| function L20A, | GFYPSDIAVEWESNGQPENNYKTTPPVLDSDGSFFLYSK | |
| L21A) | LTVDKSRWQQGBVFSCSVMHEALHNHYTQKSLSLSPGK | |

As used herein, “Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) calls, neutrophils, and macrophages) enables those cytotoxic effector cells to bind specifically to an antigen-bearing target cell and subsequently kill the target cell with cytotoxins.

In some embodiments, the fusion protein includes an Fc domain sequence has no (or reduced) ADCC and/or complement activation or effector functionality. For example, the Fc domain may comprise a naturally disabled constant region of IgG2 or IgG4 isotype or a mutated IgG1 constant region. Examples of suitable modifications are described in EP0307434. One example comprises the substitutions of alanine residues at positions 235 and 237 (EU index numbering), but are not limited thereto.

In other embodiments, the fusion protein includes an Fc domain retain some or all Fc functionality. For example if the fusion protein comprises the Fc domain from human IgG1 or IgG3, but is not limited thereto. Levels of effector function can be varied according to known techniques, for example by mutations in the CH2 domain, for example wherein the IgG1 CH2 domain has at least one mutation at positions selected from 239 and 332 and 330, for example the mutations are selected from S239D and 1332E and A330L such that the antibody has enhanced effector function, and/or for example altering the glycosylation profile of the antigen-binding protein of the disclosure such that there is a reduction in fucosylation of the Fc region.

### Albumin Fusions

In some embodiments, the fusion protein may include an albumin sequence or a fragment thereof. In some embodiments, the albumin sequence or fragment thereof may be fused or conjugated through chemical linkage other than incorporation into the polypeptide sequence including the stefin A protein variant polypeptide. In some embodiments, the albumin, albumin variant, or albumin fragment is human serum albumin (HSA), a human serum albumin variant, or a human serum albumin fragment. Albumin serum proteins comparable to HSA are found in, for example, cynomolgus monkeys, cows, dogs, rabbits and rats. Of the non-human species, bovine serum albumin (BSA) is the most structurally similar to HSA (Kosa et al., (2007) J Pharm Sci. 96(11):3117-24). In some embodiments, the albumin may be, but is not limited to, non-human serum albumin such as cyno serum albumin or bovine serum albumin.

Mature HSA, a 585 amino acid polypeptide (approx. 67 kDa) having a serum half-life of about 20 days, is primarily responsible for the maintenance of colloidal osmotic blood pressure, blood pH, and transport and distribution of numerous endogenous and exogenous ligands. The protein has three structurally homologous domains (domains I, II and III), is almost entirely in the alpha-helical conformation, and is highly stabilized by 17 disulfide bridges. In some embodiments, the antibody and/or AFFIMER® agent can be an albumin fusion protein including at least one antibody and/or AFFIMER® polypeptide sequence and the sequence for mature human serum albumin (SEQ ID NO: 559) or a variant or fragment thereof which maintains the PK and/or biodistribution properties of mature albumin to the extent desired in the fusion protein.

(SEQ ID NO: 559)
DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVELGMFLYEYARRHPDYSVVLLLRLA
KTYETTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASQAALGL

In some embodiments, the albumin sequence can be set off from the sequence or other flanking sequences in the stefin A protein variant or the fusion protein by use of linker sequences. While unless otherwise indicated, reference herein to “albumin” or to “mature albumin” is meant to refer to HSA. However, it is noted that full-length HSA has a signal peptide of 18 amino acids (MKWVTFISLLFLFSSAYS (SEQ ID NO: 486) followed by a pro-domain of 6 amino acids (RGVFRR) (SEQ ID NO: 560); these 24 amino acid residue peptides may be referred to as the pre-pro domain. In some embodiments, the fusion proteins can be expressed and secreted using the HSA pre-pro-domain in the recombinant proteins coding sequence. In some embodiments, the fusion protein comprising the stefin A protein variant and HSA can be expressed and secreted through inclusion of other secretion signal sequences, such as described above.

In alternative embodiments, the serum albumin, for example, may be covalently coupled to the stefin A protein variant or a fusion protein comprising the same by a bond other than an amide bond such as cross-linked through chemical conjugation between amino acid sidechains on each of the stefin A protein variant or a fusion protein and the albumin.

### Serum Binding Domains

In some embodiments, the fusion protein can include a serum-binding moiety—either as part of a fusion protein (if also a polypeptide) with the antibody and/or AFFIMER® polypeptide sequence or chemically conjugated through a site other than being part of a contiguous polypeptide chain.

In some embodiments, the serum-binding polypeptide is an albumin binding domain. Albumin contains multiple hydrophobic binding pockets and naturally serves as a transporter of a variety of different ligands such as fatty acids and steroids as well as different drugs. Furthermore, the surface of albumin is negatively charged making it highly water-soluble.

As used herein, the term “albumin binding domain” as used herein refers to any chemical group capable of binding to albumin, e.g., has albumin binding affinity. Albumin binds to endogenous ligands such as fatty acids; however, it also interacts with exogenous ligands such as warfarin, penicillin and diazepam. As the binding of these drugs to albumin is reversible the albumin-drug complex serves as a drug reservoir that can enhance the drug biodistribution and bioavailability. Incorporation of components that mimic endogenous albumin-binding ligands, such as fatty acids, has been used to potentiate albumin association and increase drug efficacy.

In some embodiments, a chemical modification method that can be applied in the generation of the fusion protein to increase protein half-life is lipidation, which involves the covalent binding of fatty acids to peptide side chains. Originally conceived of and developed as a method for extending the half-life of insulin, lipidation shares the same basic mechanism of half-life extension as PEGylation, namely increasing the hydrodynamic radius to reduce renal filtration. However, the lipid moiety is itself relatively small and the effect is mediated indirectly through the non-covalent binding of the lipid moiety to circulating albumin. One consequence of lipidation is that it reduces the water-solubility of the peptide but engineering of the linker between the peptide and the fatty acid can modulate this, for example by the use of glutamate or mini PEGs within the linker. Linker engineering and variation of the lipid moeity can affect self-aggregation which can contribute to increased half-life by slowing down biodistribution, independent of albumin

In some embodiments, the albumin binding domain is albumin-binding (PKE2) adnectins (See WO2011140086 “Serum Albumin Binding Molecules”, WO2015143199 “Serum albumin-binding Fibronectin Type III Domains” and WO2017053617 “Fast-off rate serum albumin binding fibronectin type iii domains”), Albumin binding domain 3 (ABD3) of Proein G of Streptococcus G148, albumin binding domain antibody (e.g. GSK2374697, AlbudAb), or albumin binding nanobody (e.g. ATN-103 (Ozoralizumab))

### Affimer® XT

In some embodiments, the fusion protein may comprise a stefin A protein variant binding to serum protein.

In some embodiments, at least one of the solvent accessible loops of “a stefin A protein variant binding to HSA (Herein after HSA AFFIMER®)” may be characterized in that it is derived from a wild-type stefin A protein capable of binding to HSA. In some embodiments, the stefin A protein variant binding to HSA binds to HSA with a Kd of 10⁻⁶M or less.

In some embodiments, the stefin A protein variant binding to HSA binds to HSA with a Kd of 1×10⁻⁹ M to 1×10⁻⁶ M at pH 7.4 to 7.6. In some embodiments, the stefin A protein variant binding to HSA binds to HSA with a Kd of 1×10⁻⁶ M or less at pH 7.4 to 7.6. In some embodiments, the stefin A protein variant binding to HSA binds to HSA with a Kd of 1×10⁻⁷ M or less at pH 7.4 to 7.6. In some embodiments, the stefin A protein variant binding to HSA binds to HSA with a Kd of 1×10⁻⁸ M or less at pH 7.4 to 7.6. In some embodiments, the stefin A protein variant binding to HSA binds to HSA with a Kd of 1×10⁻⁹ M or less at pH 7.4 to 7.6. In some embodiments, the stefin A protein variant binding to HSA binds to HSA with a Kd of 1×10⁻¹⁰ M or less at pH 7.4 to 7.6. In some embodiments, the stefin A protein variant binding to HSA binds to HSA with a Kd of 1×10⁻¹¹ M or less at pH 7.4 to 7.6. In some embodiments, the stefin A protein variant binding to HSA binds to HSA with a Kd of 1×10⁻⁹ M to 1×10⁻⁶ M at pH 7.4. In some embodiments, the stefin A protein variant binding to HSA binds to HSA with a Kd of 1×10⁻⁶ M or less at pH 7.4. In some embodiments, the stefin A protein variant binding to HSA binds to HSA with a Kd of 1×10⁻⁷ M or less at pH 7.4. In some embodiments, the stefin A protein variant binding to HSA binds to HSA with a Kd of 1×10⁻⁸ M or less at pH 7.4. In some embodiments, the stefin A protein variant binding to HSA binds to HSA with a Kd of 1×10⁻⁹ M or less at pH 7.4. In some embodiments, the stefin A protein variant binding to HSA binds to HSA with a Kd of 1×10⁻¹⁰ M or less at pH 7.4. In some embodiments, the stefin A protein variant binding to HSA binds to HSA with a Kd of 1×10⁻¹¹ M or less at pH 7.4.

In some embodiments, the stefin A protein variant binding to HSA is derived from the wild-type human Stefin A protein having a backbone sequence and in which one or both of loop 2 (designated (Xaa)n) and loop 4 (designated (Xaa)m) are replaced with alternative loop sequences (Xaa)n and (Xaa)m.

In some embodiments, the stefin A protein variant binding to HSA may comprise an amino acid sequence represented by Formula I:

[Formula I]

FR1-(Xaa)n-FR2-(Xaa)m-FR3 (I),
wherein FR1 is an amino acid sequence having at least 70% (e.g., at least 75%, at least 80%, at least 85%, at least 90%, at least 95%, or 100%) identity to MIPGGLSEAK PATPEIQEIV DKVKPQLEEK TNETYGKLEA VQYKTQVLA (SEQ ID NO: 741);
FR2 is an amino acid sequence having at least 70% (e.g., at least 75%, at least 80%, at least 85%, at least 90%, at least 95%, or 100%) identity to GTNYYIKVRA GDNKYMHLKV FKSL (SEQ ID NO: 2);
FR3 is an amino acid sequence having at least 70% (e.g., at least 75%, at least 80%, at least 85%, at least 90%, at least 95%, or 100%) identity to EDLVLTGYQV DKNKDDELTG F (SEQ ID NO: 3);
Xaa, individually for each occurrence, is an amino acid; and
n is an integer from 3 to 20, and m is an integer from 3 to 20.

In some embodiments, FR1 is a polypeptide sequence having 80%-98%, 82%-98%, 84%-98%, 86%-98%, 88%-98%, 90%-98%, 92%-98%, 94%-98%, or 96%-98% homology with SEQ ID NO: 741. In some embodiments, FR1 is a polypeptide sequence having 80%, 82%, 84%, 86%, 88%, 90%, 92%, 94%, 96%, or 95% homology with SEQ ID NO: 741. In some embodiments, FR1 is the polypeptide sequence of SEQ ID NO: 741. In some embodiments, FR2 is a polypeptide sequence having at least 80%-96%, 84%-96%, 88%-96%, or 92%-96% homology with SEQ ID NO: 2. In some embodiments, FR2 is a polypeptide sequence having at least 80%, 84%, 88%, 92%, or 96% homology with SEQ ID NO: 2. In some embodiments, FR2 is a polypeptide sequence having at least 80%, 85%, 90%, 95% or even 98% identity with SEQ ID NO: 2. In some embodiments, FR2 is the polypeptide sequence of SEQ ID NO: 2. In some embodiments, FR3 is a polypeptide sequence having at least 80%-95%, 85%-95%, or 90%-95% homology with SEQ ID NO: 3. In some embodiments, FR3 is a polypeptide sequence having at least 80%, 85%, 90%, or 95% homology with SEQ ID NO: 3. In some embodiments, FR3 is the polypeptide sequence of SEQ ID NO: 3.

In some embodiments, the stefin A protein variant binding to HSA may comprise an amino acid sequence represented by Formula II (SEQ ID NO: 4):

[Formula II]
(SEQ ID NO: 4)
MIP-Xaa1-GLSEAKPATPEIQEIVDKVKPQLEEKTGETYGKLEAVQYKTQV-
Xaa2-(Xaa)n-Xaa3-TNYYIKVRAGDNKYMHLKVF-Xaa4-Xaa5-Xaa6-(Xaa)m-
Xaa7-D-Xaa8-VLTGYQVDKNKDDELTGF,

wherein Xaa, individually for each occurrence, is any number of an amino acid residue, more suitably three or fewer (preferably, one or two) independently selected amino acids, and n and m are each, independently, an integer from 3-20.

In some embodiments, the stefin A protein variant binding to HSA may comprise an amino acid sequence represented by below:

below:

(SEQ ID NO: 742)
MIPGGLSEAKPATPEIQEIVDKVKPQLEEKTGETYGKLEAVQYKTQVV-
(Xaa)n-GTNYYIKVRAGDNKYMHLKVEKSL-(Xaa)m-
EDLVLTGYQVDKNKDDELTGF;
(SEQ ID NO: 5)
MIPGGLSEAKPATPEIQEIVDKVKPQLEEKTGETYGKLEAVQYKTQVD-
(Xaa)n-GTNYYIKVRAGDNKYMHLKVEKSL-(Xaa)m-
EDLVLTGYQVDKNKDDELTGF;
or
(SEQ ID NO: 743)
MIPGGLSEAKPATPEIQEIVDKVKPQLEEKTGETYGKLEAVQYKTQVLA-
(Xaa)n-GTNYYIKVRAGDNKYMHLKVFKSL-(Xaa) m-EDLVLTGYQVDKNKDDELTGF

wherein Xaa, individually for each occurrence, is an amino acid residue, and n and m are each, independently, an integer from 3-20.

In some embodiments, Xaa1 is Gly, Ala, Val, Arg, Lys, Asp, or Glu; Xaa2 is Val, Asp or ‘Leu-Ala’; Xaa3 is Gly, Ala, Val, Ser or Thr; Xaa4 is Arg, Lys, Asn, Gln, Ser, Thr; Xaa5 is Gly, Ala, Val, Ser or Thr; Xaa6 is Ala, Val, Ile, Leu, Gly or Pro; Xaa7 is Gly, Ala, Val, Asp or Glu; and Xaa8 is Ala, Val, Ile, Leu, Arg or Lys.

In some embodiments, Xaa1 is Gly, Ala, Arg or Lys. In some embodiments, Xaa1 is Gly or Arg. In some embodiments, Xaa2 is Val, Asp or ‘Leu-Ala’. In some embodiments, Xaa3 is Gly, Ala, Val, Ser or Thr. In some embodiments, Xaa3 is Gly or Ser. In some embodiments, Xaa4 is Arg, Lys, Asn, Gln, Ser, Thr. In some embodiments, Xaa4 is Arg, Lys, Asn or Gln. In some embodiments, Xaa4 is Lys or Asn. In some embodiments, Xaa5 is Gly, Ala, Val, Ser or Thr. In some embodiments, Xaa5 is Gly or Ser. In some embodiments, Xaa6 is Ala, Val, Ile, Leu, Gly or Pro. In some embodiments, Xaa6 is Ile, Leu or Pro. In some embodiments, Xaa6 is Leu or Pro. In some embodiments, Xaa7 is Gly, Ala, Val, Asp or Glu. In some embodiments, Xaa7 is Ala, Val, Asp or Glu. In some embodiments, Xaa7 is Ala or Glu. In some embodiments, Xaa8 is Ala, Val, Ile, Leu, Arg or Lys. In some embodiments, Xaa8 is Ile, Leu or Arg. In some embodiments, Xaa8 is Leu or Arg.

In some embodiments, n is 3, 4, 5, 6, 7, 8, 9, 10, 11, 12, 13, 14, 15, 16, 17, 18, 19, or 20. In some embodiments, n is 8 to 10, 7 to 11, 6 to 12, 5 to 13, 4 to 14, or 3 to 15. In some embodiments, m is 3, 4, 5, 6, 7, 8, 9, 10, 11, 12, 13, 14, 15, 16, 17, 18, 19, or 20. In some embodiments, m is 8 to 10, 7 to 11, 6 to 12, 5 to 13, 4 to 14, or 3 to 15.

In some embodiments, (Xaa)n is represented by Formula IV:

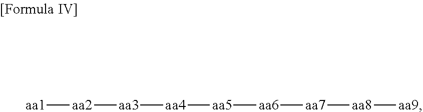

wherein aa1 is an amino acid with a neutral polar hydrophilic side chain; aa2 is an amino acid with a neutral nonpolar hydrophobic side chain; aa3 is an amino acid with a neutral nonpolar hydrophobic side chain; aa4 is an amino acid with a neutral polar hydrophilic side chain; aa5 is an amino acid with a positively charged polar hydrophilic side chain; aa6 is an amino acid with a positively charged polar hydrophilic side chain; aa7 is an amino acid with a neutral nonpolar hydrophobic side chain; aa8 is an amino acid with a neutral nonpolar hydrophobic side chain; and aa9 is an amino acid with a neutral nonpolar hydrophilic side chain.

In some embodiments, (Xaa)m is represented by Formula V:

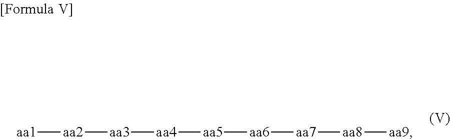

wherein aa1 is an amino acid with a neutral nonpolar hydrophobic side chain; aa2 is an amino acid with a positively charged polar hydrophilic side chain; aa3 is an amino acid with a neutral nonpolar hydrophobic side chain; aa4 is an amino acid with a positively charged polar hydrophilic side chain; aa5 is an amino acid with a neutral polar hydrophilic side chain; aa6 is an amino acid with a neutral polar hydrophilic side chain; aa7 is an amino acid with a negatively charged polar hydrophilic side chain; aa8 is an amino acid with a positively charged polar hydrophilic side chain; and aa9 is an amino acid with a neutral nonpolar hydrophilic side chain.

Examples of amino acids with a neutral nonpolar hydrophilic side chain include cysteine (Cys) and glycine (Gly). In some embodiments, the amino acid with a neutral nonpolar hydrophilic side chain is Cys. In some embodiments, the amino acid with a neutral nonpolar hydrophilic side chain is Gly.

Examples of amino acids with a neutral nonpolar hydrophobic side chain include alanine (Ala), isoleucine (Ile), leucine (Leu), methionine (Met), phenylalanine (Phe), proline (Pro), tryptophan (Trp), and valine (Val). In some embodiments, the amino acid with a neutral nonpolar hydrophobic side chain is Ala. In some embodiments, the amino acid with a neutral nonpolar hydrophobic side chain is Ile. In some embodiments, the amino acid with a neutral nonpolar hydrophobic side chain is Leu. In some embodiments, the amino acid with a neutral nonpolar hydrophobic side chain is Met. In some embodiments, the amino acid with a neutral nonpolar hydrophobic side chain is Phe. In some embodiments, the amino acid with a neutral nonpolar hydrophobic side chain is Pro. In some embodiments, the amino acid with a neutral nonpolar hydrophobic side chain is Trp. In some embodiments, the amino acid with a neutral nonpolar hydrophobic side chain is Val.

Examples of amino acids with a neutral polar hydrophilic side chain include asparagine (Asn), glutamine (Gln), serine (Ser), threonine (Thr), and tyrosine (Tyr). In some embodiments, the amino acid with a neutral polar hydrophilic side chain is Asn. In some embodiments, the amino acid with a neutral polar hydrophilic side chain is Gln. In some embodiments, the amino acid with a neutral polar hydrophilic side chain is Ser. In some embodiments, the amino acid with a neutral polar hydrophilic side chain is Thr. In some embodiments, the amino acid with a neutral polar hydrophilic side chain is Tyr.

Examples of amino acids with a positively charged polar hydrophilic side chain include arginine (Arg), histidine (His), and lysine (Lys). In some embodiments, the amino acid with a positively charged polar hydrophilic side is Arg. In some embodiments, the amino acid with a positively charged polar hydrophilic side is His. In some embodiments, the amino acid with a positively charged polar hydrophilic side is Lys.

Examples of amino acids with a negatively charged polar hydrophilic side chain include aspartate (Asp) and glutamate (Glu). In some embodiments, the amino acid with a negatively charged polar hydrophilic side chain is Asp. In some embodiments, the amino acid with a negatively charged polar hydrophilic side chain is Glu.

In some embodiments, (Xaa)n is represented by Formula IV:

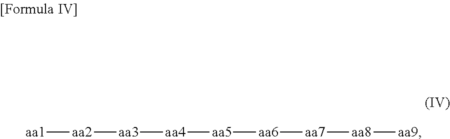

wherein aa1 is an amino acid selected from Asp, Gly, Asn, and Val; aa2 is an amino acid selected from Trp, Tyr, His, and Phe; aa3 is an amino acid selected from Trp, Tyr, Gly, Trp, and Phe; aa4 is an amino acid selected from Gln, Ala, and Pro; aa5 is an amino acid selected from Ala, Gln, Glu, Arg, and Ser; aa6 is an amino acid selected from Lys, Arg, and Tyr; aa7 is an amino acid selected from Trp and Gln; aa8 is an amino acid selected from Pro and His; and/or aa9 is an amino acid selected from His, Gly, and Gln. In some embodiments, aa1 is Asp. In some embodiments, aa1 is Gly. In some embodiments, aa1 is Asn. In some embodiments, aa2 is Trp. In some embodiments, aa2 is Tyr. In some embodiments, aa2 is His. In some embodiments, aa2 is Phe. In some embodiments, aa3 is Trp. In some embodiments, aa3 is Tyr. In some embodiments, aa3 is Gly. In some embodiments, aa3 is Trp. In some embodiments, aa3 is Phe. In some embodiments, aa4 is Gln. In some embodiments, aa4 is Ala. In some embodiments, aa4 is Pro. In some embodiments, aa5 is Ala. In some embodiments, aa5 is Gln. In some embodiments, aa5 is Glu. In some embodiments, aa5 is Arg. In some embodiments, aa5 is Ser. In some embodiments, aa6 is Lys. In some embodiments, aa6 is Arg. In some embodiments, aa6 is Tyr. In some embodiments, aa7 is Trp. In some embodiments, aa7 is Gln. In some embodiments, aa8 is Pro. In some embodiments, aa8 is His. In some embodiments, aa9 is His. In some embodiments, aa9 is Gly. In some embodiments, aa9 is Gln.

In some embodiments, (Xaa)m is represented by Formula IV:

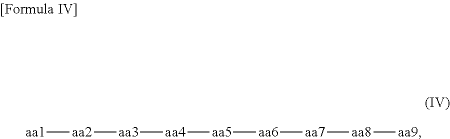

wherein aa1 is an amino acid selected from Tyr, Phe, Trp, and Asn; aa2 is an amino acid selected from Lys, Pro, His, Ala, and Thr; aa3 is an amino acid selected from Val, Asn, Gly, Gln, Ala, and Phe; aa4 is an amino acid selected from His, Thr, Lys, Trp, Lys, Val, and Arg; aa5 is an amino acid selected from Gln, Ser, Gly, Pro, and Asn; aa6 is an amino acid selected from Ser, Tyr, Glu, Leu, Lys, and Thr; aa7 is an amino acid selected from Ser, Asp, Val, and Lys; aa8 is an amino acid selected from Gly, Leu, Ser, Pro, His, Asp, and Arg; and/or aa9 is an amino acid selected from Gly, Gln, Glu, and Ala.

In some embodiments, aa1 is Tyr. In some embodiments, aa1 is Phe. In some embodiments, aa1 is Trp. In some embodiments, aa1 is Asn. In some embodiments, aa2 is Lys. In some embodiments, aa2 is Pro. In some embodiments, aa2 is His. In some embodiments, aa2 is Ala. In some embodiments, aa2 is Thr. In some embodiments, aa3 is Val. In some embodiments, aa3 is Asn. In some embodiments, aa3 is Gly. In some embodiments, aa3 is Gln. In some embodiments, aa3 is Ala. In some embodiments, aa3 is Phe. In some embodiments, aa4 is His. In some embodiments, aa4 is Thr. In some embodiments, aa4 is Lys. In some embodiments, aa4 is Trp. In some embodiments, aa4 is Lys. In some embodiments, aa4 is Val. In some embodiments, aa4 is Arg. In some embodiments, aa5 is Gln. In some embodiments, aa5 is Ser. In some embodiments, aa5 is Gly. In some embodiments, aa5 is Pro. In some embodiments, aa5 is Asn. In some embodiments, aa6 is Ser. In some embodiments, aa6 is Tyr. In some embodiments, aa6 is Glu. In some embodiments, aa6 is Leu. In some embodiments, aa6 is Lys. In some embodiments, aa6 is Thr. In some embodiments, aa7 is Ser. In some embodiments, aa7 is Asp. In some embodiments, aa7 is Val. In some embodiments, aa7 is Lys. In some embodiments, aa8 is Gly. In some embodiments, aa8 is Leu. In some embodiments, aa8 is Ser. In some embodiments, aa8 is Pro. In some embodiments, aa8 is His. In some embodiments, aa8 is Asp. In some embodiments, aa8 is Arg. In some embodiments, aa9 is Gly. In some embodiments, aa9 is Gln. In some embodiments, aa9 is Glu. In some embodiments, aa9 is Ala.

In some embodiments, (Xaa)n is represented by Formula V:

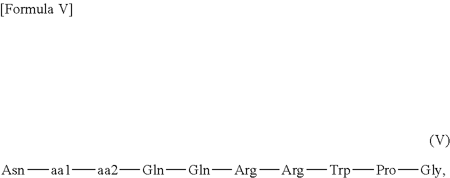

wherein aa1 is an amino acid selected from Trp and Phe; and aa2 is an amino acid selected from Tyr and Phe. In some embodiments, aa1 is Trp. In some embodiments, aa1 is Phe. In some embodiments, aa2 is Tyr. In some embodiments, aa2 is Phe.

In some embodiments, (Xaa)n is represented by Formula VI:

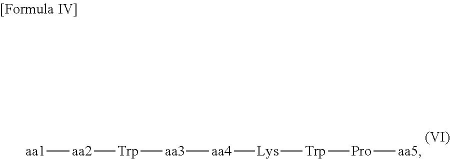

wherein aa1 is an amino acid selected from Asp and Gly; aa2 is an amino acid selected from Trp, Tyr, and Phe; aa3 is an amino acid selected from Gln and Ala; aa4 is an amino acid selected from Ala and Ser; and aa5 is an amino acid selected from His and Gly. In some embodiments, aa1 is Asp. In some embodiments, aa1 is Gly. In some embodiments, aa2 is Trp. In some embodiments, aa2 is Tyr. In some embodiments, aa2 is Phe. In some embodiments, aa3 is Gln. In some embodiments, aa3 is Ala. In some embodiments, aa4 is Ala. In some embodiments, aa4 is Ser. In some embodiments, aa5 is His. In some embodiments, aa5 is Gly.

In some embodiments, (Xaa)n is represented by Formula VII:

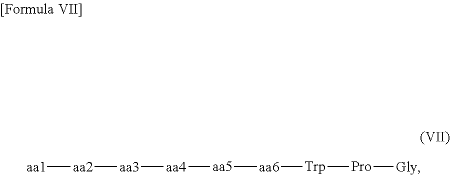

wherein aa1 is an amino acid selected from Gly and Asn; aa2 is an amino acid selected from Tyr, Phe, Trp, and His; aa3 is an amino acid selected from Trp, Tyr, and Phe; aa4 is an amino acid selected from Ala and Gln; aa5 is an amino acid selected from Ala, Ser, Gln, and Arg; and aa6 is an amino acid selected from Lys, Arg, and Tyr. In some embodiments, aa1 is Gly. In some embodiments, aa1 is Asn. In some embodiments, aa2 is Tyr. In some embodiments, aa2 is Phe. In some embodiments, aa2 is Trp. In some embodiments, aa2 is His. In some embodiments, aa3 is Trp. In some embodiments, aa3 is Tyr. In some embodiments, aa3 is Phe. In some embodiments, aa4 is Ala. In some embodiments, aa4 is Gln. In some embodiments, aa5 is Ala. In some embodiments, aa5 is Ser. In some embodiments, aa5 is Gln. In some embodiments, aa5 is Arg. In some embodiments, aa6 is Lys. In some embodiments, aa6 is Arg. In some embodiments, aa6 is Tyr.

In some embodiments, (Xaa)n is represented by Formula VIII:

[Formula VIII]
(SEQ ID NO: 561)
Gly-aal-aa2-Ala-aa3-aa4-Trp-Pro-Gly (VIII),

wherein aa1 is an amino acid selected from Tyr, Phe, and His; aa2 is an amino acid selected from Trp and Tyr; aa3 is an amino acid selected from Ala, Ser, and Arg; and aa4 is an amino acid selected from Lys and Tyr. In some embodiments, aa1 is Tyr. In some embodiments, aa1 is Phe His. In some embodiments, aa1 is His. In some embodiments, aa2 is Trp. In some embodiments, aa2 is Tyr. In some embodiments, aa3 is Ala. In some embodiments, aa3 is Ser. In some embodiments, aa3 is Arg. In some embodiments, aa4 is Lys. In some embodiments, aa4 is Tyr.

In some embodiments, (Xaa)n is represented by Formula IX:

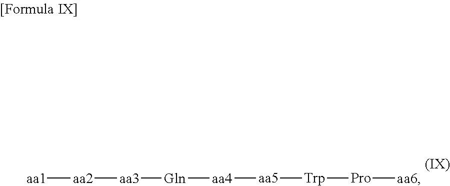

wherein aa1 is an amino acid selected from Asp and Asn; aa2 is an amino acid selected from Trp and Phe; aa3 is an amino acid selected from Trp, Tyr, and Phe; aa4 is an amino acid selected from Ala, Gln, and Arg; aa5 is an amino acid selected from Lys and Arg; and aa6 is an amino acid selected from His and Gly. In some embodiments, aa1 is Asp. In some embodiments, aa1 is Asn. In some embodiments, aa2 is Trp. In some embodiments, aa2 is Phe. In some embodiments, aa3 is Trp. In some embodiments, aa3 is Tyr. In some embodiments, aa3 is Phe. In some embodiments, aa4 is Ala. In some embodiments, aa4 is Gln. In some embodiments, aa4 is Arg. In some embodiments, aa5 is Lys. In some embodiments, aa5 is Arg. In some embodiments, aa6 is His. In some embodiments, aa6 is Gly.

In some embodiments, the stefin A protein variant binding to HSA comprises a loop 2 amino acid sequence selected from any one of SEQ ID NOs: 562 to 614 (Table 8). In some embodiments, the stefin A protein variant binding to HSA comprises a loop 4 amino acid sequence selected from any one of SEQ ID NOs: 615 to 667 (Table 8).

TABLE 8
Exemplary HSA AFFIMER ® Polypeptide Loop Sequences

| Name | Loop 2 | SEQ ID NO: | Loop 4 | SEQ ID NO: |
|---|---|---|---|---|
| HSA-00 | [symbol] | 562 | [symbol] | 615 |
| HSA-01 | [symbol] | 563 | [symbol] | 616 |
| HSA-02 | [symbol] | 564 | [symbol] | 617 |
| HSA-03 | [symbol] | 565 | [symbol] | 618 |
| HSA-04 | [symbol] | 566 | [symbol] | 619 |
| HSA-05 | [symbol] | 567 | [symbol] | 620 |
| HSA-06 | [symbol] | 568 | [symbol] | 621 |
| HSA-07 | [symbol] | 569 | [symbol] | 622 |
| HSA-08 | [symbol] | 570 | [symbol] | 623 |
| HSA-09 | [symbol] | 571 | [symbol] | 624 |
| HSA-10 | [symbol] | 572 | [symbol] | 625 |
| HSA-11 | [symbol] | 573 | [symbol] | 626 |
| HSA-12 | [symbol] | 574 | [symbol] | 627 |
| HSA-13 | [symbol] | 575 | [symbol] | 628 |
| HSA-14 | [symbol] | 576 | [symbol] | 629 |
| HSA-15 | [symbol] | 577 | [symbol] | 630 |
| HSA-16 | [symbol] | 578 | [symbol] | 631 |
| HSA-17 | [symbol] | 579 | [symbol] | 632 |
| HSA-18 | [symbol] | 580 | [symbol] | 633 |
| HSA-19 | [symbol] | 581 | [symbol] | 634 |
| HSA-20 | [symbol] | 582 | [symbol] | 635 |
| HSA-21 | [symbol] | 583 | [symbol] | 636 |
| HSA-22 | [symbol] | 584 | [symbol] | 637 |
| HSA-23 | [symbol] | 585 | [symbol] | 638 |
| HSA-24 | [symbol] | 586 | [symbol] | 639 |
| HSA-25 | [symbol] | 587 | [symbol] | 640 |
| HSA-26 | [symbol] | 588 | [symbol] | 641 |
| HSA-27 | [symbol] | 589 | [symbol] | 642 |
| HSA-28 | [symbol] | 590 | [symbol] | 643 |
| HSA-29 | [symbol] | 591 | [symbol] | 644 |
| HSA-30 | [symbol] | 592 | [symbol] | 645 |
| HSA-31 | [symbol] | 593 | [symbol] | 646 |
| HSA-32 | [symbol] | 594 | [symbol] | 647 |
| HSA-33 | [symbol] | 595 | [symbol] | 648 |
| HSA-34 | [symbol] | 596 | [symbol] | 649 |
| HSA-35 | [symbol] | 597 | [symbol] | 650 |
| HSA-36 | [symbol] | 598 | [symbol] | 651 |
| HSA-37 | [symbol] | 599 | [symbol] | 652 |
| HSA-38 | [symbol] | 600 | [symbol] | 653 |
| HSA-39 | [symbol] | 601 | [symbol] | 654 |
| HSA-40 | [symbol] | 602 | [symbol] | 655 |
| HSA-41 | [symbol] | 603 | [symbol] | 656 |
| HSA-42 | [symbol] | 604 | [symbol] | 657 |
| HSA-43 | [symbol] | 605 | [symbol] | 658 |
| HSA-44 | [symbol] | 606 | [symbol] | 659 |
| HSA-45 | [symbol] | 607 | [symbol] | 660 |
| HSA-46 | [symbol] | 608 | [symbol] | 661 |
| HSA-47 | [symbol] | 609 | [symbol] | 662 |
| HSA-48 | [symbol] | 610 | [symbol] | 663 |
| HSA-49 | [symbol] | 611 | [symbol] | 664 |
| HSA-50 | [symbol] | 612 | [symbol] | 665 |
| HSA-51 | [symbol] | 613 | [symbol] | 666 |
| Consensus | [symbol] | 614 | [symbol] | 667 |

[symbol]

indicates data missing or illegible when filed

In some embodiments, (Xaa)n comprises an amino acid sequence having at least 80% or at least 90% identity to the amino acid sequence of any one of SEQ ID NOs: 562 to 614. In some embodiments, (Xaa)n comprises an amino acid sequence having 80% to 90% identity to the amino acid sequence of any one of SEQ ID NOs: 562 to 614. In some embodiments, (Xaa)n comprises the amino acid sequence of any one SEQ ID NOs: 562 to 614.

In some embodiments, (Xaa)m comprises an amino acid sequence having at least 80% or at least 90% identity to the amino acid sequence of any one of SEQ ID NOs: 615 to 667. In some embodiments, (Xaa)m comprises an amino acid sequence having 80% to 90% identity to the amino acid sequence of any one of SEQ ID NOs: 615 to 667. In some embodiments, (Xaa)m comprises the amino acid sequence of any one of SEQ ID NOs: 615 to 667.

In some embodiments, the stefin A protein variant binding to HSA comprises an amino acid sequence selected from any one of SEQ ID NOs: 668 to 674 (Table 9).

TABLE 9
Exemplary the stefin A protein variant binding
to HSA

| | SEQ ID NO: |
|---|---|
| [symbol] | 668 |
| [symbol] | |
| [symbol] | 669 |
| [symbol] | |
| [symbol] | 670 |
| [symbol] | |
| [symbol] | 671 |
| [symbol] | |
| [symbol] | 672 |
| [symbol] | |
| [symbol] | 673 |
| [symbol] | |
| [symbol] | 674 |
| [symbol] | |

[symbol]

indicates data missing or illegible when filed

In some embodiments, the stefin A protein variant binding to HSA comprises an amino acid sequence having at least 80% or at least 90% identity to the amino acid sequence of any one of SEQ ID NOs: 668 to 674.

In some embodiments, the stefin A protein variant binding to HSA comprises an amino acid sequence having 80%- to 90% identity to the amino acid sequence of any one of SEQ ID NOs: 668 to 674.

The stefin A protein variant binding to HSA provided herein, in some embodiments, is linked to another molecule and extend the half-life of that molecule (e.g., a therapeutic polypeptide or the stefin A protein variant specifically binding to CD40L).

Provided herein is a range of the stefin A protein variant binding to HSA, with a range of binding affinities, for example, that cross-react with other species such as mouse and cynomolgus (cyno) monkey. These stefin A protein variants binding to HSA, in some embodiments, make up what is referred to as the AFFIMER XT™ platform. In some embodiments, these the stefin A protein variants binding to HSA have been shown in in vivo pharmacokinetic (PK) studies to extend, in a controlled manner, the serum half-life of any other stefin A protein variants therapeutic to which it is conjugated in a single genetic fusion. In some embodiments, AFFIMER XT™ polypeptides can also be used to extend the half-life of other peptide or protein therapeutics.

In some embodiments, the stefin A protein variants binding to HSA may increase the serum half-life of the therapeutic protein in vivo such as the stefin A protein variant specifically binding to CD40L or the fusion protein comprising the same. For example, the stefin A protein variants binding to HSA may extend the half-life of a molecule by at least 2-fold, relative to the half-life of the molecule not linked to the stefin A protein variants binding to HSA.

In some embodiments, the stefin A protein variants binding to HSA extends the half-life of a molecule by at least 3-fold, at least 4-fold, at least 5-fold, at least 6-fold, at least 7-fold, at least 8-fold, at least 9-fold, at least 10-fold, at least 20-fold, or at least 30-fold, relative to the half-life of the molecule not linked to an the stefin A protein variants binding to HSA. In some embodiments, the stefin A protein variants binding to HSA extends the half-life of a molecule by 2-fold to 5-fold, 2-fold to 10-fold, 3-fold to 5-fold, 3-fold to 10-fold, 15-fold to 5-fold, 4-fold to 10-fold, or 5-fold to 10-fold, relative to the half-life of the molecule not linked to the stefin A protein variants binding to HSA. In some embodiments, the stefin A protein variants binding to HSA extends the half-life of a molecule by at least 6 hours, at least 12 hours, at least 24 hours, at least 48 hours, at least 72 hours, at least 96 hours, for example, at least 1 week after in vivo administration, relative to the half-life of the molecule not linked to the stefin A protein variants binding to HSA.

In some embodiments, the stefin A protein variant specifically binding to CD40L has an extended serum half-life and comprises an amino acid sequence that is at least 70%, 75% 80%, 85%, 90%, 95% or even 98% identical with a sequence selected from SEQ ID NOs: 667-702 (Table 10). In some embodiments, the stefin A protein variant specifically binding to CD40L has an extended serum half-life and comprises an amino acid sequence encoded by a nucleotide sequence that is at least 70%, 75% 80%, 85%, 90%, 95% or even 98% identical with a sequence selected from SEQ ID NOs: 667-702 (Table 10). SEQ ID NOs: 703-728 (Table 11).

### PEGylation, XTEN, PAS and Other Polymers

A wide variety of macromolecular polymers and other molecules can be linked to a CD40L binding agent of the present disclosure (e.g., a stefin A protein variant specifically binding to CD40L) to modulate biological properties, and/or provide new biological properties.

In some embodiments, these macromolecular polymers can be linked to a CD40L binding agent of the present disclosure (e.g., a stefin A protein variant specifically binding to CD40L) via a naturally encoded amino acid, via a non-naturally encoded amino acid, or any functional substituent of a natural or non-natural amino acid, or any substituent or functional group added to a natural or non-natural amino acid.

In some embodiments, the molecular weight of the polymer may be of a wide range, including but not limited to, between about 100 Da and about 100,000 Da or more. The molecular weight of the polymer may be between about 100 Da and about 100,000 Da, including but not limited to, 100,000 Da, 95,000 Da, 90,000 Da, 85,000 Da, 80,000 Da, 75,000 Da, 70,000 Da, 65,000 Da, 60,000 Da, 55,000 Da, 50,000 Da, 45,000 Da, 40,000 Da, 35,000 Da, 30,000 Da, 25,000 Da, 20,000 Da, 15,000 Da, 10,000 Da, 9,000 Da, 8,000 Da, 7,000 Da, 6,000 Da, 5,000 Da, 4,000 Da, 3,000 Da, 2,000 Da, 1,000 Da, 900 Da, 800 Da, 700 Da, 600 Da, 500 Da, 400 Da, 300 Da, 200 Da, and 100 Da. In some embodiments, the molecular weight of the polymer is between about 100 Da and about 50,000 Da. In some embodiments, the molecular weight of the polymer is between about 100 Da and about 40,000 Da. In some embodiments, the molecular weight of the polymer is between about 1,000 Da and about 40,000 Da. In some embodiments, the molecular weight of the polymer is between about 5,000 Da and about 40,000 Da. In some embodiments, the molecular weight of the polymer is between about 10,000 Da and about 40,000 Da.

For this purpose, various methods including pegylation, polysialylation, HESylation, glycosylation, or recombinant PEG analogue fused to flexible and hydrophilic amino acid chain (500 to 600 amino acids) have been developed (See Chapman, (2002) Adv Drug Deliv Rev. 54. 531-545; Schlapschy et al., (2007) Prot Eng Des Sel. 20, 273-283; Contermann (2011) Curr Op Biotechnol. 22, 868-876; Jevsevar et al., (2012) Methods Mol Biol. 901, 233-246).

In some embodiments, examples of polymers include, but are not limited to, polyalkyl ethers and alkoxy-capped analogs thereof (e.g., polyoxyethylene glycol, polyoxyethylene/propylene glycol, and methoxy or ethoxy-capped analogs thereof, especially polyoxyethylene glycol, the latter is also known as polyethylene glycol or PEG); discrete PEG (dPEG); polyvinylpyrrolidones; polyvinylalkyl ethers; polyoxazolines, polyalkyl oxazolines and polyhydroxyalkyl oxazolines; polyacrylamides, polyalkyl acrylamides, and polyhydroxyalkyl acrylamides (e.g., polyhydroxypropylmethacrylamide and derivatives thereof); polyhydroxyalkyl acrylates; polysialic acids and analogs thereof; hydrophilic peptide sequences; polysaccharides and their derivatives, including dextran and dextran derivatives, e.g., carboxymethyldextran, dextran sulfates, aminodextran; cellulose and its derivatives, e.g., carboxymethyl cellulose, hydroxyalkyl celluloses; chitin and its derivatives, e.g., chitosan, succinyl chitosan, carboxymethylchitin, carboxymethylchitosan; hyaluronic acid and its derivatives; starches; alginates; chondroitin sulfate; albumin; pullulan and carboxymethyl pullulan; polyaminoacids and derivatives thereof, e.g., polyglutamic acids, polylysines, polyaspartic acids, polyaspartamides; maleic anhydride copolymers such as: styrene maleic anhydride copolymer, divinylethyl ether maleic anhydride copolymer; polyvinyl alcohols; copolymers thereof; terpolymers thereof; mixtures thereof; and derivatives of the foregoing.

In some embodiments, the polymer selected may be water soluble so that the fusion protein to which it is attached does not precipitate in an aqueous environment, such as a physiological environment. In some embodiments, the water-soluble polymer may be any structural form including but not limited to linear, forked or branched. Typically, the water-soluble polymer is a poly(alkylene glycol), such as poly(ethylene glycol) (PEG), but other water-soluble polymers can also be employed. By way of example, PEG is used to describe some embodiments of this disclosure. In some embodiments, the polymer may be pharmaceutically acceptable

As used herein, the term “PEG” is used broadly to encompass any polyethylene glycol molecule, without regard to size or to modification at an end of the PEG, and can be represented as linked to the stefin A protein variant by the formula below:

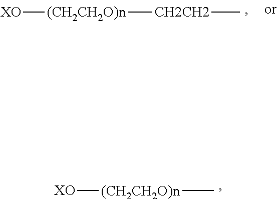

where n is 2 to 10,000 and X is H or a terminal modification, including but not limited to, a C1-4 alkyl, a protecting group, or a terminal functional group. In some cases, a PEG used in the polypeptides of the disclosure terminates on one end with hydroxy or methoxy, e.g., X is H or CH3 (“methoxy PEG”).

It is noted that the other end of the PEG, which is shown in the above formulas by a terminal “-”, may attach to the stefin A protein variant containing polypeptide via a naturally-occurring or non-naturally encoded amino acid. For instance, the attachment may be through an amide, carbamate or urea linkage to an amine group (including but not limited to, the epsilon amine of lysine or the N-terminus) of the polypeptide. Alternatively, the polymer is linked by a maleimide linkage to a thiol group (including but not limited to, the thiol group of cysteine)—which in the case of attachment to the fusion protein sequence per se requires altering a residue in the stefin A protein variant sequence to a cysteine.

The number of water-soluble polymers linked to a CD40L binding agent of the present disclosure (e.g., a stefin A protein variant or fusion protein), including, the extent of PEGylation or glycosylation, can be adjusted to provide an altered (including but not limited to, increased or decreased) pharmacologic, pharmacokinetic or pharmacodynamic characteristic such as in vivo half-life. In some embodiments, the half-life of the resulting CD40L binding agent of the present disclosure (e.g., stefin A protein variant or fusion protein) is increased at least about 10, 20, 30, 40, 50, 60, 70, 80, 90 percent, 2-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 50-fold, or at least about 100-fold over an unmodified polypeptide.

Another variation of polymer system useful to modify the PK or other biological properties of a CD40L binding agent of the present disclosure (e.g., a stefin A protein variant or fusion protein) are the use of unstructured, hydrophilic amino acid polymers that are functional analogs of PEG. For example, hydrophilic amino acid polymers that are functional analogs of PEG may be particularly useful as part of a fusion protein with the stefin A protein variant. The inherent biodegradability of the polypeptide platform makes it attractive as a potentially more benign alternative to PEG. Another advantage is the precise molecular structure of the recombinant molecule in contrast to the polydispersity of PEG.

Unlike HSA and Fc peptide fusions, in which the three-dimensional folding of the fusion partner needs to be maintained, the recombinant fusions to unstructured partners can, in many cases, be subjected to higher temperatures or harsh conditions such as HPLC purification.

One of the more advanced of this class of polypeptides is termed XTEN (Amunix) and is 864 amino acids long and comprised of six amino acids (A, E, G, P, S and T). See Schellenberger et al. “A recombinant polypeptide extends the in vivo half-life of peptides and proteins in a tunable manner” 2009 Nat Biotechnol. 27(12):1186-90. Enabled by the biodegradable nature of the polymer, this is much larger than the 40 KDa PEGs typically used and confers a concomitantly greater half-life extension. The fusion of XTEN to a CD40L binding fusion protein (e.g., an AFFIMER® containing polypeptide) should result in halflife extension of the fusion protein by 60- to 130-fold over the unmodified polypeptide. A second polymer based on similar conceptual considerations is PAS (XL-Protein GmbH). Schlapschy et al. “PASYlation: a biological alternative to PEGylation for extending the plasma half-life of pharmaceutically active proteins” 2013 Protein Eng Des Sel. 26(8):489-501. A random coil polymer comprised of an even more restricted set of only three small uncharged amino acids, proline, alanine and serine. As with Fc, HSA and XTEN, the PAS modification can be genetically encoded with a CD40L binding agent of the present disclosure (e.g., a stefin A protein variant) to produce an inline fusion protein when expressed.

### In-Line Fusions

In some embodiments, the fusion protein may be selected from any one of the polypeptides provided in Table 10 or encoded by any one of the polynucleotides provided in Table 11.

TABLE 10
Exemplary Encoded Half-Life Extension In-Line Fusion
Anti-CD40L AFFIMER® Polypeptide Sequences

| | Amino Acid | SEQ ID |
|---|---|---|
| [symbol] | [symbol] | 677 |
| | [symbol] | |
| | [symbol] | |
| | [symbol] | |
| | [symbol] | |
| | [symbol] | |
| [symbol] | [symbol] | 678 |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 679 |
| | [symbol] | |
| | [symbol] | |
| | [symbol] | |
| | [symbol] | |
| | [symbol] | |
| [symbol] | [symbol] | 680 |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 681 |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 682 |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 683 |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 684 |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 685 |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 686 |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 687 |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 688 |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 689 |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 690 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 691 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 692 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 693 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 694 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 695 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 696 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 697 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 698 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 699 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 700 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 701 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 702 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |

[symbol]

indicates data missing or illegible when filed

In some embodiments, the nucleic acid encoding the fusion protein may be characterized in that it is selected from the nucleic acids provided in Table 11, but is not limited thereto. Based on the amino acid sequence of the stefin A protein specifically binding to CD40L of the present invention and the nucleic acid sequence encoding the same, the polypeptide sequence of the fusion domain included in the fusion protein of the present invention and the nucleic acid sequence encoding the same, It will be apparent that the nucleic acid sequence encoding the fusion protein can be designed and predicted.

TABLE 11
Exemplary Nucleic Acids Encoding Fusion Proteins
Comprising Inline Fused Stefin A Protein Variants for Half-
Life Extension

| Clone# | Nucleic Acid | SEQ ID No: |
|---|---|---|
| [symbol] | [symbol] | 703 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 704 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 708 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 709 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 710 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 711 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 712 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 713 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 714 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 715 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 716 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 717 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 718 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 719 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 720 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 721 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 722 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 723 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 724 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 725 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 726 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 727 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| [symbol] | [symbol] | 728 |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |
| | [symbol] | |

[symbol]

indicates data missing or illegible when filed

### Host Cells and Genetically Modified Cells

In some embodiments, a nucleic acid encoding a CD40L binding agent of the present disclosure (e.g., a stefin A protein variant specifically binding to CD40L described above) and/or the fusion protein including the same is introduced into a host cell.

As used herein, the term “host cell” refers to a cell before introduction, for introducing a nucleic acid encoding a CD40L binding agent (e.g., a stefin A protein variant specifically binding to CD40L) and/or the fusion protein including the same.

As used herein, the term “genetically modified cell” refers to a cell that has been engineered to include an exogenous nucleic acid. Genetically modified cells include cells that contain an exogenous nucleic acid, whether or not such exogenous nucleic acid is integrated into the genome of the cell. In some embodiments, a genetically modified cell is engineered to express a CD40L binding agent (e.g., a cell expressing a stefin A protein variant and/or a fusion protein including the same). In some embodiments, a genetically modified cell is produced by introducing a nucleic acid encoding a CD40L binding agent (e.g., Stefin A protein variant that binds CD40L) of the present disclosure and/or fusion protein including the same.

However, the present invention excludes from the scope of rights genetically engineered cells for use in the manufacturing of Stefin A protein variants that specifically bind to CD40L and/or fusion proteins containing the same.

In some embodiments, the genetically modified cell may include additional genetic modification in addition to the introduction of a nucleic acid encoding a CD40L binding agent of the present disclosure (e.g., a stefin A protein variant that specifically binds to CD40L) and/or the fusion protein including the same.

In some embodiments, host cells may be genetically modified to include a nucleic acid encoding a CD40L binding agent of the present disclosure (e.g., a stefin A protein variant specifically binding to CD40L) and/or the fusion protein including the same, in order to produce genetically modified cells that express the CD40L binding agent and/or the fusion protein including the same.

Genetically modified cells in accordance with the present disclosure includes all types of cells such as eukaryotic cells, prokaryotic cells, etc. may be used, and examples of the host cell may include, but are not limited to, bacterial cells such as Escherichia coli, Streptomyces, Salmonella typhimurium, etc., yeast cells, fungal cells such as Pichia pastoris, etc., insect cells such as Drosophila, Spodoptera Sf9 cells, etc., animal cells such as CHO, COS, NSO, 293, and bow melanoma cells, and plant cells.

In some embodiments, the host cell may be selected from the group consisting of a stem cell, an immune cell, and a somatic cell.

In some embodiments, the host cell is a cell derived from nature, for example, an animal, preferably a mammal, more preferably a human, or a cell engineered through cell engineering or genetic engineering.

As used herein, the term “stem cell” refers to a cell capable of differentiating into various types of cells constituting a biological tissue, and collectively refers to undifferentiated cells in the pre-differentiation stage which may be obtained from each tissue of embryo, fetus, and adult body. Stem cells are differentiated into specific cells by differentiation stimuli (environment), and unlike cells in which differentiation is completed and cell division is stopped, stem cells are capable of self-renewal by cell division to thus enable proliferation (expansion), and may be differentiated into other cells by different environments or different differentiation stimuli, meaning they have plasticity in differentiation.

In some embodiments, examples of the stem cells may include, but are not limited to, pluripotent stem cells, multipotent stem cells, and unipotent stem cells, depending on the differentiation potential thereof.

In some embodiments, the pluripotent stem cells are stem cells capable of differentiating into three germ layers constituting a living body, and examples thereof may include, but are not limited to, embryonic stem cells, induced pluripotent stem cells (iPS), and the like.

In some embodiments, the present disclosure provides genetically modified pluripotent stem (PS) cells that express a CD40L binding agent and/or fusion thereof. In some embodiments, provided PS cells comprise a nucleic acid encoding a CD40L binding agent and/or fusion thereof. In some embodiments, provided genetically modified PS cells are iPS cells. In some embodiments, provided genetically modified PS cells are ES cells.

In some embodiments of the present disclosure, a genetically modified PS cell that expresses a CD40L binding agent (e.g., a stefin A protein variant) or the fusion protein thereof is produced by introducing a nucleic acid encoding the CD40L binding agent or the fusion protein including the same into a PS cell (e.g., an iPS cell or ES cell).

Multipotent stem cells are cells having the potential to differentiate progenitor cells into cells belonging to a certain family. Examples of the multipotent stem cells may include, but are not limited to, hematopoietic stem cells, mesenchymal stem cells, neural stem cells, and the like.

In some embodiments, the stem cells may be mesenchymal stem cells. As used herein, the terms “mesenchymal stem cell” and “mesenchymal stromal cell” are used interchangeably to refer to a cell capable of differentiating into osteoblasts, adipocytes, chondrocytes, and the like, which may be differentiated from mesoderm among the three germ layers of embryonic tissue. The mesenchymal stem cells may be extracted from bone marrow, adipose tissue, umbilical cord blood, synovial membrane, trabecular bone, subpatellar fat pad, etc. The mesenchymal stem cells are known to 1) inhibit the activity and proliferation of T lymphocytes and B lymphocytes, 2) inhibit the activity of natural killer cells (NK cells), and 3) enable allotransplantation and xenotransplantation by virtue of immunomodulatory activity of regulating the functions of dendritic cells and macrophages.

In some embodiments, the present disclosure provides genetically modified mesenchymal stromal cells (MSCs) that express a CD40L binding agent and/or fusion thereof. In some embodiments, provided MSCs comprise a nucleic acid encoding a CD40L binding agent and/or fusion thereof.

In some embodiments of the present disclosure, a genetically modified mesenchymal stromal cell that expresses a CD40L binding agent (e.g., a stefin A protein variant) or the fusion protein thereof is produced by introducing a nucleic acid encoding the CD40L binding agent or the fusion protein including the same into a mesenchymal stem cell.

In some embodiments, a CD40L binding agent (e.g., a stefin A protein variant that specifically binds to CD40L) exhibits a CD40L antagonistic effect, and thus an immunosuppressive effect such as inhibition of T-cell and/or B-cell activity. Therefore, in some embodiments, by introducing a nucleic acid encoding a CD40L binding agent (e.g., a stefin A protein variant) into a mesenchymal stem cell (MSC) as the host cell, novel immunomodulatory activity was acquired through the CD40L antagonistic effect by the stefin A protein variant while maintaining the immunomodulatory effect of the mesenchymal stem cell (e.g. T-cell activation inhibitory effect), confirming that immunity was very effectively inhibited and also that a vastly superior therapeutic effect on immune-related diseases such as GVHD or autoimmune diseases was exhibited.

In some embodiments, the mesenchymal stem cell may be derived from a pluripotent stem cell. In some embodiments, the mesenchymal stem cell enables long-term subculture. The method of producing the mesenchymal stem cell from the pluripotent stem cell and the long-term subculture method are well known in the art, and for example, Korean Patent Application Publication No. 10-2021-0072734, Korean Patent No. 10-1135636, etc. disclose a method of maintaining undifferentiation potency and marker expression characteristics even after tens of passages.

In some embodiments, the mesenchymal stem cell may express at least one selected from among CD29, CD44, CD73, CD90, CD90, and CD105. In some embodiments, the mesenchymal stem cell may not express at least one cell surface marker selected from among CD11b, CD14, CD34, CD45, CD79, HLA-DR, TRA-1-60, and TRA-1-81. In some embodiments, the mesenchymal stem cell may not express at least one cell surface marker selected from among CD14, CD19, CD34, CD45, HLA-DR, SSEA-3, TRA-1-60, TRA-1-81, Nanog and Oct3/4.

In some embodiments, the mesenchymal stem cell may express at least one cell surface marker selected from among CD29, CD44, CD73, CD90, and CD105.

In some embodiments, the MSCs express CD90. In some embodiments, at least 85%, at least 90%, at least 91%, at least 92%, at least 93%, at least 94%, at least 95%, at least 96%, at least 97%, or at least 98% of the MSCs express CD90.

In some embodiments, the mesenchymal stem cell is capable of maintaining at least 70%, at least 75%, at least 80%, at least 85%, at least 90%, or at least 95% of expression of the cell surface marker after at least 10 passages, at least 11 passages, at least 12 passages, at least 13 passages, at least 14 passages, at least 15 passages, at least 16 passages, at least 17 passages, at least 18 passages, at least 19 passages, or at least 20 passages.

In some embodiments, the passage may be based on an increase in the number of cells by a certain fold or more, and for example, proliferation of the number of cells at least 2-fold, 3-fold or more, 4-fold or more, 5-fold or more, 6-fold or more, 7-fold or more, or 8-fold or more as compared to the start of the previous passage may be determined to be 1 passage, but the present invention is not limited thereto.

In some embodiments, the mesenchymal stem cell may not express at least one cell surface marker selected from among CD14, CD19, CD34, CD45, HLA-DR, SSEA-3, TRA-1-60, TRA-1-81, Nanog and Oct3/4. In some embodiments, the mesenchymal stem cell may not express at least one cell surface marker selected from among CD11b, CD14, CD34, CD45, CD79, HLA-DR, TRA-1-60, and TRA-1-81. In some embodiments, the mesenchymal stem cell may not express at least one cell surface marker selected from among CD34, CD45, HLA-DR, TRA-1-60, and TRA-1-81.

In some embodiments, the host cell may be an immune cell.

As used herein, the term “immune cell” refers to all types of cells constituting the immune system. A cell therapeutic agent using the immunomodulatory activity of immune cells is used for the treatment of various diseases such as cancer and autoimmune diseases, but due to the non-specific effects thereof, immune cell therapeutic agents engineered to enable target-specific immune regulation, such as chimeric antigen receptors, are of great interest.

A CD40L binding agent (e.g., a stefin A protein variant) targets CD40L involved in immune regulation, and in particular, since it has an antagonistic ability to CD40L, when a nucleic acid encoding the CD40L binding agent (e.g., stefin A protein variant specifically binding to CD40L) and/or the fusion protein including the same is introduced into immune cells, a vastly superior immunomodulatory effect may be exhibited.

In some embodiments, the immune cells may be selected from the group consisting of T cells, B cells, natural killer (NK) cells, nkT cells, and dendritic cells, but are not limited thereto.

In some embodiments, the immune cells are those isolated from the human body, blood, or peripheral blood mononuclear cells (PBMCs), or those differentiated from stem cells (preferably pluripotent stem cells), but are not limited thereto.

In some embodiments, when a nucleic acid encoding a fusion protein (e.g. a chimeric antigen receptor) including a CD40L binding agent (e.g., a stefin A protein variant specifically binding to CD40L) as an extracellular binding domain is introduced into the immune cells, the resulting cells may be used as a cell therapeutic agent, such as CAR-T or CAR-NK.

In some embodiments, the host cell may be a somatic cell.

As used herein, the term “somatic cell” refers to any type of cells except gametes, constituting the body of an animal, preferably a human. Examples of cell therapeutic agents using somatic cells are well known in the art. For example, epidermal cells such as keratinocytes, fibroblasts, and mucous membranes may be used for the treatment of skin burns, scars, cosmetic purposes, etc., and chondrocytes, adipocytes, islet cells, and skeletal myoblasts may be used for the treatment of diseases such as degenerative arthritis, subcutaneous fat obliteration, etc., but the present invention is not limited thereto.

When a gene encoding a CD40L binding agent (e.g., a stefin A protein variant specifically binding to CD40L) and/or the fusion protein including the same is introduced into a somatic cell as the host cell of the present invention, an immunomodulatory effect through inhibition of CD40L activity may be exhibited.

In some embodiments, the genetically modified cell enables secretion of a CD40L binding agent (e.g., a stefin A protein variant specifically binding to CD40L) and/or the fusion protein including the same, expression thereof on the cell membrane, and/or localization thereof to a specific site in the cell.

Method of Introducing Nucleic Acid into Host Cell

As used herein, the term “introduction” refers to allowing the host cell to receive a foreign gene (nucleic acid) that the host cell does not have.

In some embodiments, a nucleic acid encoding a CD40L binding agent (e.g., a stefin A protein variant specifically binding to CD40L) or a fusion protein including the same may be introduced into a host cell using a vector including the same.

As used herein, a “vector” is a means for expressing a target gene in a host cell, and examples of the vector may include, but are not limited to, viral vectors such as adenoviral vector, retroviral vector, adeno-associated viral vector, and vectors derived from viruses such as vaccinia virus (Puhlmann M. et al., Human Gene Therapy, 10:649-657 (1999); Ridgeway, 467-492 (1988); Baichwal and Sugden, In: Kucherlapati R., ed. Gene transfer. New York: Plenum Press, 117-148 (1986) and Coupar et al., Gene, 68:1-10(1988)), lentivirus (Wang G. et al., J. Clin. Invest., 104(11):R55-62(1999)), herpes simplex virus (Chamber R., et al., Proc. Natl. Acad. Sci USA, 92:1411-1415 (1995)), poxvirus (GCE, NJL, Krupa M., Esteban M., Curr. Gene Ther. 8(2):97-120 (2008)), reovirus, measles virus, Semliki Forest virus, and poliovirus, and non-viral vectors such as plasmid vectors (Sambrook et al., 1989) and mini circles (Yew et al. 2000 Mol. Ther. 1(3), 255-62).

The vector may typically include at least one component selected from among a signal sequence, an origin of replication, at least one antibiotic resistance marker gene, an enhancer element, a promoter, and a transcription termination sequence, but the present invention is not limited thereto. The nucleic acid encoding the stefin A protein variant of the present invention or the fusion protein including the same may be operably linked with a promoter and a transcription termination sequence.

As used herein, the term “operably linked” means a functional linkage between a nucleic acid expression control sequence (e.g., a promoter, a signal sequence, or an array of transcriptional regulator binding sites) and a different nucleic acid sequence, whereby the control sequence serves to control the transcription and/or translation of the different nucleic acid sequence.

When a prokaryotic cell is used as a host, a strong promoter capable of promoting transcription (e.g., a tac promoter, lac promoter, lacUV5 promoter, lpp promoter, pLλ promoter, pRλ promoter, rac5 promoter, amp promoter, recA promoter, SP6 promoter, trp promoter, or T7 promoter), a ribosome-binding site for initiation of translation, and a transcription/translation termination sequence are generally included. In addition, for example, when a eukaryotic cell is used as a host, a promoter derived from the genome of a mammalian cell (e.g. a metallothionine promoter, R-actin promoter, human hemoglobin promoter or human muscle creatine promoter) or a promoter derived from a mammalian virus (e.g. an adenovirus late promoter, vaccinia virus 7.5k promoter, SV40 promoter, cytomegalovirus (CMV) promoter, tk promoter of HSV, mouse mammary tumor virus (MMTV) promoter, LTR promoter of HIV, promoter of Moloney virus, promoter of Epstein-Barr virus (EBV), or promoter of Rous sarcoma virus (RSV)) may be used, and a polyadenylation sequence is generally used as a transcription termination sequence

In some embodiments, the promoter may be a eukaryotic promoter, is preferably selected from among a cytomegalovirus (CMV) promoter, a PGK promoter, an EF1α promoter, an EFS promoter, a CBh promoter, an MSCV promoter, an SFFV promoter, and a UbC promoter, and is most preferably selected from among a CMV promoter, an EF1α promoter, and a CBh promoter, but is not limited thereto.

In some embodiments, the promoter may further include an enhancer sequence, but is not limited thereto.

In some cases, the vector may be fused with another sequence in order to facilitate purification of the antibody expressed therefrom. Examples of the sequence that is fused therewith include glutathione S-transferase (Pharmacia, USA), maltose-binding protein (NEB, USA), FLAG (IBI, USA), and 6×His (hexa-histidine; Qiagen, USA)).

The vector may include, as a selective marker, an antibiotic resistance gene that is commonly used in the art, for example, a gene conferring resistance to ampicillin, gentamicin, carbenicillin, chloramphenicol, streptomycin, kanamycin, puromycin, blasticidin, hygromycin, geneticin, neomycin, and tetracycline, but is not limited thereto.

In some embodiments, a nucleic acid encoding the stefin A protein variant specifically binding to CD40L or the fusion protein including the same may be incorporated and introduced into the gene of the host cell.

In some embodiments, the nucleic acid encoding a CD40L binding agent (e.g., a stefin A protein variant specifically binding to CD40L) or a fusion protein including the same may be constructed through chemical synthesis using an oligonucleotide synthesizer. Oligonucleotides may be designed based on the amino acid sequence of the desired polypeptide and by selecting codons that are favored in the host cell in which the recombinant polypeptide of interest will be produced. A polynucleotide sequence encoding the isolated polypeptide of interest may be synthesized using standard methods. For example, a reverse-translated gene may be constructed using a complete amino acid sequence. In addition, a DNA oligomer containing a nucleotide sequence encoding a particular isolated polypeptide may be synthesized. For example, several small oligonucleotides encoding portions of a desired polypeptide may be synthesized and then ligated. Individual oligonucleotides generally contain 5′ or 3′ overhangs for complementary assembly.

In some embodiments, when the nucleic acid sequence encoding a CD40L binding agent (e.g., a stefin A protein variant specifically binding to CD40L) or a fusion protein including the same is obtained, a vector including the same may be produced through recombinant DNA technology using a technique well known in the art. An expression vector containing a sequence encoding a CD40L binding agent (e.g., a stefin A protein variant) or a fusion protein including the same and appropriate transcriptional and translational control signals may be constructed using methods well known to those skilled in the art. Examples of such methods may include in-vitro recombinant DNA techniques, synthesis techniques, and in-vivo genetic recombination (e.g. Sambrook et al., 1990, MOLECULAR CLONING, A LABORATORY MANUAL, 2d Ed., Cold Spring Harbor Laboratory, Cold Spring Harbor, N.Y. and Ausubel et al. eds., 1998, CURRENT PROTOCOLS IN Molecular Biology, John Wiley & Sons, NY).

In some embodiments, the nucleic acid encoding a CD40L binding agent (e.g., a stefin A protein variant specifically binding to CD40L) or aa fusion protein including the same or the non-viral expression vector including the same may be delivered to the host cell using typical techniques (e.g. electroporation, liposome transfection, and calcium phosphate precipitation).

The vector may be introduced into the host cell through a method such as transduction or transfection. As used herein, the term “transduction” refers to introduction of DNA into a host such that the DNA becomes replicable either as an extrachromosomal factor or through chromosomal integration. As used herein, the term “transfection” means that an expression vector is accommodated by the host cell, regardless of whether or not any coding sequence is actually expressed. In order to introduce the vector, a variety of techniques commonly used to introduce exogenous nucleic acids (DNA or RNA) into prokaryotic or eukaryotic host cells, for example, electrophoresis, calcium phosphate precipitation, DEAE-dextran transfection, or lipofection may be used, but the present invention is not limited thereto.

It is to be understood that not all vectors and expression control sequences function equally in expressing the DNA sequence of the present invention. Likewise, not all hosts function equally for the same expression system. However, those skilled in the art will be able to make an appropriate selection from among various vectors, expression control sequences, and hosts without undue experimentation and without departing from the scope of the present invention. For example, a vector may be selected in consideration of the host. This is because the vector has to be able to replicate in the host. Also, the number of copies of a vector, ability to control the number of copies, and expression of another protein encoded by the vector, for example, an antibiotic marker, have to be taken into consideration. In selecting the expression control sequence, various factors have to be considered. For example, the relative strength of the sequences, controllability thereof, compatibility with the DNA sequences of the present invention, etc., should be taken into account, particularly with regard to possible secondary structures. The single-celled host should be selected in consideration of factors such as the selected vector, the toxicity and secretory properties of the product encoded by the DNA sequence of the invention, the ability to correctly fold the protein, culture and fermentation requirements, ease of purification of the product encoded by the DNA sequence of the present invention from the host, and the like. Within the scope of these parameters, those skilled in the art may select various vector/expression control sequence/host combinations capable of expressing the DNA sequence of the present invention in fermentation or large-scale animal culture. Examples of a screening method of cloning cDNA by expression cloning may include a binding method, a panning method, a film emulsion method, etc.

In some embodiments, a nucleic acid encoding a CD40L binding agent or a fusion protein including the same is introduced into a host cell using a lentivirus.

In some embodiments, a nucleic acid encoding the stefin A protein variant specifically binding to CD40L or the fusion protein including the same was introduced into a host cell using a lentivirus.

In some particular embodiments, a transduction enhancer may be used when introducing a gene using a lentivirus.

In some embodiments, the transduction enhancer may be selected from among, for example, polybrene, protamine sulfate, and LentiBOOST from Sirion, and is most preferably polybrene, but is not limited thereto.

In some embodiments, the host cell that is attached or not attached may be infected with both a transduction enhancer and a lentivirus, and infection before cell attachment is preferable, but the present invention is not limited thereto.

Another aspect of the present invention pertains to a genetically modified cell expressing a CD40L binding agent (e.g., a stefin A protein variant specifically binding to CD40L) and/or a fusion protein including the same.

### Method of Producing Genetically Modified Cell and Culture Fluid of Genetically Modified Cell

Still another aspect of the present invention pertains to a method of producing a genetically modified cell into which a nucleic acid encoding a CD40L binding agent (e.g., a stefin A protein variant specifically binding to CD40L) and/or a fusion protein including the same is introduced, including:
(a) introducing a nucleic acid encoding a stefin A protein variant specifically binding to CD40L and/or a fusion protein including the same into a host cell; and
(b) selecting and obtaining the host cell into which the nucleic acid encoding the CD40L binding agent (e.g., stefin A protein variant specifically binding to CD40L) and/or the fusion protein including the same is introduced.

In some embodiments, step (a) may be performed through various means known in the art.

In some embodiments, step (a) may be performed using a lentivirus containing the nucleic acid encoding the CD40L binding agent (e.g., the stefin A protein variant specifically binding to CD40L) and/or the fusion protein including the same.

In some embodiments, the lentivirus may be introduced with a vector including the nucleic acid encoding the CD40L binding agent (e.g., the stefin A protein variant specifically binding to CD40L) and/or the fusion protein including the same.

In some embodiments, the vector may further include at least one selected from among a signal sequence, an origin of replication, at least one antibiotic resistance marker gene, an enhancer element, a promoter, and a transcription termination sequence.

In some embodiments, the promoter may be a eukaryotic promoter, is preferably selected from among a CMV promoter, a PGK promoter, an EF1α promoter, an EFS promoter, a CBh promoter, an MSCV promoter, an SFFV promoter, and a UbC promoter, and is most preferably selected from among a CMV promoter, an EF1α promoter, and a CBh promoter, but is not limited thereto.

In some embodiments, the promoter may further include an enhancer sequence, but is not limited thereto.

In some embodiments, the enhancer is a short DNA region of about 50 to 1500 bp in length that may bind to a transcriptional regulatory protein. The enhancer may be located in the transcription start site or upstream or downstream of the promoter. Enhancers for various promoters are well known in the art, and may be selected and applied without limitation by those skilled in the art.

In some embodiments, step (a) may include transducing the host cell by infecting the host cell with the lentivirus.

In some embodiments, transducing the host cell by infecting the host cell with the lentivirus may be performed by adding a transduction enhancer.

In some embodiments, the transduction enhancer is preferably a cationic polymer, making it easy to incorporate a negatively charged nucleic acid or gene into the host cell.

In some embodiments, the transduction enhancer may be a cationic polymer. For example, the transduction enhancer may be selected from among polybrene, protamine sulfate, and LentiBOOST from Sirion, and is most preferably polybrene, but is not limited thereto.

In some embodiments, transducing the host cell by infecting the host cell with the lentivirus may be performed by treating the host cell with the lentivirus after attaching the host cell, or by treating and infecting the host cell with the lentivirus before attaching the host cell.

In some embodiments, a method of infecting the host cell with the lentivirus during the process of attaching the host cell by treating the host cell with the lentivirus before attaching the host cell is also known as reverse transduction.

In some embodiments, in the transduction of the host cell by infecting the host cell with the lentivirus, it is preferable to infect the host cell through treatment with the lentivirus before attaching the host cell, but the present invention is not limited thereto.

In some embodiments, step (b) may be characterized in that the host cell into which the nucleic acid encoding the CD40L binding agent (e.g., the stefin A protein variant specifically binding to CD40L) and/or the fusion protein including the same is introduced is selected using an antibiotic and a resistance gene thereto.

The method of selecting the transduced cell into which the gene is introduced using a vector including an antibiotic and a resistance gene thereto is well known in the art.

In some embodiments, step (b) may be characterized in that the genetically modified cell into which the nucleic acid is introduced is selected through treatment with an aminoglycoside-based antibiotic.

In some embodiments, examples of the antibiotic may include, but are not limited to, ampicillin, gentamycin, carbenicillin, chloramphenicol, streptomycin, kanamycin, puromycin, blasticidin, hygromycin, geneticin, neomycin, tetracycline, and the like.

In some embodiments, the genetically modified cell may further include a neomycin resistance gene introduced thereto, in addition to the nucleic acid encoding the CD40L binding agent (e.g., the stefin A protein variant specifically binding to CD40L) and/or the fusion protein including the same.

In some embodiments, treatment with the antibiotic for 3 to 7 days at a concentration of 250 to 500 μg/mL, for 5 days at a concentration of about 125 μg/mL, or for 7 days at a concentration of about 62.5 μg/mL is possible, but the present invention is not limited thereto.

Yet another aspect of the present invention pertains to a culture fluid of the genetically modified cell. In some embodiments, the culture fluid may be prepared by culturing the genetically modified cell using suitable conditions and media depending on the type of host cell.

In some embodiments, the genetically modified cell is capable of expressing and secreting the CD40L binding agent (e.g., the stefin A protein variant specifically binding to CD40L) and/or the fusion protein including the same. As such, the culture fluid of the genetically modified cell may include not only the genetically modified cell, but also the CD40L binding agent (e.g., the stefin A protein variant specifically binding to CD40L) and/or the fusion protein including the same, which are secreted thereby.

In some embodiments, provided are genetically modified mesenchymal stem cells (MSCs) that comprise a nucleic acid encoding a CD40L binding agent as described herein. In some embodiments, provided are genetically modified MSCs that express a CD40L binding agent, wherein the MSCs are derived from induced pluripotent stem cells. In some embodiments, provided are genetically modified MSCs that express a CD40L binding agent, wherein the MSCs possess multipotency capable of differentiating into cells selected from the group consisting of adipocytes, osteocytes, chondrocytes, myocytes, nerve cells and cardiomyocytes. In some embodiments, provided are genetically modified MSCs that express a CD40L binding agent, wherein the MSCs that are capable of long term storage and/or repeated passages. In some embodiments, provided are genetically modified MSCs that express a CD40L binding agent, wherein expression of MSC cell surface markers is maintained at 90% or more in mesenchymal stem cells of 20 or more passages.

In some embodiments, where the CD40L binding agent is secreted extracellularly, the population of cells express and secrete CD40L binding agent at an average level of 200 fg/cell/day or greater. In some embodiments, where the CD40L binding agent is secreted extracellularly, the population of cells express and secrete CD40L binding agent at an average level of 300 fg/cell/day or greater. In some embodiments, where the CD40L binding agent is secreted extracellularly, the population of cells express and secrete CD40L binding agent at an average level of 400 fg/cell/day or greater.

In some embodiments, where the CD40L binding agent is secreted extracellularly, the population of cells express and secrete CD40L binding agent at an average level of 200 to 1500 fg/cell/day. In some embodiments, where the CD40L binding agent is secreted extracellularly, the population of cells express and secrete CD40L binding agent at an average level of 300 to 1000 fg/cell/day. In some embodiments, where the CD40L binding agent is secreted extracellularly, the population of cells express and secrete CD40L binding agent at an average level of 400 to 800 fg/cell/day.

In some embodiments, when the host cell is a mesenchymal stem cell, a long-term subculture method thereof is described in the art, and, for example, Korean Patent Application Publication No. 10-2021-0072734, Korean Patent No. 10-No. 1135636, and the like disclose a method of maintaining undifferentiation potency and marker expression characteristics even after tens of passages.

### Usage

The present invention excludes from the scope of rights the use for the manufacturing of Stepin A protein variants and/or fusion proteins containing them that specifically bind to CD40L of the genetically engineered cells.

Uses of the genetically engineered cells of the present invention include, without limitation, all uses except the use for manufacturing stepin A protein variants and/or fusion proteins containing the same. Preferred examples include, but are not limited to, medical, pharmaceutical, and clinical uses.

### Use—Cell Therapeutic Agent or Pharmaceutical Composition

In particular, it is well known in the art that the activation of T cells and B cells by interaction of CD40L/CD40 acts as a pathogenic factor for autoimmune diseases or inflammatory diseases that have a major influence on pathology. Specifically, it is a pathogenic factor of various diseases such as type 1 diabetes, thyroiditis, psoriasis, lupus (systemic lupus erythematosus (SLE)), rheumatoid arthritis (RA), multiple sclerosis (MS), and the like. Various compounds or antibodies targeting CD40L have been developed for the treatment of these diseases (Semin Immunol. 2009; 21(5):293-300; Advanced Drug Delivery Reviews Volume 141, 15 Feb. 2019, Pages 92-103).

In some embodiments, it has been confirmed that the genetically modified cell of the present invention has very high ability to inhibit T-cell activity and B-cell activity.

In some embodiments, it has been confirmed that, when the CD40L binding agent (e.g., the stefin A protein variant specifically binding to CD40L) is administered to an animal model having graft-versus-host disease (GVHD), a significant therapeutic effect is exhibited.

The present disclosure provides further aspects that pertain to a cell therapeutic agent including the genetically modified cell.

The present disclosure provides further aspects that pertain to a pharmaceutical composition for preventing or treating an immune disease including the genetically modified cell or the culture fluid thereof.

In some embodiments, provided are compositions comprising genetically modified cells (e.g., MSCs) that include or express a CD40L binding agent. In some embodiments, provided are pharmaceutical compositions comprising the genetically modified MSCs.

In some embodiments, when the genetically modified cell includes a CD40L binding agent (e.g., a stefin A protein variant specifically binding to CD40L) and/or a fusion protein including the same, it may be contained in the pharmaceutical composition in the form of a culture fluid including not only the genetically modified cell but also the CD40L binding agent (e.g., stefin A protein variant specifically binding to CD40L) and/or the fusion protein including the same, which are secreted thereby.

As used herein, the term “prevention” refers to any action that inhibits or delays the onset of an immune disease by administering the pharmaceutical composition provided in the present invention to a subject who is expected to develop an immune disease.

As used herein, the term “treatment” refers to any action that clinically intervenes to alter the natural process of a subject or cell to be treated, and may be performed during the course of or to prevent a clinical pathology. The desired therapeutic effect includes prevention of occurrence or recurrence of disease, alleviation of symptoms, inhibition of all direct or indirect pathological consequences of disease, prevention of metastasis, reduction of disease progression rate, alleviation or temporary alleviation of disease state, and prognosis improvement. For the purpose of the present invention, the treatment may be interpreted as including all actions of ameliorating the symptoms of an autoimmune disease by administering the pharmaceutical composition of the present invention to a patient suffering from an autoimmune disease including psoriasis, but the present invention is not particularly limited thereto.

In some embodiments, the pharmaceutical composition may further include at least one pharmaceutically acceptable carrier.

The pharmaceutically acceptable carrier included in the composition of the present invention is commonly used in formulations, and includes lactose, dextrose, sucrose, sorbitol, mannitol, starch, acacia gum, calcium phosphate, alginate, gelatin, calcium silicate, microcrystalline cellulose, polyvinylpyrrolidone, water, syrup, methyl cellulose, methyl hydroxybenzoate, propyl hydroxybenzoate, talc, magnesium stearate, mineral oil, and the like, but is not limited thereto. The composition of the present invention may further include a lubricant, a wetting agent, a sweetening agent, a flavoring agent, an emulsifying agent, a suspension agent, a preservative, and the like, in addition to the above components.

The pharmaceutical composition of the present invention may be administered orally or parenterally, and parenteral administration may include intravenous injection, subcutaneous injection, intramuscular injection, intraperitoneal injection, endothelial administration, topical administration, intranasal administration, intrapulmonary administration, and intrarectal administration.

When administered orally, the protein or peptide is digestible and therefore oral compositions should be formulated to coat the active agent or to protect the same from degradation in the stomach. In addition, the pharmaceutical composition may be administered by any device capable of transporting the active material to a target cell.

A suitable dosage of the composition according to the present invention may vary depending on factors such as formulation method, administration mode, patient's age, body weight, gender, and pathological condition, food, administration time, administration route, excretion rate, and reaction sensitivity. Here, an effective dosage thereof for the desired treatment or prevention may be easily determined and prescribed by skilled doctors. As used herein, the term “pharmaceutically effective amount” refers to an amount sufficient to prevent or treat an immune disease.

The pharmaceutical composition of the present invention may be formulated into a unit dosage form or placed in a multi-dose container using a pharmaceutically acceptable carrier and/or excipient according to a method that may be easily carried out by a person of ordinary skill in the art to which the present invention belongs. Here, the formulation may be in the form of a solution, suspension, or emulsion in oil or aqueous medium, or may be in the form of an extract, powder, suppository, powder, granule, tablet, or capsule, and may additionally include a dispersant or stabilizer.

The pharmaceutical composition according to the present invention may be administered in combination with a known drug or pharmaceutical composition having an effect of preventing, ameliorating, or eliminating symptoms of an immune disease, and may be co-administered with, for example, one or more other immunotherapeutic agents, chemotherapeutic agents, antibody therapeutic agents, and the like.

Specifically, the pharmaceutical composition of the present invention may be used in combination with a drug selected from the group consisting of disease-modifying antirheumatic drugs (DMARDs), nonsteroidal anti-inflammatory drugs (NSAIDs), corticosteroids, Janus kinase inhibitors, calcineurin inhibitors, mTOR inhibitors, IMDH inhibitors, and biological agents, but the present invention is not limited thereto.

In some embodiments, examples of the disease-modifying antirheumatic drugs (DMARDs) may include, but are not limited to, actarit, auranofin, azathioprine, bucillamine, cyclophosphamide, D-penicillamine, leflunomide, lobenzarit disodium, methotrexate, minocycline hydrochloride, mizoribine, salazosulfapyridine, and the like.

In some embodiments, examples of the nonsteroidal anti-inflammatory drugs (NSAIDs) may include, but are not limited to, celecoxib, diclofenac sodium, ibuprofen, ketoprofen, meloxicam, naproxen, piroxicam, and the like.

In some embodiments, examples of the corticosteroids may include, but are not limited to, prednisone (Deltasone, Orasone), budesonide (Entocort EC), prednisolone (Millipred), methylprednisolone, and the like.

In some embodiments, examples of the Janus kinase inhibitors may include, but are not limited to, approved drugs such as tofacitinib, abrocitinib, baricitinib, delgocitinib, fedratinib, filgotinib, oclacitinib, peficitinib, ruxolitinib, and upadacitinib, drugs in clinical trials such as cerdulatinib, gandotinib, lestaurtinib, momelotinib, pacritinib, and deucravacitinib, and the like.

In some embodiments, examples of the calcineurin inhibitors include, but are not limited to, cyclosporine, tacrolimus, and the like.

In some embodiments, examples of the mTOR inhibitors may include, but are not limited to, sirolimus (Rapamune), everolimus (Afinitor, Zortress), and the like.

In some embodiments, examples of the IMDH inhibitors may include, but are not limited to, azathioprine (Azasan, Imuran), mycophenolate (CellCept, Myfortic), and the like.

In some embodiments, examples of the biological agents includes abatacept, adalimumab, anakinra, certolizumab, etanercept, golimumab, infliximab, ixekizumab, natalizumab, rituximab, secukinumab, tocilizumab, ustekinumab, vedolizumab, basiliximab, daclizumab, and the like.

In some embodiments, the immune disease may be an autoimmune disease or an inflammatory disease.

In some embodiments, the immune disease may be selected from the group consisting of lupus (SLE), lupus nephritis (e.g. drug-induced lupus nephritis), immune thrombocytopenia (ITP), rheumatoid arthritis (RA), multiple sclerosis (MS), inflammatory bowel disease (IBD) (e.g. Crohn's disease and colitis/ulcerative colitis), graft-versus-host disease (GVHD) or allograft rejection, transplantation/solid organ transplantation (SOT), primary biliary cholangitis (PBC), psoriasis, psoriatic arthritis, collagen-induced arthritis, experimental allergic encephalomyelitis (EAE), oophoritis, allergic rhinitis, asthma, Sjogren's syndrome, atopic eczema, myasthenia gravis, Graves' disease, and/or glomerulosclerosis, but is not limited thereto.

### Lupus

Systemic lupus erythematosus (SLE), also called lupus, is a chronic autoimmune disease that may cause swelling (inflammation) and pain throughout the body. There are several types of lupus, and systemic lupus erythematosus is the most common. Other types of lupus are described below.

Cutaneous lupus erythematosus: This type of lupus affects the skin. “Cutaneous” is a term that means skin. People with cutaneous lupus erythematosus may experience skin problems such as sensitivity to the sun and rashes. Hair loss may also be a symptom of the disease.

Drug-induced lupus: This lupus is caused by certain drugs. People with drug-induced lupus may have many of the same symptoms as systemic lupus erythematosus, but these symptoms are usually temporary.

Neonatal lupus: Neonatal lupus, which is a rare type of lupus, is a disease found in infants at birth. Children born with neonatal lupus inherit antibodies from their mother, and the mother may have had lupus during pregnancy or may develop the disease later. Not all babies born to mothers with lupus will develop lupus.

Examples of therapies that may be used in combination with the pharmaceutical composition of the present invention may include, but are not limited to, steroids (including corticosteroids, prednisone); hydroxychloroquine (Plaquenil®); azathioprine (Imuran®); methotrexate (Rheumatrex®); cyclophosphamide (Cytoxan®) and mycophenolate mofetil (CellCept®); belimumab (Benlysta®); and/or rituximab (Rituxan®).

### Lupus Nephritis

Lupus nephritis develops as a complication of lupus. Lupus nephritis occurs when lupus autoantibodies affect the structure of the kidneys, which filter waste. This causes kidney inflammation and may lead to blood in the urine, protein in the urine, high blood pressure, kidney dysfunction, or kidney failure. About half of adults with systemic lupus develop lupus nephritis. In systemic lupus, immune system proteins damage the kidneys, impairing the ability to filter waste.

### Rheumatoid Arthritis

Rheumatoid arthritis is a type of chronic (progressive) arthritis that occurs in both joints of the body, such as the hands, wrists, and knees. The short-term goal of drugs for rheumatoid arthritis is to reduce joint pain and swelling and improve joint function. The long-term goal thereof is to slow or stop disease progression, particularly joint damage.

Arthritis is a general term that describes inflammation of the joints. Rheumatoid arthritis is a type of chronic (progressive) arthritis (causing pain and swelling) that usually occurs symmetrically in the joints (on both sides of the body, such as the hands, wrists, and knees). This involvement of multiple joints helps to distinguish rheumatoid arthritis from other types of arthritis.

In addition to affecting the joints, rheumatoid arthritis may sometimes affect the skin, eyes, lungs, heart, blood, nerves, or kidneys.

Therapies that may be used in combination with a pharmaceutical composition of the present invention to treat rheumatoid arthritis may include the following examples.

Pain relievers: These products include nonsteroidal anti-inflammatory drugs (NSAIDs) such as ibuprofen (MOTRINR), naproxen (ALEVE®), celecoxib, diclofenac sodium, ketoprofen, meloxicam, and piroxicam. A COX-2 inhibitor, which is another type of drug, also falls in this category and relieves the signs and symptoms of rheumatoid arthritis. Celecoxib (CELEBREX®), which is an example of the COX-2 inhibitor, is available in the United States. A COX-2 inhibitor is designed to have fewer side effects of gastrointestinal bleeding.

Disease-modifying antirheumatic drugs (DMARDs): Unlike other NSAIDs, DMARDs may actually slow disease progression by modifying the immune system. Previous DMARDs include methotrexate (TREXALL®), gold salts, penicillamine (CUPRIMINE®), hydroxychloroquine (PLAQUENIL®), sulfasalazine (AZULFIDINE®), cyclosporine (SANDIMMUNE®), cyclophosphamide (CYTOXAN®), and leflunomide (ARAVA®). Currently available examples thereof may include methotrexate, leflunomide, hydroxychloroquine, and sulfasalazine (cyclosporine, cyclophosphamide, gold salt, and penicillamine are no longer commonly used).

Biological agents: In addition to these “traditional” DMARDs, new drugs are being approved. 7 classes of drugs are currently available, and in some cases, there are different types for each class (some of which are anti-TNF series and have been in use since 2000). Collectively, these DMARDs are known by other names: biological agents (or biological materials). Compared to traditional DMARDs, these products target molecules that cause inflammation in rheumatoid arthritis. Inflammatory cells in the joints are involved in the onset of rheumatoid arthritis itself. Biological agents reduce the inflammatory process that ultimately induces joint damage in rheumatoid arthritis. By attacking cells at a specific level rather than inflammation itself, biological agents are considered to be more effective and more specifically targeted. The biological agents include etanercept (ENBREL®), infliximab (REMICADE®), adalimumab (HUMIRA®), anakinra (KINARET®), abatacept (ORENCIA®), rituximab (RITUXAN®), certolizumab pegol (CIMZIA®), golimumab (SYMPON®), tocilizumab (ACTEMRA®), and tofacitinib (XELJANJ®). Some biological agents are used in combination with traditional DMARDs, especially methotrexate.

### Multiple Sclerosis

Multiple sclerosis (MS) is an autoimmune disease. Under such conditions, the immune system mistakenly attacks healthy cells. In patients with multiple sclerosis, the immune system attacks myelin cells, which are the protective sheath that surrounds nerves in the brain and spinal cord. When myelin is damaged, nerve signals from the brain to other parts of the body are blocked. Damage may cause symptoms that affect the brain, spinal cord, and eyes.

There are four types of multiple sclerosis.
Clinically isolated syndrome (CIS): When there are the first symptoms of MS, health care providers often classify the same as CIS. Not all cases of CIS progress to multiple sclerosis.
Relapse-remitting MS (RRMS): This is the most common form of multiple sclerosis. People with RRMS have flares of new or worsening symptoms—also called relapses or exacerbations. A period of remission continues (when symptoms stabilize or disappear).
Primary-progressive MS (PPMS): People diagnosed with PPMS have symptoms that gradually worsen over time without relapse or remission.
Secondary-progressive MS (SPMS): In most cases, people originally diagnosed with RRMS eventually progress to SPMS.

In secondary-progressive multiple sclerosis, nerve damage continues to accumulate. Symptoms gradually worsen. Such people may experience some relapse or flare-up (when symptoms increase), but there is no longer a period of remission after that (when symptoms stabilize or disappear).

Therapies that may be used in combination with the pharmaceutical composition of the present invention may include the following examples.

Disease-modifying therapy (DMT) Several drugs have received FDA approval for the treatment of long-term MS. These drugs help reduce relapses (also called flares or seizures). They slow the progression of the disease, and may prevent the formation of new lesions in the brain and spinal cord.
Relapse management medication: If there are severe seizures, the neurologist may recommend high-dose corticosteroids. Medications may reduce inflammation quickly, and may slow damage to the myelin sheaths surrounding nerve cells.
Physical rehabilitation: Multiple sclerosis may affect bodily functions. Staying physically healthy and strong will help a patient stay mobile.
Mental health counseling: Coping with chronic illness may be emotionally challenging. MS may sometimes affect mood and memory. Working with a neuropsychologist or getting other emotional support is an essential part of disease management.

### Inflammatory Bowel Disease

Inflammatory bowel disease (IBD) is a group of disorders that cause chronic inflammation (pain and swelling) of the intestine.

Crohn's disease and ulcerative colitis are the main types of IBD. The types thereof are described below.

Crohn's disease causes pain and swelling in the digestive tract, and may affect everything from the mouth to the anus. It most commonly affects the small intestine and upper part of the large intestine.

Ulcerative colitis causes boils and wounds (ulcers) in the large intestine (colon and rectum).

Microscopic colitis causes intestinal inflammation that may only be detected under a microscope.

Examples of therapies that may be used in combination with the pharmaceutical composition of the present invention may include aminosalicylates (anti-inflammatory agents such as sulfasalazine, mesalamine, or balsalazide) that minimize irritation to the intestines; antibiotics that treat infections and abscesses; biological agents that block signals from the immune system that cause inflammation; corticosteroids, such as prednisone, which suppress the immune system and manage flares; immunomodulators that calm the overactive immune system; antidiarrheal drugs; nonsteroidal anti-inflammatory drugs (NSAIDs); supplements such as vitamins and probiotics.

### Graft-Versus-Host Disease (GVHD)

Graft-versus-host disease (GVHD) is a disease that may occur after allotransplantation. In GVHD, donated bone marrow or peripheral blood stem cells treat the recipient's body as a foreign object and the donated cells/bone marrow attack the body.

GVHD may include acute graft-versus-host disease (aGVHD) and chronic graft-versus-host disease (cGVHD).

### Psoriasis

Psoriasis is a chronic skin disorder meaning a skin disease with no cure. People with psoriasis have thick patches of pink or red skin covered with white or silvery scales. Thick scaly patches are called plaques. Psoriasis usually begins in early adulthood, but it may also start later. In addition to red scaly patches, symptoms of psoriasis include itching, cracking, dry skin, scaly scalp, skin pain, pitted nails, cracked or brittle nails, and joint pain.

Examples of therapies that may be used in combination with the pharmaceutical composition of the present invention may include steroid creams, moisturizers for dry skin, anthralin (drugs that slow the production of skin cells), medicated lotions, shampoos, and bath solutions to ameliorate scalp psoriasis, vitamin D3 ointment, vitamin A or retinoid creams, phototherapy, PUVA (treatment including both a drug called psoralen and UV exposure in a special form), methotrexate, retinoids, cyclosporine, and/or immunotherapy, but are not limited thereto.

### Sjogren's Syndrome

Sjogren's syndrome is a lifelong autoimmune disease that reduces the amount of water produced by the sweat glands of the eyes and mouth. This disease is named after Henrik Sjogren, a Swedish ophthalmologist who first described the condition. Dry mouth and dry eyes are the main symptoms, but most people with these problems do not have Sjogren's syndrome. Dry mouth is also called xerostomia.

There are two forms of Sjogren's syndrome: primary Sjogren's syndrome, which occurs without other autoimmune diseases, and secondary Sjogren's syndrome, which occurs in people who already have other autoimmune diseases, such as rheumatoid arthritis, lupus, and psoriatic arthritis.

Examples of therapies that may be used in combination with the pharmaceutical composition of the present invention may include dry eye treatment (e.g. artificial tears, prescription eye drops, punctual plugs, surgery, and autologous serum eye drops), dry mouth treatment (e.g. saliva-forming agents), and treatment for joint or organ problems (e.g. analgesic agents, antirheumatic agents, immunosuppressive agents, steroids, antifungal agents, and vaginal dryness therapeutic agents).

### Myasthenia Gravis

Myasthenia gravis (MG) is an autoimmune disease in which the body's immune system inadvertently attacks parts of itself. MG affects signal transmission between nerves and muscles (neuromuscular junctions).

Patients with myasthenia gravis lose the ability to voluntarily control their muscles. They experience muscle weakness and fatigue of varying severity, and may be unable to move the muscles of eyes, face, neck, or limbs. MG is a lifelong neuromuscular disease.

Myasthenia gravis affects about 20 out of 100,000 people. Experts estimate that between 36,000 and 60,000 Americans have this neuromuscular disease. The actual number of people affected may be higher because some people with mild symptoms may not know they have the disease. MG mainly affects women between the ages of 20 and 40 and men between the ages of 50 and 80. About 1 in 10 cases of MG occurs in teens (juvenile MG). The disease may affect people of all ages, but is rare in children.

Autoimmune MG is the most common form of this neuromuscular disease. Autoimmune MG may include the following examples.

Ocular MG: The muscles that move the eyes and eyelids weaken. People with ocular MG have droopy eyelids or may not be able to open their eyes, and may have double vision in some cases. Weakness of vision is often the first sign of MG.

Nearly half of people with ocular MG evolve to a systemic form within 2 years after their first symptoms.

Systemic MG: Muscle weakness affects the eyes, face, and other parts of the body, such as the neck, arms, and legs. Patients may have difficulty talking, swallowing, raising their arms above the head, standing from a sitting position, walking long distances, or climbing stairs.

Examples of therapies that may be used in combination with the pharmaceutical composition of the present invention may include drugs, monoclonal antibodies, IV immunoglobulin (IVIG), plasma exchange, and/or surgery.

Yet a further aspect of the present invention pertains to a method of preventing or treating an immune disease including administering the genetically modified cell or the culture fluid thereof.

Still yet a further aspect of the present invention pertains to the use of the genetically modified cell or the culture fluid thereof for the prevention or treatment of an immune disease.

Even still a further aspect of the present invention pertains to the use of the genetically modified cell or the culture fluid thereof for the manufacture of a pharmaceutical composition for the prevention or treatment of an immune disease.

### Use—Composition for Drug Delivery

Even yet a further aspect of the present invention pertains to a composition for drug delivery including the genetically modified cell described above.

In some embodiments, the genetically modified cell may include at least one drug that is supported thereby or attached to the surface thereof.

In some embodiments, the drug may be additionally loaded with at least one selected from the group consisting of a gene, a virus, and a small molecule compound.

In some embodiments, the drug may have immunomodulatory activity, preferably an immunosuppressive effect.

In some embodiments, a genetically modified cell of the present disclosure is capable of expressing a CD40L binding agent (e.g., a stefin A protein variant that specifically binds to CD40L), thereby inhibiting the CD40L-targeted attachment and activity of immune cells.

In some embodiments, the genetically modified cell is a mesenchymal stem cell. When the host cell of the present invention is a mesenchymal stem cell, the mesenchymal stem cell has a homing function to biologically search for a damaged or infected site in the body, and thus has very high targeting ability, making it possible to effectively and accurately deliver the drug to a desired site of the body.

In some embodiments, the drug may be supported inside the mesenchymal stem cell, attached to the surface thereof, or supported inside the cell and attached to the cell surface, but the present invention may not be limited thereto. The drug may be directly supported inside the mesenchymal stem cells and/or attached to the surface thereof, but the present invention is not limited thereto. The drug may be loaded to a nanostructure, supported inside the mesenchymal stem cell in a state of being attached to a specific molecule, and/or attached to the cell surface, but the present invention is not limited thereto. In some embodiments, the nanostructure may include inorganic nanoparticles, polymer nanoparticles, proteins, or liposomes. Examples of the inorganic nanoparticles may include, but are not limited to, iron oxide nanoparticles, quantum dot nanoparticles, metal oxide nanoparticles, and the like. The nanostructure may include, but is not limited to, a porous nanostructure. For example, the drug may be supported by the pores in the porous nanostructure or loaded to the mesenchymal stem cell in a form of being attached to the surface of the nanostructure, but the present invention is not limited thereto. The polymer nanoparticles are nanoparticles frequently used for drug delivery, and are mainly made of polymers and fats.

In some embodiments, the genetically modified cell may be characterized in that the drug is released at a target site. In some embodiments, the drug carried in the genetically modified cell may be released. In some embodiments, the drug attached to the surface of the genetically modified cell may be detached and released from the surface depending on the target environment. In some embodiments, when the drug is supported by the nanostructure, the drug may be released by temperature-specific or pH-specific structural change of the nanostructure.

### EXAMPLE NUMBERED EMBODIMENTS

Embodiment 1. A genetically modified cell in which a nucleic acid encoding a stefin A protein variant specifically binding to CD40L or a fusion protein comprising the stefin A protein variant is introduced into a host cell.

Embodiment 2. The genetically modified cell according to embodiment 1, wherein the stefin A protein variant exhibits a Kd value of 1×10⁻⁶ M or less for CD40L.

Embodiment 3. The genetically modified cell according to embodiment 1, wherein the stefin A protein variant comprises an amino acid sequence represented below:
MIPGGLSEAKPATPEIQEIVDKVKPQLEEKTGETYGKLEAVQYKTQVV-(Xaa)n-GTNYYIKVRAGDNKYMHLKVFKSL-(Xaa)m-EDLVLTGYQVDKNKDDELTGF;
MIPGGLSEAKPATPEIQEIVDKVKPQLEEKTGETYGKLEAVQYKTQVD-(Xaa)n-GTNYYIKVRAGDNKYMHLKVFKSL-(Xaa)m-EDLVLTGYQVDKNKDDELTGF; or
MI PGGLSEAKPATPEIQEIVDKVKPQLEEKTGETYGKLEAVQYKTQVLA-(Xaa)n-GTNYYIKVRAGDNKYMHLKVFKSL-(Xaa)m-EDLVLTGYQVDKNKDDELTGF
wherein Xaa is an amino acid residue, and n and m are each independently an integer from 3 to 20.

Embodiment 4. The genetically modified cell according to embodiment 1, wherein the stefin A protein variant comprises an amino acid sequence selected from the group consisting of SEQ ID NOs: 246 to 365.

Embodiment 5. The genetically modified cell according to embodiment 3, wherein (Xaa)n comprises an amino acid sequence selected from the group consisting of SEQ ID NOs: 6 to 125.

Embodiment 6. The genetically modified cell according to embodiment 3, wherein (Xaa)m comprises an amino acid sequence selected from the group consisting of SEQ ID NOs: 126 to 245.

Embodiment 7. The genetically modified cell according to embodiment 1, wherein the stefin A protein variant or the fusion protein comprising the stefin A protein variant further comprises a signal peptide.

Embodiment 8. The genetically modified cell according to embodiment 1, wherein the fusion protein comprises a trimer or tetramer of stefin A protein variants.

Embodiment 9. The genetically modified cell according to embodiment 8, wherein the fusion protein comprises an amino acid sequence of SEQ ID NO: 681, 691, 694, or 695.

Embodiment 10. The genetically modified cell according to embodiment 8, wherein the trimer or tetramer is configured such that stefin A protein variants are linked by a linker.

Embodiment 11. The genetically modified cell according to embodiment 10, wherein the linker is a rigid linker or a flexible linker.

Embodiment 12. The genetically modified cell according to embodiment 10, wherein the linker is selected from the group consisting of SEQ ID NOs: 508 to 534.

Embodiment 13. The genetically modified cell according to embodiment 1, wherein the fusion protein further comprises at least one selected from the group consisting of a binding domain, a cytokine, a half-life extension domain, a growth factor, an enzyme, and a cell-penetrating domain.

Embodiment 14. The genetically modified cell according to embodiment 1, wherein the fusion protein further comprises at least one selected from the group consisting of a transmembrane domain, a hinge domain, a coiled coil domain, a virus-derived domain, an intracellular signaling domain, and a localization domain.

Embodiment 15. The genetically modified cell according to embodiment 1, wherein the fusion protein further comprises a therapeutic peptide or protein.

Embodiment 16. The genetically modified cell according to embodiment 1, wherein the host cell is selected from the group consisting of a stem cell, an immune cell, and a somatic cell.

Embodiment 17. The genetically modified cell according to embodiment 16, wherein the stem cell is selected from the group consisting of a pluripotent stem cell, a multipotent stem cell, and a unipotent stem cell.

Embodiment 18. The genetically modified cell according to embodiment 16, wherein the host cell is a mesenchymal stem cell.

Embodiment 19. The genetically modified cell according to embodiment 18, wherein the mesenchymal stem cell is differentiated from a human pluripotent stem cell.

Embodiment 20. The genetically modified cell according to embodiment 18, wherein the mesenchymal stem cell expresses at least one cell surface marker selected from among CD29, CD44, CD73, and CD105.

Embodiment 21. The genetically modified cell according to embodiment 20, wherein at least 90% of expression of the cell surface marker is maintained in the mesenchymal stem cell after at least 15 passages.

Embodiment 22. The genetically modified cell according to embodiment 18, wherein the mesenchymal stem cell does not express at least one cell surface marker selected from among CD34, CD45, HLA-DR, TRA-1-60, and TRA-1-81.

Embodiment 23. The genetically modified cell according to embodiment 1, wherein the stefin A protein variant or the fusion protein comprising the stefin A protein variant is expressed intracellularly.

Embodiment 24. The genetically modified cell according to embodiment 1, wherein the stefin A protein variant or the fusion protein comprising the stefin A protein variant is expressed on a cell surface.

Embodiment 25. The genetically modified cell according to any one of embodiments 1 to 22, wherein the stefin A protein variant or the fusion protein comprising the stefin A protein variant is secreted extracellularly.

Embodiment 26. A culture fluid of the genetically modified cell according to embodiment 25.

Embodiment 27. The culture fluid according to embodiment 26, wherein the culture fluid comprises a stefin A protein variant that is secreted by the genetically modified cell or a fusion protein comprising the stefin A protein variant.

Embodiment 28. A cell therapeutic agent for preventing or treating an immune disease comprising the genetically modified cell according to any one of embodiments 1 to 24.

Embodiment 29. A pharmaceutical composition for preventing or treating an immune disease comprising the culture fluid according to embodiment 26.

Embodiment 30. The pharmaceutical composition according to embodiment 29, wherein the immune disease is selected from the group consisting of lupus (SLE), lupus nephritis (e.g. drug-induced lupus nephritis), immune thrombocytopenia (ITP), rheumatoid arthritis (RA), multiple sclerosis (MS), inflammatory bowel disease (IBD) (e.g., Crohn's disease and colitis/ulcerative colitis), graft-versus-host disease (GVHD) or allograft rejection, transplantation/solid organ transplantation (SOT), primary biliary cholangitis (PBC), psoriasis, psoriatic arthritis, collagen-induced arthritis, oophoritis, allergic rhinitis, asthma, Sjogren's syndrome, atopic eczema, myasthenia gravis, Graves' disease, and glomerulosclerosis.

Embodiment 31. A composition for drug delivery comprising the genetically modified cell according to any one of embodiments 1 to 24.

Embodiment 32. The composition according to embodiment 31, wherein the genetically modified cell is configured such that a gene, a virus, or a small molecule compound is further loaded thereto.

### Examples

Hereinafter, the present invention will be described in more detail with reference to the following examples. However, it will be obvious to those skilled in the art that the following examples are provided only for illustration of the present invention and should not be construed as limiting the scope of the present invention.

### Example 1: Selection of Anti-CD40L Stefin A Protein Variant from Phage Display Library

### Identification of Candidate Clones

Example anti-CD40L stefin A protein variants were identified by selection from a library of stefin A protein variant with two random loop sequences, each loop having a length of about 9 amino acids displayed in a constant stefin A protein variant framework backbone based on the amino acid sequence of Stefin A. Such selection procedures have been described (see, e.g., Tiede et al. Protein Eng Des Sel. 2014. 27(5): 145-155 and Hughes et al. Sci Signal. 2018. 10(505): eaaj2005). According to such procedures, suspensions of phage expressing stefin A protein variant were incubated with human or mouse CD40L as required. In some cases the CD40L was biotinylated and captured on alternating streptavidin and neutravidin beads, alternatively the CD40L was passively absorbed to a surface. Unbound phage particles were then washed away and, following washing, bound phage were eluted. Elution of bound phage was accomplished by incubating the antigen with low pH solution, followed by high pH solution and exposure to trypsin. Eluted phage particles were then used to infect Escherichia coli (E. coli), and infected bacteria were incubated under conditions suitable for replication of the bacteriophage. Following release of bacteriophage particles from these infected bacteria, the cycle of allowing phage particles to bind to the target antigen, eluting bound phage particles, propagating eluted phage particles in bacteria, and isolating released phage particles from infected bacteria was repeated to enrich the bacteriophage population for phage particles displaying proteins that bind the target antigen. Specific conditions were modified in these cycles, such as increasing the number of wash steps, reducing the amount of available antigen, or adding a blocking reagent, to select for phage particles displaying proteins that bind more tightly or specifically to the target antigen.

Following multiple rounds of phage display library selection and amplification, proteins expressed by phages were expressed and screened by enzyme-linked immunosorbent assay (ELISA). Briefly, stefin A protein variant were overexpressed from phagemid vectors, the bacterial cells were lysed, and lysates were used as substrates in ELISAs. In these ELISAs, human CD40L was immobilized on a plate, lysates were added, and the amount of anti-CD40L stefin A protein variant in each plate was measured using a detector antibody specific to the 6×Myc tag expressed on the candidate Stefin A protein variant. The phagemid vectors encoding the stefin A protein variant with the best human CD40L-binding activity were sequenced to identify DNA sequences of candidate clones for further development. The loop 2 and loop 4 amino acid sequences of each of these candidate clones are shown in Table 1 and Table 2, respectively.

### Example 2: Screening of Anti-CD40L Stefin A Protein Variant by Direct ELISA

A binding ELISA was performed to measure the affinity of different monomer DAW01 clones for hCD40L.

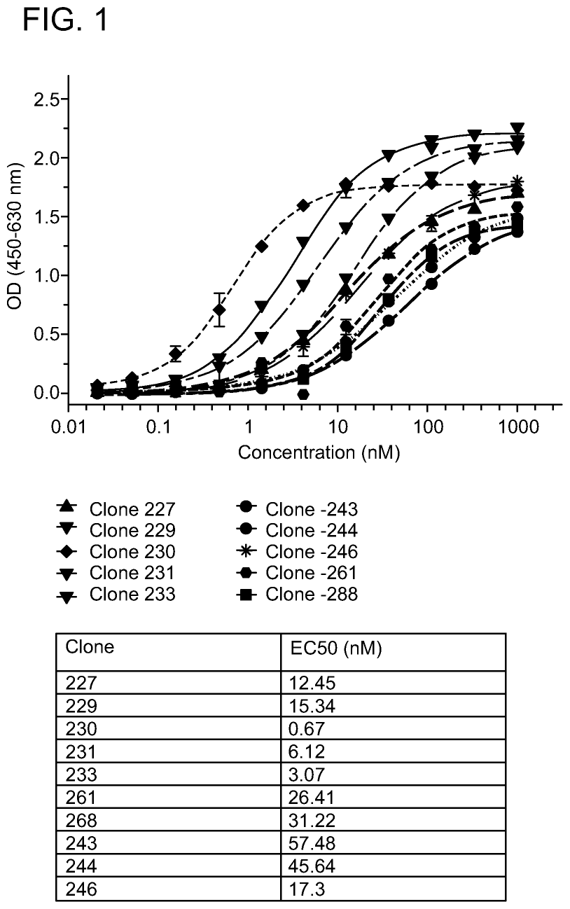

Briefly, plates were coated with hCD40L antigen at 5 μg/ml and incubated overnight at 4° C. Plates were washed 2 times with 150 μl of washing buffer (PBS, Tween 20 0.1%) with a plate washer and saturated with Casein 5% (Sigma) in PBS for 90 minutes at room temperature (25±1° C.). For binding, each DAW01 clone and rhCD40 Fc were added to the plate, starting from 1 μM, 1 in 3 and 300 nM, 1 in 3 respectively. Plates were washed 3 times as described previously. Anti-human CD40 biotinylated polyclonal antibody was then added, and the plate was incubated 90 min. Then, cystatin A biotinylated polyclonal antibody (BAF1407) was diluted to a concentration of 0.05 μg/ml in dilution buffer (PBS, 1% casein, 0.01% Tween 20) and the plate was incubated for 90 minutes at room temperature (25±1° C.). PolyHRP-streptavidin was then diluted in dilution buffer and the plates were incubated for 90 minutes at room temperature (25±1° C.). The plates were then washed 3 times as described previously, and the substrate (TMB, Pierce Thermo-Scientific) was added to the plates for 10 minutes. The reaction was stopped using an acidic solution, and plates were read at 450-630 nm. An example of the results is shown in FIG. 1. The EC50 values ranged between 0.67 to 60 nM.

### Example 3: Screening of Anti-CD40L Stefin A Protein Variant on hCD40L-HEK293 Cells by Flow Cytometry

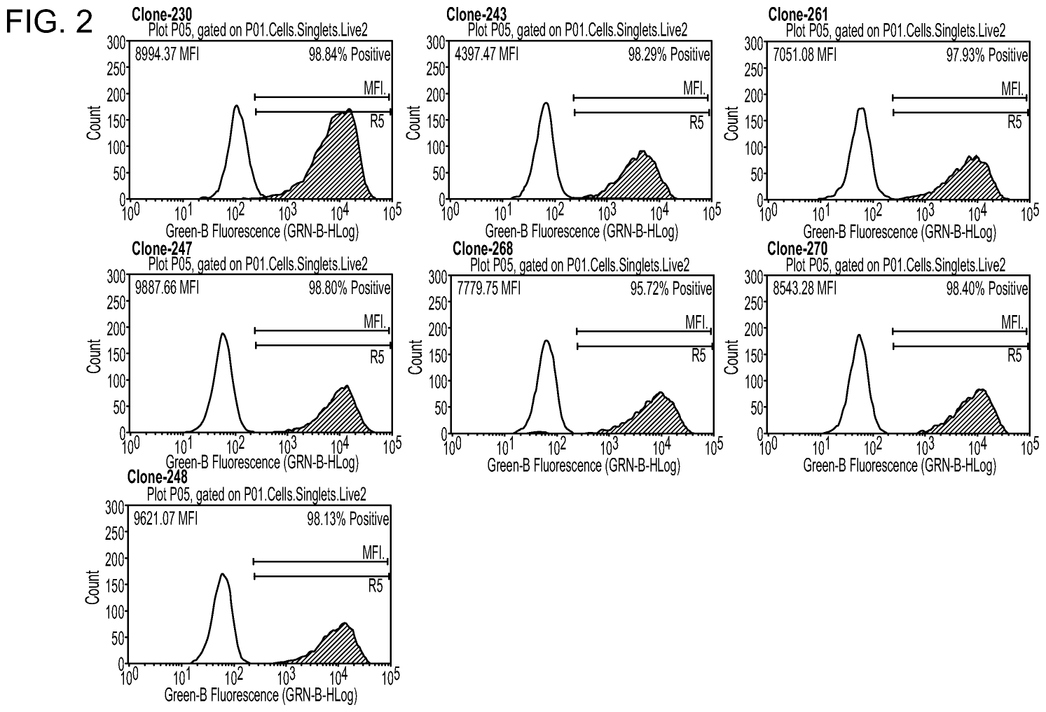

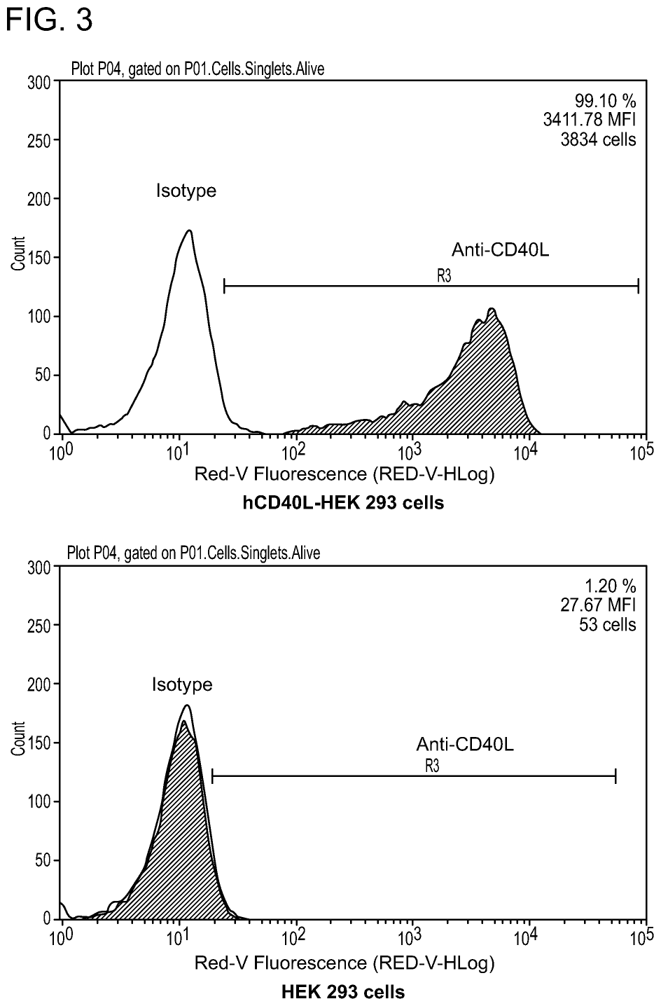

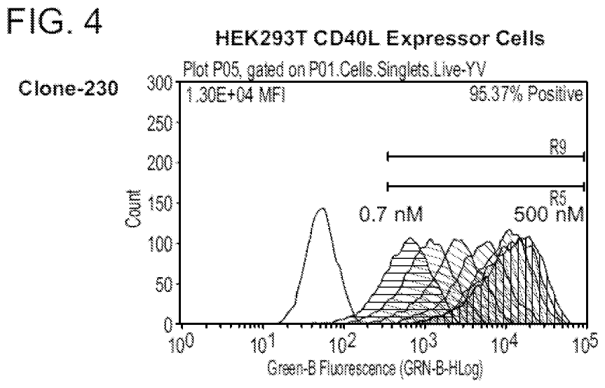

To examine the binding capacity of DAW01 monomer stefin A protein variant for hCD40L expressed on the surface of cells, a flow cytometry cell binding assay was performed. Briefly, hCD40L-HEK-293 cells (Crown Biosciences, C2041) were collected by centrifugation at 300 rpm for 5 min. The cells were resuspended in PBS and 200,000 cells per well were dispatched in a round bottom 96 well plate. Cells were washed with PBS. The stefin A protein variant and controls were diluted in staining buffer containing 1% BSA, 0.01% Sodium Azide (NaN3), 2 mM EDTA in DPBS in duplicate and added on cells for staining for approximately 60 min at 4±1° c. Cells were washed and the secondary anti-Cystatin A (R&D, AF1407) was diluted 0.2 mg/ml in staining buffer and added on cells for staining for approximately 45 min at 4±1° c. Cells were washed again and the detection antibody A488 anti-goat (ThermoFisher, A21467) was diluted 1:500 in staining buffer and added to the cells for staining (approximately 30 min at 4±1° C.). Finally, the cells were washed and live and dead cells were stained using L/D stain Zombie Yellow (Biolegend, 423103) diluted in staining buffer for 10 min at 4±1° C. Cells were washed again and fixation buffer (R&D) was added to each well for 10 min at 4±1° C. then PBS with EDTA (Lonza) was added prior reading the plate on the flow cytometer (Guava 12 HT, Millipore). Dead cells were excluded, and the fluorescent green channel (488 nm/525/30) was acquired. Results were analyzed using Incyte and data were plotted using GraphPad. An example of results at 1 μM is shown in FIG. 2. stefin A protein variant were shown to bind specifically to hCD40L-HEK293 cells (dark gray). HEK-293 negatives cells were also used in the experiment to assess non-specific binding (light gray). The huCD40L HEK293 and HEK293 control cells were assessed for their expression of hCD40L using a BV711 mAb (clone 24-31). The results are shown in FIG. 3. The binding of clone 230 (SEQ ID NO: 249) to hCD40L-HEK293 cells was assessed at different doses ranging from 0.7 to 500 nM. The binding of clone 230 was shown to be dose-dependent between 0.7-55 nM and reached saturation above 55 nM as seen in FIG. 4.

### Example 4: Screening of Anti-CD40L Stefin A Protein Variant in a CD40-HEK Blue Reporter Assay

HEK-Blue CD40 expressor cells (Invivogen), allow detection of bioactive CD40L through activation of NF-κB following CD40 stimulation. Activation of the NF-κB pathway can be determined by measuring levels of secreted embryonic alkaline phosphatase (SEAP) in the cell medium. The assay was performed according to the manufacturer's instructions.

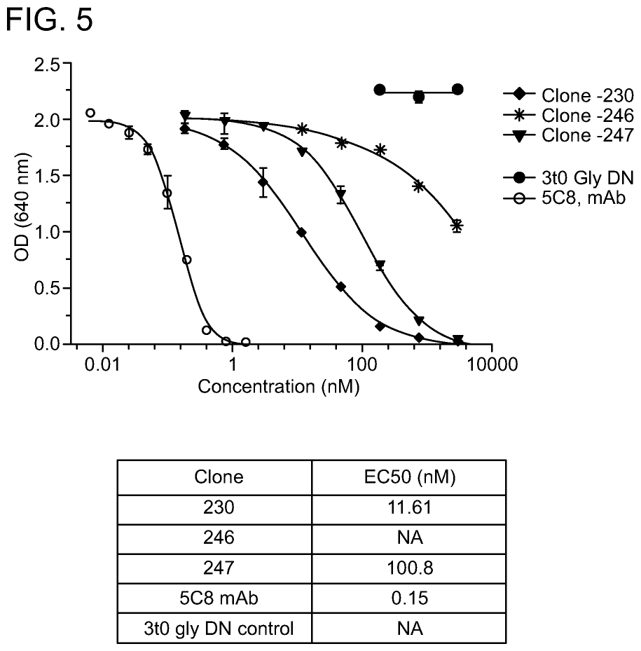

Briefly, cells were seeded at 20000 cells/well in (100 μl) in test medium (DMEM High glucose with Blasticin and Zeocin) in a 96-well flat bottom tissue culture plate and grown overnight at 37° C., 5% C02. Cells were then treated by removing 50 μl of test medium and adding 50 μl 4× dilution of test the stefin A protein variant or controls with hCD40L (0.8 nM final concentration). Plates were then incubated for 22 h at 37° C., 5% CO2. The following day, the supernatant was collected and SEAP activity was detected by mixing 30 ul of each well with 200 ul of HEK-Blue detection reagent (Invivogen). The mixture was incubated 37° C. and any color change was monitored periodically. Absorbance (640 nm) was measured at 3 hours using Pherastar plate reader. The data were plotted. The IC50 was then calculated using the interpolated non-linear four-parameters curve as OD=f(log concentration). 5C8, a clinical grade monoclonal anti-hCD40L antibody, was used as a positive control in this assay. The calculated IC50 ranged from 11.6 to 100 nM (e.g., clone 230 (SEQ ID NO: 249) and clone 248 (SEQ ID NO: 267)). An example of the results is shown in FIG. 5.

### Example 5: Formatting the Stefin A Protein Variant in Dimeric or Trimeric Configurations to Increase Avidity to hCD40L

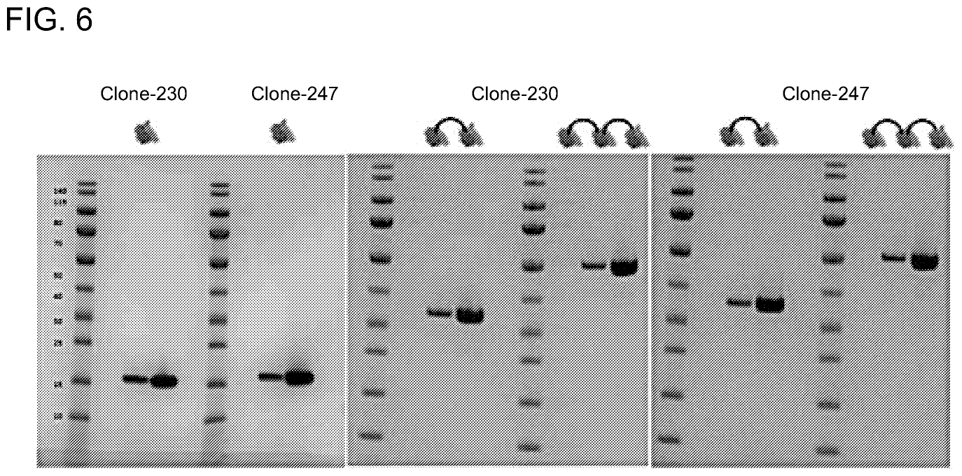

The stefin A protein variant can be engineered to assemble into stable multimeric oligomers to increase avidity. The resulting stefin A protein variant, which are In Line Fusion (ILF) proteins, can be dimers or trimers as pictured in FIG. 6 with various linkers (rigid linkers, see e.g., SEQ ID NOs: 508, 510-514; or flexible linkers, see e.g., SEQ ID NOs: 509, 515-518).

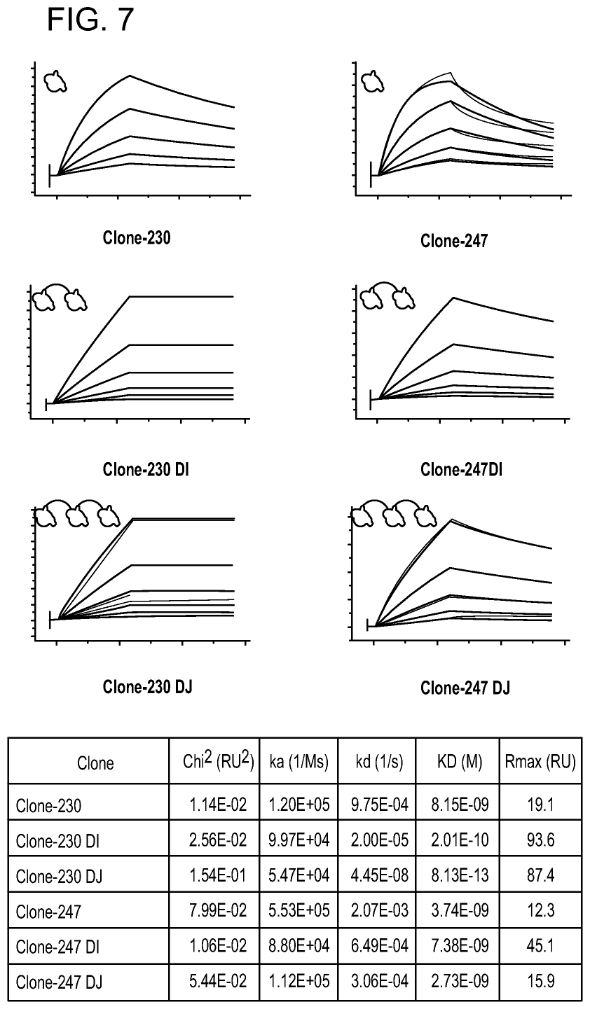

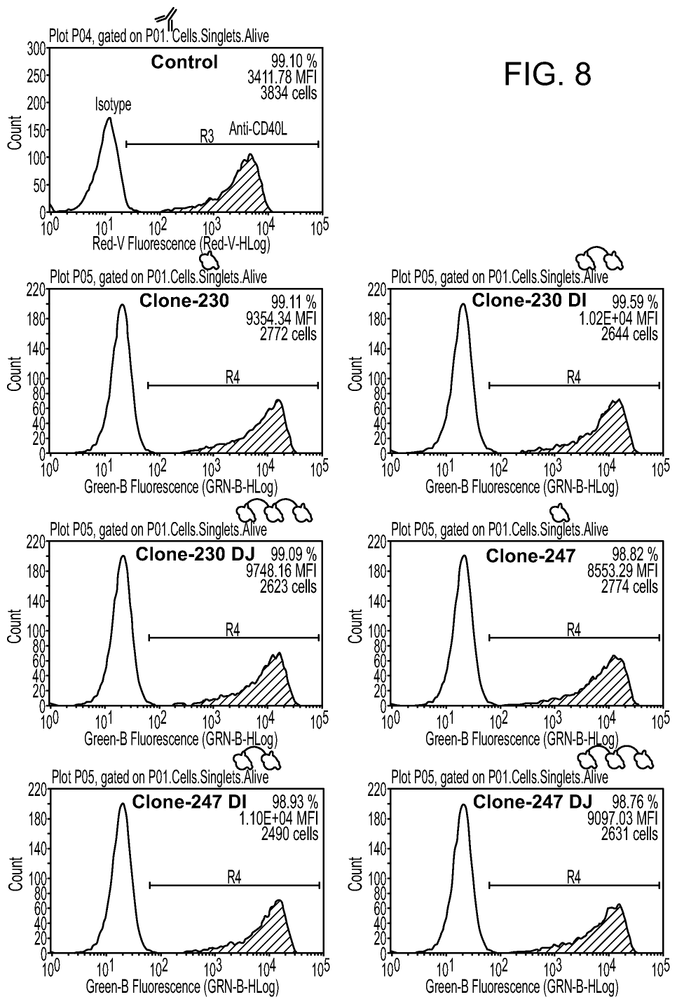

The binding of the different stefin A protein variant (monomeric, dimeric, trimeric) to hCD40L was examined as described above. The results are shown in FIG. 7 and demonstrate that the ILF proteins had comparable or greater binding affinities for the target ligand than the monomeric formulations. The binding was further examined with flow cytometry (FIG. 8), and it was demonstrated that the stefin A protein variant (monomeric, dimeric, and trimeric formats) bound hCD40L at levels comparable to an anti-CD40L antibody.

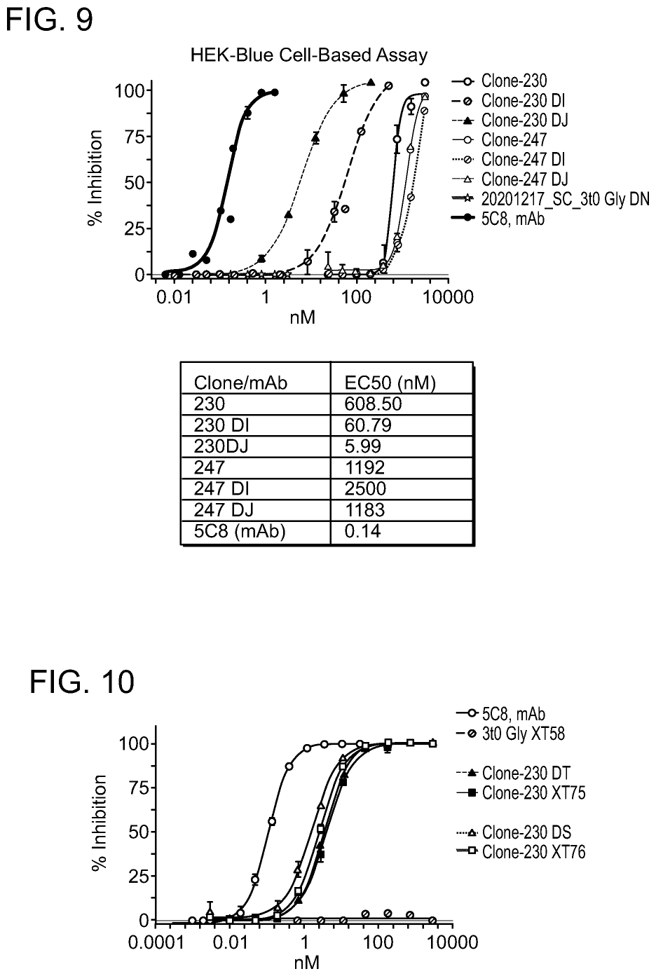

The different formats were also screened using a HEK-Blue cell-based assay, as described above. The results are shown in FIG. 9, and demonstrate that the clone 230 DJ format (trimeric) performed the best, with an EC50 of 5.99 nM.

### Example 6: Characterization of in Line Fusion (ILF) Stefin A Protein Variant in Trimeric or Tetrameric Configurations with HSA Binding Stefin A Protein Variants

In a further experiment, stefin A protein variants were engineered to assemble into stable multimeric oligomers. The resulting stefin A protein variants, which are In Line Fusion (ILF) proteins, can be trimers or tetramers with various linkers (rigid linkers, see e.g., SEQ ID NOs: 508, 510-514; or flexible linkers, see e.g., SEQ ID NOs: 509, 515-518). Four stefin A protein variants were tested: clone-230 DT (trimer with a rigid linker), clone-230 XT75 (trimer with a rigid linker and an HSA binding stefin A protein variants), clone-230 DS (tetramer with a rigid linker), and clone-230 XT76 (tetramer with a rigid linker and an HSA binding stefin A protein variants).

To evaluate the avidity of the different formats, a CD40L competitive ELISA was performed. Briefly, the plates were coated with rhCD40 Fc at 1 μg/ml and incubated overnight at 4° C. The plates were washed twice with 150 μl of washing buffer (PBS, Tween 20 0.1%) with a plate washer and saturated with Casein 5% (Sigma) in PBS for 90 minutes at room temperature (25±1° C.). Human CD40L at 2×EC80 (1 nM) was combined with anti-hCD40L monoclonal antibody starting from 10 nM or with each tested ILF clone. The resulting solution was then mixed and added to the plates. The plates were washed 3 times as described previously. Biotinylated anti-hCD40L polyclonal antibody was then diluted in dilution buffer and added to the plates. The plates were incubated for 90 minutes at room temperature (25±1° C.). The plates were then washed. PolyHRP-Streptavidin was then diluted in dilution buffer and added to the plates. The plates were incubated an additional 90 minutes at room temperature (25±1° C.). Plates were washed 3 times as described previously and the substrate (TMB, Pierce Thermo-Scientific) was added in the plate for 10 minutes. The reaction was stopped using an acidic solution and the plates were read at 450-630 nm and the resulting percent inhibition was calculated. The results are shown in FIG. 10 and demonstrate that the tetrameric formats slightly outcompete the trimeric formats.

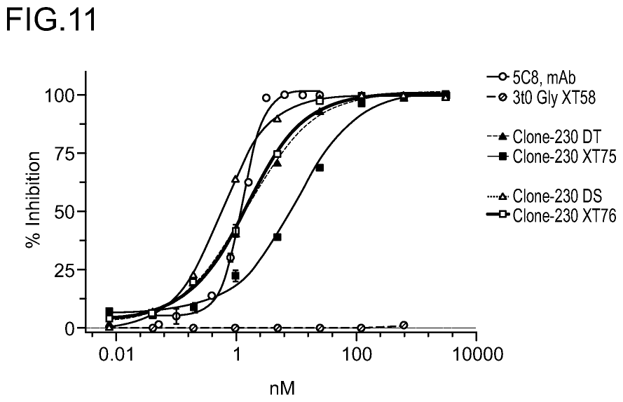

The different formats were also screened using a HEK-Blue cell-based assay, as described above. The results are shown in FIG. 11, and demonstrate that the tetrameric formats outperformed the trimeric formats.

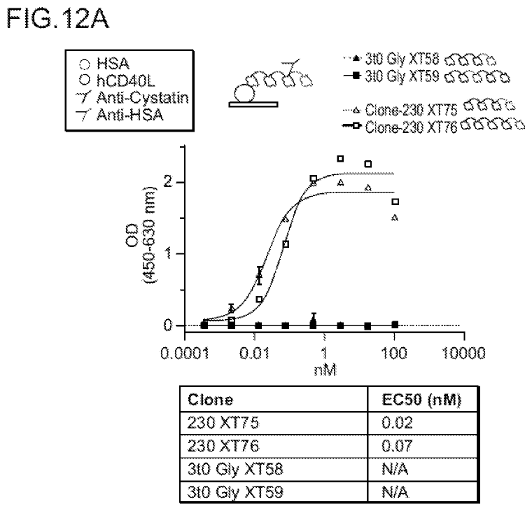

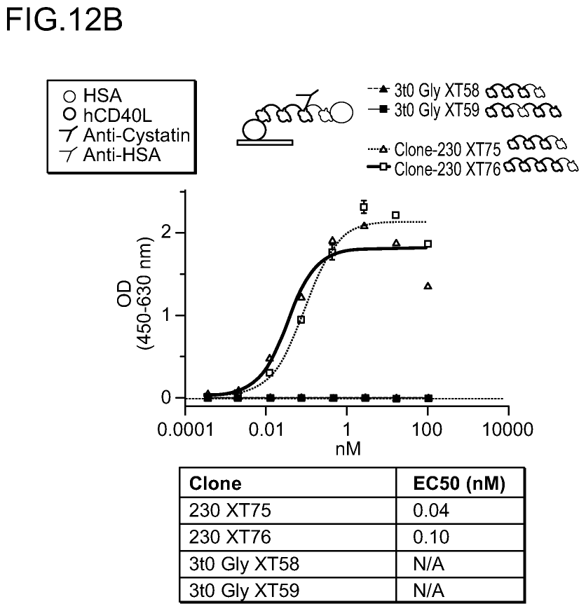

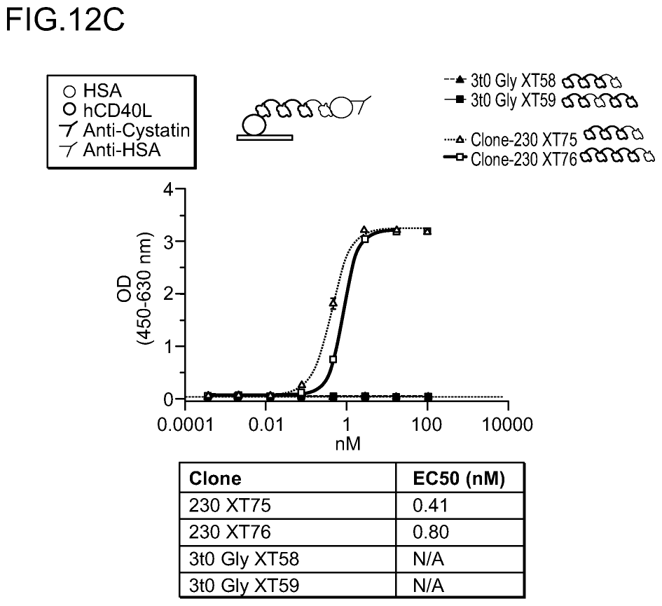

To demonstrate that the different stefin A protein variants ILF proteins were able to engage both targets (human CD40L and HSA) simultaneously, a bridging ELISA was performed. The assay captured the bispecific Stefin A protein variant using hCD40L and detecting the Stefin A protein variant using an anti-HSA antibody, that is, permitting the detection of the HSA binding stefin A protein variants. Briefly, human CD40L was coated on 96 well plates at 0.5 mg/ml in carbonate buffer. After saturation with 5% casein/PBS buffer, the plates were washed and a dilution of Stefin A protein variant or controls was incubated with HSA at a final concentration of 10 μM for 90 minutes. Plates were then washed, and a biotinylated polyclonal antibody, anti-HSA (HRP-conjugated) (Abcam), was added for 90 minutes. After a last washing step, TMB was added for the development of the experiment and the plates were read at 450 nm. The EC50 was then calculated using the interpolated non-linear four-parameters standard curve (FIG. 12C). In control experiments, an anti-cystatin antibody, which binds to the stefin A framework of the AFFIMER® polypeptide was added in place of the anti-HSA antibody in the absence (FIG. 12A) or presence (FIG. 12B) of HSA, demonstrating that the Stefin A protein variant bind hCD40L under both conditions. The bridging ELISA data showed that engagement with HSA does not impact CD40L binding for either of the two AFFIMER® polypeptide formats tested.

### Example 7: Introduction of Anti-CD40L Stefin A Protein Variant Gene Via Lentivirus

### Example 7-1: Selection of Transduction Enhancer

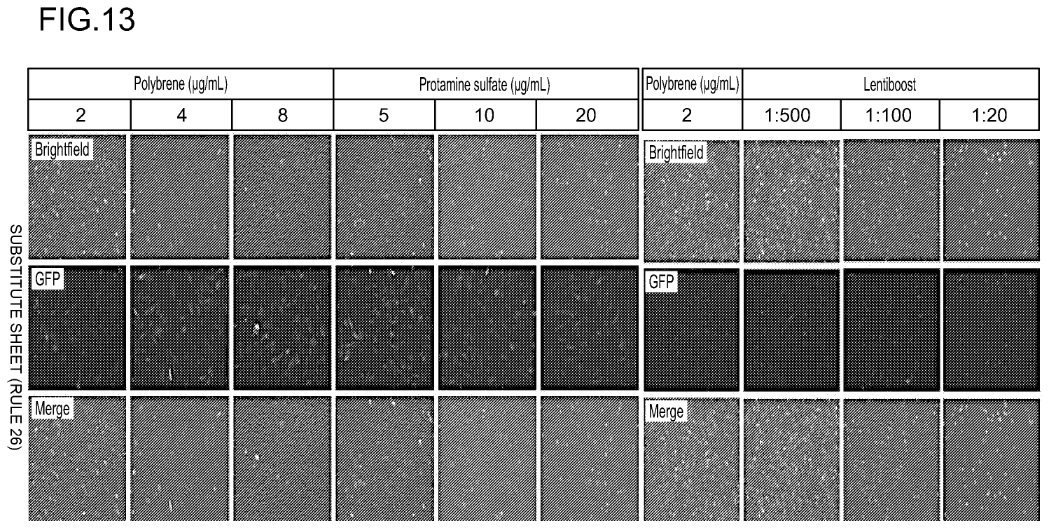

When introducing a gene into PSC-derived MSC (obtained from the Institute of Reproductive Medicine and Population, Seoul National University Medical Research Center) using a lentivirus, a transduction enhancer was selected to increase the introduction efficiency. The transduction enhancer is a cationic polymer and helps the lentivirus to bind to cells through ion neutralization, thereby increasing gene introduction efficiency. Representative transduction enhancers include polybrene and protamine sulfate, which have different efficiencies and sensitivities depending on cell characteristics, so it is necessary to select a suitable transduction enhancer. Hence, in order to select a transduction enhancer suitable for PSC-derived MSC, three types, namely polybrene (Sigma-Aldrich, TR-1003-G), protamine sulfate (Sigma-Aldrich, P3369), and LentiBOOST (SIRION Biotech, SB-P-LV-101-02), were used at different concentrations and introduction efficiency was compared. The cells were treated with 5 MOI of a lentivirus (SIRION Biotech, SEQ ID NO: 729) constructed using a vector including the eGFP gene. Here, polybrene at 2, 4, and 8 μg/mL, protamine sulfate at 5, 10, and 20 μg/mL, and LentiBOOST at concentrations of 1:500, 1:100, and 1:20 according to the manufacturer's recommendations were also used for cell treatment. 16 to 20 hours after treatment with the lentivirus, the culture medium containing the lentivirus was removed and replaced with a fresh culture medium (FUJIFILM Irvine Scientific, 991333), followed by culture for 48 hours. After 48 hours, the cells were harvested and gene introduction efficiency was compared based on the GFP fluorescence-introduced cell population through flow cytometry. Consequently, as shown in FIG. 13, polybrene showed an introduction efficiency of 66.27% at a concentration of 2 μg/mL, protamine sulfate showed an introduction efficiency of 63.90% at a concentration of 20 μg/mL, and LentiBOOST showed an introduction efficiency of 21.98% at 1:500, confirming a tendency of increasing cell death with an increase in the treatment concentration. Based on the results thereof, when introducing the gene into PSC-derived MSC using the lentivirus, polybrene, showing the highest introduction efficiency at a low concentration, exhibited vastly superior introduction efficiency.

The vectors used for the construction of the lentivirus used in Examples of the present invention are as shown in SEQ ID NOs: 729 to 738 of Table 12 below.

TABLE 12

| | | SEQ |
|---|---|---|
| | | ID |
| Vector | Sequence | NO. |
| 7958_pcLV- | aatattttgaagcatttatcagggttattgtctcatgagcggatacatatt | 729 |
| CMV-eGFP- | tgaatgtatttagaaaaataaacaaataggggttccgcgcacatttccccg | |
| IRES-Neo- | aaaagtgccacctgacgtctaagaaaccattattatcatgacattaaccta | |
| WPRE | taaaaataggcgtatcacgaggccctttcgtctcgcgcgtttcggtgatga | |
| (8566 bp) | cggtgaaaacctctgacacatgcagctcccggagacggtcacagcttgtct | |
| | gtaagcggatgccgggagcagacaagcccgtcagggcgcgtcagcgggtgt | |
| | tggcgggtgtcggggctggcttaactatgcggcatcagagcagattgtact | |
| | gagagtgcaccatatgcggtgtgaaataccgcacagatgcgtaaggagaaa | |
| | ataccgcatcaggcgccattcgccattcaggctgcgcaactgttgggaagg | |
| | gcgatcggtgcgggcctcttcgctattacgccagctggcgaaagggggatg | |
| | tgctgcaaggcgattaagttgggtaacgccagggttttcccagtcacgacg | |
| | ttgtaaaacgacggccagtgccaagctgacgcgtgtagtcttatgcaatac | |
| | tcttgtagtcttgcaacatggtaacgatgagttagcaacatgccttacaag | |
| | gagagaaaaagcaccgtgcatgccgattggtggaagtaaggtggtacgatc | |
| | gtgccttattaggaaggcaacagacgggtctgacatggattggacgaacca | |
| | ctgaattgccgcattgcagagatattgtatttaagtgcctagctcgataca | |
| | ataaacgggtctctctggttagaccagatctgagcctgggagctctctggc | |
| | taactagggaacccactgcttaagcctcaataaagcttgccttgagtgctt | |
| | caagtagtgtgtgcccgtctgttgtgtgactctggtaactagagatccctc | |
| | agacccttttagtcagtgtggaaaatctctagcagtggcgcccgaacaggg | |
| | acctgaaagcgaaagggaaaccagagctctctcgacgcaggactcggcttg | |
| | ctgaagcgcgcacggcaagaggcgaggggcggcgactggtgagtacgccaa | |
| | aaattttgactagcggaggctagaaggagagagatgggtgcgagagcgtca | |
| | gtattaagcgggggagaattagatcgcgatgggaaaaaattcggttaaggc | |
| | cagggggaaagaaaaaatataaattaaaacatatagtatgggcaagcaggg | |
| | agctagaacgattcgcagttaatcctggcctgttagaaacatcagaaggct | |
| | gtagacaaatactgggacagctacaaccatcccttcagacaggatcagaag | |
| | aacttagatcattatataatacagtagcaaccctctattgtgtgcatcaaa | |
| | ggatagagataaaagacaccaaggaagctttagacaagatagaggaagagc | |
| | aaaacaaaagtaagaccaccgcacagcaagcggccactgatcttcagacct | |
| | ggaggaggagatatgagggacaattggagaagtgaattatataaatataaa | |
| | gtagtaaaaattgaaccattaggagtagcacccaccaaggcaaagagaaga | |
| | gtggtgcagagagaaaaaagagcagtgggaataggagctttgttccttggg | |
| | ttcttgggagcagcaggaagcactatgggcgcagcctcaatgacgctgacg | |
| | gtacaggccagacaattattgtctggtatagtgcagcagcagaacaatttg | |
| | ctgagggctattgaggcgcaacagcatctgttgcaactcacagtctggggc | |
| | atcaagcagctccaggcaagaatcctggctgtggaaagatacctaaaggat | |
| | caacagctcctggggatttggggttgctctggaaaactcatttgcaccact | |
| | gctgtgccttggaatgctagttggagtaataaatctctggaacagattgga | |
| | atcacacgacctggatggagtgggacagagaaattaacaattacacaagct | |
| | taatacactccttaattgaagaatcgcaaaaccagcaagaaaagaatgaac | |
| | aagaattattggaattagataaatgggcaagtttgtggaattggtttaaca | |
| | taacaaattggctgtggtatataaaattattcataatgatagtaggaggct | |
| | tggtaggtttaagaatagtttttgctgtactttctatagtgaatagagtta | |
| | ggcagggatattcaccattatcgtttcagacccacctcccaaccccgaggg | |
| | gacccgacaggcccgaaggaatagaagaagaaggtggagagagagacagag | |
| | acagatccattcgattagtgaacggatctcgacggtatcggttaactttta | |
| | aaagaaaaggggggattggggggtacagtgcaggggaaagaatagtagaca | |
| | taatagcaacagacatacaaactaaagaattacaaaaacaaattacaaaat | |
| | tcaaaattttatcgatactagtggccctcctatagtgagtcgtattatact | |
| | atgccgatatactatgccgatgattaattgtcaacacgtgctgcagactag | |
| | tattatgcccagtacatgaccttatgggactttcctacttggcagtacatc | |
| | tacgtattagtcatcgctattaccatggtgatgcggttttggcagtacatc | |
| | aatgggcgtggatagcggtttgactcacggggatttccaagtctccacccc | |
| | attgacgtcaatgggagtttgttttggcaccaaaatcaacgggactttcca | |
| | aaatgtcgtaacaactccgccccattgacgcaaatgggcggtaggcgtgta | |
| | cggtgggaggtttatataagcagagctcgtttagtgaaccgtcagatcgcc | |
| | tggagacgccatccacgctgttttgacctccatagaagagctagcctagcg | |
| | gatccgaattctcgatcacaagtttgtacaaaaaagcaggctttaaaggaa | |
| | ccaattcagtcgccgccaccatggtgagcaagggcgaggagctgttcaccg | |
| | gggtggtgcccatcctggtcgagctggacggcgacgtaaacggccacaagt | |
| | tcagcgtgtccggcgagggcgagggcgatgccacctacggcaagctgaccc | |
| | tgaagttcatctgcaccaccggcaagctgcccgtgccctggcccaccctcg | |
| | tgaccaccctgacctacggcgtgcagtgcttcagccgctaccccgaccaca | |
| | tgaagcagcacgacttcttcaagtccgccatgcccgaaggctacgtccagg | |
| | agcgcaccatcttcttcaaggacgacggcaactacaagacccgcgccgagg | |
| | tgaagttcgagggcgacaccctggtgaaccgcatcgagctgaagggcatcg | |
| | acttcaaggaggacggcaacatcctggggcacaagctggagtacaactaca | |
| | acagccacaacgtctatatcatggccgacaagcagaagaacggcatcaagg | |
| | tgaacttcaagatccgccacaacatcgaggacggcagcgtgcagctcgccg | |
| | accactaccagcagaacacccccatcggcgacggccccgtgctgctgcccg | |
| | acaaccactacctgagcacccagtccgccctgagcaaagaccccaacgaga | |
| | agcgcgatcacatggtcctgctggagttcgtgaccgccgccgggatcactc | |
| | tcggcatggacgagctgtacaagaattagcgctactagtgatatcaagatg | |
| | atctagacccagctttcttgtacaaagtggtatcgagatatctgcatttaa | |
| | ttaatctagagtttaaacgcggccgcaaattccgcccctctccctcccccc | |
| | cccctaacgttactggccgaagccgcttggaataaggccggtgtgcgtttg | |
| | tctatatgttattttccaccatattgccgtcttttggcaatgtgagggccc | |
| | ggaaacctggccctgtcttcttgacgagcattcctaggggtctttcccctc | |
| | tcgccaaaggaatgcaaggtctgttgaatgtcgtgaaggaagcagttcctc | |
| | tggaagcttcttgaagacaaacaacgtctgtagcgaccctttgcaggcagc | |
| | ggaaccccccacctggcgacaggtgcctctgcggccaaaagccacgtgtat | |
| | aagatacacctgcaaaggcggcacaaccccagtgccacgttgtgagttgga | |
| | tagttgtggaaagagtcaaatggctctcctcaagcgtattcaacaaggggc | |
| | tgaaggatgcccagaaggtaccccattgtatgggatctgatctggggcctc | |
| | ggtgcacatgctttacatgtgtttagtcgaggttaaaaaaacgtctaggcc | |
| | ccccgaaccacggggacgtggttttcctttgaaaaacacgatgataatatg | |
| | gccacaaccatggttattgaacaagatggattgcacgcaggttctccggcc | |
| | gcttgggtggagaggctattcggctatgactgggcacaacagacaatcggc | |
| | tgctctgatgccgccgtgttccggctgtcagcgcaggggcgcccggttctt | |
| | tttgtcaagaccgacctgtccggtgccctgaatgaactgcaggacgaggca | |
| | gcgcggctatcgtggctggccacgacgggcgttccttgcgcagctgtgctc | |
| | gacgttgtcactgaagcgggaagggactggctgctattgggcgaagtgccg | |
| | gggcaggatctcctgtcatctcaccttgctcctgccgagaaagtatccatc | |
| | atggctgatgcaatgcggcggctgcatacgcttgatccggctacctgccca | |
| | ttcgaccaccaagcgaaacatcgcatcgagcgagcacgtactcggatggaa | |
| | gccggtcttgtcgatcaggatgatctggacgaagagcatcaggggctcgcg | |
| | ccagccgaactgttcgccaggctcaaggcgcgcatgcccgacggcgaggat | |
| | ctcgtcgtgacccatggcgatgcctgcttgccgaatatcatggtggaaaat | |
| | ggccgcttttctggattcatcgactgtggccggctgggtgtggcggaccgc | |
| | tatcaggacatagcgttggctacccgtgatattgctgaagagcttggcggc | |
| | gaatgggctgaccgcttcctcgtgctttacggtatcgccgctcccgattcg | |
| | cagcgcatcgccttctatcgccttcttgacgagttcttctgagtcgacaat | |
| | caacctctggattacaaaatttgtgaaagattgactggtattcttaactat | |
| | gttgctccttttacgctatgtggatacgctgctttaatgcctttgtatcat | |
| | gctattgcttcccgtatggctttcattttctcctccttgtataaatcctgg | |
| | ttgctgtctctttatgaggagttgtggcccgttgtcaggcaacgtggcgtg | |
| | gtgtgcactgtgtttgctgacgcaacccccactggttggggcattgccacc | |
| | acctgtcagctcctttccgggactttcgctttccccctccctattgccacg | |
| | gcggaactcatcgccgcctgccttgcccgctgctggacaggggctcggctg | |
| | ttgggcactgacaattccgtggtgttgtcggggaaatcatcgtcctttcct | |
| | tggctgctcgcctgtgttgccacctggattctgcgcgggacgtccttctgc | |
| | tacgtcccttcggccctcaatccagcggaccttccttcccgcggcctgctg | |
| | ccggctctgcggcctcttccgcgtcttcgccttcgccctcagacgagtcgg | |
| | atctccctttgggccgcctccccgcctggtacctttaagaccaatgactta | |
| | caaggcagctgtagatcttagccactttttaaaagaaaaggggggactgga | |
| | agggctaattcactcccaacgaaaataagatctgctttttgcttgtactgg | |
| | gtctctctggttagaccagatctgagcctgggagctctctggctaactagg | |
| | gaacccactgcttaagcctcaataaagcttgccttgagtgcttcaagtagt | |
| | gtgtgcccgtctgttgtgtgactctggtaactagagatccctcagaccctt | |
| | ttagtcagtgtggaaaatctctagcagtagtagttcatgtcatcttattat | |
| | tcagtatttataacttgcaaagaaatgaatatcagagagtgagaggaactt | |
| | gtttattgcagcttataatggttacaaataaagcaatagcatcacaaattt | |
| | cacaaataaagcatttttttcactgcattctagttgtggtttgtccaaact | |
| | catcaatgtatcttatcatgtctggctctagctatcccgcccctaactccg | |
| | cccagttccgcccattctccgccccatggctgactaattttttttatttat | |
| | gcagaggccgaggccgcctcggcctctgagctattccagaagtagtgagga | |
| | ggcttttttggaggcctagacttttgcagagacggcccaaattcgtaatca | |
| | tggtcatagctgtttcctgtgtgaaattgttatccgctcacaattccacac | |
| | aacatacgagccggaagcataaagtgtaaagcctggggtgcctaatgagtg | |
| | agctaactcacattaattgcgttgcgctcactgcccgctttccagtcggga | |
| | aacctgtcgtgccagctgcattaatgaatcggccaacgcgcggggagaggc | |
| | ggtttgcgtattgggcgctcttccgcttcctcgctcactgactcgctgcgc | |
| | tcggtcgttcggctgcggcgagcggtatcagctcactcaaaggcggtaata | |
| | cggttatccacagaatcaggggataacgcaggaaagaacatgtgagcaaaa | |
| | ggccagcaaaaggccaggaaccgtaaaaaggccgcgttgctggcgtttttc | |
| | cataggctccgcccccctgacgagcatcacaaaaatcgacgctcaagtcag | |
| | aggtggcgaaacccgacaggactataaagataccaggcgtttccccctgga | |
| | agctccctcgtgcgctctcctgttccgaccctgccgcttaccggatacctg | |
| | tccgcctttctcccttcgggaagcgtggcgctttctcatagctcacgctgt | |
| | aggtatctcagttcggtgtaggtcgttcgctccaagctgggctgtgtgcac | |
| | gaaccccccgttcagcccgaccgctgcgccttatccggtaactatcgtctt | |
| | gagtccaacccggtaagacacgacttatcgccactggcagcagccactggt | |
| | aacaggattagcagagcgaggtatgtaggcggtgctacagagttcttgaag | |
| | tggtggcctaactacggctacactagaaggacagtatttggtatctgcgct | |
| | ctgctgaagccagttaccttcggaaaaagagttggtagctcttgatccggc | |
| | aaacaaaccaccgctggtagcggtggtttttttgtttgcaagcagcagatt | |
| | acgcgcagaaaaaaaggatctcaagaagatcctttgatcttttctacgggg | |
| | tctgacgctcagtggaacgaaaactcacgttaagggattttggtcatgaga | |
| | ttatcaaaaaggatcttcacctagatccttttaaattaaaaatgaagtttt | |
| | aaatcaatctaaagtatatatgagtaaacttggtctgacagttaccaatgc | |
| | ttaatcagtgaggcacctatctcagcgatctgtctatttcgttcatccata | |
| | gttgcctgactccccgtcgtgtagataactacgatacgggagggcttacca | |
| | tctggccccagtgctgcaatgataccgcgagacccacgctcaccggctcca | |
| | gatttatcagcaataaaccagccagccggaagggccgagcgcagaagtggt | |
| | cctgcaactttatccgcctccatccagtctattaattgttgccgggaagct | |
| | agagtaagtagttcgccagttaatagtttgcgcaacgttgttgccattgct | |
| | acaggcatcgtggtgtcacgctcgtcgtttggtatggcttcattcagctcc | |
| | ggttcccaacgatcaaggcgagttacatgatcccccatgttgtgcaaaaaa | |
| | gcggttagctccttcggtcctccgatcgttgtcagaagtaagttggccgca | |
| | gtgttatcactcatggttatggcagcactgcataattctcttactgtcatg | |
| | ccatccgtaagatgcttttctgtgactggtgagtactcaaccaagtcattc | |
| | tgagaatagtgtatgcggcgaccgagttgctcttgcccggcgtcaatacgg | |
| | gataataccgcgccacatagcagaactttaaaagtgctcatcattggaaaa | |
| | cgttcttcggggcgaaaactctcaaggatcttaccgctgttgagatccagt | |
| | tcgatgtaacccactcgtgcacccaactgatcttcagcatcttttactttc | |
| | accagcgtttctgggtgagcaaaaacaggaaggcaaaatgccgcaaaaaag | |
| | ggaataagggcgacacggaaatgttgaatactcatactcttcctttttc | |
| 8461_pcLV- | aatattttgaagcatttatcagggttattgtctcatgagcggatacatatt | 730 |
| CMV-AFX01- | tgaatgtatttagaaaaataaacaaataggggttccgcgcacatttccccg | |
| 230 | aaaagtgccacctgacgtctaagaaaccattattatcatgacattaaccta | |
| XT75(C)- | taaaaataggcgtatcacgaggccctttcgtctcgcgcgtttcggtgatga | |
| IRES-Neo | cggtgaaaacctctgacacatgcagctcccggagacggtcacagcttgtct | |
| (9590 bp) | gtaagcggatgccgggagcagacaagcccgtcagggcgcgtcagcgggtgt | |
| | tggcgggtgtcggggctggcttaactatgcggcatcagagcagattgtact | |
| | gagagtgcaccatatgcggtgtgaaataccgcacagatgcgtaaggagaaa | |
| | ataccgcatcaggcgccattcgccattcaggctgcgcaactgttgggaagg | |
| | gcgatcggtgcgggcctcttcgctattacgccagctggcgaaagggggatg | |
| | tgctgcaaggcgattaagttgggtaacgccagggttttcccagtcacgacg | |
| | ttgtaaaacgacggccagtgccaagctgacgcgtgtagtcttatgcaatac | |
| | tcttgtagtcttgcaacatggtaacgatgagttagcaacatgccttacaag | |
| | gagagaaaaagcaccgtgcatgccgattggtggaagtaaggtggtacgatc | |
| | gtgccttattaggaaggcaacagacgggtctgacatggattggacgaacca | |
| | ctgaattgccgcattgcagagatattgtatttaagtgcctagctcgataca | |
| | taaacgggtctctctggttagaccagatctgagcctgggagctctctggct | |
| | aactagggaacccactgcttaagcctcaataaagcttgccttgagtgcttc | |
| | aagtagtgtgtgcccgtctgttgtgtgactctggtaactagagatccctca | |
| | gacccttttagtcagtgtggaaaatctctagcagtggcgcccgaacaggga | |
| | cttgaaagcgaaagggaaaccagaggagctctctcgacgcaggactcggct | |
| | tgctgaagcgcgcacggcaagaggcgaggggcggcgactggtgagtacgcc | |
| | aaaaattttgactagcggaggctagaaggagagagatgggtgcgagagcgt | |
| | cagtattaagcgggggagaattagatcgcgatgggaaaaaattcggttaag | |
| | gccagggggaaagaaaaaatataaattaaaacatatagtatgggcaagcag | |
| | ggagctagaacgattcgcagttaatcctggcctgttagaaacatcagaagg | |
| | ctgtagacaaatactgggacagctacaaccatcccttcagacaggatcaga | |
| | agaacttagatcattatataatacagtagcaaccctctattgtgtgcatca | |
| | aaggatagagataaaagacaccaaggaagctttagacaagatagaggaaga | |
| | gcaaaacaaaagtaagaccaccgcacagcaagcggccactgatcttcagac | |
| | ctggaggaggagatatgagggacaattggagaagtgaattatataaatata | |
| | aagtagtaaaaattgaaccattaggagtagcacccaccaaggcaaagagaa | |
| | gagtggtgcagagagaaaaaagagcagtgggaataggagctttgttccttg | |
| | ggttcttgggagcagcaggaagcactatgggcgcagcgtcaatgacgctga | |
| | cggtacaggccagacaattattgtctggtatagtgcagcagcagaacaatt | |
| | tgctgagggctattgaggcgcaacagcatctgttgcaactcacagtctggg | |
| | gcatcaagcagctccaggcaagaatcctggctgtggaaagatacctaaagg | |
| | atcaacagctcctggggatttggggttgctctggaaaactcatttgcacca | |
| | ctgctgtgccttggaatgctagttggagtaataaatctctggaacagattt | |
| | ggaatcacacgacctggatggagtgggacagagaaattaacaattacacaa | |
| | gcttaatacactccttaattgaagaatcgcaaaaccagcaagaaaagaatg | |
| | aacaagaattattggaattagataaatgggcaagtttgtggaattggttta | |
| | acataacaaattggctgtggtatataaaattattcataatgatagtaggag | |
| | gcttggtaggtttaagaatagtttttgctgtactttctatagtgaatagag | |
| | ttaggcagggatattcaccattatcgtttcagacccacctcccaaccccga | |
| | ggggacccgacaggcccgaaggaatagaagaagaaggtggagagagagaca | |
| | gagacagatccattcgattagtgaacggatctcgacggtatcggttaactt | |
| | ttaaaagaaaaggggggattggggggtacagtgcaggggaaagaatagtag | |
| | acataatagcaacagacatacaaactaaagaattacaaaaacaaattacaa | |
| | aattcaaaattttatttccgcgttacataacttacggtaaatggcccgcct | |
| | ggctgaccgcccaacgacccccgcccattgacgtcaataatgacgtatgtt | |
| | cccatagtaacgccaatagggactttccattgacgtcaatgggtggagtat | |
| | ttacggtaaactgcccacttggcagtacatcaagtgtatcatatgccaagt | |
| | acgccccctattgacgtcaatgacggtaaatggcccgcctggcattatgcc | |
| | cagtacatgaccttatgggactttcctacttggcagtacatctacgtatta | |
| | gtcatcgctattaccatggtgatgcggttttggcagtacatcaatgggcgt | |
| | ggatagcggtttgactcacggggatttccaagtctccaccccattgacgtc | |
| | aatgggagtttgttttggcaccaaaatcaacgggactttccaaaatgtcgt | |
| | aacaactccgccccattgacgcaaatgggcggtaggcgtgtacggtgggag | |
| | gtctatataagcagagctggtttagtgaaccgtcagatccgctagcctagc | |
| | ggatccgaattctcgatcacaagtttgtacaaaaaagcaggcttcgctagc | |
| | cgccaccatgcccctgctcctgctgctgcctctgctgtgggccggtgcatt | |
| | ggccatccctggagggctgtccgaggccaagcctgctacgccggaaattca | |
| | agaaatcgtcgataaggttaagccacagctcgaagagaaaactggggaaac | |
| | atacggcaaacttgaggcggttcagtacaaaactcaggtcgtgcactacta | |
| | cgtgcactacaacgatcaagggaccaactactatatcaaggtccgcgcggg | |
| | agacaacaaatacatgcatctcaaagtgttcaaatcgctgtggggcgagaa | |
| | ccttttcgcgaagtgggaggatttggtgctgactggatatcaggtcgataa | |
| | aaacaaggatgacgagcttactggctttgccgaggctgcagccaaagaagc | |
| | cgcggcgaaggaagcggcggcaaaggaggcagccgcaaaggaagctgccgc | |
| | taaagaggccgccgcaaagatgattcccggaggcctctcagaagccaaacc | |
| | agccactcctgagattcaggaaattgtcgacaaggtcaagccgcagctgga | |
| | agagaagactggcgaaacctacgggaagctagaggcggtccagtacaagac | |
| | ccaagtcgtccattactatgtacattacaacgaccagggaaccaattacta | |
| | cattaaagtgcgggccggagataataagtatatgcatcttaaggtgtttaa | |
| | gtcgttgtggggcgaaaacctgtttgccaagtgggaagatcttgtgctcac | |
| | cggttaccaagtggacaagaacaaggacgacgagctgaccgggttcgctga | |
| | ggctgccgccaaggaagccgcagcgaaggaggccgctgcgaaagaagcggc | |
| | agccaaggaggcggctgccaaagaggcggccgcgaagatgatacctggcgg | |
| | actgtcggaggcgaaaccggccacccctgaaatccaggagattgtggacaa | |
| | ggtgaaaccccaactcgaggaaaaaaccggcgaaacgtacggtaaactcga | |
| | agccgtgcagtataagacccaggtcgtacactactatgtgcactataacga | |
| | tcagggaacgaactactacatcaaggtcagagctggagacaataagtacat | |
| | gcacctcaaggttttcaagtcgttatggggagaaaacctcttcgcgaaatg | |
| | ggaggatctcgtgctgaccggctaccaggtggacaaaaataaggatgatga | |
| | actcaccggtttcgctgaagcagcggcgaaagaggcagcggccaaggaagc | |
| | cgctgccaaggaagccgccgctaaggaggctgcggctaaggaagcagccgc | |
| | caagatgatcccacggggtctgtccgaagccaagccagccaccccagagat | |
| | ccaagaaatagtggataaggtgaagccccagcttgaggaaaagactggaga | |
| | gacttacggaaagctcgaggctgtccaatacaaaacacaggtgctggcgaa | |
| | cttcttccaaaggagatggccaggcagtactaactattacatcaaagtgcg | |
| | cgcaggagataacaaatatatgcacttgaaggtgttcaatggaccgtggaa | |
| | gttccggaacaccgacagaggcgccgacagggtcttgaccgggtaccaagt | |
| | cgataagaacaaagacgacgaactgaccggattctgaatctagagacccag | |
| | ctttcttgtacaaagtggtatcgagatatctgcatttaattaatctagagt | |
| | ttaaacgcggccgcaaattccgcccctctccctcccccccccctaacgtta | |
| | ctggccgaagccgcttggaataaggccggtgtgcgtttgtctatatgttat | |
| | tttccaccatattgccgtcttttggcaatgtgagggcccggaaacctggcc | |
| | ctgtcttcttgacgagcattcctaggggtctttcccctctcgccaaaggaa | |
| | tgcaaggtctgttgaatgtcgtgaaggaagcagttcctctggaagcttctt | |
| | gaagacaaacaacgtctgtagcgaccctttgcaggcagcggaaccccccac | |
| | ctggcgacaggtgcctctgcggccaaaagccacgtgtataagatacacctg | |
| | caaaggcggcacaaccccagtgccacgttgtgagttggatagttgtggaaa | |
| | gagtcaaatggctctcctcaagcgtattcaacaaggggctgaaggatgccc | |
| | agaaggtaccccattgtatgggatctgatctggggcctcggtgcacatgct | |
| | ttacatgtgtttagtcgaggttaaaaaaacgtctaggccccccgaaccacg | |
| | gggacgtggttttcctttgaaaaacacgatgataatatggccacaaccatg | |
| | gttattgaacaagatggattgcacgcaggttctccggccgcttgggtggag | |
| | aggctattcggctatgactgggcacaacagacaatcggctgctctgatgcc | |
| | gccgtgttccggctgtcagcgcaggggcgcccggttctttttgtcaagacc | |
| | gacctgtccggtgccctgaatgaactgcaggacgaggcagcgcggctatcg | |
| | tggctggccacgacgggcgttccttgcgcagctgtgctcgacgttgtcact | |
| | gaagcgggaagggactggctgctattgggcgaagtgccggggcaggatctc | |
| | ctgtcatctcaccttgctcctgccgagaaagtatccatcatggctgatgca | |
| | atgcggcggctgcatacgcttgatccggctacctgcccattcgaccaccaa | |
| | gcgaaacatcgcatcgagcgagcacgtactcggatggaagccggtcttgtc | |
| | gatcaggatgatctggacgaagagcatcaggggctcgcgccagccgaactg | |
| | ttcgccaggctcaaggcgcgcatgcccgacggcgaggatctcgtcgtgacc | |
| | catggcgatgcctgcttgccgaatatcatggtggaaaatggccgcttttct | |
| | ggattcatcgactgtggccggctgggtgtggcggaccgctatcaggacata | |
| | gcgttggctacccgtgatattgctgaagagcttggcggcgaatgggctgac | |
| | cgcttcctcgtgctttacggtatcgccgctcccgattcgcagcgcatcgcc | |
| | ttctatcgccttcttgacgagttcttctgagtcgacaatcaacctctggat | |
| | tacaaaatttgtgaaagattgactggtattcttaactatgttgctcctttt | |
| | acgctatgtggatacgctgctttaatgcctttgtatcatgctattgcttcc | |
| | cgtatggctttcattttctcctccttgtataaatcctggttgctgtctctt | |
| | tatgaggagttgtggcccgttgtcaggcaacgtggcgtggtgtgcactgtg | |
| | tttgctgacgcaacccccactggttggggcattgccaccacctgtcagctc | |
| | ctttccgggactttcgctttccccctccctattgccacggcggaactcatc | |
| | gccgcctgccttgcccgctgctggacaggggctcggctgttgggcactgac | |
| | aattccgtggtgttgtcggggaaatcatcgtcctttccttggctgctcgcc | |
| | tgtgttgccacctggattctgcgcgggacgtccttctgctacgtcccttcg | |
| | gccctcaatccagcggaccttccttcccgcggcctgctgccggctctgcgg | |
| | cctcttccgcgtcttcgccttcgccctcagacgagtcggatctccctttgg | |
| | gccgcctccccgcctggtacctttaagaccaatgacttacaaggcagctgt | |
| | agatcttagccactttttaaaagaaaaggggggactggaagggctaattca | |
| | ctcccaacgaagataagatctgctttttgcttgtactgggtctctctggtt | |
| | agaccagatctgagcctgggagctctctggctaactagggaacccactgct | |
| | taagcctcaataaagcttgccttgagtgcttcaagtagtgtgtgcccgtct | |
| | gttgtgtgactctggtaactagagatccctcagacccttttagtcagtgtg | |
| | gaaaatctctagcagtagtagttcatgtcatcttattattcagtatttata | |
| | acttgcaaagaaatgaatatcagagagtgagaggaacttgtttattgcagc | |
| | ttataatggttacaaataaagcaatagcatcacaaatttcacaaataaagc | |
| | atttttttcactgcattctagttgtggtttgtccaaactcatcaatgtatc | |
| | ttatcatgtctggctctagctatcccgcccctaactccgcccatcccgccc | |
| | ctaactccgcccagttccgcccattctccgccccatggctgactaattttt | |
| | tttatttatgcagaggccgaggccgcctcggcctctgagctattccagaag | |
| | tagtgaggaggcttttttggaggcctagacttttgcagagaccaaattcgt | |
| | aatcatgtcatagctgtttcctgtgtgaaattgttatccgctcacaattcc | |
| | acacaacatacgagccggaagcataaagtgtaaagcctggggtgcctaatg | |
| | agtgagctaactcacattaattgcgttgcgctcactgcccgctttccagtc | |
| | gggaaacctgtcgtgccagctgcattaatgaatcggccaacgcgcggggag | |
| | aggcggtttgcgtattgggcgctcttccgcttcctcgctcactgactcgct | |
| | gcgctcggtcgttcggctgcggcgagcggtatcagctcactcaaaggcggt | |
| | aatacggttatccacagaatcaggggataacgcaggaaagaacatgtgagc | |
| | aaaaggccagcaaaaggccaggaaccgtaaaaaggccgcgttgctggcgtt | |
| | tttccataggctccgcccccctgacgagcatcacaaaaatcgacgctcaag | |
| | tcagaggtggcgaaacccgacaggactataaagataccaggcgtttccccc | |
| | tggaagctccctcgtgcgctctcctgttccgaccctgccgcttaccggata | |
| | cctgtccgcctttctcccttcgggaagcgtggcgctttctcatagctcacg | |
| | ctgtaggtatctcagttcggtgtaggtcgttcgctccaagctgggctgtgt | |
| | gcacgaaccccccgttcagcccgaccgctgcgccttatccggtaactatcg | |
| | tcttgagtccaacccggtaagacacgacttatcgccactggcagcagccac | |
| | tggtaacaggattagcagagcgaggtatgtaggcggtgctacagagttctt | |
| | gaagtggtggcctaactacggctacactagaaggacagtatttggtatctg | |
| | cgctctgctgaagccagttaccttcggaaaaagagttggtagctcttgatc | |
| | cggcaaacaaaccaccgctggtagcggtggtttttttgtttgcaagcagca | |
| | gattacgcgcagaaaaaaaggatctcaagaagatcctttgatcttttctac | |
| | ggggtctgacgctcagtggaacgaaaactcacgttaagggattttggtcat | |
| | gagattatcaaaaaggatcttcacctagatccttttaaattaaaaatgaag | |
| | ttttaaatcaatctaaagtatatatgagtaaacttggtctgacagttacca | |
| | atgcttaatcagtgaggcacctatctcagcgatctgtctatttcgttcatc | |
| | catagttgcctgactccccgtcgtgtagataactacgatacgggagggctt | |
| | accatctggccccagtgctgcaatgataccgcgagacccacgctcaccggc | |
| | tccagatttatcagcaataaaccagccagccggaagggccgagcgcagaag | |
| | tggtcctgcaactttatccgcctccatccagtctattaattgttgccggga | |
| | agctagagtaagtagttcgccagttaatagtttgcgcaacgttgttgccat | |
| | tgctacaggcatcgtggtgtcacgctcgtcgtttggtatggcttcattcag | |
| | ctccggttcccaacgatcaaggcgagttacatgatcccccatgttgtgcaa | |
| | aaaagcggttagctccttcggtcctccgatcgttgtcagaagtaagttggc | |
| | cgcagtgttatcactcatggttatggcagcactgcataattctcttactgt | |
| | catgccatccgtaagatgcttttctgtgactggtgagtactcaaccaagtc | |
| | attctgagaatagtgtatgcggcgaccgagttgctcttgcccggcgtcaat | |
| | acgggataataccgcgccacatagcagaactttaaaagtgctcatcattgg | |
| | aaaacgttcttcggggcgaaaactctcaaggatcttaccgctgttgagatc | |
| | cagttcgatgtaacccactcgtgcacccaactgatcttcagcatcttttac | |
| | tttcaccagcgtttctgggtgagcaaaaacaggaaggcaaaatgccgcaaa | |
| | aaagggaataagggcgacacggaaatgttgaatactcatactcttcctttt | |
| | tc | |
| VB211001- | aatgtagtcttatgcaatactcttgtagtcttgcaacatggtaacgatgag | 731 |
| 1274vjf | ttagcaacatgccttacaaggagagaaaaagcaccgtgcatgccgattggt | |
| (9895 bp) | ggaagtaaggtggtacgatcgtgccttattaggaaggcaacagacgggtct | |
| | gacatggattggacgaaccactgaattgccgcattgcagagatattgtatt | |
| | taagtgcctagctcgatacataaacgggtctctctggttagaccagatctg | |
| | agcctgggagctctctggctaactagggaacccactgcttaagcctcaata | |
| | aagcttgccttgagtgcttcaagtagtgtgtgcccgtctgttgtgtgactc | |
| | tggtaactagagatccctcagacccttttagtcagtgtggaaaatctctag | |
| | cagtggcgcccgaacagggacttgaaagcgaaagggaaaccagaggagctc | |
| | tctcgacgcaggactcggcttgctgaagcgcgcacggcaagaggcgagggg | |
| | cggcgactggtgagtacgccaaaaattttgactagcggaggctagaaggag | |
| | agagatgggtgcgagagcgtcagtattaagcgggggagaattagatcgcga | |
| | tgggaaaaaattcggttaaggccagggggaaagaaaaaatataaattaaaa | |
| | catatagtatgggcaagcagggagctagaacgattcgcagttaatcctggc | |
| | ctgttagaaacatcagaaggctgtagacaaatactgggacagctacaacca | |
| | tcccttcagacaggatcagaagaacttagatcattatataatacagtagca | |
| | accctctattgtgtgcatcaaaggatagagataaaagacaccaaggaagct | |
| | ttagacaagatagaggaagagcaaaacaaaagtaagaccaccgcacagcaa | |
| | gcggccgctgatcttcagacctggaggaggagatatgagggacaattggag | |
| | aagtgaattatataaatataaagtagtaaaaattgaaccattaggagtagc | |
| | acccaccaaggcaaagagaagagtggtgcagagagaaaaaagagcagtggg | |
| | aataggagctttgttccttgggttcttgggagcagcaggaagcactatggg | |
| | cgcagcgtcaatgacgctgacggtacaggccagacaattattgtctggtat | |
| | agtgcagcagcagaacaatttgctgagggctattgaggcgcaacagcatct | |
| | gttgcaactcacagtctggggcatcaagcagctccaggcaagaatcctggc | |
| | tgtggaaagatacctaaaggatcaacagctcctggggatttggggttgctc | |
| | tggaaaactcatttgcaccactgctgtgccttggaatgctagttggagtaa | |
| | taaatctctggaacagatttggaatcacacgacctggatggagtgggacag | |
| | agaaattaacaattacacaagcttaatacactccttaattgaagaatcgca | |
| | aaaccagcaagaaaagaatgaacaagaattattggaattagataaatgggc | |
| | aagtttgtggaattggtttaacataacaaattggctgtggtatataaaatt | |
| | attcataatgatagtaggaggcttggtaggtttaagaatagtttttgctgt | |
| | actttctatagtgaatagagttaggcagggatattcaccattatcgtttca | |
| | gacccacctcccaaccccgaggggacccgacaggcccgaaggaatagaaga | |
| | agaaggtggagagagagacagagacagatccattcgattagtgaacggatc | |
| | tcgacggtatcgctagcttttaaaagaaaaggggggattggggggtacagt | |
| | gcaggggaaagaatagtagacataatagcaacagacatacaaactaaagaa | |
| | ttacaaaaacaaattacaaaaattcaaaattttactagtatcaactttgta | |
| | tagaaaagttgtagttattaatagtaatcaattacggggtcattagttcat | |
| | agcccatatatggagttccgcgttacataacttacggtaaatggcccgcct | |
| | ggctgaccgcccaacgacccccgcccattgacgtcaataatgacgtatgtt | |
| | cccatagtaacgccaatagggactttccattgacgtcaatgggtggagtat | |
| | ttacggtaaactgcccacttggcagtacatcaagtgtatcatatgccaagt | |
| | acgccccctattgacgtcaatgacggtaaatggcccgcctggcattatgcc | |
| | cagtacatgaccttatgggactttcctacttggcagtacatctacgtatta | |
| | gtcatcgctattaccatggtgatgcggttttggcagtacatcaatgggcgt | |
| | ggatagcggtttgactcacggggatttccaagtctccaccccattgacgtc | |
| | aatgggagtttgttttggcaccaaaatcaacgggactttccaaaatgtcgt | |
| | aacaactccgccccattgacgcaaatgggcggtaggcgtgtacggtgggag | |
| | gtctatataagcagagctggtttagtgaaccgtcagatccaagtttgtaca | |
| | aaaaagcaggctgccaccatgccattgcttcttctgctgccactcctttgg | |
| | gctggtgcgctggctattccggggggtttgtcagaagcaaagcccgcgacc | |
| | ccagaaattcaggagatcgtggataaagtaaaacctcagcttgaagaaaaa | |
| | accggggagacatacggcaagctggaagcagtacaatataaaacgcaggta | |
| | gtacattactacgtccattataacgatcaaggtacaaattattatataaaa | |
| | gtacgggcaggggacaacaaatacatgcatctgaaagtatttaagagtctg | |
| | tggggtgagaacttgtttgcaaagtgggaagatttggtgcttaccggctac | |
| | caggtcgataagaataaggatgacgagcttacaggttttgcagaagctgca | |
| | gcgaaagaggctgccgcaaaagaagcagcggctaaggaagcggccgctaaa | |
| | gaagcggccgcgaaggaagcggccgctaagatgatcccgggtggtctgtcc | |
| | gaagccaaaccggcgacgccggaaatccaggagatagtcgataaggtcaag | |
| | ccacaactcgaagagaagactggagaaacatatggaaaacttgaggccgtt | |
| | caatacaaaacgcaggtggtccactactacgttcactataacgatcaagga | |
| | acaaactattacattaaagtcagggcgggagacaacaagtacatgcatctt | |
| | aaagtgtttaaatctctttggggggagaatcttttcgccaagtgggaagac | |
| | ttggtcctcaccggataccaggtcgacaaaaacaaagacgatgaactcacc | |
| | ggctttgccgaagcggctgcgaaagaagcggctgccaaagaggctgcagcc | |
| | aaagaagctgcggcaaaggaggccgccgccaaagaagcggcggccaaaatg | |
| | atccccggaggacttagcgaggctaaacccgcgacacctgaaatacaggag | |
| | atcgtggataaggttaaaccacaattggaggagaaaactggtgagacctat | |
| | ggcaagctggaagcggttcagtataaaacgcaagttgtacactactatgta | |
| | cattacaacgatcaggggacgaactactatattaaggtcagggcaggagat | |
| | aataaatatatgcacttgaaagttttcaaaagcctgtggggggaaaacttg | |
| | ttcgccaagtgggaagatctcgtcttgactgggtaccaggttgataagaac | |
| | aaagacgatgaactgacaggcttcgctgaagcggctgcaaaggaggcggca | |
| | gcgaaggaggctgccgcgaaagaagcggcggctaaagaagcggccgcaaag | |
| | gaagcagcagcgaagatgataccgaggggtttgagcgaagcaaagcccgca | |
| | acgcctgagatacaagaaatcgttgacaaagtaaaaccacaattggaagag | |
| | aagacgggagaaacgtatggaaagcttgaggccgtacaatacaagacacag | |
| | gtgttggcaaattttttccaaaggcgatggcccggatcaacgaattactat | |
| | atcaaggtacgcgcgggggataacaagtacatgcatctcaaggttttcaac | |
| | ggtccgtggaaattccgaaatacagaccgcggagccgacagggtcctcacc | |
| | ggctatcaggtcgacaagaataaggatgatgagttgacaggtttcgccgcc | |
| | gccggtgagcagaaattgatctccgaagaggatctcggtgccgcgcaccac | |
| | caccaccatcattaaacccagctttcttgtacaaagtgggcccctctccct | |
| | cccccccccctaacgttactggccgaagccgcttggaataaggccggtgtg | |
| | cgtttgtctatatgttattttccaccatattgccgtcttttggcaatgtga | |
| | gggcccggaaacctggccctgtcttcttgacgagcattcctaggggtcttt | |
| | cccctctcgccaaaggaatgcaaggtctgttgaatgtcgtgaaggaagcag | |
| | ttcctctggaagcttcttgaagacaaacaacgtctgtagcgaccctttgca | |
| | ggcagcggaaccccccacctggcgacaggtgcctctgcggccaaaagccac | |
| | gtgtataagatacacctgcaaaggcggcacaaccccagtgccacgttgtga | |
| | gttggatagttgtggaaagagtcaaatggctctcctcaagcgtattcaaca | |
| | aggggctgaaggatgcccagaaggtaccccattgtatgggatctgatctgg | |
| | ggcctcggtgcacatgctttacatgtgtttagtcgaggttaaaaaaacgtc | |
| | taggccccccgaaccacggggacgtggttttcctttgaaaaacacgatgat | |
| | aatatggccacaaccatgattgaacaagatggattgcacgcaggttctccg | |
| | gccgcttgggtggagaggctattcggctatgactgggcacaacagacaatc | |
| | ggctgctctgatgccgccgtgttccggctgtcagcgcaggggcgcccggtt | |
| | ctttttgtcaagaccgacctgtccggtgccctgaatgaactgcaagacgag | |
| | gcagcgcggctatcgtggctggccacgacgggcgttccttgcgcagctgtg | |
| | ctcgacgttgtcactgaagcgggaagggactggctgctattgggcgaagtg | |
| | ccggggcaggatctcctgtcatctcaccttgctcctgccgagaaagtatcc | |
| | atcatggctgatgcaatgcggcggctgcatacgcttgatccggctacctgc | |
| | ccattcgaccaccaagcgaaacatcgcatcgagcgagcacgtactcggatg | |
| | gaagccggtcttgtcgatcaggatgatctggacgaagagcatcaggggctc | |
| | gcgccagccgaactgttcgccaggctcaaggcgagcatgcccgacggcgag | |
| | gatctcgtcgtgacccatggcgatgcctgcttgccgaatatcatggtggaa | |
| | aatggccgcttttctggattcatcgactgtggccggctgggtgtggcggac | |
| | cgctatcaggacatagcgttggctacccgtgatattgctgaagagcttggc | |
| | ggcgaatgggctgaccgcttcctcgtgctttacggtatcgccgctcccgat | |
| | tcgcagcgcatcgccttctatcgccttcttgacgagttcttctgacaactt | |
| | tattatacatagttgatcaattccgataatcaacctctggattacaaaatt | |
| | tgtgaaagattgactggtattcttaactatgttgctccttttacgctatgt | |
| | ggatacgctgctttaatgcctttgtatcatgctattgcttcccgtatggct | |
| | ttcattttctcctccttgtataaatcctggttgctgtctctttatgaggag | |
| | ttgtggcccgttgtcaggcaacgtggcgtggtgtgcactgtgtttgctgac | |
| | gcaacccccactggttggggcattgccaccacctgtcagctcctttccggg | |
| | actttcgctttccccctccctattgccacggcggaactcatcgccgcctgc | |
| | cttgcccgctgctggacaggggctcggctgttgggcactgacaattccgtg | |
| | gtgttgtcggggaagctgacgtcctttccatggctgctcgcctgtgttgcc | |
| | acctggattctgcgcgggacgtccttctgctacgtcccttcggccctcaat | |
| | ccagcggaccttccttcccgcggcctgctgccggctctgcggcctcttccg | |
| | cgtcttcgccttcgccctcagacgagtcggatctccctttgggccgcctcc | |
| | ccgcatcgggaattcccgcggttcgctttaagaccaatgacttacaaggca | |
| | gctgtagatcttagccactttttaaaagaaaaggggggactggaagggcta | |
| | attcactcccaacgaagacaagatctgctttttgcttgtactgggtctctc | |
| | tggttagaccagatctgagcctgggagctctctggctaactagggaaccca | |
| | ctgcttaagcctcaataaagcttgccttgagtgcttcaagtagtgtgtgcc | |
| | cgtctgttgtgtgactctggtaactagagatccctcagacccttttagtca | |
| | gtgtggaaaatctctagcagtagtagttcatgtcatcttattattcagtat | |
| | ttataacttgcaaagaaatgaatatcagagagtgagaggaacttgtttatt | |
| | gcagcttataatggttacaaataaagcaatagcatcacaaatttcacaaat | |
| | aaagcatttttttcactgcattctagttgtggtttgtccaaactcatcaat | |
| | gtatcttatcatgtctggctctagctatcccgcccctaactccgcccatcc | |
| | cgcccctaactccgcccagttccgcccattctccgccccatggctgactaa | |
| | ttttttttatttatgcagaggccgaggccgcctcggcctctgagctattcc | |
| | agaagtagtgaggaggcttttttggaggcctagggacgtacccaattcgcc | |
| | ctatagtgagtcgtattacgcgcgctcactggccgtcgttttacaacgtcg | |
| | tgactgggaaaaccctggcgttacccaacttaatcgccttgcagcacatcc | |
| | ccctttcgccagctggcgtaatagcgaagaggcccgcaccgatcgcccttc | |
| | ccaacagttgcgcagcctgaatggcgaatgggacgcgccctgtagcggcgc | |
| | attaagcgcggcgggtgtggtggttacgcgcagcgtgaccgctacacttgc | |
| | cagcgccctagcgcccgctcctttcgctttcttcccttcctttctcgccac | |
| | gttcgccggctttccccgtcaagctctaaatcgggggctccctttagggtt | |
| | ccgatttagtgctttacggcacctcgaccccaaaaaacttgattagggtga | |
| | tggttcacgtagtgggccatcgccctgatagacggtttttcgccctttgac | |
| | gttggagtccacgttctttaatagtggactcttgttccaaactggaacaac | |
| | actcaaccctatctcggtctattcttttgatttataagggattttgccgat | |
| | ttcggcctattggttaaaaaatgagctgatttaacaaaaatttaacgcgaa | |
| | ttttaacaaaatattaacgcttacaatttaggtggcacttttcggggaaat | |
| | gtgcgcggaacccctatttgtttatttttctaaatacattcaaatatgtat | |
| | ccgctcatgagacaataaccctgataaatgcttcaataatattgaaaaagg | |
| | aagagtatgagtattcaacatttccgtgtcgcccttattcccttttttgcg | |
| | gcattttgccttcctgtttttgctcacccagaaacgctggtgaaagtaaaa | |
| | gatgctgaagatcagttgggtgcacgagtgggttacatcgaactggatctc | |
| | aacagcggtaagatccttgagagttttcgccccgaagaacgttttccaatg | |
| | atgagcacttttaaagttctgctatgtggcgcggtattatcccgtattgac | |
| | gccgggcaagagcaactcggtcgccgcatacactattctcagaatgacttg | |
| | gttgagtactcaccagtcacagaaaagcatcttacggatggcatgacagta | |
| | agagaattatgcagtgctgccataaccatgagtgataacactgcggccaac | |
| | ttacttctgacaacgatcggaggaccgaaggagctaaccgcttttttgcac | |
| | aacatgggggatcatgtaactcgccttgatcgttgggaaccggagctgaat | |
| | gaagccataccaaacgacgagcgtgacaccacgatgcctgtagcaatggca | |
| | acaacgttgcgcaaactattaactggcgaactacttactctagcttcccgg | |
| | caacaattaatagactggatggaggcggataaagttgcaggaccacttctg | |
| | cgctcggcccttccggctggctggtttattgctgataaatctggagccggt | |
| | gagcgtgggtctcgcggtatcattgcagcactggggccagatggtaagccc | |
| | tcccgtatcgtagttatctacacgacggggagtcaggcaactatggatgaa | |
| | cgaaatagacagatcgctgagataggtgcctcactgattaagcattggtaa | |
| | ctgtcagaccaagtttactcatatatactttagattgatttaaaacttcat | |
| | ttttaatttaaaaggatctaggtgaagatcctttttgataatctcatgacc | |
| | aaaatcccttaacgtgagttttcgttccactgagcgtcagaccccgtagaa | |
| | aagatcaaaggatcttcttgagatcctttttttctgcgcgtaatctgctgc | |
| | ttgcaaacaaaaaaaccaccgctaccagcggtggtttgtttgccggatcaa | |
| | gagctaccaactctttttccgaaggtaactggcttcagcagagcgcagata | |
| | ccaaatactgttcttctagtgtagccgtagttaggccaccacttcaagaac | |
| | tctgtagcaccgcctacatacctcgctctgctaatcctgttaccagtggct | |
| | gctgccagtggcgataagtcgtgtcttaccgggttggactcaagacgatag | |
| | ttaccggataaggcgcagcggtcgggctgaacggggggttcgtgcacacag | |
| | cccagcttggagcgaacgacctacaccgaactgagatacctacagcgtgag | |
| | ctatgagaaagcgccacgcttcccgaagagagaaaggcggacaggtatccg | |
| | gtaagcggcagggtcggaacaggagagcgcacgagggagcttccaggggga | |
| | aacgcctggtatctttatagtcctgtcgggtttcgccacctctgacttgag | |
| | cgtcgatttttgtgatgctcgtcaggggggcggagcctatggaaaaacgcc | |
| | agcaacgcggcctttttacggttcctggccttttgctggccttttgctcac | |
| | atgttctttcctgcgttatcccctgattctgtggataaccgtattaccgcc | |
| | tttgagtgagctgataccgctcgccgcagccgaacgaccgagcgcagcgag | |
| | tcagtgagcgaggaagcggaagagcgcccaatacgcaaaccgcctctcccc | |
| | gcgcgttggccgattcattaatgcagctggcacgacaggtttcccgactgg | |
| | aaagcgggcagtgagcgcaacgcaattaatgtgagttagctcactcattag | |
| | gcaccccaggctttacactttatgcttccggctcgtatgttgtgtggaatt | |
| | gtgagcggataacaatttcacacaggaaacagctatgaccatgattacgcc | |
| | aagcgcgcaattaaccctcactaaagggaacaaaagctggagctgcaagct | |
| | t | |
| VB211001- | aatgtagtcttatgcaatactcttgtagtcttgcaacatggtaacgatgag | 732 |
| 1279pfe | ttagcaacatgccttacaaggagagaaaaagcaccgtgcatgccgattggt | |
| (9361 bp) | ggaagtaaggtggtacgatcgtgccttattaggaaggcaacagacgggtct | |
| | gacatggattggacgaaccactgaattgccgcattgcagagatattgtatt | |
| | taagtgcctagctcgatacataaacgggtctctctggttagaccagatctg | |
| | agcctgggagctctctggctaactagggaacccactgcttaagcctcaata | |
| | aagcttgccttgagtgcttcaagtagtgtgtgcccgtctgttgtgtgactc | |
| | tggtaactagagatccctcagacccttttagtcagtgtggaaaatctctag | |
| | cagtggcgcccgaacagggacttgaaagcgaaagggaaaccagaggagctc | |
| | tctcgacgcaggactcggcttgctgaagcgcgcacggcaagaggcgagggg | |
| | cggcgactggtgagtacgccaaaaattttgactagcggaggctagaaggag | |
| | agagatgggtgcgagagcgtcagtattaagcgggggagaattagatcgcga | |
| | tgggaaaaaattcggttaaggccagggggaaagaaaaaatataaattaaaa | |
| | catatagtatgggcaagcagggagctagaacgattcgcagttaatcctggc | |
| | ctgttagaaacatcagaaggctgtagacaaatactgggacagctacaacca | |
| | tcccttcagacaggatcagaagaacttagatcattatataatacagtagca | |
| | accctctattgtgtgcatcaaaggatagagataaaagacaccaaggaagct | |
| | ttagacaagatagaggaagagcaaaacaaaagtaagaccaccgcacagcaa | |
| | gcggccgctgatcttcagacctggaggaggagatatgagggacaattggag | |
| | aagtgaattatataaatataaagtagtaaaaattgaaccattaggagtagc | |
| | acccaccaaggcaaagagaagagtggtgcagagagaaaaaagagcagtggg | |
| | aataggagctttgttccttgggttcttgggagcagcaggaagcactatggg | |
| | cgcagcgtcaatgacgctgacggtacaggccagacaattattgtctggtat | |
| | agtgcagcagcagaacaatttgctgagggctattgaggcgcaacagcatct | |
| | gttgcaactcacagtctggggcatcaagcagctccaggcaagaatcctggc | |
| | tgtggaaagatacctaaaggatcaacagctcctggggatttggggttgctc | |
| | tggaaaactcatttgcaccactgctgtgccttggaatgctagttggagtaa | |
| | taaatctctggaacagatttggaatcacacgacctggatggagtgggacag | |
| | agaaattaacaattacacaagcttaatacactccttaattgaagaatcgca | |
| | aaaccagcaagaaaagaatgaacaagaattattggaattagataaatgggc | |
| | aagtttgtggaattggtttaacataacaaattggctgtggtatataaaatt | |
| | attcataatgatagtaggaggcttggtaggtttaagaatagtttttgctgt | |
| | actttctatagtgaatagagttaggcagggatattcaccattatcgtttca | |
| | gacccacctcccaaccccgaggggacccgacaggcccgaaggaatagaaga | |
| | agaaggtggagagagagacagagacagatccattcgattagtgaacggatc | |
| | tcgacggtatcgctagcttttaaaagaaaaggggggattggggggtacagt | |
| | gcaggggaaagaatagtagacataatagcaacagacatacaaactaaagaa | |
| | ttacaaaaacaaattacaaaaattcaaaattttactagtgattatcggatc | |
| | aactttgtatagaaaagttgtagttattaatagtaatcaattacggggtca | |
| | ttagttcatagcccatatatggagttccgcgttacataacttacggtaaat | |
| | ggcccgcctggctgaccgcccaacgacccccgcccattgacgtcaataatg | |
| | acgtatgttcccatagtaacgccaatagggactttccattgacgtcaatgg | |
| | gtggagtatttacggtaaactgcccacttggcagtacatcaagtgtatcat | |
| | atgccaagtacgccccctattgacgtcaatgacggtaaatggcccgcctgg | |
| | cattatgcccagtacatgaccttatgggactttcctacttggcagtacatc | |
| | tacgtattagtcatcgctattaccatggtgatgcggttttggcagtacatc | |
| | aatgggcgtggatagcggtttgactcacggggatttccaagtctccacccc | |
| | attgacgtcaatgggagtttgttttggcaccaaaatcaacgggactttcca | |
| | aaatgtcgtaacaactccgccccattgacgcaaatgggcggtaggcgtgta | |
| | cggtgggaggtctatataagcagagctggtttagtgaaccgtcagatccaa | |
| | gtttgtacaaaaaagcaggctgccaccatgccattgcttcttctgctgcca | |
| | ctcctttgggctggtgcgctggctattccggggggtttgtcagaagcaaag | |
| | cccgcgaccccagaaattcaggagatcgtggataaagtaaaacctcagctt | |
| | gaagaaaaaaccggggagacatacggcaagctggaagcagtacaatataaa | |
| | acgcaggtagtacattactacgtccattataacgatcaaggtacaaattat | |
| | tatataaaagtacgggcaggggacaacaaatacatgcatctgaaagtattt | |
| | aagagtctgtggggtgagaacttgtttgcaaagtgggaagatttggtgctt | |
| | accggctaccaggtcgataagaataaggatgacgagcttacaggttttgca | |
| | gaagctgcagcgaaagaggctgccgcaaaagaagcagcggctaaggaagcg | |
| | gccgctaaagaagcggccgcgaaggaagcggccgctaagatgatcccgggt | |
| | ggtctgtccgaagccaaaccggcgacgccggaaatccaggagatagtcgat | |
| | aaggtcaagccacaactcgaagagaagactggagaaacatatggaaaactt | |
| | gaggccgttcaatacaaaacgcaggtggtccactactacgttcactataac | |
| | gatcaaggaacaaactattacattaaagtcagggcgggagacaacaagtac | |
| | atgcatcttaaagtgtttaaatctctttggggggagaatcttttcgccaag | |
| | tgggaagacttggtcctcaccggataccaggtcgacaaaaacaaagacgat | |
| | gaactcaccggctttgccgaagcggctgcgaaagaagcggctgccaaagag | |
| | gctgcagccaaagaagctgcggcaaaggaggccgccgccaaagaagcggcg | |
| | gccaaaatgatccccggaggacttagcgaggctaaacccgcgacacctgaa | |
| | atacaggagatcgtggataaggttaaaccacaattggaggagaaaactggt | |
| | gagacctatggcaagctggaagcggttcagtataaaacgcaagttgtacac | |
| | tactatgtacattacaacgatcaggggacgaactactatattaaggtcagg | |
| | gcaggagataataaatatatgcacttgaaagttttcaaaagcctgtggggg | |
| | gaaaacttgttcgccaagtgggaagatctcgtcttgactgggtaccaggtt | |
| | gataagaacaaagacgatgaactgacaggcttcgctgaagcggctgcaaag | |
| | gaggcggcagcgaaggaggctgccgcgaaagaagcggcggctaaagaagcg | |
| | gccgcaaaggaagcagcagcgaagatgataccgaggggtttgagcgaagca | |
| | aagcccgcaacgcctgagatacaagaaatcgttgacaaagtaaaaccacaa | |
| | ttggaagagaagacgggagaaacgtatggaaagcttgaggccgtacaatac | |
| | aagacacaggtgttggcaaattttttccaaaggcgatggcccggatcaacg | |
| | aattactatatcaaggtacgcgcgggggataacaagtacatgcatctcaag | |
| | gttttcaacggtccgtggaaattccgaaatacagaccgcggagccgacagg | |
| | gtcctcaccggctatcaggtcgacaagaataaggatgatgagttgacaggt | |
| | ttcgccgccgccggtgagcagaaattgatctccgaagaggatctcggtgcc | |
| | gcgcaccaccaccaccatcatggaagcggagagggcaggggaagtcttcta | |
| | acatgcggggacgtggaggaaaatcccggccccatgattgaacaagatgga | |
| | ttgcacgcaggttctccggccgcttgggtggagaggctattcggctatgac | |
| | tgggcacaacagacaatcggctgctctgatgccgccgtgttccggctgtca | |
| | gcgcaggggcgcccggttctttttgtcaagaccgacctgtccggtgccctg | |
| | aatgaactgcaagacgaggcagcgcggctatcgtggctggccacgacgggc | |
| | gttccttgcgcagctgtgctcgacgttgtcactgaagcgggaagggactgg | |
| | ctgctattgggcgaagtgccggggcaggatctcctgtcatctcaccttgct | |
| | cctgccgagaaagtatccatcatggctgatgcaatgcggcggctgcatacg | |
| | cttgatccggctacctgcccattcgaccaccaagcgaaacatcgcatcgag | |
| | cgagcacgtactcggatggaagccggtcttgtcgatcaggatgatctggac | |
| | gaagagcatcaggggctcgcgccagccgaactgttcgccaggctcaaggcg | |
| | agcatgcccgacggcgaggatctcgtcgtgacccatggcgatgcctgcttg | |
| | ccgaatatcatggtggaaaatggccgcttttctggattcatcgactgtggc | |
| | cggctgggtgtggcggaccgctatcaggacatagcgttggctacccgtgat | |
| | attgctgaagagcttggcggcgaatgggctgaccgcttcctcgtgctttac | |
| | ggtatcgccgctcccgattcgcagcgcatcgccttctatcgccttcttgac | |
| | gagttcttctgaacccagctttcttgtacaaagtggtgataatcgaattcc | |
| | gataatcaacctctggattacaaaatttgtgaaagattgactggtattctt | |
| | aactatgttgctccttttacgctatgtggatacgctgctttaatgcctttg | |
| | tatcatgctattgcttcccgtatggctttcattttctcctccttgtataaa | |
| | tcctggttgctgtctctttatgaggagttgtggcccgttgtcaggcaacgt | |
| | ggcgtggtgtgcactgtgtttgctgacgcaacccccactggttggggcatt | |
| | gccaccacctgtcagctcctttccgggactttcgctttccccctccctatt | |
| | gccacggcggaactcatcgccgcctgccttgcccgctgctggacaggggct | |
| | cggctgttgggcactgacaattccgtggtgttgtcggggaagctgacgtcc | |
| | tttccatggctgctcgcctgtgttgccacctggattctgcgcgggacgtcc | |
| | ttctgctacgtcccttcggccctcaatccagcggaccttccttcccgcggc | |
| | ctgctgccggctctgcggcctcttccgcgtcttcgccttcgccctcagacg | |
| | agtcggatctccctttgggccgcctccccgcatcgggaattcccgcggttc | |
| | gctttaagaccaatgacttacaaggcagctgtagatcttagccacttttta | |
| | aaagaaaaggggggactggaagggctaattcactcccaacgaagacaagat | |
| | ctgctttttgcttgtactgggtctctctggttagaccagatctgagcctgg | |
| | gagctctctggctaactagggaacccactgcttaagcctcaataaagcttg | |
| | ccttgagtgcttcaagtagtgtgtgcccgtctgttgtgtgactctggtaac | |
| | tagagatccctcagacccttttagtcagtgtggaaaatctctagcagtagt | |
| | agttcatgtcatcttattattcagtatttataacttgcaaagaaatgaata | |
| | tcagagagtgagaggaacttgtttattgcagcttataatggttacaaataa | |
| | agcaatagcatcacaaatttcacaaataaagcatttttttcactgcattct | |
| | agttgtggtttgtccaaactcatcaatgtatcttatcatgtctggctctag | |
| | ctatcccgcccctaactccgcccatcccgcccctaactccgcccagttccg | |
| | cccattctccgccccatggctgactaattttttttatttatgcagaggccg | |
| | aggccgcctcggcctctgagctattccagaagtagtgaggaggcttttttg | |
| | gaggcctagggacgtacccaattcgccctatagtgagtcgtattacgcgcg | |
| | ctcactggccgtcgttttacaacgtcgtgactgggaaaaccctggcgttac | |
| | ccaacttaatcgccttgcagcacatccccctttcgccagctggcgtaatag | |
| | cgaagaggcccgcaccgatcgcccttcccaacagttgcgcagcctgaatgg | |
| | cgaatgggacgcgccctgtagcggcgcattaagcgcggcgggtgtggtggt | |
| | tacgcgcagcgtgaccgctacacttgccagcgccctagcgcccgctccttt | |
| | cgctttcttcccttcctttctcgccacgttcgccggctttccccgtcaagc | |
| | tctaaatcgggggctccctttagggttccgatttagtgctttacggcacct | |
| | cgaccccaaaaaacttgattagggtgatggttcacgtagtgggccatcgcc | |
| | ctgatagacggtttttcgccctttgacgttggagtccacgttctttaatag | |
| | tggactcttgttccaaactggaacaacactcaaccctatctcggtctattc | |
| | ttttgatttataagggattttgccgatttcggcctattggttaaaaaatga | |
| | gctgatttaacaaaaatttaacgcgaattttaacaaaatattaacgcttac | |
| | aatttaggtggcacttttcggggaaatgtgcgcggaacccctatttgttta | |
| | tttttctaaatacattcaaatatgtatccgctcatgagacaataaccctga | |
| | taaatgcttcaataatattgaaaaaggaagagtatgagtattcaacatttc | |
| | cgtgtcgcccttattcccttttttgcggcattttgccttcctgtttttgct | |
| | cacccagaaacgctggtgaaagtaaaagatgctgaagatcagttgggtgca | |
| | cgagtgggttacatcgaactggatctcaacagcggtaagatccttgagagt | |
| | tttcgccccgaagaacgttttccaatgatgagcacttttaaagttctgcta | |
| | tgtggcgcggtattatcccgtattgacgccgggcaagagcaactcggtcgc | |
| | cgcatacactattctcagaatgacttggttgagtactcaccagtcacagaa | |
| | aagcatcttacggatggcatgacagtaagagaattatgcagtgctgccata | |
| | accatgagtgataacactgcggccaacttacttctgacaacgatcggagga | |
| | ccgaaggagctaaccgcttttttgcacaacatgggggatcatgtaactcgc | |
| | cttgatcgttgggaaccggagctgaatgaagccataccaaacgacgagcgt | |
| | gacaccacgatgcctgtagcaatggcaacaacgttgcgcaaactattaact | |
| | ggcgaactacttactctagcttcccggcaacaattaatagactggatggag | |
| | gcggataaagttgcaggaccacttctgcgctcggcccttccggctggctgg | |
| | tttattgctgataaatctggagccggtgagcgtgggtctcgcggtatcatt | |
| | gcagcactggggccagatggtaagccctcccgtatcgtagttatctacacg | |
| | acggggagtcaggcaactatggatgaacgaaatagacagatcgctgagata | |
| | ggtgcctcactgattaagcattggtaactgtcagaccaagtttactcatat | |
| | atactttagattgatttaaaacttcatttttaatttaaaaggatctaggtg | |
| | aagatcctttttgataatctcatgaccaaaatcccttaacgtgagttttcg | |
| | ttccactgagcgtcagaccccgtagaaaagatcaaaggatcttcttgagat | |
| | cctttttttctgcgcgtaatctgctgcttgcaaacaaaaaaaccaccgcta | |
| | ccagcggtggtttgtttgccggatcaagagctaccaactctttttccgaag | |
| | gtaactggcttcagcagagcgcagataccaaatactgttcttctagtgtag | |
| | ccgtagttaggccaccacttcaagaactctgtagcaccgcctacatacctc | |
| | gctctgctaatcctgttaccagtggctgctgccagtggcgataagtcgtgt | |
| | cttaccgggttggactcaagacgatagttaccggataaggcgcagcggtcg | |
| | ggctgaacggggggttcgtgcacacagcccagcttggagcgaacgacctac | |
| | accgaactgagatacctacagcgtgagctatgagaaagcgccacgcttccc | |
| | gaagagagaaaggcggacaggtatccggtaagcggcagggtcggaacagga | |
| | gagcgcacgagggagcttccagggggaaacgcctggtatctttatagtcct | |
| | gtcgggtttcgccacctctgacttgagcgtcgatttttgtgatgctcgtca | |
| | ggggggcggagcctatggaaaaacgccagcaacgcggcctttttacggttc | |
| | ctggccttttgctggccttttgctcacatgttctttcctgcgttatcccct | |
| | gattctgtggataaccgtattaccgcctttgagtgagctgataccgctcgc | |
| | cgcagccgaacgaccgagcgcagcgagtcagtgagcgaggaagcggaagag | |
| | cgcccaatacgcaaaccgcctctccccgcgcgttggccgattcattaatgc | |
| | agctggcacgacaggtttcccgactggaaagcgggcagtgagcgcaacgca | |
| | attaatgtgagttagctcactcattaggcaccccaggctttacactttatg | |
| | cttccggctcgtatgttgtgtggaattgtgagcggataacaatttcacaca | |
| | ggaaacagctatgaccatgattacgccaagcgcgcaattaaccctcactaa | |
| | agggaacaaaagctggagctgcaagctt | |
| VB211001- | aatgtagtcttatgcaatactcttgtagtcttgcaacatggtaacgatgag | 733 |
| 1280ykc | ttagcaacatgccttacaaggagagaaaaagcaccgtgcatgccgattggt | |
| (9951 bp) | ggaagtaaggtggtacgatcgtgccttattaggaaggcaacagacgggtct | |
| | gacatggattggacgaaccactgaattgccgcattgcagagatattgtatt | |
| | taagtgcctagctcgatacataaacgggtctctctggttagaccagatctg | |
| | agcctgggagctctctggctaactagggaacccactgcttaagcctcaata | |
| | aagcttgccttgagtgcttcaagtagtgtgtgcccgtctgttgtgtgactc | |
| | tggtaactagagatccctcagacccttttagtcagtgtggaaaatctctag | |
| | cagtggcgcccgaacagggacttgaaagcgaaagggaaaccagaggagctc | |
| | tctcgacgcaggactcggcttgctgaagcgcgcacggcaagaggcgagggg | |
| | cggcgactggtgagtacgccaaaaattttgactagcggaggctagaaggag | |
| | agagatgggtgcgagagcgtcagtattaagcgggggagaattagatcgcga | |
| | tgggaaaaaattcggttaaggccagggggaaagaaaaaatataaattaaaa | |
| | catatagtatgggcaagcagggagctagaacgattcgcagttaatcctggc | |
| | ctgttagaaacatcagaaggctgtagacaaatactgggacagctacaacca | |
| | tcccttcagacaggatcagaagaacttagatcattatataatacagtagca | |
| | accctctattgtgtgcatcaaaggatagagataaaagacaccaaggaagct | |
| | ttagacaagatagaggaagagcaaaacaaaagtaagaccaccgcacagcaa | |
| | gcggccgctgatcttcagacctggaggaggagatatgagggacaattggag | |
| | aagtgaattatataaatataaagtagtaaaaattgaaccattaggagtagc | |
| | acccaccaaggcaaagagaagagtggtgcagagagaaaaaagagcagtggg | |
| | aataggagctttgttccttgggttcttgggagcagcaggaagcactatggg | |
| | cgcagcgtcaatgacgctgacggtacaggccagacaattattgtctggtat | |
| | agtgcagcagcagaacaatttgctgagggctattgaggcgcaacagcatct | |
| | gttgcaactcacagtctggggcatcaagcagctccaggcaagaatcctggc | |
| | tgtggaaagatacctaaaggatcaacagctcctggggatttggggttgctc | |
| | tggaaaactcatttgcaccactgctgtgccttggaatgctagttggagtaa | |
| | taaatctctggaacagatttggaatcacacgacctggatggagtgggacag | |
| | agaaattaacaattacacaagcttaatacactccttaattgaagaatcgca | |
| | aaaccagcaagaaaagaatgaacaagaattattggaattagataaatgggc | |
| | aagtttgtggaattggtttaacataacaaattggctgtggtatataaaatt | |
| | attcataatgatagtaggaggcttggtaggtttaagaatagtttttgctgt | |
| | actttctatagtgaatagagttaggcagggatattcaccattatcgtttca | |
| | gacccacctcccaaccccgaggggacccgacaggcccgaaggaatagaaga | |
| | agaaggtggagagagagacagagacagatccattcgattagtgaacggatc | |
| | tcgacggtatcgctagcttttaaaagaaaaggggggattggggggtacagt | |
| | gcaggggaaagaatagtagacataatagcaacagacatacaaactaaagaa | |
| | ttacaaaaacaaattacaaaaattcaaaattttactagtgattatcggatc | |
| | aactttgtatagaaaagttgggctccggtgcccgtcagtgggcagagcgca | |
| | catcgcccacagtccccgagaagttggggggaggggtcggcaattgaaccg | |
| | gtgcctagagaaggtggcgcggggtaaactgggaaagtgatgtcgtgtact | |
| | ggctccgcctttttcccgagggtgggggagaaccgtatataagtgcagtag | |
| | tcgccgtgaacgttctttttcgcaacgggtttgccgccagaacacaggtaa | |
| | gtgccgtgtgtggttcccgcgggcctggcctctttacgggttatggccctt | |
| | gcgtgccttgaattacttccacctggctgcagtacgtgattcttgatcccg | |
| | agcttcgggttggaagtgggtgggagagttcgaggccttgcgcttaaggag | |
| | ccccttcgcctcgtgcttgagttgaggcctggcctgggcgctggggccgcc | |
| | gcgtgcgaatctggtggcaccttcgcgcctgtctcgctgctttcgataagt | |
| | ctctagccatttaaaatttttgatgacctgctgcgacgctttttttctggc | |
| | aagatagtcttgtaaatgcgggccaagatctgcacactggtatttcggttt | |
| | ttggggccgcgggcggcgacggggcccgtgcgtcccagcgcacatgttcgg | |
| | cgaggcggggcctgcgagcgcggccaccgagaatcggacgggggtagtctc | |
| | aagctggccggcctgctctggtgcctggtctcgcgccgccgtgtatcgccc | |
| | cgccctgggcggcaaggctggcccggtcggcaccagttgcgtgagcggaaa | |
| | gatggccgcttcccggccctgctgcagggagctcaaaatggaggacgcggc | |
| | gctcgggagagcgggcgggtgagtcacccacacaaaggaaaagggcctttc | |
| | cgtcctcagccgtcgcttcatgtgactccacggagtaccgggcgccgtcca | |
| | ggcacctcgattagttctcgagcttttggagtacgtcgtctttaggttggg | |
| | gggaggggttttatgcgatggagtttccccacactgagtgggtggagactg | |
| | aagttaggccagcttggcacttgatgtaattctccttggaatttgcccttt | |
| | ttgagtttggatcttggttcattctcaagcctcagacagtggttcaaagtt | |
| | tttttcttccatttcaggtgtcgtgacaagtttgtacaaaaaagcaggctg | |
| | ccaccatgccattgcttcttctgctgccactcctttgggctggtgcgctgg | |
| | ctattccggggggtttgtcagaagcaaagcccgcgaccccagaaattcagg | |
| | agatcgtggataaagtaaaacctcagcttgaagaaaaaaccggggagacat | |
| | acggcaagctggaagcagtacaatataaaacgcaggtagtacattactacg | |
| | tccattataacgatcaaggtacaaattattatataaaagtacgggcagggg | |
| | acaacaaatacatgcatctgaaagtatttaagagtctgtggggtgagaact | |
| | tgtttgcaaagtgggaagatttggtgcttaccggctaccaggtcgataaga | |
| | ataaggatgacgagcttacaggttttgcagaagctgcagcgaaagaggctg | |
| | ccgcaaaagaagcagcggctaaggaagcggccgctaaagaagcggccgcga | |
| | aggaagcggccgctaagatgatcccgggtggtctgtccgaagccaaaccgg | |
| | cgacgccggaaatccaggagatagtcgataaggtcaagccacaactcgaag | |
| | agaagactggagaaacatatggaaaacttgaggccgttcaatacaaaacgc | |
| | aggtggtccactactacgttcactataacgatcaaggaacaaactattaca | |
| | ttaaagtcagggcgggagacaacaagtacatgcatcttaaagtgtttaaat | |
| | ctctttggggggagaatcttttcgccaagtgggaagacttggtcctcaccg | |
| | gataccaggtcgacaaaaacaaagacgatgaactcaccggctttgccgaag | |
| | cggctgcgaaagaagcggctgccaaagaggctgcagccaaagaagctgcgg | |
| | caaaggaggccgccgccaaagaagcggcggccaaaatgatccccggaggac | |
| | ttagcgaggctaaacccgcgacacctgaaatacaggagatcgtggataagg | |
| | ttaaaccacaattggaggagaaaactggtgagacctatggcaagctggaag | |
| | cggttcagtataaaacgcaagttgtacactactatgtacattacaacgatc | |
| | aggggacgaactactatattaaggtcagggcaggagataataaatatatgc | |
| | acttgaaagttttcaaaagcctgtggggggaaaacttgttcgccaagtggg | |
| | aagatctcgtcttgactgggtaccaggttgataagaacaaagacgatgaac | |
| | tgacaggcttcgctgaagcggctgcaaaggaggcggcagcgaaggaggctg | |
| | ccgcgaaagaagcggcggctaaagaagcggccgcaaaggaagcagcagcga | |
| | agatgataccgaggggtttgagcgaagcaaagcccgcaacgcctgagatac | |
| | aagaaatcgttgacaaagtaaaaccacaattggaagagaagacgggagaaa | |
| | cgtatggaaagcttgaggccgtacaatacaagacacaggtgttggcaaatt | |
| | ttttccaaaggcgatggcccggatcaacgaattactatatcaaggtacgcg | |
| | cgggggataacaagtacatgcatctcaaggttttcaacggtccgtggaaat | |
| | tccgaaatacagaccgcggagccgacagggtcctcaccggctatcaggtcg | |
| | acaagaataaggatgatgagttgacaggtttcgccgccgccggtgagcaga | |
| | aattgatctccgaagaggatctcggtgccgcgcaccaccaccaccatcatg | |
| | gaagcggagagggcaggggaagtcttctaacatgcggggacgtggaggaaa | |
| | atcccggccccatgattgaacaagatggattgcacgcaggttctccggccg | |
| | cttgggtggagaggctattcggctatgactgggcacaacagacaatcggct | |
| | gctctgatgccgccgtgttccggctgtcagcgcaggggcgcccggttcttt | |
| | ttgtcaagaccgacctgtccggtgccctgaatgaactgcaagacgaggcag | |
| | cgcggctatcgtggctggccacgacgggcgttccttgcgcagctgtgctcg | |
| | acgttgtcactgaagcgggaagggactggctgctattgggcgaagtgccgg | |
| | ggcaggatctcctgtcatctcaccttgctcctgccgagaaagtatccatca | |
| | tggctgatgcaatgcggcggctgcatacgcttgatccggctacctgcccat | |
| | tcgaccaccaagcgaaacatcgcatcgagcgagcacgtactcggatggaag | |
| | ccggtcttgtcgatcaggatgatctggacgaagagcatcaggggctcgcgc | |
| | cagccgaactgttcgccaggctcaaggcgagcatgcccgacggcgaggatc | |
| | tcgtcgtgacccatggcgatgcctgcttgccgaatatcatggtggaaaatg | |
| | gccgcttttctggattcatcgactgtggccggctgggtgtggcggaccgct | |
| | atcaggacatagcgttggctacccgtgatattgctgaagagcttggcggcg | |
| | aatgggctgaccgcttcctcgtgctttacggtatcgccgctcccgattcgc | |
| | agcgcatcgccttctatcgccttcttgacgagttcttctgaacccagcttt | |
| | cttgtacaaagtggtgataatcgaattccgataatcaacctctggattaca | |
| | aaatttgtgaaagattgactggtattcttaactatgttgctccttttacgc | |
| | tatgtggatacgctgctttaatgcctttgtatcatgctattgcttcccgta | |
| | tggctttcattttctcctccttgtataaatcctggttgctgtctctttatg | |
| | aggagttgtggcccgttgtcaggcaacgtggcgtggtgtgcactgtgtttg | |
| | ctgacgcaacccccactggttggggcattgccaccacctgtcagctccttt | |
| | ccgggactttcgctttccccctccctattgccacggcggaactcatcgccg | |
| | cctgccttgcccgctgctggacaggggctcggctgttgggcactgacaatt | |
| | ccgtggtgttgtcggggaagctgacgtcctttccatggctgctcgcctgtg | |
| | ttgccacctggattctgcgcgggacgtccttctgctacgtcccttcggccc | |
| | tcaatccagcggaccttccttcccgcggcctgctgccggctctgcggcctc | |
| | ttccgcgtcttcgccttcgccctcagacgagtcggatctccctttgggccg | |
| | cctccccgcatcgggaattcccgcggttcgctttaagaccaatgacttaca | |
| | aggcagctgtagatcttagccactttttaaaagaaaaggggggactggaag | |
| | ggctaattcactcccaacgaagacaagatctgctttttgcttgtactgggt | |
| | ctctctggttagaccagatctgagcctgggagctctctggctaactaggga | |
| | acccactgcttaagcctcaataaagcttgccttgagtgcttcaagtagtgt | |
| | gtgcccgtctgttgtgtgactctggtaactagagatccctcagaccctttt | |
| | agtcagtgtggaaaatctctagcagtagtagttcatgtcatcttattattc | |
| | agtatttataacttgcaaagaaatgaatatcagagagtgagaggaacttgt | |
| | ttattgcagcttataatggttacaaataaagcaatagcatcacaaatttca | |
| | caaataaagcatttttttcactgcattctagttgtggtttgtccaaactca | |
| | tcaatgtatcttatcatgtctggctctagctatcccgcccctaactccgcc | |
| | catcccgcccctaactccgcccagttccgcccattctccgccccatggctg | |
| | actaattttttttatttatgcagaggccgaggccgcctcggcctctgagct | |
| | attccagaagtagtgaggaggcttttttggaggcctagggacgtacccaat | |
| | tcgccctatagtgagtcgtattacgcgcgctcactggccgtcgttttacaa | |
| | cgtcgtgactgggaaaaccctggcgttacccaacttaatcgccttgcagca | |
| | catccccctttcgccagctggcgtaatagcgaagaggcccgcaccgatcgc | |
| | ccttcccaacagttgcgcagcctgaatggcgaatgggacgcgccctgtagc | |
| | ggcgcattaagcgcggcgggtgtggtggttacgcgcagcgtgaccgctaca | |
| | cttgccagcgccctagcgcccgctcctttcgctttcttcccttcctttctc | |
| | gccacgttcgccggctttccccgtcaagctctaaatcgggggctcccttta | |
| | gggttccgatttagtgctttacggcacctcgaccccaaaaaacttgattag | |
| | ggtgatggttcacgtagtgggccatcgccctgatagacggtttttcgccct | |
| | ttgacgttggagtccacgttctttaatagtggactcttgttccaaactgga | |
| | acaacactcaaccctatctcggtctattcttttgatttataagggattttg | |
| | ccgatttcggcctattggttaaaaaatgagctgatttaacaaaaatttaac | |
| | gcgaattttaacaaaatattaacgcttacaatttaggtggcacttttcggg | |
| | gaaatgtgcgcggaacccctatttgtttatttttctaaatacattcaaata | |
| | tgtatccgctcatgagacaataaccctgataaatgcttcaataatattgaa | |
| | aaaggaagagtatgagtattcaacatttccgtgtcgcccttattccctttt | |
| | ttgcggcattttgccttcctgtttttgctcacccagaaacgctggtgaaag | |
| | taaaagatgctgaagatcagttgggtgcacgagtgggttacatcgaactgg | |
| | atctcaacagcggtaagatccttgagagttttcgccccgaagaacgttttc | |
| | caatgatgagcacttttaaagttctgctatgtggcgcggtattatcccgta | |
| | ttgacgccgggcaagagcaactcggtcgccgcatacactattctcagaatg | |
| | acttggttgagtactcaccagtcacagaaaagcatcttacggatggcatga | |
| | cagtaagagaattatgcagtgctgccataaccatgagtgataacactgcgg | |
| | ccaacttacttctgacaacgatcggaggaccgaaggagctaaccgcttttt | |
| | tgcacaacatgggggatcatgtaactcgccttgatcgttgggaaccggagc | |
| | tgaatgaagccataccaaacgacgagcgtgacaccacgatgcctgtagcaa | |
| | tggcaacaacgttgcgcaaactattaactggcgaactacttactctagctt | |
| | cccggcaacaattaatagactggatggaggcggataaagttgcaggaccac | |
| | ttctgcgctcggcccttccggctggctggtttattgctgataaatctggag | |
| | ccggtgagcgtgggtctcgcggtatcattgcagcactggggccagatggta | |
| | agccctcccgtatcgtagttatctacacgacggggagtcaggcaactatgg | |
| | atgaacgaaatagacagatcgctgagataggtgcctcactgattaagcatt | |
| | ggtaactgtcagaccaagtttactcatatatactttagattgatttaaaac | |
| | ttcatttttaatttaaaaggatctaggtgaagatcctttttgataatctca | |
| | tgaccaaaatcccttaacgtgagttttcgttccactgagcgtcagaccccg | |
| | tagaaaagatcaaaggatcttcttgagatcctttttttctgcgcgtaatct | |
| | gctgcttgcaaacaaaaaaaccaccgctaccagcggtggtttgtttgccgg | |
| | atcaagagctaccaactctttttccgaaggtaactggcttcagcagagcgc | |
| | agataccaaatactgttcttctagtgtagccgtagttaggccaccacttca | |
| | agaactctgtagcaccgcctacatacctcgctctgctaatcctgttaccag | |
| | tggctgctgccagtggcgataagtcgtgtcttaccgggttggactcaagac | |
| | gatagttaccggataaggcgcagcggtcgggctgaacggggggttcgtgca | |
| | cacagcccagcttggagcgaacgacctacaccgaactgagatacctacagc | |
| | gtgagctatgagaaagcgccacgcttcccgaagagagaaaggcggacaggt | |
| | atccggtaagcggcagggtcggaacaggagagcgcacgagggagcttccag | |
| | ggggaaacgcctggtatctttatagtcctgtcgggtttcgccacctctgac | |
| | ttgagcgtcgatttttgtgatgctcgtcaggggggcggagcctatggaaaa | |
| | acgccagcaacgcggcctttttacggttcctggccttttgctggccttttg | |
| | ctcacatgttctttcctgcgttatcccctgattctgtggataaccgtatta | |
| | ccgcctttgagtgagctgataccgctcgccgcagccgaacgaccgagcgca | |
| | gcgagtcagtgagcgaggaagcggaagagcgcccaatacgcaaaccgcctc | |
| | tccccgcgcgttggccgattcattaatgcagctggcacgacaggtttcccg | |
| | actggaaagcgggcagtgagcgcaacgcaattaatgtgagttagctcactc | |
| | attaggcaccccaggctttacactttatgcttccggctcgtatgttgtgtg | |
| | gaattgtgagcggataacaatttcacacaggaaacagctatgaccatgatt | |
| | acgccaagcgcgcaattaaccctcactaaagggaacaaaagctggagctgc | |
| | aagctt | |
| VB211001- | aatgtagtcttatgcaatactcttgtagtcttgcaacatggtaacgatgag | 734 |
| 1281ptb | ttagcaacatgccttacaaggagagaaaaagcaccgtgcatgccgattggt | |
| (9004 bp) | ggaagtaaggtggtacgatcgtgccttattaggaaggcaacagacgggtct | |
| | gacatggattggacgaaccactgaattgccgcattgcagagatattgtatt | |
| | taagtgcctagctcgatacataaacgggtctctctggttagaccagatctg | |
| | agcctgggagctctctggctaactagggaacccactgcttaagcctcaata | |
| | aagcttgccttgagtgcttcaagtagtgtgtgcccgtctgttgtgtgactc | |
| | tggtaactagagatccctcagacccttttagtcagtgtggaaaatctctag | |
| | cagtggcgcccgaacagggacttgaaagcgaaagggaaaccagaggagctc | |
| | tctcgacgcaggactcggcttgctgaagcgcgcacggcaagaggcgagggg | |
| | cggcgactggtgagtacgccaaaaattttgactagcggaggctagaaggag | |
| | agagatgggtgcgagagcgtcagtattaagcgggggagaattagatcgcga | |
| | tgggaaaaaattcggttaaggccagggggaaagaaaaaatataaattaaaa | |
| | catatagtatgggcaagcagggagctagaacgattcgcagttaatcctggc | |
| | ctgttagaaacatcagaaggctgtagacaaatactgggacagctacaacca | |
| | tcccttcagacaggatcagaagaacttagatcattatataatacagtagca | |
| | accctctattgtgtgcatcaaaggatagagataaaagacaccaaggaagct | |
| | ttagacaagatagaggaagagcaaaacaaaagtaagaccaccgcacagcaa | |
| | gcggccgctgatcttcagacctggaggaggagatatgagggacaattggag | |
| | aagtgaattatataaatataaagtagtaaaaattgaaccattaggagtagc | |
| | acccaccaaggcaaagagaagagtggtgcagagagaaaaaagagcagtggg | |
| | aataggagctttgttccttgggttcttgggagcagcaggaagcactatggg | |
| | cgcagcgtcaatgacgctgacggtacaggccagacaattattgtctggtat | |
| | agtgcagcagcagaacaatttgctgagggctattgaggcgcaacagcatct | |
| | gttgcaactcacagtctggggcatcaagcagctccaggcaagaatcctggc | |
| | tgtggaaagatacctaaaggatcaacagctcctggggatttggggttgctc | |
| | tggaaaactcatttgcaccactgctgtgccttggaatgctagttggagtaa | |
| | taaatctctggaacagatttggaatcacacgacctggatggagtgggacag | |
| | agaaattaacaattacacaagcttaatacactccttaattgaagaatcgca | |
| | aaaccagcaagaaaagaatgaacaagaattattggaattagataaatgggc | |
| | aagtttgtggaattggtttaacataacaaattggctgtggtatataaaatt | |
| | attcataatgatagtaggaggcttggtaggtttaagaatagtttttgctgt | |
| | actttctatagtgaatagagttaggcagggatattcaccattatcgtttca | |
| | gacccacctcccaaccccgaggggacccgacaggcccgaaggaatagaaga | |
| | agaaggtggagagagagacagagacagatccattcgattagtgaacggatc | |
| | tcgacggtatcgctagcttttaaaagaaaaggggggattggggggtacagt | |
| | gcaggggaaagaatagtagacataatagcaacagacatacaaactaaagaa | |
| | ttacaaaaacaaattacaaaaattcaaaattttactagtgattatcggatc | |
| | aactttgtatagaaaagttgggctccggtgcccgtcagtgggcagagcgca | |
| | catcgcccacagtccccgagaagttggggggaggggtcggcaattgatccg | |
| | gtgcctagagaaggtggcgcggggtaaactgggaaagtgatgtcgtgtact | |
| | ggctccgcctttttcccgagggtgggggagaaccgtatataagtgcagtag | |
| | tcgccgtgaacgttctttttcgcaacgggtttgccgccagaacacaggcaa | |
| | gtttgtacaaaaaagcaggctgccaccatgccattgcttcttctgctgcca | |
| | ctcctttgggctggtgcgctggctattccggggggtttgtcagaagcaaag | |
| | cccgcgaccccagaaattcaggagatcgtggataaagtaaaacctcagctt | |
| | gaagaaaaaaccggggagacatacggcaagctggaagcagtacaatataaa | |
| | acgcaggtagtacattactacgtccattataacgatcaaggtacaaattat | |
| | tatataaaagtacgggcaggggacaacaaatacatgcatctgaaagtattt | |
| | aagagtctgtggggtgagaacttgtttgcaaagtgggaagatttggtgctt | |
| | accggctaccaggtcgataagaataaggatgacgagcttacaggttttgca | |
| | gaagctgcagcgaaagaggctgccgcaaaagaagcagcggctaaggaagcg | |
| | gccgctaaagaagcggccgcgaaggaagcggccgctaagatgatcccgggt | |
| | ggtctgtccgaagccaaaccggcgacgccggaaatccaggagatagtcgat | |
| | aaggtcaagccacaactcgaagagaagactggagaaacatatggaaaactt | |
| | gaggccgttcaatacaaaacgcaggtggtccactactacgttcactataac | |
| | gatcaaggaacaaactattacattaaagtcagggcgggagacaacaagtac | |
| | atgcatcttaaagtgtttaaatctctttggggggagaatcttttcgccaag | |
| | tgggaagacttggtcctcaccggataccaggtcgacaaaaacaaagacgat | |
| | gaactcaccggctttgccgaagcggctgcgaaagaagcggctgccaaagag | |
| | gctgcagccaaagaagctgcggcaaaggaggccgccgccaaagaagcggcg | |
| | gccaaaatgatccccggaggacttagcgaggctaaacccgcgacacctgaa | |
| | atacaggagatcgtggataaggttaaaccacaattggaggagaaaactggt | |
| | gagacctatggcaagctggaagcggttcagtataaaacgcaagttgtacac | |
| | tactatgtacattacaacgatcaggggacgaactactatattaaggtcagg | |
| | gcaggagataataaatatatgcacttgaaagttttcaaaagcctgtggggg | |
| | gaaaacttgttcgccaagtgggaagatctcgtcttgactgggtaccaggtt | |
| | gataagaacaaagacgatgaactgacaggcttcgctgaagcggctgcaaag | |
| | gaggcggcagcgaaggaggctgccgcgaaagaagcggcggctaaagaagcg | |
| | gccgcaaaggaagcagcagcgaagatgataccgaggggtttgagcgaagca | |
| | aagcccgcaacgcctgagatacaagaaatcgttgacaaagtaaaaccacaa | |
| | ttggaagagaagacgggagaaacgtatggaaagcttgaggccgtacaatac | |
| | aagacacaggtgttggcaaattttttccaaaggcgatggcccggatcaacg | |
| | aattactatatcaaggtacgcgcgggggataacaagtacatgcatctcaag | |
| | gttttcaacggtccgtggaaattccgaaatacagaccgcggagccgacagg | |
| | gtcctcaccggctatcaggtcgacaagaataaggatgatgagttgacaggt | |
| | ttcgccgccgccggtgagcagaaattgatctccgaagaggatctcggtgcc | |
| | gcgcaccaccaccaccatcatggaagcggagagggcaggggaagtcttcta | |
| | acatgcggggacgtggaggaaaatcccggccccatgattgaacaagatgga | |
| | ttgcacgcaggttctccggccgcttgggtggagaggctattcggctatgac | |
| | tgggcacaacagacaatcggctgctctgatgccgccgtgttccggctgtca | |
| | gcgcaggggcgcccggttctttttgtcaagaccgacctgtccggtgccctg | |
| | aatgaactgcaagacgaggcagcgcggctatcgtggctggccacgacgggc | |
| | gttccttgcgcagctgtgctcgacgttgtcactgaagcgggaagggactgg | |
| | ctgctattgggcgaagtgccggggcaggatctcctgtcatctcaccttgct | |
| | cctgccgagaaagtatccatcatggctgatgcaatgcggcggctgcatacg | |
| | cttgatccggctacctgcccattcgaccaccaagcgaaacatcgcatcgag | |
| | cgagcacgtactcggatggaagccggtcttgtcgatcaggatgatctggac | |
| | gaagagcatcaggggctcgcgccagccgaactgttcgccaggctcaaggcg | |
| | agcatgcccgacggcgaggatctcgtcgtgacccatggcgatgcctgcttg | |
| | ccgaatatcatggtggaaaatggccgcttttctggattcatcgactgtggc | |
| | cggctgggtgtggcggaccgctatcaggacatagcgttggctacccgtgat | |
| | attgctgaagagcttggcggcgaatgggctgaccgcttcctcgtgctttac | |
| | ggtatcgccgctcccgattcgcagcgcatcgccttctatcgccttcttgac | |
| | gagttcttctgaacccagctttcttgtacaaagtggtgataatcgaattcc | |
| | gataatcaacctctggattacaaaatttgtgaaagattgactggtattctt | |
| | aactatgttgctccttttacgctatgtggatacgctgctttaatgcctttg | |
| | tatcatgctattgcttcccgtatggctttcattttctcctccttgtataaa | |
| | tcctggttgctgtctctttatgaggagttgtggcccgttgtcaggcaacgt | |
| | ggcgtggtgtgcactgtgtttgctgacgcaacccccactggttggggcatt | |
| | gccaccacctgtcagctcctttccgggactttcgctttccccctccctatt | |
| | gccacggcggaactcatcgccgcctgccttgcccgctgctggacaggggct | |
| | cggctgttgggcactgacaattccgtggtgttgtcggggaagctgacgtcc | |
| | tttccatggctgctcgcctgtgttgccacctggattctgcgcgggacgtcc | |
| | ttctgctacgtcccttcggccctcaatccagcggaccttccttcccgcggc | |
| | ctgctgccggctctgcggcctcttccgcgtcttcgccttcgccctcagacg | |
| | agtcggatctccctttgggccgcctccccgcatcgggaattcccgcggttc | |
| | gctttaagaccaatgacttacaaggcagctgtagatcttagccacttttta | |
| | aaagaaaaggggggactggaagggctaattcactcccaacgaagacaagat | |
| | ctgctttttgcttgtactgggtctctctggttagaccagatctgagcctgg | |
| | gagctctctggctaactagggaacccactgcttaagcctcaataaagcttg | |
| | ccttgagtgcttcaagtagtgtgtgcccgtctgttgtgtgactctggtaac | |
| | tagagatccctcagacccttttagtcagtgtggaaaatctctagcagtagt | |
| | agttcatgtcatcttattattcagtatttataacttgcaaagaaatgaata | |
| | tcagagagtgagaggaacttgtttattgcagcttataatggttacaaataa | |
| | agcaatagcatcacaaatttcacaaataaagcatttttttcactgcattct | |
| | agttgtggtttgtccaaactcatcaatgtatcttatcatgtctggctctag | |
| | ctatcccgcccctaactccgcccatcccgcccctaactccgcccagttccg | |
| | cccattctccgccccatggctgactaattttttttatttatgcagaggccg | |
| | aggccgcctcggcctctgagctattccagaagtagtgaggaggcttttttg | |
| | gaggcctagggacgtacccaattcgccctatagtgagtcgtattacgcgcg | |
| | ctcactggccgtcgttttacaacgtcgtgactgggaaaaccctggcgttac | |
| | ccaacttaatcgccttgcagcacatccccctttcgccagctggcgtaatag | |
| | cgaagaggcccgcaccgatcgcccttcccaacagttgcgcagcctgaatgg | |
| | cgaatgggacgcgccctgtagcggcgcattaagcgcggcgggtgtggtggt | |
| | tacgcgcagcgtgaccgctacacttgccagcgccctagcgcccgctccttt | |
| | cgctttcttcccttcctttctcgccacgttcgccggctttccccgtcaagc | |
| | tctaaatcgggggctccctttagggttccgatttagtgctttacggcacct | |
| | cgaccccaaaaaacttgattagggtgatggttcacgtagtgggccatcgcc | |
| | ctgatagacggtttttcgccctttgacgttggagtccacgttctttaatag | |
| | tggactcttgttccaaactggaacaacactcaaccctatctcggtctattc | |
| | ttttgatttataagggattttgccgatttcggcctattggttaaaaaatga | |
| | gctgatttaacaaaaatttaacgcgaattttaacaaaatattaacgcttac | |
| | aatttaggtggcacttttcggggaaatgtgcgcggaacccctatttgttta | |
| | tttttctaaatacattcaaatatgtatccgctcatgagacaataaccctga | |
| | taaatgcttcaataatattgaaaaaggaagagtatgagtattcaacatttc | |
| | cgtgtcgcccttattcccttttttgcggcattttgccttcctgtttttgct | |
| | cacccagaaacgctggtgaaagtaaaagatgctgaagatcagttgggtgca | |
| | cgagtgggttacatcgaactggatctcaacagcggtaagatccttgagagt | |
| | tttcgccccgaagaacgttttccaatgatgagcacttttaaagttctgcta | |
| | tgtggcgcggtattatcccgtattgacgccgggcaagagcaactcggtcgc | |
| | cgcatacactattctcagaatgacttggttgagtactcaccagtcacagaa | |
| | aagcatcttacggatggcatgacagtaagagaattatgcagtgctgccata | |
| | accatgagtgataacactgcggccaacttacttctgacaacgatcggagga | |
| | ccgaaggagctaaccgcttttttgcacaacatgggggatcatgtaactcgc | |
| | cttgatcgttgggaaccggagctgaatgaagccataccaaacgacgagcgt | |
| | gacaccacgatgcctgtagcaatggcaacaacgttgcgcaaactattaact | |
| | ggcgaactacttactctagcttcccggcaacaattaatagactggatggag | |
| | gcggataaagttgcaggaccacttctgcgctcggcccttccggctggctgg | |
| | tttattgctgataaatctggagccggtgagcgtgggtctcgcggtatcatt | |
| | gcagcactggggccagatggtaagccctcccgtatcgtagttatctacacg | |
| | acggggagtcaggcaactatggatgaacgaaatagacagatcgctgagata | |
| | ggtgcctcactgattaagcattggtaactgtcagaccaagtttactcatat | |
| | atactttagattgatttaaaacttcatttttaatttaaaaggatctaggtg | |
| | aagatcctttttgataatctcatgaccaaaatcccttaacgtgagttttcg | |
| | ttccactgagcgtcagaccccgtagaaaagatcaaaggatcttcttgagat | |
| | cctttttttctgcgcgtaatctgctgcttgcaaacaaaaaaaccaccgcta | |
| | ccagcggtggtttgtttgccggatcaagagctaccaactctttttccgaag | |
| | gtaactggcttcagcagagcgcagataccaaatactgttcttctagtgtag | |
| | ccgtagttaggccaccacttcaagaactctgtagcaccgcctacatacctc | |
| | gctctgctaatcctgttaccagtggctgctgccagtggcgataagtcgtgt | |
| | cttaccgggttggactcaagacgatagttaccggataaggcgcagcggtcg | |
| | ggctgaacggggggttcgtgcacacagcccagcttggagcgaacgacctac | |
| | accgaactgagatacctacagcgtgagctatgagaaagcgccacgcttccc | |
| | gaagagagaaaggcggacaggtatccggtaagcggcagggtcggaacagga | |
| | gagcgcacgagggagcttccagggggaaacgcctggtatctttatagtcct | |
| | gtcgggtttcgccacctctgacttgagcgtcgatttttgtgatgctcgtca | |
| | ggggggcggagcctatggaaaaacgccagcaacgcggcctttttacggttc | |
| | ctggccttttgctggccttttgctcacatgttctttcctgcgttatcccct | |
| | gattctgtggataaccgtattaccgcctttgagtgagctgataccgctcgc | |
| | cgcagccgaacgaccgagcgcagcgagtcagtgagcgaggaagcggaagag | |
| | cgcccaatacgcaaaccgcctctccccgcgcgttggccgattcattaatgc | |
| | agctggcacgacaggtttcccgactggaaagcgggcagtgagcgcaacgca | |
| | attaatgtgagttagctcactcattaggcaccccaggctttacactttatg | |
| | cttccggctcgtatgttgtgtggaattgtgagcggataacaatttcacaca | |
| | ggaaacagctatgaccatgattacgccaagcgcgcaattaaccctcactaa | |
| | agggaacaaaagctggagctgcaagctt | |
| VB211001- | aatgtagtcttatgcaatactcttgtagtcttgcaacatggtaacgatgag | 735 |
| 1282mht | ttagcaacatgccttacaaggagagaaaaagcaccgtgcatgccgattggt | |
| (9272 bp) | ggaagtaaggtggtacgatcgtgccttattaggaaggcaacagacgggtct | |
| | gacatggattggacgaaccactgaattgccgcattgcagagatattgtatt | |
| | taagtgcctagctcgatacataaacgggtctctctggttagaccagatctg | |
| | agcctgggagctctctggctaactagggaacccactgcttaagcctcaata | |
| | aagcttgccttgagtgcttcaagtagtgtgtgcccgtctgttgtgtgactc | |
| | tggtaactagagatccctcagacccttttagtcagtgtggaaaatctctag | |
| | cagtggcgcccgaacagggacttgaaagcgaaagggaaaccagaggagctc | |
| | tctcgacgcaggactcggcttgctgaagcgcgcacggcaagaggcgagggg | |
| | cggcgactggtgagtacgccaaaaattttgactagcggaggctagaaggag | |
| | agagatgggtgcgagagcgtcagtattaagcgggggagaattagatcgcga | |
| | tgggaaaaaattcggttaaggccagggggaaagaaaaaatataaattaaaa | |
| | catatagtatgggcaagcagggagctagaacgattcgcagttaatcctggc | |
| | ctgttagaaacatcagaaggctgtagacaaatactgggacagctacaacca | |
| | tcccttcagacaggatcagaagaacttagatcattatataatacagtagca | |
| | accctctattgtgtgcatcaaaggatagagataaaagacaccaaggaagct | |
| | ttagacaagatagaggaagagcaaaacaaaagtaagaccaccgcacagcaa | |
| | gcggccgctgatcttcagacctggaggaggagatatgagggacaattggag | |
| | aagtgaattatataaatataaagtagtaaaaattgaaccattaggagtagc | |
| | acccaccaaggcaaagagaagagtggtgcagagagaaaaaagagcagtggg | |
| | aataggagctttgttccttgggttcttgggagcagcaggaagcactatggg | |
| | cgcagcgtcaatgacgctgacggtacaggccagacaattattgtctggtat | |
| | agtgcagcagcagaacaatttgctgagggctattgaggcgcaacagcatct | |
| | gttgcaactcacagtctggggcatcaagcagctccaggcaagaatcctggc | |
| | tgtggaaagatacctaaaggatcaacagctcctggggatttggggttgctc | |
| | tggaaaactcatttgcaccactgctgtgccttggaatgctagttggagtaa | |
| | taaatctctggaacagatttggaatcacacgacctggatggagtgggacag | |
| | agaaattaacaattacacaagcttaatacactccttaattgaagaatcgca | |
| | aaaccagcaagaaaagaatgaacaagaattattggaattagataaatgggc | |
| | aagtttgtggaattggtttaacataacaaattggctgtggtatataaaatt | |
| | attcataatgatagtaggaggcttggtaggtttaagaatagtttttgctgt | |
| | actttctatagtgaatagagttaggcagggatattcaccattatcgtttca | |
| | gacccacctcccaaccccgaggggacccgacaggcccgaaggaatagaaga | |
| | agaaggtggagagagagacagagacagatccattcgattagtgaacggatc | |
| | tcgacggtatcgctagcttttaaaagaaaaggggggattggggggtacagt | |
| | gcaggggaaagaatagtagacataatagcaacagacatacaaactaaagaa | |
| | ttacaaaaacaaattacaaaaattcaaaattttactagtgattatcggatc | |
| | aactttgtatagaaaagttgctgcagccccgataaaataaaagattttatt | |
| | tagtctccagaaaaaggggggaatgaaagaccccacctgtaggtttggcaa | |
| | gctagctgcagtaacgccattttgcaaggcatggaaaaataccaaaccaag | |
| | aatagagaagttcagatcaagggcgggtacatgaaaatagctaacgttggg | |
| | ccaaacaggatatctgcggtgagcagtttcggccccggcccggggccaaga | |
| | acagatggtcaccgcagtttcggccccggcccgaggccaagaacagatggt | |
| | ccccagatatggcccaaccctcagcagtttcttaagacccatcagatgttt | |
| | ccaggctcccccaaggacctgaaatgaccctgcgccttatttgaattaacc | |
| | aatcagcctgcttctcgcttctgttcgcgcgcttctgcttcccgagctcta | |
| | taaaagagctcacaacccctcactcggcgcgccagtcctccgacagactga | |
| | gtcgcccgggcaagtttgtacaaaaaagcaggctgccaccatgccattgct | |
| | tcttctgctgccactcctttgggctggtgcgctggctattccggggggttt | |
| | gtcagaagcaaagcccgcgaccccagaaattcaggagatcgtggataaagt | |
| | aaaacctcagcttgaagaaaaaaccggggagacatacggcaagctggaagc | |
| | agtacaatataaaacgcaggtagtacattactacgtccattataacgatca | |
| | aggtacaaattattatataaaagtacgggcaggggacaacaaatacatgca | |
| | tctgaaagtatttaagagtctgtggggtgagaacttgtttgcaaagtggga | |
| | agatttggtgcttaccggctaccaggtcgataagaataaggatgacgagct | |
| | tacaggttttgcagaagctgcagcgaaagaggctgccgcaaaagaagcagc | |
| | ggctaaggaagcggccgctaaagaagcggccgcgaaggaagcggccgctaa | |
| | gatgatcccgggtggtctgtccgaagccaaaccggcgacgccggaaatcca | |
| | ggagatagtcgataaggtcaagccacaactcgaagagaagactggagaaac | |
| | atatggaaaacttgaggccgttcaatacaaaacgcaggtggtccactacta | |
| | cgttcactataacgatcaaggaacaaactattacattaaagtcagggcggg | |
| | agacaacaagtacatgcatcttaaagtgtttaaatctctttggggggagaa | |
| | tcttttcgccaagtgggaagacttggtcctcaccggataccaggtcgacaa | |
| | aaacaaagacgatgaactcaccggctttgccgaagcggctgcgaaagaagc | |
| | ggctgccaaagaggctgcagccaaagaagctgcggcaaaggaggccgccgc | |
| | caaagaagcggcggccaaaatgatccccggaggacttagcgaggctaaacc | |
| | cgcgacacctgaaatacaggagatcgtggataaggttaaaccacaattgga | |
| | ggagaaaactggtgagacctatggcaagctggaagcggttcagtataaaac | |
| | gcaagttgtacactactatgtacattacaacgatcaggggacgaactacta | |
| | tattaaggtcagggcaggagataataaatatatgcacttgaaagttttcaa | |
| | aagcctgtggggggaaaacttgttcgccaagtgggaagatctcgtcttgac | |
| | tgggtaccaggttgataagaacaaagacgatgaactgacaggcttcgctga | |
| | agcggctgcaaaggaggcggcagcgaaggaggctgccgcgaaagaagcggc | |
| | ggctaaagaagcggccgcaaaggaagcagcagcgaagatgataccgagggg | |
| | tttgagcgaagcaaagcccgcaacgcctgagatacaagaaatcgttgacaa | |
| | agtaaaaccacaattggaagagaagacgggagaaacgtatggaaagcttga | |
| | ggccgtacaatacaagacacaggtgttggcaaattttttccaaaggcgatg | |
| | gcccggatcaacgaattactatatcaaggtacgcgcgggggataacaagta | |
| | catgcatctcaaggttttcaacggtccgtggaaattccgaaatacagaccg | |
| | cggagccgacagggtcctcaccggctatcaggtcgacaagaataaggatga | |
| | tgagttgacaggtttcgccgccgccggtgagcagaaattgatctccgaaga | |
| | ggatctcggtgccgcgcaccaccaccaccatcatggaagcggagagggcag | |
| | gggaagtcttctaacatgcggggacgtggaggaaaatcccggccccatgat | |
| | tgaacaagatggattgcacgcaggttctccggccgcttgggtggagaggct | |
| | attcggctatgactgggcacaacagacaatcggctgctctgatgccgccgt | |
| | gttccggctgtcagcgcaggggcgcccggttctttttgtcaagaccgacct | |
| | gtccggtgccctgaatgaactgcaagacgaggcagcgcggctatcgtggct | |
| | ggccacgacgggcgttccttgcgcagctgtgctcgacgttgtcactgaagc | |
| | gggaagggactggctgctattgggcgaagtgccggggcaggatctcctgtc | |
| | atctcaccttgctcctgccgagaaagtatccatcatggctgatgcaatgcg | |
| | gcggctgcatacgcttgatccggctacctgcccattcgaccaccaagcgaa | |
| | acatcgcatcgagcgagcacgtactcggatggaagccggtcttgtcgatca | |
| | ggatgatctggacgaagagcatcaggggctcgcgccagccgaactgttcgc | |
| | caggctcaaggcgagcatgcccgacggcgaggatctcgtcgtgacccatgg | |
| | cgatgcctgcttgccgaatatcatggtggaaaatggccgcttttctggatt | |
| | catcgactgtggccggctgggtgtggcggaccgctatcaggacatagcgtt | |
| | ggctacccgtgatattgctgaagagcttggcggcgaatgggctgaccgctt | |
| | cctcgtgctttacggtatcgccgctcccgattcgcagcgcatcgccttcta | |
| | tcgccttcttgacgagttcttctgaacccagctttcttgtacaaagtggtg | |
| | ataatcgaattccgataatcaacctctggattacaaaatttgtgaaagatt | |
| | gactggtattcttaactatgttgctccttttacgctatgtggatacgctgc | |
| | tttaatgcctttgtatcatgctattgcttcccgtatggctttcattttctc | |
| | ctccttgtataaatcctggttgctgtctctttatgaggagttgtggcccgt | |
| | tgtcaggcaacgtggcgtggtgtgcactgtgtttgctgacgcaacccccac | |
| | tggttggggcattgccaccacctgtcagctcctttccgggactttcgcttt | |
| | ccccctccctattgccacggcggaactcatcgccgcctgccttgcccgctg | |
| | ctggacaggggctcggctgttgggcactgacaattccgtggtgttgtcggg | |
| | gaagctgacgtcctttccatggctgctcgcctgtgttgccacctggattct | |
| | gcgcgggacgtccttctgctacgtcccttcggccctcaatccagcggacct | |
| | tccttcccgcggcctgctgccggctctgcggcctcttccgcgtcttcgcct | |
| | tcgccctcagacgagtcggatctccctttgggccgcctccccgcatcggga | |
| | attcccgcggttcgctttaagaccaatgacttacaaggcagctgtagatct | |
| | tagccactttttaaaagaaaaggggggactggaagggctaattcactccca | |
| | acgaagacaagatctgctttttgcttgtactgggtctctctggttagacca | |
| | gatctgagcctgggagctctctggctaactagggaacccactgcttaagcc | |
| | tcaataaagcttgccttgagtgcttcaagtagtgtgtgcccgtctgttgtg | |
| | tgactctggtaactagagatccctcagacccttttagtcagtgtggaaaat | |
| | ctctagcagtagtagttcatgtcatcttattattcagtatttataacttgc | |
| | aaagaaatgaatatcagagagtgagaggaacttgtttattgcagcttataa | |
| | tggttacaaataaagcaatagcatcacaaatttcacaaataaagcattttt | |
| | ttcactgcattctagttgtggtttgtccaaactcatcaatgtatcttatca | |
| | tgtctggctctagctatcccgcccctaactccgcccatcccgcccctaact | |
| | ccgcccagttccgcccattctccgccccatggctgactaattttttttatt | |
| | tatgcagaggccgaggccgcctcggcctctgagctattccagaagtagtga | |
| | ggaggcttttttggaggcctagggacgtacccaattcgccctatagtgagt | |
| | cgtattacgcgcgctcactggccgtcgttttacaacgtcgtgactgggaaa | |
| | accctggcgttacccaacttaatcgccttgcagcacatccccctttcgcca | |
| | gctggcgtaatagcgaagaggcccgcaccgatcgcccttcccaacagttgc | |
| | gcagcctgaatggcgaatgggacgcgccctgtagcggcgcattaagcgcgg | |
| | cgggtgtggtggttacgcgcagcgtgaccgctacacttgccagcgccctag | |
| | cgcccgctcctttcgctttcttcccttcctttctcgccacgttcgccggct | |
| | ttccccgtcaagctctaaatcgggggctccctttagggttccgatttagtg | |
| | ctttacggcacctcgaccccaaaaaacttgattagggtgatggttcacgta | |
| | gtgggccatcgccctgatagacggtttttcgccctttgacgttggagtcca | |
| | cgttctttaatagtggactcttgttccaaactggaacaacactcaacccta | |
| | tctcggtctattcttttgatttataagggattttgccgatttcggcctatt | |
| | ggttaaaaaatgagctgatttaacaaaaatttaacgcgaattttaacaaaa | |
| | tattaacgcttacaatttaggtggcacttttcggggaaatgtgcgcggaac | |
| | ccctatttgtttatttttctaaatacattcaaatatgtatccgctcatgag | |
| | acaataaccctgataaatgcttcaataatattgaaaaaggaagagtatgag | |
| | tattcaacatttccgtgtcgcccttattcccttttttgcggcattttgcct | |
| | tcctgtttttgctcacccagaaacgctggtgaaagtaaaagatgctgaaga | |
| | tcagttgggtgcacgagtgggttacatcgaactggatctcaacagcggtaa | |
| | gatccttgagagttttcgccccgaagaacgttttccaatgatgagcacttt | |
| | taaagttctgctatgtggcgcggtattatcccgtattgacgccgggcaaga | |
| | gcaactcggtcgccgcatacactattctcagaatgacttggttgagtactc | |
| | accagtcacagaaaagcatcttacggatggcatgacagtaagagaattatg | |
| | cagtgctgccataaccatgagtgataacactgcggccaacttacttctgac | |
| | aacgatcggaggaccgaaggagctaaccgcttttttgcacaacatggggga | |
| | tcatgtaactcgccttgatcgttgggaaccggagctgaatgaagccatacc | |
| | aaacgacgagcgtgacaccacgatgcctgtagcaatggcaacaacgttgcg | |
| | caaactattaactggcgaactacttactctagcttcccggcaacaattaat | |
| | agactggatggaggcggataaagttgcaggaccacttctgcgctcggccct | |
| | tccggctggctggtttattgctgataaatctggagccggtgagcgtgggtc | |
| | tcgcggtatcattgcagcactggggccagatggtaagccctcccgtatcgt | |
| | agttatctacacgacggggagtcaggcaactatggatgaacgaaatagaca | |
| | gatcgctgagataggtgcctcactgattaagcattggtaactgtcagacca | |
| | agtttactcatatatactttagattgatttaaaacttcatttttaatttaa | |
| | aaggatctaggtgaagatcctttttgataatctcatgaccaaaatccctta | |
| | acgtgagttttcgttccactgagcgtcagaccccgtagaaaagatcaaagg | |
| | atcttcttgagatcctttttttctgcgcgtaatctgctgcttgcaaacaaa | |
| | aaaaccaccgctaccagcggtggtttgtttgccggatcaagagctaccaac | |
| | tctttttccgaaggtaactggcttcagcagagcgcagataccaaatactgt | |
| | tcttctagtgtagccgtagttaggccaccacttcaagaactctgtagcacc | |
| | gcctacatacctcgctctgctaatcctgttaccagtggctgctgccagtgg | |
| | cgataagtcgtgtcttaccgggttggactcaagacgatagttaccggataa | |
| | ggcgcagcggtcgggctgaacggggggttcgtgcacacagcccagcttgga | |
| | gcgaacgacctacaccgaactgagatacctacagcgtgagctatgagaaag | |
| | cgccacgcttcccgaagagagaaaggcggacaggtatccggtaagcggcag | |
| | ggtcggaacaggagagcgcacgagggagcttccagggggaaacgcctggta | |
| | tctttatagtcctgtcgggtttcgccacctctgacttgagcgtcgattttt | |
| | gtgatgctcgtcaggggggcggagcctatggaaaaacgccagcaacgcggc | |
| | ctttttacggttcctggccttttgctggccttttgctcacatgttctttcc | |
| | tgcgttatcccctgattctgtggataaccgtattaccgcctttgagtgagc | |
| | tgataccgctcgccgcagccgaacgaccgagcgcagcgagtcagtgagcga | |
| | ggaagcggaagagcgcccaatacgcaaaccgcctctccccgcgcgttggcc | |
| | gattcattaatgcagctggcacgacaggtttcccgactggaaagcgggcag | |
| | tgagcgcaacgcaattaatgtgagttagctcactcattaggcaccccaggc | |
| | tttacactttatgcttccggctcgtatgttgtgtggaattgtgagcggata | |
| | acaatttcacacaggaaacagctatgaccatgattacgccaagcgcgcaat | |
| | taaccctcactaaagggaacaaaagctggagctgcaagctt | |
| VB211001- | aatgtagtcttatgcaatactcttgtagtcttgcaacatggtaacgatgag | 736 |
| 1283mjc | ttagcaacatgccttacaaggagagaaaaagcaccgtgcatgccgattggt | |
| (9287 bp) | ggaagtaaggtggtacgatcgtgccttattaggaaggcaacagacgggtct | |
| | gacatggattggacgaaccactgaattgccgcattgcagagatattgtatt | |
| | taagtgcctagctcgatacataaacgggtctctctggttagaccagatctg | |
| | agcctgggagctctctggctaactagggaacccactgcttaagcctcaata | |
| | aagcttgccttgagtgcttcaagtagtgtgtgcccgtctgttgtgtgactc | |
| | tggtaactagagatccctcagacccttttagtcagtgtggaaaatctctag | |
| | cagtggcgcccgaacagggacttgaaagcgaaagggaaaccagaggagctc | |
| | tctcgacgcaggactcggcttgctgaagcgcgcacggcaagaggcgagggg | |
| | cggcgactggtgagtacgccaaaaattttgactagcggaggctagaaggag | |
| | agagatgggtgcgagagcgtcagtattaagcgggggagaattagatcgcga | |
| | tgggaaaaaattcggttaaggccagggggaaagaaaaaatataaattaaaa | |
| | catatagtatgggcaagcagggagctagaacgattcgcagttaatcctggc | |
| | ctgttagaaacatcagaaggctgtagacaaatactgggacagctacaacca | |
| | tcccttcagacaggatcagaagaacttagatcattatataatacagtagca | |
| | accctctattgtgtgcatcaaaggatagagataaaagacaccaaggaagct | |
| | ttagacaagatagaggaagagcaaaacaaaagtaagaccaccgcacagcaa | |
| | gcggccgctgatcttcagacctggaggaggagatatgagggacaattggag | |
| | aagtgaattatataaatataaagtagtaaaaattgaaccattaggagtagc | |
| | acccaccaaggcaaagagaagagtggtgcagagagaaaaaagagcagtggg | |
| | aataggagctttgttccttgggttcttgggagcagcaggaagcactatggg | |
| | cgcagcgtcaatgacgctgacggtacaggccagacaattattgtctggtat | |
| | agtgcagcagcagaacaatttgctgagggctattgaggcgcaacagcatct | |
| | gttgcaactcacagtctggggcatcaagcagctccaggcaagaatcctggc | |
| | tgtggaaagatacctaaaggatcaacagctcctggggatttggggttgctc | |
| | tggaaaactcatttgcaccactgctgtgccttggaatgctagttggagtaa | |
| | taaatctctggaacagatttggaatcacacgacctggatggagtgggacag | |
| | agaaattaacaattacacaagcttaatacactccttaattgaagaatcgca | |
| | aaaccagcaagaaaagaatgaacaagaattattggaattagataaatgggc | |
| | aagtttgtggaattggtttaacataacaaattggctgtggtatataaaatt | |
| | attcataatgatagtaggaggcttggtaggtttaagaatagtttttgctgt | |
| | actttctatagtgaatagagttaggcagggatattcaccattatcgtttca | |
| | gacccacctcccaaccccgaggggacccgacaggcccgaaggaatagaaga | |
| | agaaggtggagagagagacagagacagatccattcgattagtgaacggatc | |
| | tcgacggtatcgctagcttttaaaagaaaaggggggattggggggtacagt | |
| | gcaggggaaagaatagtagacataatagcaacagacatacaaactaaagaa | |
| | ttacaaaaacaaattacaaaaattcaaaattttactagtgattatcggatc | |
| | aactttgtatagaaaagttgtgaaagaccccacctgtaggtttggcaagct | |
| | agcttaagtaacgccattttgcaaggcatggaaaatacataactgagaata | |
| | gagaagttcagatcaaggttaggaacagagagacagcagaatatgggccaa | |
| | acaggatatctgtggtaagcagttcctgccccggctcagggccaagaacag | |
| | atggtccccagatgcggtcccgccctcagcagtttctagagaaccatcaga | |
| | tgtttccagggtgccccaaggacctgaaatgaccctgtgccttatttgaac | |
| | taaccaatcagttcgcttctcgcttctgttcgcgcgcttctgctccccgag | |
| | ctcaataaaagagcccacaacccctcactcggcgcgccagtcctccgatag | |
| | actgcgtcgcccgggtacccgtattcccaataaagcctcttgctgtttgca | |
| | tccgaatcgtggactcgctgatccttgggagggtctcctcagattgattga | |
| | ctgcccacctcgggggtctttcattcaagtttgtacaaaaaagcaggctgc | |
| | caccatgccattgcttcttctgctgccactcctttgggctggtgcgctggc | |
| | tattccggggggtttgtcagaagcaaagcccgcgaccccagaaattcagga | |
| | gatcgtggataaagtaaaacctcagcttgaagaaaaaaccggggagacata | |
| | cggcaagctggaagcagtacaatataaaacgcaggtagtacattactacgt | |
| | ccattataacgatcaaggtacaaattattatataaaagtacgggcagggga | |
| | caacaaatacatgcatctgaaagtatttaagagtctgtggggtgagaactt | |
| | gtttgcaaagtgggaagatttggtgcttaccggctaccaggtcgataagaa | |
| | taaggatgacgagcttacaggttttgcagaagctgcagcgaaagaggctgc | |
| | cgcaaaagaagcagcggctaaggaagcggccgctaaagaagcggccgcgaa | |
| | ggaagcggccgctaagatgatcccgggtggtctgtccgaagccaaaccggc | |
| | gacgccggaaatccaggagatagtcgataaggtcaagccacaactcgaaga | |
| | gaagactggagaaacatatggaaaacttgaggccgttcaatacaaaacgca | |
| | ggtggtccactactacgttcactataacgatcaaggaacaaactattacat | |
| | taaagtcagggcgggagacaacaagtacatgcatcttaaagtgtttaaatc | |
| | tctttggggggagaatcttttcgccaagtgggaagacttggtcctcaccgg | |
| | ataccaggtcgacaaaaacaaagacgatgaactcaccggctttgccgaagc | |
| | ggctgcgaaagaagcggctgccaaagaggctgcagccaaagaagctgcggc | |
| | aaaggaggccgccgccaaagaagcggcggccaaaatgatccccggaggact | |
| | tagcgaggctaaacccgcgacacctgaaatacaggagatcgtggataaggt | |
| | taaaccacaattggaggagaaaactggtgagacctatggcaagctggaagc | |
| | ggttcagtataaaacgcaagttgtacactactatgtacattacaacgatca | |
| | ggggacgaactactatattaaggtcagggcaggagataataaatatatgca | |
| | cttgaaagttttcaaaagcctgtggggggaaaacttgttcgccaagtggga | |
| | agatctcgtcttgactgggtaccaggttgataagaacaaagacgatgaact | |
| | gacaggcttcgctgaagcggctgcaaaggaggcggcagcgaaggaggctgc | |
| | cgcgaaagaagcggcggctaaagaagcggccgcaaaggaagcagcagcgaa | |
| | gatgataccgaggggtttgagcgaagcaaagcccgcaacgcctgagataca | |
| | agaaatcgttgacaaagtaaaaccacaattggaagagaagacgggagaaac | |
| | gtatggaaagcttgaggccgtacaatacaagacacaggtgttggcaaattt | |
| | tttccaaaggcgatggcccggatcaacgaattactatatcaaggtacgcgc | |
| | gggggataacaagtacatgcatctcaaggttttcaacggtccgtggaaatt | |
| | ccgaaatacagaccgcggagccgacagggtcctcaccggctatcaggtcga | |
| | caagaataaggatgatgagttgacaggtttcgccgccgccggtgagcagaa | |
| | attgatctccgaagaggatctcggtgccgcgcaccaccaccaccatcatgg | |
| | aagcggagagggcaggggaagtcttctaacatgcggggacgtggaggaaaa | |
| | tcccggccccatgattgaacaagatggattgcacgcaggttctccggccgc | |
| | ttgggtggagaggctattcggctatgactgggcacaacagacaatcggctg | |
| | ctctgatgccgccgtgttccggctgtcagcgcaggggcgcccggttctttt | |
| | tgtcaagaccgacctgtccggtgccctgaatgaactgcaagacgaggcagc | |
| | gcggctatcgtggctggccacgacgggcgttccttgcgcagctgtgctcga | |
| | cgttgtcactgaagcgggaagggactggctgctattgggcgaagtgccggg | |
| | gcaggatctcctgtcatctcaccttgctcctgccgagaaagtatccatcat | |
| | ggctgatgcaatgcggcggctgcatacgcttgatccggctacctgcccatt | |
| | cgaccaccaagcgaaacatcgcatcgagcgagcacgtactcggatggaagc | |
| | cggtcttgtcgatcaggatgatctggacgaagagcatcaggggctcgcgcc | |
| | agccgaactgttcgccaggctcaaggcgagcatgcccgacggcgaggatct | |
| | cgtcgtgacccatggcgatgcctgcttgccgaatatcatggtggaaaatgg | |
| | ccgcttttctggattcatcgactgtggccggctgggtgtggcggaccgcta | |
| | tcaggacatagcgttggctacccgtgatattgctgaagagcttggcggcga | |
| | atgggctgaccgcttcctcgtgctttacggtatcgccgctcccgattcgca | |
| | gcgcatcgccttctatcgccttcttgacgagttcttctgaacccagctttc | |
| | ttgtacaaagtggtgataatcgaattccgataatcaacctctggattacaa | |
| | aatttgtgaaagattgactggtattcttaactatgttgctccttttacgct | |
| | atgtggatacgctgctttaatgcctttgtatcatgctattgcttcccgtat | |
| | ggctttcattttctcctccttgtataaatcctggttgctgtctctttatga | |
| | ggagttgtggcccgttgtcaggcaacgtggcgtggtgtgcactgtgtttgc | |
| | tgacgcaacccccactggttggggcattgccaccacctgtcagctcctttc | |
| | cgggactttcgctttccccctccctattgccacggcggaactcatcgccgc | |
| | ctgccttgcccgctgctggacaggggctcggctgttgggcactgacaattc | |
| | cgtggtgttgtcggggaagctgacgtcctttccatggctgctcgcctgtgt | |
| | tgccacctggattctgcgcgggacgtccttctgctacgtcccttcggccct | |
| | caatccagcggaccttccttcccgcggcctgctgccggctctgcggcctct | |
| | tccgcgtcttcgccttcgccctcagacgagtcggatctccctttgggccgc | |
| | ctccccgcatcgggaattcccgcggttcgctttaagaccaatgacttacaa | |
| | ggcagctgtagatcttagccactttttaaaagaaaaggggggactggaagg | |
| | gctaattcactcccaacgaagacaagatctgctttttgcttgtactgggtc | |
| | tctctggttagaccagatctgagcctgggagctctctggctaactagggaa | |
| | cccactgcttaagcctcaataaagcttgccttgagtgcttcaagtagtgtg | |
| | tgcccgtctgttgtgtgactctggtaactagagatccctcagaccctttta | |
| | gtcagtgtggaaaatctctagcagtagtagttcatgtcatcttattattca | |
| | gtatttataacttgcaaagaaatgaatatcagagagtgagaggaacttgtt | |
| | tattgcagcttataatggttacaaataaagcaatagcatcacaaatttcac | |
| | aaataaagcatttttttcactgcattctagttgtggtttgtccaaactcat | |
| | caatgtatcttatcatgtctggctctagctatcccgcccctaactccgccc | |
| | atcccgcccctaactccgcccagttccgcccattctccgccccatggctga | |
| | ctaattttttttatttatgcagaggccgaggccgcctcggcctctgagcta | |
| | ttccagaagtagtgaggaggcttttttggaggcctagggacgtacccaatt | |
| | cgccctatagtgagtcgtattacgcgcgctcactggccgtcgttttacaac | |
| | gtcgtgactgggaaaaccctggcgttacccaacttaatcgccttgcagcac | |
| | atccccctttcgccagctggcgtaatagcgaagaggcccgcaccgatcgcc | |
| | cttcccaacagttgcgcagcctgaatggcgaatgggacgcgccctgtagcg | |
| | gcgcattaagcgcggcgggtgtggtggttacgcgcagcgtgaccgctacac | |
| | ttgccagcgccctagcgcccgctcctttcgctttcttcccttcctttctcg | |
| | ccacgttcgccggctttccccgtcaagctctaaatcgggggctccctttag | |
| | ggttccgatttagtgctttacggcacctcgaccccaaaaaacttgattagg | |
| | gtgatggttcacgtagtgggccatcgccctgatagacggtttttcgccctt | |
| | tgacgttggagtccacgttctttaatagtggactcttgttccaaactggaa | |
| | caacactcaaccctatctcggtctattcttttgatttataagggattttgc | |
| | cgatttcggcctattggttaaaaaatgagctgatttaacaaaaatttaacg | |
| | cgaattttaacaaaatattaacgcttacaatttaggtggcacttttcgggg | |
| | aaatgtgcgcggaacccctatttgtttatttttctaaatacattcaaatat | |
| | gtatccgctcatgagacaataaccctgataaatgcttcaataatattgaaa | |
| | aaggaagagtatgagtattcaacatttccgtgtcgcccttattcccttttt | |
| | tgcggcattttgccttcctgtttttgctcacccagaaacgctggtgaaagt | |
| | aaaagatgctgaagatcagttgggtgcacgagtgggttacatcgaactgga | |
| | tctcaacagcggtaagatccttgagagttttcgccccgaagaacgttttcc | |
| | aatgatgagcacttttaaagttctgctatgtggcgcggtattatcccgtat | |
| | tgacgccgggcaagagcaactcggtcgccgcatacactattctcagaatga | |
| | cttggttgagtactcaccagtcacagaaaagcatcttacggatggcatgac | |
| | agtaagagaattatgcagtgctgccataaccatgagtgataacactgcggc | |
| | caacttacttctgacaacgatcggaggaccgaaggagctaaccgctttttt | |
| | gcacaacatgggggatcatgtaactcgccttgatcgttgggaaccggagct | |
| | gaatgaagccataccaaacgacgagcgtgacaccacgatgcctgtagcaat | |
| | ggcaacaacgttgcgcaaactattaactggcgaactacttactctagcttc | |
| | ccggcaacaattaatagactggatggaggcggataaagttgcaggaccact | |
| | tctgcgctcggcccttccggctggctggtttattgctgataaatctggagc | |
| | cggtgagcgtgggtctcgcggtatcattgcagcactggggccagatggtaa | |
| | gccctcccgtatcgtagttatctacacgacggggagtcaggcaactatgga | |
| | tgaacgaaatagacagatcgctgagataggtgcctcactgattaagcattg | |
| | gtaactgtcagaccaagtttactcatatatactttagattgatttaaaact | |
| | tcatttttaatttaaaaggatctaggtgaagatcctttttgataatctcat | |
| | gaccaaaatcccttaacgtgagttttcgttccactgagcgtcagaccccgt | |
| | agaaaagatcaaaggatcttcttgagatcctttttttctgcgcgtaatctg | |
| | ctgcttgcaaacaaaaaaaccaccgctaccagcggtggtttgtttgccgga | |
| | tcaagagctaccaactctttttccgaaggtaactggcttcagcagagcgca | |
| | gataccaaatactgttcttctagtgtagccgtagttaggccaccacttcaa | |
| | gaactctgtagcaccgcctacatacctcgctctgctaatcctgttaccagt | |
| | ggctgctgccagtggcgataagtcgtgtcttaccgggttggactcaagacg | |
| | atagttaccggataaggcgcagcggtcgggctgaacggggggttcgtgcac | |
| | acagcccagcttggagcgaacgacctacaccgaactgagatacctacagcg | |
| | tgagctatgagaaagcgccacgcttcccgaagagagaaaggcggacaggta | |
| | tccggtaagcggcagggtcggaacaggagagcgcacgagggagcttccagg | |
| | gggaaacgcctggtatctttatagtcctgtcgggtttcgccacctctgact | |
| | tgagcgtcgatttttgtgatgctcgtcaggggggcggagcctatggaaaaa | |
| | cgccagcaacgcggcctttttacggttcctggccttttgctggccttttgc | |
| | tcacatgttctttcctgcgttatcccctgattctgtggataaccgtattac | |
| | cgcctttgagtgagctgataccgctcgccgcagccgaacgaccgagcgcag | |
| | cgagtcagtgagcgaggaagcggaagagcgcccaatacgcaaaccgcctct | |
| | ccccgcgcgttggccgattcattaatgcagctggcacgacaggtttcccga | |
| | ctggaaagcgggcagtgagcgcaacgcaattaatgtgagttagctcactca | |
| | ttaggcaccccaggctttacactttatgcttccggctcgtatgttgtgtgg | |
| | aattgtgagcggataacaatttcacacaggaaacagctatgaccatgatta | |
| | cgccaagcgcgcaattaaccctcactaaagggaacaaaagctggagctgca | |
| | agctt | |
| VB211012- | aatgtagtcttatgcaatactcttgtagtcttgcaacatggtaacgatgag | 737 |
| 1093gfx | ttagcaacatgccttacaaggagagaaaaagcaccgtgcatgccgattggt | |
| (9570 bp) | ggaagtaaggtggtacgatcgtgccttattaggaaggcaacagacgggtct | |
| | gacatggattggacgaaccactgaattgccgcattgcagagatattgtatt | |
| | taagtgcctagctcgatacataaacgggtctctctggttagaccagatctg | |
| | agcctgggagctctctggctaactagggaacccactgcttaagcctcaata | |
| | aagcttgccttgagtgcttcaagtagtgtgtgcccgtctgttgtgtgactc | |
| | tggtaactagagatccctcagacccttttagtcagtgtggaaaatctctag | |
| | cagtggcgcccgaacagggacttgaaagcgaaagggaaaccagaggagctc | |
| | tctcgacgcaggactcggcttgctgaagcgcgcacggcaagaggcgagggg | |
| | cggcgactggtgagtacgccaaaaattttgactagcggaggctagaaggag | |
| | agagatgggtgcgagagcgtcagtattaagcgggggagaattagatcgcga | |
| | tgggaaaaaattcggttaaggccagggggaaagaaaaaatataaattaaaa | |
| | catatagtatgggcaagcagggagctagaacgattcgcagttaatcctggc | |
| | ctgttagaaacatcagaaggctgtagacaaatactgggacagctacaacca | |
| | tcccttcagacaggatcagaagaacttagatcattatataatacagtagca | |
| | accctctattgtgtgcatcaaaggatagagataaaagacaccaaggaagct | |
| | ttagacaagatagaggaagagcaaaacaaaagtaagaccaccgcacagcaa | |
| | gcggccgctgatcttcagacctggaggaggagatatgagggacaattggag | |
| | aagtgaattatataaatataaagtagtaaaaattgaaccattaggagtagc | |
| | acccaccaaggcaaagagaagagtggtgcagagagaaaaaagagcagtggg | |
| | aataggagctttgttccttgggttcttgggagcagcaggaagcactatggg | |
| | cgcagcgtcaatgacgctgacggtacaggccagacaattattgtctggtat | |
| | agtgcagcagcagaacaatttgctgagggctattgaggcgcaacagcatct | |
| | gttgcaactcacagtctggggcatcaagcagctccaggcaagaatcctggc | |
| | tgtggaaagatacctaaaggatcaacagctcctggggatttggggttgctc | |
| | tggaaaactcatttgcaccactgctgtgccttggaatgctagttggagtaa | |
| | taaatctctggaacagatttggaatcacacgacctggatggagtgggacag | |
| | agaaattaacaattacacaagcttaatacactccttaattgaagaatcgca | |
| | aaaccagcaagaaaagaatgaacaagaattattggaattagataaatgggc | |
| | aagtttgtggaattggtttaacataacaaattggctgtggtatataaaatt | |
| | attcataatgatagtaggaggcttggtaggtttaagaatagtttttgctgt | |
| | actttctatagtgaatagagttaggcagggatattcaccattatcgtttca | |
| | gacccacctcccaaccccgaggggacccgacaggcccgaaggaatagaaga | |
| | agaaggtggagagagagacagagacagatccattcgattagtgaacggatc | |
| | tcgacggtatcgctagcttttaaaagaaaaggggggattggggggtacagt | |
| | gcaggggaaagaatagtagacataatagcaacagacatacaaactaaagaa | |
| | ttacaaaaacaaattacaaaaattcaaaattttactagtgattatcggatc | |
| | aactttgtatagaaaagttgcgttacataacttacggtaaatggcccgcct | |
| | ggctgaccgcccaacgacccccgcccattgacgtcaatagtaacgccaata | |
| | gggactttccattgacgtcaatgggtggagtatttacggtaaactgcccac | |
| | ttggcagtacatcaagtgtatcatatgccaagtacgccccctattgacgtc | |
| | aatgacggtaaatggcccgcctggcattgtgcccagtacatgaccttatgg | |
| | gactttcctacttggcagtacatctacgtattagtcatcgctattaccatg | |
| | gtcgaggtgagccccacgttctgcttcactctccccatctcccccccctcc | |
| | ccacccccaattttgtatttatttattttttaattattttgtgcagcgatg | |
| | ggggcggggggggggggggggcgcgcgccaggcggggcggggcggggcgag | |
| | gggcggggcggggcgaggcggagaggtgcggcggcagccaatcagagcggc | |
| | gcgctccgaaagtttccttttatggcgaggcggcggcggcggcggccctat | |
| | aaaaagcgaagcgcgcggcgggcgggagtcgctgcgcgctgccttcgcccc | |
| | gtgccccgctccgccgccgcctcgcgccgcccgccccggctctgactgacc | |
| | gcgttactcccacaggtgagcgggcgggacggcccttctcctccgggctgt | |
| | aattagctgagcaagaggtaagggtttaagggatggttggttggtggggta | |
| | ttaatgtttaattacctggagcacctgcctgaaatcactttttttcaggtt | |
| | ggcaagtttgtacaaaaaagcaggctgccaccatgccattgcttcttctgc | |
| | tgccactcctttgggctggtgcgctggctattccggggggtttgtcagaag | |
| | caaagcccgcgaccccagaaattcaggagatcgtggataaagtaaaacctc | |
| | agcttgaagaaaaaaccggggagacatacggcaagctggaagcagtacaat | |
| | ataaaacgcaggtagtacattactacgtccattataacgatcaaggtacaa | |
| | attattatataaaagtacgggcaggggacaacaaatacatgcatctgaaag | |
| | tatttaagagtctgtggggtgagaacttgtttgcaaagtgggaagatttgg | |
| | tgcttaccggctaccaggtcgataagaataaggatgacgagcttacaggtt | |
| | ttgcagaagctgcagcgaaagaggctgccgcaaaagaagcagcggctaagg | |
| | aagcggccgctaaagaagcggccgcgaaggaagcggccgctaagatgatcc | |
| | cgggtggtctgtccgaagccaaaccggcgacgccggaaatccaggagatag | |
| | tcgataaggtcaagccacaactcgaagagaagactggagaaacatatggaa | |
| | aacttgaggccgttcaatacaaaacgcaggtggtccactactacgttcact | |
| | ataacgatcaaggaacaaactattacattaaagtcagggcgggagacaaca | |
| | agtacatgcatcttaaagtgtttaaatctctttggggggagaatcttttcg | |
| | ccaagtgggaagacttggtcctcaccggataccaggtcgacaaaaacaaag | |
| | acgatgaactcaccggctttgccgaagcggctgcgaaagaagcggctgcca | |
| | aagaggctgcagccaaagaagctgcggcaaaggaggccgccgccaaagaag | |
| | cggcggccaaaatgatccccggaggacttagcgaggctaaacccgcgacac | |
| | ctgaaatacaggagatcgtggataaggttaaaccacaattggaggagaaaa | |
| | ctggtgagacctatggcaagctggaagcggttcagtataaaacgcaagttg | |
| | tacactactatgtacattacaacgatcaggggacgaactactatattaagg | |
| | tcagggcaggagataataaatatatgcacttgaaagttttcaaaagcctgt | |
| | ggggggaaaacttgttcgccaagtgggaagatctcgtcttgactgggtacc | |
| | aggttgataagaacaaagacgatgaactgacaggcttcgctgaagcggctg | |
| | caaaggaggcggcagcgaaggaggctgccgcgaaagaagcggcggctaaag | |
| | aagcggccgcaaaggaagcagcagcgaagatgataccgaggggtttgagcg | |
| | aagcaaagcccgcaacgcctgagatacaagaaatcgttgacaaagtaaaac | |
| | cacaattggaagagaagacgggagaaacgtatggaaagcttgaggccgtac | |
| | aatacaagacacaggtgttggcaaattttttccaaaggcgatggcccggat | |
| | caacgaattactatatcaaggtacgcgcgggggataacaagtacatgcatc | |
| | tcaaggttttcaacggtccgtggaaattccgaaatacagaccgcggagccg | |
| | acagggtcctcaccggctatcaggtcgacaagaataaggatgatgagttga | |
| | caggtttcgccgccgccggtgagcagaaattgatctccgaagaggatctcg | |
| | gtgccgcgcaccaccaccaccatcatggaagcggagagggcaggggaagtc | |
| | ttctaacatgcggggacgtggaggaaaatcccggccccatgattgaacaag | |
| | atggattgcacgcaggttctccggccgcttgggtggagaggctattcggct | |
| | atgactgggcacaacagacaatcggctgctctgatgccgccgtgttccggc | |
| | tgtcagcgcaggggcgcccggttctttttgtcaagaccgacctgtccggtg | |
| | ccctgaatgaactgcaagacgaggcagcgcggctatcgtggctggccacga | |
| | cgggcgttccttgcgcagctgtgctcgacgttgtcactgaagcgggaaggg | |
| | actggctgctattgggcgaagtgccggggcaggatctcctgtcatctcacc | |
| | ttgctcctgccgagaaagtatccatcatggctgatgcaatgcggcggctgc | |
| | atacgcttgatccggctacctgcccattcgaccaccaagcgaaacatcgca | |
| | tcgagcgagcacgtactcggatggaagccggtcttgtcgatcaggatgatc | |
| | tggacgaagagcatcaggggctcgcgccagccgaactgttcgccaggctca | |
| | aggcgagcatgcccgacggcgaggatctcgtcgtgacccatggcgatgcct | |
| | gcttgccgaatatcatggtggaaaatggccgcttttctggattcatcgact | |
| | gtggccggctgggtgtggcggaccgctatcaggacatagcgttggctaccc | |
| | gtgatattgctgaagagcttggcggcgaatgggctgaccgcttcctcgtgc | |
| | tttacggtatcgccgctcccgattcgcagcgcatcgccttctatcgccttc | |
| | ttgacgagttcttctgaacccagctttcttgtacaaagtggtgataatcga | |
| | attccgataatcaacctctggattacaaaatttgtgaaagattgactggta | |
| | ttcttaactatgttgctccttttacgctatgtggatacgctgctttaatgc | |
| | ctttgtatcatgctattgcttcccgtatggctttcattttctcctccttgt | |
| | ataaatcctggttgctgtctctttatgaggagttgtggcccgttgtcaggc | |
| | aacgtggcgtggtgtgcactgtgtttgctgacgcaacccccactggttggg | |
| | gcattgccaccacctgtcagctcctttccgggactttcgctttccccctcc | |
| | ctattgccacggcggaactcatcgccgcctgccttgcccgctgctggacag | |
| | gggctcggctgttgggcactgacaattccgtggtgttgtcggggaagctga | |
| | cgtcctttccatggctgctcgcctgtgttgccacctggattctgcgcggga | |
| | cgtccttctgctacgtcccttcggccctcaatccagcggaccttccttccc | |
| | gcggcctgctgccggctctgcggcctcttccgcgtcttcgccttcgccctc | |
| | agacgagtcggatctccctttgggccgcctccccgcatcgggaattcccgc | |
| | ggttcgctttaagaccaatgacttacaaggcagctgtagatcttagccact | |
| | ttttaaaagaaaaggggggactggaagggctaattcactcccaacgaagac | |
| | aagatctgctttttgcttgtactgggtctctctggttagaccagatctgag | |
| | cctgggagctctctggctaactagggaacccactgcttaagcctcaataaa | |
| | gcttgccttgagtgcttcaagtagtgtgtgcccgtctgttgtgtgactctg | |
| | gtaactagagatccctcagacccttttagtcagtgtggaaaatctctagca | |
| | gtagtagttcatgtcatcttattattcagtatttataacttgcaaagaaat | |
| | gaatatcagagagtgagaggaacttgtttattgcagcttataatggttaca | |
| | aataaagcaatagcatcacaaatttcacaaataaagcatttttttcactgc | |
| | attctagttgtggtttgtccaaactcatcaatgtatcttatcatgtctggc | |
| | tctagctatcccgcccctaactccgcccatcccgcccctaactccgcccag | |
| | ttccgcccattctccgccccatggctgactaattttttttatttatgcaga | |
| | ggccgaggccgcctcggcctctgagctattccagaagtagtgaggaggctt | |
| | ttttggaggcctagggacgtacccaattcgccctatagtgagtcgtattac | |
| | gcgcgctcactggccgtcgttttacaacgtcgtgactgggaaaaccctggc | |
| | gttacccaacttaatcgccttgcagcacatccccctttcgccagctggcgt | |
| | aatagcgaagaggcccgcaccgatcgcccttcccaacagttgcgcagcctg | |
| | aatggcgaatgggacgcgccctgtagcggcgcattaagcgcggcgggtgtg | |
| | gtggttacgcgcagcgtgaccgctacacttgccagcgccctagcgcccgct | |
| | cctttcgctttcttcccttcctttctcgccacgttcgccggctttccccgt | |
| | caagctctaaatcgggggctccctttagggttccgatttagtgctttacgg | |
| | cacctcgaccccaaaaaacttgattagggtgatggttcacgtagtgggcca | |
| | tcgccctgatagacggtttttcgccctttgacgttggagtccacgttcttt | |
| | aatagtggactcttgttccaaactggaacaacactcaaccctatctcggtc | |
| | tattcttttgatttataagggattttgccgatttcggcctattggttaaaa | |
| | aatgagctgatttaacaaaaatttaacgcgaattttaacaaaatattaacg | |
| | cttacaatttaggtggcacttttcggggaaatgtgcgcggaacccctattt | |
| | gtttatttttctaaatacattcaaatatgtatccgctcatgagacaataac | |
| | cctgataaatgcttcaataatattgaaaaaggaagagtatgagtattcaac | |
| | atttccgtgtcgcccttattcccttttttgcggcattttgccttcctgttt | |
| | ttgctcacccagaaacgctggtgaaagtaaaagatgctgaagatcagttgg | |
| | gtgcacgagtgggttacatcgaactggatctcaacagcggtaagatccttg | |
| | agagttttcgccccgaagaacgttttccaatgatgagcacttttaaagttc | |
| | tgctatgtggcgcggtattatcccgtattgacgccgggcaagagcaactcg | |
| | gtcgccgcatacactattctcagaatgacttggttgagtactcaccagtca | |
| | cagaaaagcatcttacggatggcatgacagtaagagaattatgcagtgctg | |
| | ccataaccatgagtgataacactgcggccaacttacttctgacaacgatcg | |
| | gaggaccgaaggagctaaccgcttttttgcacaacatgggggatcatgtaa | |
| | ctcgccttgatcgttgggaaccggagctgaatgaagccataccaaacgacg | |
| | agcgtgacaccacgatgcctgtagcaatggcaacaacgttgcgcaaactat | |
| | taactggcgaactacttactctagcttcccggcaacaattaatagactgga | |
| | tggaggcggataaagttgcaggaccacttctgcgctcggcccttccggctg | |
| | gctggtttattgctgataaatctggagccggtgagcgtgggtctcgcggta | |
| | tcattgcagcactggggccagatggtaagccctcccgtatcgtagttatct | |
| | acacgacggggagtcaggcaactatggatgaacgaaatagacagatcgctg | |
| | agataggtgcctcactgattaagcattggtaactgtcagaccaagtttact | |
| | catatatactttagattgatttaaaacttcatttttaatttaaaaggatct | |
| | aggtgaagatcctttttgataatctcatgaccaaaatcccttaacgtgagt | |
| | tttcgttccactgagcgtcagaccccgtagaaaagatcaaaggatcttctt | |
| | gagatcctttttttctgcgcgtaatctgctgcttgcaaacaaaaaaaccac | |
| | cgctaccagcggtggtttgtttgccggatcaagagctaccaactctttttc | |
| | cgaaggtaactggcttcagcagagcgcagataccaaatactgttcttctag | |
| | tgtagccgtagttaggccaccacttcaagaactctgtagcaccgcctacat | |
| | acctcgctctgctaatcctgttaccagtggctgctgccagtggcgataagt | |
| | cgtgtcttaccgggttggactcaagacgatagttaccggataaggcgcagc | |
| | ggtcgggctgaacggggggttcgtgcacacagcccagcttggagcgaacga | |
| | cctacaccgaactgagatacctacagcgtgagctatgagaaagcgccacgc | |
| | ttcccgaagagagaaaggcggacaggtatccggtaagcggcagggtcggaa | |
| | caggagagcgcacgagggagcttccagggggaaacgcctggtatctttata | |
| | gtcctgtcgggtttcgccacctctgacttgagcgtcgatttttgtgatgct | |
| | cgtcaggggggcggagcctatggaaaaacgccagcaacgcggcctttttac | |
| | ggttcctggccttttgctggccttttgctcacatgttctttcctgcgttat | |
| | cccctgattctgtggataaccgtattaccgcctttgagtgagctgataccg | |
| | ctcgccgcagccgaacgaccgagcgcagcgagtcagtgagcgaggaagcgg | |
| | aagagcgcccaatacgcaaaccgcctctccccgcgcgttggccgattcatt | |
| | aatgcagctggcacgacaggtttcccgactggaaagcgggcagtgagcgca | |
| | acgcaattaatgtgagttagctcactcattaggcaccccaggctttacact | |
| | ttatgcttccggctcgtatgttgtgtggaattgtgagcggataacaatttc | |
| | acacaggaaacagctatgaccatgattacgccaagcgcgcaattaaccctc | |
| | actaaagggaacaaaagctggagctgcaagctt | |
| 8459_pcLV- | aatattttgaagcatttatcagggttattgtctcatgagcggatacatatt | 738 |
| CMV-AFX01- | tgaatgtatttagaaaaataaacaaataggggttccgcgcacatttccccg | |
| 230 | aaaagtgccacctgacgtctaagaaaccattattatcatgacattaaccta | |
| XT73(C)- | taaaaataggcgtatcacgaggccctttcgtctcgcgcgtttcggtgatga | |
| IRES-Neo | cggtgaaaacctctgacacatgcagctcccggagacggtcacagcttgtct | |
| (9581 bp) | gtaagcggatgccgggagcagacaagcccgtcagggcgcgtcagcgggtgt | |
| | tggcgggtgtcggggctggcttaactatgcggcatcagagcagattgtact | |
| | gagagtgcaccatatgcggtgtgaaataccgcacagatgcgtaaggagaaa | |
| | ataccgcatcaggcgccattcgccattcaggctgcgcaactgttgggaagg | |
| | gcgatcggtgcgggcctcttcgctattacgccagctggcgaaagggggatg | |
| | tgctgcaaggcgattaagttgggtaacgccagggttttcccagtcacgacg | |
| | ttgtaaaacgacggccagtgccaagctgacgcgtgtagtcttatgcaatac | |
| | tcttgtagtcttgcaacatggtaacgatgagttagcaacatgccttacaag | |
| | gagagaaaaagcaccgtgcatgccgattggtggaagtaaggtggtacgatc | |
| | gtgccttattaggaaggcaacagacgggtctgacatggattggacgaacca | |
| | ctgaattgccgcattgcagagatattgtatttaagtgcctagctcgataca | |
| | taaacgggtctctctggttagaccagatctgagcctgggagctctctggct | |
| | aactagggaacccactgcttaagcctcaataaagcttgccttgagtgcttc | |
| | aagtagtgtgtgcccgtctgttgtgtgactctggtaactagagatccctca | |
| | gacccttttagtcagtgtggaaaatctctagcagtggcgcccgaacaggga | |
| | cttgaaagcgaaagggaaaccagaggagctctctcgacgcaggactcggct | |
| | tgctgaagcgcgcacggcaagaggcgaggggcggcgactggtgagtacgcc | |
| | aaaaattttgactagcggaggctagaaggagagagatgggtgcgagagcgt | |
| | cagtattaagcgggggagaattagatcgcgatgggaaaaaattcggttaag | |
| | gccagggggaaagaaaaaatataaattaaaacatatagtatgggcaagcag | |
| | ggagctagaacgattcgcagttaatcctggcctgttagaaacatcagaagg | |
| | ctgtagacaaatactgggacagctacaaccatcccttcagacaggatcaga | |
| | agaacttagatcattatataatacagtagcaaccctctattgtgtgcatca | |
| | aaggatagagataaaagacaccaaggaagctttagacaagatagaggaaga | |
| | gcaaaacaaaagtaagaccaccgcacagcaagcggccactgatcttcagac | |
| | ctggaggaggagatatgagggacaattggagaagtgaattatataaatata | |
| | aagtagtaaaaattgaaccattaggagtagcacccaccaaggcaaagagaa | |
| | gagtggtgcagagagaaaaaagagcagtgggaataggagctttgttccttg | |
| | ggttcttgggagcagcaggaagcactatgggcgcagcgtcaatgacgctga | |
| | cggtacaggccagacaattattgtctggtatagtgcagcagcagaacaatt | |
| | tgctgagggctattgaggcgcaacagcatctgttgcaactcacagtctggg | |
| | gcatcaagcagctccaggcaagaatcctggctgtggaaagatacctaaagg | |
| | atcaacagctcctggggatttggggttgctctggaaaactcatttgcacca | |
| | ctgctgtgccttggaatgctagttggagtaataaatctctggaacagattt | |
| | ggaatcacacgacctggatggagtgggacagagaaattaacaattacacaa | |
| | gcttaatacactccttaattgaagaatcgcaaaaccagcaagaaaagaatg | |
| | aacaagaattattggaattagataaatgggcaagtttgtggaattggttta | |
| | acataacaaattggctgtggtatataaaattattcataatgatagtaggag | |
| | gcttggtaggtttaagaatagtttttgctgtactttctatagtgaatagag | |
| | ttaggcagggatattcaccattatcgtttcagacccacctcccaaccccga | |
| | ggggacccgacaggcccgaaggaatagaagaagaaggtggagagagagaca | |
| | gagacagatccattcgattagtgaacggatctcgacggtatcggttaactt | |
| | ttaaaagaaaaggggggattggggggtacagtgcaggggaaagaatagtag | |
| | acataatagcaacagacatacaaactaaagaattacaaaaacaaattacaa | |
| | aattcaaaattttatttccgcgttacataacttacggtaaatggcccgcct | |
| | ggctgaccgcccaacgacccccgcccattgacgtcaataatgacgtatgtt | |
| | cccatagtaacgccaatagggactttccattgacgtcaatgggtggagtat | |
| | ttacggtaaactgcccacttggcagtacatcaagtgtatcatatgccaagt | |
| | acgccccctattgacgtcaatgacggtaaatggcccgcctggcattatgcc | |
| | cagtacatgaccttatgggactttcctacttggcagtacatctacgtatta | |
| | gtcatcgctattaccatggtgatgcggttttggcagtacatcaatgggcgt | |
| | ggatagcggtttgactcacggggatttccaagtctccaccccattgacgtc | |
| | aatgggagtttgttttggcaccaaaatcaacgggactttccaaaatgtcgt | |
| | aacaactccgccccattgacgcaaatgggcggtaggcgtgtacggtgggag | |
| | gtctatataagcagagctggtttagtgaaccgtcagatccgctagcctagc | |
| | ggatccgaattctcgatcacaagtttgtacaaaaaagcaggcttcgctagc | |
| | cgccaccatgccgctcctgctgctgctgcccctgctgtgggcaggagctct | |
| | ggccatccctggagggctgtccgaggccaagcctgctacgccggaaattca | |
| | agaaatcgtcgataaggttaagccacagctcgaagagaaaactggggaaac | |
| | atacggcaaacttgaggcggttcagtacaaaactcaggtcgtgcactacta | |
| | cgtgcactacaacgatcaagggaccaactactatatcaaggtccgcgcggg | |
| | agacaacaaatacatgcatctcaaagtgttcaaatcgctgtggggcgagaa | |
| | ccttttcgcgaagtgggaggatttggtgctgactggatatcaggtcgataa | |
| | aaacaaggatgacgagcttactggctttggaggaggagggtccggtggagg | |
| | aggctctggtggaggcggaagcggcggaggaggatcgggtggcggaggaag | |
| | cggtggtggaggctccatgattcccggaggcctctcagaagccaaaccagc | |
| | cactcctgagattcaggaaattgtcgacaaggtcaagccgcagctggaaga | |
| | gaagactggcgaaacctacgggaagctagaggcggtccagtacaagaccca | |
| | agtcgtccattactatgtacattacaacgaccagggaaccaattactacat | |
| | taaagtgcgggccggagataataagtatatgcatcttaaggtgtttaagtc | |
| | gttgtggggcgaaaacctgtttgccaagtgggaagatcttgtgctcaccgg | |
| | ttaccaagtggacaagaacaaggacgacgagctgaccgggttcggaggagg | |
| | cggatcgggaggtggaggatccggtggcggtggaagcggaggtggcggatc | |
| | tgggggagggggatcaggaggcggcggatccatgatacctggcggactgtc | |
| | ggaggcgaaaccggccacccctgaaatccaggagattgtggacaaggtgaa | |
| | accccaactcgaggaaaaaaccggcgaaacgtacggtaaactcgaagccgt | |
| | gcagtataagacccaggtcgtacactactatgtgcactataacgatcaggg | |
| | aacgaactactacatcaaggtcagagctggagacaataagtacatgcacct | |
| | caaggttttcaagtcgttatggggagaaaacctcttcgcgaaatgggagga | |
| | tctcgtgctgaccggctaccaggtggacaaaaataaggatgatgaactcac | |
| | cggtttcggaggcggaggatcaggcggagggggttcaggagggggtggctc | |
| | aggaggagggggtagtggaggcggtggatccggaggaggtggcagcatgat | |
| | cccacggggtctgtccgaagccaagccagccaccccagagatccaagaaat | |
| | agtggataaggtgaagccccagcttgaggaaaagactggagagacttacgg | |
| | aaagctcgaggctgtccaatacaaaacacaggtgctggcgaacttcttcca | |
| | aaggagatggccaggcagtactaactattacatcaaagtgcgcgcaggaga | |
| | taacaaatatatgcacttgaaggtgttcaatggaccgtggaagttccggaa | |
| | caccgacagaggcgccgacagggtcttgaccgggtaccaagtcgataagaa | |
| | caaagacgacgaactgaccggtttctgaatctagagacccagctttcttgt | |
| | acaaagtggtatcgagatatctgcatttaattaatctagagtttaaacgcg | |
| | gccgcaaattccgcccctctccctcccccccccctaacgttactggccgaa | |
| | gccgcttggaataaggccggtgtgcgtttgtctatatgttattttccacca | |
| | tattgccgtcttttggcaatgtgagggcccggaaacctggccctgtcttct | |
| | tgacgagcattcctaggggtctttcccctctcgccaaaggaatgcaaggtc | |
| | tgttgaatgtcgtgaaggaagcagttcctctggaagcttcttgaagacaaa | |
| | caacgtctgtagcgaccctttgcaggcagcggaaccccccacctggcgaca | |
| | ggtgcctctgcggccaaaagccacgtgtataagatacacctgcaaaggcgg | |
| | cacaaccccagtgccacgttgtgagttggatagttgtggaaagagtcaaat | |
| | ggctctcctcaagcgtattcaacaaggggctgaaggatgcccagaaggtac | |
| | cccattgtatgggatctgatctggggcctcggtgcacatgctttacatgtg | |
| | tttagtcgaggttaaaaaaacgtctaggccccccgaaccacggggacgtgg | |
| | ttttcctttgaaaaacacgatgataatatggccacaaccatggttattgaa | |
| | caagatggattgcacgcaggttctccggccgcttgggtggagaggctattc | |
| | ggctatgactgggcacaacagacaatcggctgctctgatgccgccgtgttc | |
| | cggctgtcagcgcaggggcgcccggttctttttgtcaagaccgacctgtcc | |
| | ggtgccctgaatgaactgcaggacgaggcagcgcggctatcgtggctggcc | |
| | acgacgggcgttccttgcgcagctgtgctcgacgttgtcactgaagcggga | |
| | agggactggctgctattgggcgaagtgccggggcaggatctcctgtcatct | |
| | caccttgctcctgccgagaaagtatccatcatggctgatgcaatgcggcgg | |
| | ctgcatacgcttgatccggctacctgcccattcgaccaccaagcgaaacat | |
| | cgcatcgagcgagcacgtactcggatggaagccggtcttgtcgatcaggat | |
| | gatctggacgaagagcatcaggggctcgcgccagccgaactgttcgccagg | |
| | ctcaaggcgcgcatgcccgacggcgaggatctcgtcgtgacccatggcgat | |
| | gcctgcttgccgaatatcatggtggaaaatggccgcttttctggattcatc | |
| | gactgtggccggctgggtgtggcggaccgctatcaggacatagcgttggct | |
| | acccgtgatattgctgaagagcttggcggcgaatgggctgaccgcttcctc | |
| | gtgctttacggtatcgccgctcccgattcgcagcgcatcgccttctatcgc | |
| | cttcttgacgagttcttctgagtcgacaatcaacctctggattacaaaatt | |
| | tgtgaaagattgactggtattcttaactatgttgctccttttacgctatgt | |
| | ggatacgctgctttaatgcctttgtatcatgctattgcttcccgtatggct | |
| | ttcattttctcctccttgtataaatcctggttgctgtctctttatgaggag | |
| | ttgtggcccgttgtcaggcaacgtggcgtggtgtgcactgtgtttgctgac | |
| | gcaacccccactggttggggcattgccaccacctgtcagctcctttccggg | |
| | actttcgctttccccctccctattgccacggcggaactcatcgccgcctgc | |
| | cttgcccgctgctggacaggggctcggctgttgggcactgacaattccgtg | |
| | gtgttgtcggggaaatcatcgtcctttccttggctgctcgcctgtgttgcc | |
| | acctggattctgcgcgggacgtccttctgctacgtcccttcggccctcaat | |
| | ccagcggaccttccttcccgcggcctgctgccggctctgcggcctcttccg | |
| | cgtcttcgccttcgccctcagacgagtcggatctccctttgggccgcctcc | |
| | ccgcctggtacctttaagaccaatgacttacaaggcagctgtagatcttag | |
| | ccactttttaaaagaaaaggggggactggaagggctaattcactcccaacg | |
| | aagataagatctgctttttgcttgtactgggtctctctggttagaccagat | |
| | ctgagcctgggagctctctggctaactagggaacccactgcttaagcctca | |
| | ataaagcttgccttgagtgcttcaagtagtgtgtgcccgtctgttgtgtga | |
| | ctctggtaactagagatccctcagacccttttagtcagtgtggaaaatctc | |
| | tagcagtagtagttcatgtcatcttattattcagtatttataacttgcaaa | |
| | gaaatgaatatcagagagtgagaggaacttgtttattgcagcttataatgg | |
| | ttacaaataaagcaatagcatcacaaatttcacaaataaagcatttttttc | |
| | actgcattctagttgtggtttgtccaaactcatcaatgtatcttatcatgt | |
| | ctggctctagctatcccgcccctaactccgcccatcccgcccctaactccg | |
| | cccagttccgcccattctccgccccatggctgactaattttttttatttat | |
| | gcagaggccgaggccgcctcggcctctgagctattccagaagtagtgagga | |
| | ggcttttttggaggcctagacttttgcagagaccaaattcgtaatcatgtc | |
| | atagctgtttcctgtgtgaaattgttatccgctcacaattccacacaacat | |
| | acgagccggaagcataaagtgtaaagcctggggtgcctaatgagtgagcta | |
| | actcacattaattgcgttgcgctcactgcccgctttccagtcgggaaacct | |
| | gtcgtgccagctgcattaatgaatcggccaacgcgcggggagaggcggttt | |
| | gcgtattgggcgctcttccgcttcctcgctcactgactcgctgcgctcggt | |
| | cgttcggctgcggcgagcggtatcagctcactcaaaggcggtaatacggtt | |
| | atccacagaatcaggggataacgcaggaaagaacatgtgagcaaaaggcca | |
| | gcaaaaggccaggaaccgtaaaaaggccgcgttgctggcgtttttccatag | |
| | gctccgcccccctgacgagcatcacaaaaatcgacgctcaagtcagaggtg | |
| | gcgaaacccgacaggactataaagataccaggcgtttccccctggaagctc | |
| | cctcgtgcgctctcctgttccgaccctgccgcttaccggatacctgtccgc | |
| | ctttctcccttcgggaagcgtggcgctttctcatagctcacgctgtaggta | |
| | tctcagttcggtgtaggtcgttcgctccaagctgggctgtgtgcacgaacc | |
| | ccccgttcagcccgaccgctgcgccttatccggtaactatcgtcttgagtc | |
| | caacccggtaagacacgacttatcgccactggcagcagccactggtaacag | |
| | gattagcagagcgaggtatgtaggcggtgctacagagttcttgaagtggtg | |
| | gcctaactacggctacactagaaggacagtatttggtatctgcgctctgct | |
| | gaagccagttaccttcggaaaaagagttggtagctcttgatccggcaaaca | |
| | aaccaccgctggtagcggtggtttttttgtttgcaagcagcagattacgcg | |
| | cagaaaaaaaggatctcaagaagatcctttgatcttttctacggggtctga | |
| | cgctcagtggaacgaaaactcacgttaagggattttggtcatgagattatc | |
| | aaaaaggatcttcacctagatccttttaaattaaaaatgaagttttaaatc | |
| | aatctaaagtatatatgagtaaacttggtctgacagttaccaatgcttaat | |
| | cagtgaggcacctatctcagcgatctgtctatttcgttcatccatagttgc | |
| | ctgactccccgtcgtgtagataactacgatacgggagggcttaccatctgg | |
| | ccccagtgctgcaatgataccgcgagacccacgctcaccggctccagattt | |
| | atcagcaataaaccagccagccggaagggccgagcgcagaagtggtcctgc | |
| | aactttatccgcctccatccagtctattaattgttgccgggaagctagagt | |
| | aagtagttcgccagttaatagtttgcgcaacgttgttgccattgctacagg | |
| | catcgtggtgtcacgctcgtcgtttggtatggcttcattcagctccggttc | |
| | ccaacgatcaaggcgagttacatgatcccccatgttgtgcaaaaaagcggt | |
| | tagctccttcggtcctccgatcgttgtcagaagtaagttggccgcagtgtt | |
| | atcactcatggttatggcagcactgcataattctcttactgtcatgccatc | |
| | cgtaagatgcttttctgtgactggtgagtactcaaccaagtcattctgaga | |
| | atagtgtatgcggcgaccgagttgctcttgcccggcgtcaatacgggataa | |
| | taccgcgccacatagcagaactttaaaagtgctcatcattggaaaacgttc | |
| | ttcggggcgaaaactctcaaggatcttaccgctgttgagatccagttcgat | |
| | gtaacccactcgtgcacccaactgatcttcagcatcttttactttcaccag | |
| | cgtttctgggtgagcaaaaacaggaaggcaaaatgccgcaaaaaagggaat | |
| | aagggcgacacggaaatgttgaatactcatactcttcctttttc | |

### Example 7-2: Establishment of Cell Introduction Conditions

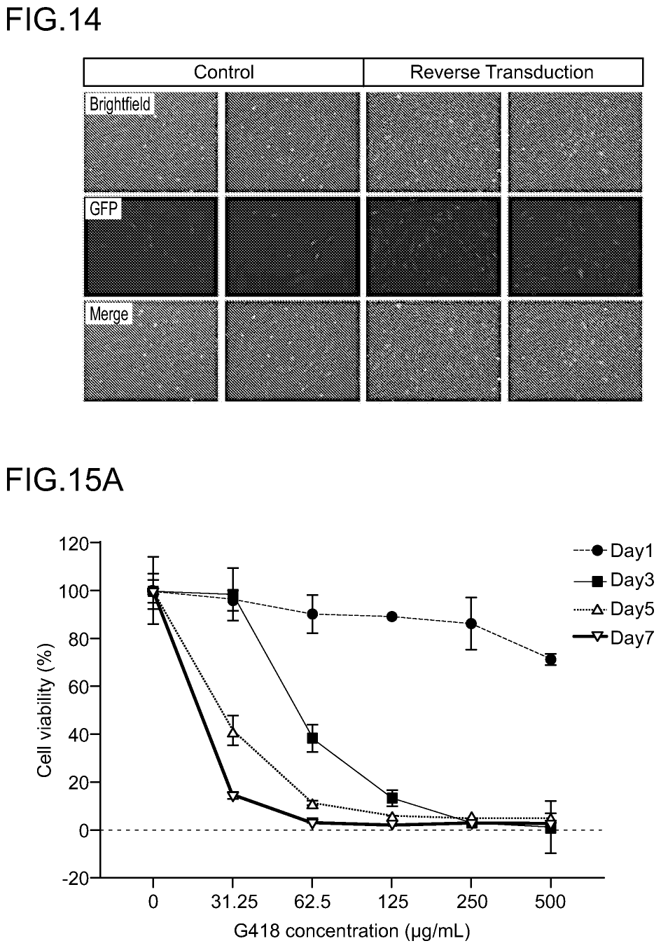

When introducing a gene into PSC-derived MSC using a lentivirus, it is necessary to select an appropriate introduction method in order to increase the introduction efficiency. A typical method of treating cells with a lentivirus is to infect the attached cells with both lentiviral particles and a transduction enhancer. Another method is a reverse transduction method of infecting the cells with the lentivirus during cell attachment by treating the cells with both lentiviral particles and a transduction enhancer before cell attachment. Among these two methods, in order to select a method capable of further increasing the efficiency when introducing the lentivirus into PSC-derived MSC, introduction efficiency was compared using the lentivirus constructed with a vector (SIRION Biotech, SEQ ID NO: 729) including eGFP fluorescence. The frozen PSC-derived MSC was thawed, counted equally, and inoculated. Cell attachment was confirmed the next day, and the cells were then treated with 1-5 MOI of lentivirus and 2-8 μg/mL of polybrene for 16 to 20 hours. Under different conditions, 1-5 MOI of lentivirus and 2-8 μg/mL of polybrene were mixed and placed in a plate, and the same number of cells was inoculated into the plate and treated therewith for 16 to 20 hours. In both conditions, 16 to 20 hours after treatment with the lentivirus, the culture medium containing the lentivirus was removed and replaced with a fresh culture medium, followed by culture for 48 hours. After 48 hours, the cells were harvested and gene introduction efficiency was compared based on the GFP fluorescence-introduced cell population through flow cytometry. Consequently, as shown in FIG. 14, when the attached cells were treated with the lentivirus, the introduction efficiency was 6.61%, whereas when the cells were also treated with the lentivirus during cell inoculation, the introduction efficiency was 21.57%, which was increased about 3.26-fold. Based on these results, it was confirmed that, when introducing a gene into PSC-derived MSC using a lentivirus, treatment with the lentivirus during cell attachment was a method capable of increasing the efficiency.

### Example 7-3: Selection of Genetically Modified Cell into which Gene was Introduced

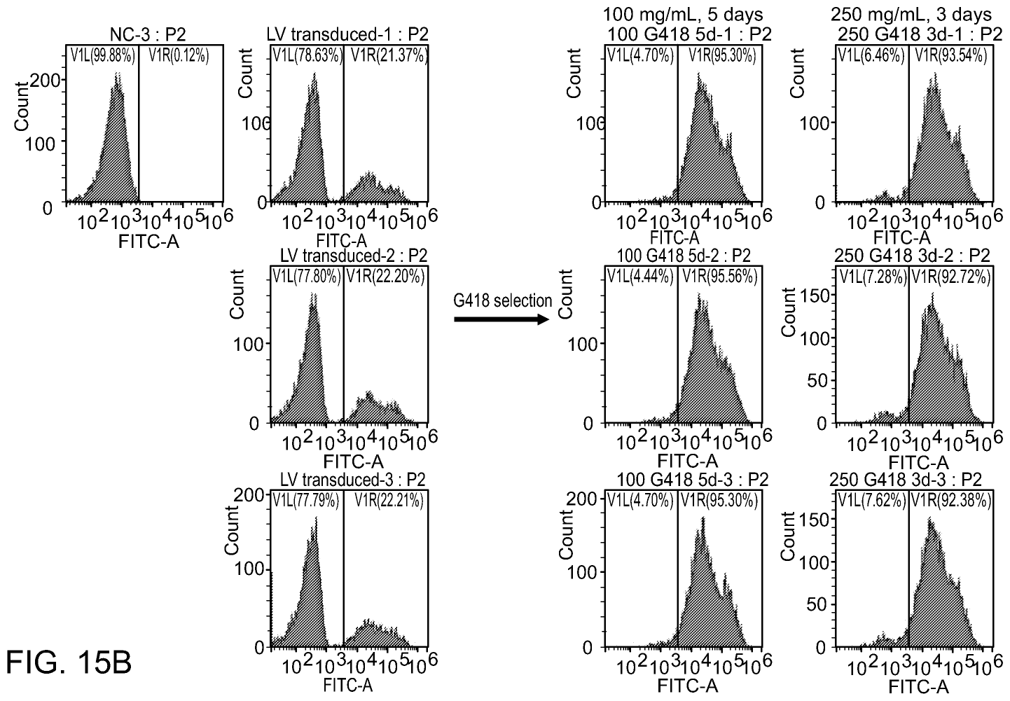

In order to select gene-introduced cells alone after gene introduction through the lentivirus in PSC-derived MSC, the lentivirus was constructed such that a neomycin resistance gene was expressed downstream of the stefin A protein variant (AFFIMERC) gene to be introduced. When the stefin A protein variant (AFFIMER®) gene is introduced into cells, the neomycin resistance gene is also expressed, resulting in antibiotic resistance. However, since treatment with high concentrations of antibiotics for a long time may cause genetic mutation in cells, it is necessary to determine the minimum concentration capable of selecting the gene-introduced cells alone. Since such a concentration varies depending on cell characteristics, in order to determine the minimum G418 concentration that kills PSC-derived MSC, the extent of cell death after cell culture through treatment with the antibiotic G418 at different concentrations was evaluated. The next day after uniform inoculation of PSC-derived MSC into a 96-well plate, the cells were treated with a culture medium containing G418 at 500, 250, 125, 62.5, 31.25, 15.625, and 7.8125 μg/mL. The culture medium containing G418 was thoroughly replaced every 2 days, and cell death was observed after 1, 3, 5, and 7 days from the first treatment day. In order to evaluate cell viability, Cell Counting kit-8 (CCK-8) was diluted 1:10 in the culture medium, and the culture medium containing G418 was thoroughly removed, followed by treatment with 100 μL thereof and then reaction at 37° C. for 1 hour. After reaction, absorbance was measured at 460 nm using a multimode microplate reader. Consequently, as shown in FIG. 15A, when the cells were treated with G418 at a concentration of 500 μg/mL, cell viability was 71.40% on the 1 t day but was 1.34% on the 3^{rd} day, indicating that most cells were killed. When the cells were treated with C418 at a concentration of 250 μg/mL, cell viability was 86.29% on the 1^{st} day but was 3.07% on the 3^{rd} day, indicating that most cells were killed, as with 500 μg/mL. In addition, when the cells were treated with G418 at a concentration of 125 μg/mL, cell viability was 89.14% on the 1^{st} day, 13.43% on the 3^{rd} day, and 5.82% on the 5^{th} day. When the cells were treated with G418 at a concentration of 62.5 μg/mL, cell viability was 90.38% on the 1^{st} day, 38.51% on the 3^{rd} day, 11.52% on the 5^{th} day, and 2.84% on the 7^{th} day.

After setting the G418 concentration and the treatment time, the proportion of the selected gene-introduced cells before and after treatment with G418 was measured. In order to compare suitability for G418 treatment conditions, a lentivirus was constructed (SEQ ID NO: 729) such that a neomycin resistance gene was expressed downstream of the eGFP fluorescent gene. PSC-derived MSC was treated with a mixture of 5 MOI of lentivirus and 2 μg/mL of polybrene, followed by culture for 16 to 20 hours. The culture medium containing the lentivirus was thoroughly removed and then replaced with a fresh culture medium, followed by culture for 48 hours. When cell confluency reached 90% or more, the cells were harvested and then inoculated again at a cell density of 0.4-1.0×10⁴ cells/cm². After culture for 18 to 24 hours, the culture medium was replaced with a culture medium containing G418 at 100 μg/mL and 250 μg/mL, and then replaced with a culture medium containing G418 at each concentration every 2 days. The cells were treated for 5 days in the presence of G418 at 100 μg/mL and for 3 days in the presence of G418 at 250 μg/mL, after which the culture medium was replaced with a culture medium not containing G418. Thereafter, when cell confluency reached 90% or more, the cells were harvested and then the proportion of the eGFP fluorescent gene-introduced cell line was measured through flow cytometry. Consequently, as shown in FIG. 15B, the proportion of the eGFP gene-introduced cell line was 22.00% before treatment with G418, but was 96.01% after treatment for 5 days in the presence of G418 at 100 μg/mL and was 93.47% after treatment for 3 days in the presence of G418 at 250 μg/mL.

### Example 7-4: Promoter Screening

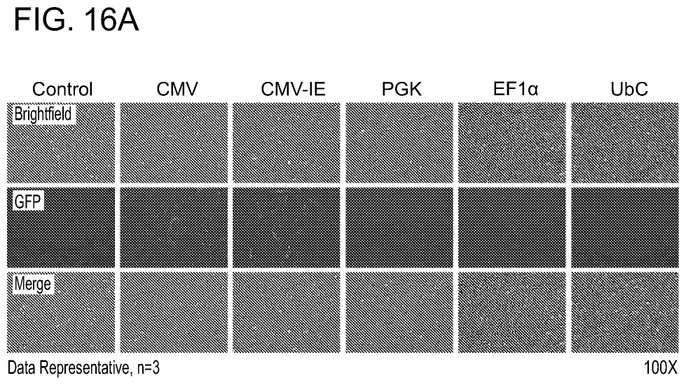

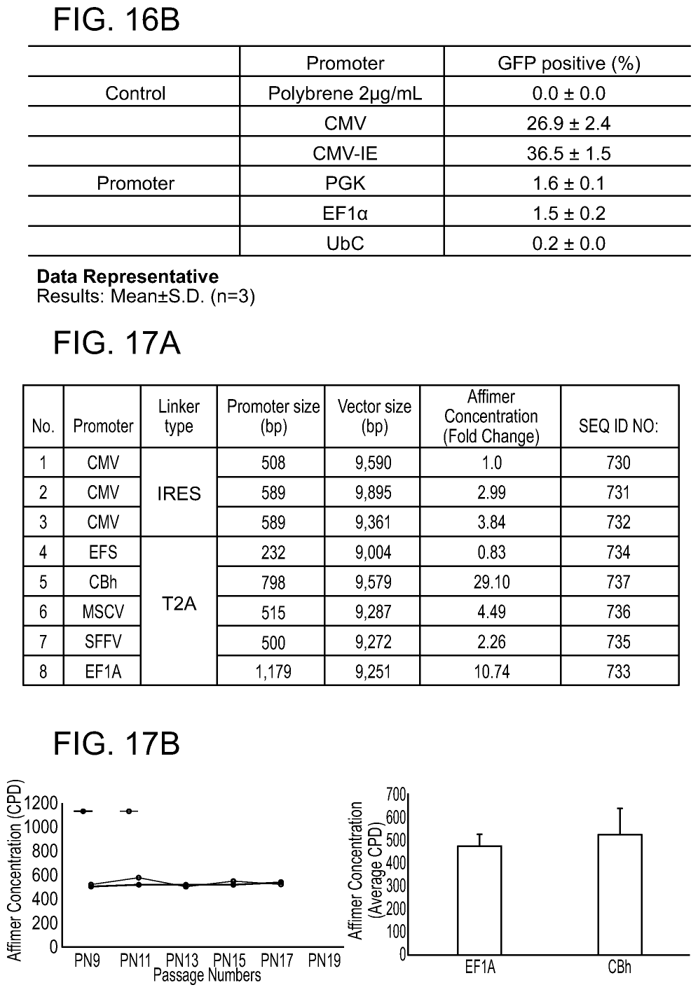

When introducing the stefin A protein variant into PSC-derived MSC, four types of promoters that induce constitutive expression were evaluated in order to select a promoter capable of maintaining stable and high expression. A cell line was constructed with lentiviral particles including a vector (Applied Biological Materials Inc. LV950)) designed to express the eGFP fluorescent protein downstream of four promoters: CMV, PGK, EF1A, and UbC. The cells were treated with a mixture of 1-5 MOI of lentivirus and 2-8 μg/mL of polybrene, followed by culture at 37° C. and 5% CO₂ for 16 to 20 hours. As a positive control, a cell line was constructed and compared under the same conditions using a lentivirus (SIRION Biotech, Table 12) in which an eGFP fluorescent protein was fused downstream of the CMV promoter. The culture medium containing the lentivirus was thoroughly removed and then replaced with a fresh culture medium, followed by culture at 37° C. and 5% CO₂ for 48 hours. After 48 hours, the cells were harvested and gene introduction efficiency was compared based on the GFP fluorescence-introduced cell population through flow cytometry. As shown in FIGS. 16A and 16B, the positive control showed an eGFP gene-introduced cell proportion of 26.9%, whereas the CMV-IE promoter showed 36.5%, which is a 1.4-fold increase. In addition, when using the other PGK, EF1A, and UbC promoters, fluorescence introduction was very low to the levels of 1.6%, 1.5%, and 0.2%, respectively. Therefore, the use of the CMV promoter among the four promoters and the enhancer sequence was confirmed to induce protein expression with the highest efficiency.

### Examples 7-5: Confirmation of Passage Stability of MSC into which Anti-CD40L Stefin A Protein Variant Gene was Introduced

A cell line in which anti-CD40L stefin A protein variant expression was regulated by a vector (VB211001-1274, SEQ ID NO: 731) containing a CMV promoter (CMV-IE) including an enhancer was constructed and compared for long-term subculture stability with a non-introduced cell line (Naïve MSC). A lentivirus was constructed using a vector including an anti-CD40L stefin A protein variant gene and a neomycin resistance gene. The frozen Naïve MSC was thawed, mixed with 1-5 MOI of lentivirus and 2-8 μg/mL of polybrene, and then inoculated into a cell culture dish. After culture at 37° C. and 5% CO₂ for 16 to 20 hours, the culture medium containing the lentivirus was thoroughly removed and then replaced with a fresh culture medium, followed by culture at 37° C. and 5% CO₂ for 48 hours. Thereafter, the cells were harvested and inoculated again at a cell density of 0.4-1.0×10⁴ cells/cm², followed by culture at 37° C. and 5% CO₂ for 18 to 24 hours. The culture medium was replaced with a culture medium containing 100 μg/mL of G418, followed by culture for 5 days, and the culture medium was replaced with a culture medium containing G418 every 2 days. When cell confluency reached 90% or more, the cells were harvested and then frozen. Naïve MSC of the same passage number as the frozen gene-introduced cell line was thawed and inoculated into a T175 flask. After culture for 18 to 24 hours, the culture medium was thoroughly replaced with a fresh culture medium every 2 days, followed by culture. When cell confluency reached 90% or more, the cells were harvested and inoculated again into a T175 flask, followed by culture at 37° C. and 5% CO₂. Continuous culture was carried out until the PDL of the cells dropped to 3.0 or less. The cells remaining after inoculation in each passage were frozen, and after termination of continuous culture, the frozen cells at each passage were thawed and analyzed for purity and immune markers through flow cytometry. Expression of mesenchymal stem cell surface markers CD29, CD44, CD73, and CD105, and expression of cell surface markers for hematopoietic stem cell-specific marker CD45, embryonic stem cell-specific markers SSEA-3, TRA-1-60, and TRA-1-81, and immune marker HLA-DR were comparatively analyzed from passages 10 to 18. As shown in Tables 13 and 14 below, it was confirmed that the expression of the mesenchymal stem cell surface markers CD29, CD44, CD73, and CD105 was maintained at 95% or more until PN18, regardless of gene introduction. Also, expression of CD45, SSEA-3, TRA-1-60, TRA-1-81, and HLA-DR was maintained at less than 1% until PN18, indicating that the characteristics of mesenchymal stem cells were maintained well. Based on these results, it was confirmed that the important characteristics and long-term passage stability of mesenchymal stem cells were maintained even when the anti-CD40L stefin A protein variant gene was introduced.

TABLE 13

| | | Naïve MSC | | | | | | | | |
|---|---|---|---|---|---|---|---|---|---|---|
| Marker | | PN10 | PN11 | PN12 | PN13 | PN14 | PN15 | PN16 | PN17 | PN18 |
| Identification | CD29 | 99.9 | 99.9 | 99.9 | 98.7 | 98.3 | 99.7 | 94.3 | 99.8 | 99.5 |
| Markers | CD44 | 99.9 | 99.9 | 99.9 | 99.8 | 99.9 | 99.9 | 99.9 | 99.9 | 99.9 |
| | CD73 | 99.9 | 99.9 | 99.9 | 99.6 | 98.7 | 99.9 | 98.7 | 99.8 | 99.5 |
| | CD105 | 99.9 | 99.9 | 99.9 | 99.8 | 99.8 | 99.9 | 99.8 | 99.9 | 99.7 |
| Purity | CD45 | 0.8 | 0.9 | 1.3 | 0.6 | 0.6 | 0.5 | 0.6 | 0.5 | 0.5 |
| Markers | SSEA-3 | 0.2 | 0.1 | 0.1 | 0.3 | 0.3 | 0.3 | 0.4 | 0.4 | 0.4 |
| | TRA-1-60 | 0.4 | 0.5 | 0.4 | 0.5 | 0.5 | 0.5 | 0.4 | 0.5 | 0.4 |
| | TRA-1-81 | 0.5 | 0.4 | 0.4 | 0.4 | 0.4 | 0.5 | 0.4 | 0.5 | 0.4 |
| Immune | HLA-DR | 0.3 | 0.3 | 0.4 | 0.4 | 0.5 | 0.5 | 0.5 | 0.4 | 0.4 |
| Makers | | | | | | | | | | |

TABLE 14

| | | eMSC | | | | | | | | |
|---|---|---|---|---|---|---|---|---|---|---|
| Marker | | PN10 | PN11 | PN12 | PN13 | PN14 | PN15 | PN16 | PN17 | PN18 |
| Identification | CD29 | 99.8 | 99.7 | 98.6 | 98.8 | 96.4 | 99.0 | 99.2 | 97.7 | 94.7 |
| Markers | CD44 | 100.0 | 100.0 | 99.9 | 99.7 | 99.9 | 99.9 | 99.9 | 100.0 | 99.9 |
| | CD73 | 100.0 | 100.0 | 99.9 | 100.0 | 99.9 | 99.9 | 99.9 | 99.8 | 99.0 |
| | CD105 | 100.0 | 99.9 | 99.7 | 99.7 | 99.6 | 99.7 | 99.9 | 99.7 | 99.9 |
| Purity | CD45 | 0.1 | 0.6 | 0.3 | 0.4 | 0.4 | 0.3 | 0.2 | 0.1 | 0.5 |
| Markers | SSEA-3 | 0.3 | 0.3 | 0.3 | 0.3 | 0.3 | 0.3 | 0.2 | 0.2 | 0.3 |
| | TRA-1-60 | 0.2 | 0.4 | 0.1 | 0.3 | 0.2 | 0.3 | 0.4 | 0.1 | 0.4 |
| | TRA-1-81 | 0.2 | 0.3 | 0.1 | 0.2 | 0.2 | 0.1 | 0.3 | 0.1 | 0.3 |
| Immune | HLA-DR | 0.7 | 0.4 | 0.3 | 0.3 | 0.2 | 0.1 | 0.3 | 0.1 | 0.4 |
| Makers | | | | | | | | | | |

### Examples 7-6: Additional Screening of Promoter for Expression of Anti-CD40L Stefin A Protein Variant

In order to introduce the stefin A protein variant gene into PSC-derived MSC, eight types of lentiviral vectors having characteristics shown in FIG. 17A were constructed, and a promoter capable of inducing stable and high expression was selected.

A cell line was constructed with lentiviral particles including a vector designed such that the stefin A protein variant (AFFIMER®) was expressed downstream of each promoter and in which an antibiotic gene was inserted downstream of the IRES or T2A sequence. The cells were treated with a mixture of 1-5 MCI of lentivirus and 2-8 μg/mL of polybrene, followed by culture at 37° C. and 5% CO₂ for 16 to 20 hours. Thereafter, the culture medium containing the lentivirus was thoroughly removed and then replaced with a fresh culture medium, followed by culture at 37° C. and 5% CO₂ for 48 hours. After 48 hours, the cells were harvested and expression efficiency of the stefin A protein variant was evaluated through ELISA. As shown in FIG. 17A, two promoters (EF1A and CBh) in which the expression level of the stefin A protein variant was increased 10-fold or more compared to the control (SEQ ID NO: 730) were confirmed.

Each cell line in which the expression of the stefin A protein variant (AFFIMER®) was regulated by the EF1A promoter (SEQ ID NO: 733) or the CBh promoter (SEQ ID NO: 737) was constructed, followed by long-term subculture, and culture stability, expression level, and activity of the stefin A protein variant (AFFIMER®) were evaluated (through binding ELISA, competition ELISA, or functional cell assay). As shown in FIG. 17B, by confirming that expression of the stefin A protein variant was maintained stable from PN9 to PN17 in both cell lines, an expression cassette combination suitable for expression of the stefin A protein variant in PSC-derived MSC was identified.

In order to confirm maintenance of stable stefin A protein variant expression depending on long-term passage, each gene-introduced cell line in which the expression of the stefin A protein variant was regulated by the EF1A promoter (SEQ ID NO: 733) or CBh promoter (SEQ ID NO: 737) was constructed using the method described in Example 7-5. Each of the two gene-introduced cell lines thus constructed was thawed and then inoculated into a T175 flask using a culture medium. After culture at 37° C. and 5% CO₂ for 18 to 24 hours, the culture medium was thoroughly replaced with a fresh culture medium every 2 days, followed by culture. When cell confluency reached 70-80%, the cells were harvested and inoculated again into a T175 flask, followed by continuous subculture. Continuous culture was carried out until the PDL of the cells dropped to 3.0 or less. The cell culture fluid was harvested and frozen during subculture at each passage, and the cells remaining after subculture were frozen. After termination of continuous culture, the frozen cell culture fluid was completely thawed and diluted, after which the stefin A protein variant in the cell culture fluid was quantified through sandwich ELISA. Since the secreted amount thereof may vary depending on the number of cells and the culture period, the daily amount of stefin A protein variant that was secreted per cell was calculated by division by the number of cells harvested during subculture and the time required to harvest the culture fluid after medium replacement. As shown in Table 15 below, it was confirmed that expression of the stefin A protein variant was stably maintained even when long-term subculture was continued.

TABLE 15

| | Passage | Affimer | Affimer secretion |
|---|---|---|---|
| ID | # | (ng/mL) | (fg/cells/day) |
| eMSC | PN9 | 219.2 | 407.18 |
| (w/EF1A) | PN11 | 196.9 | 462.19 |
| | PN13 | 211.5 | 445.78 |
| | PN15 | 214.0 | 492.88 |
| | PN17 | 188.2 | 548.66 |
| eMSC | PN9 | 216.0 | 462.12 |
| (w/CBh) | PN11 | 296.4 | 696.16 |
| | PN13 | 176.5 | 400.73 |
| | PN15 | 229.5 | 577.59 |
| | PN17 | 177.7 | 486.39 |

### Examples 7-7: Confirmation of Long-Term Passage Stability when Using Additional Promoter

The anti-CD40L stefin A protein variant gene-introduced cell lines (eMSC) including two promoters were constructed and then compared for long-term subculture stability with Naïve MSC in the same manner as in Example 7-6.

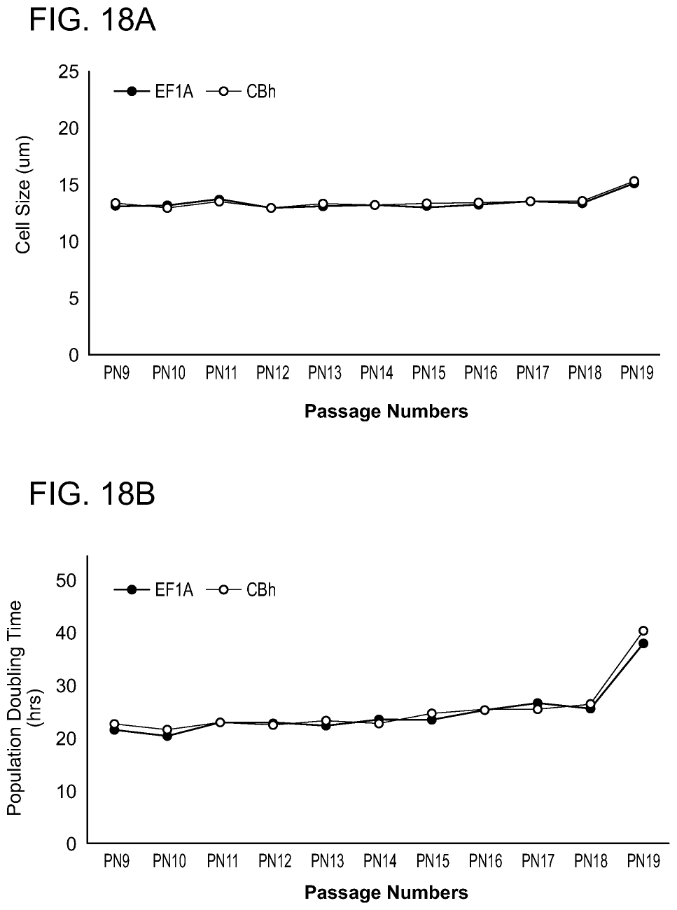

During subculture, cell size, viability, total cell number, PDT, and PDL were measured, and cell morphology was observed using a phase-contrast microscope. Based on the results of morphological observation, two gene-introduced cell lines and Naïve MSC were confirmed to maintain the spindle shape from PN9 to PN19. Cell viability was maintained at 95% or more from PN9 to PN19 in both gene-introduced cell lines, and the amount of the stefin A protein variant that was secreted, PDT, and PDL levels were similar (FIGS. 18A to 16). PDT was gradually increased from 19.21 hours at PN9 to 37.23 hours at PN19 in Naïve MSC, and showed a tendency to increase from 21.78 hours and 22.86 hours at PN9 to 38.11 hours and 40.67 hours at PN19 in the two gene-introduced cell lines. Also, PDL was maintained at 3.0 or more until PN18 in all of Naïve MSC and the two gene-introduced cell lines, but was decreased to 3.0 or less at PN19. Therefore, the long-term subculture stability of PSC-derived MSC was maintained even when the stefin A protein variant gene was introduced, confirming that the present invention is platform technology suitable for the development of cell gene therapies (Table 16).

TABLE 16

| | Passage | Total Cell | Viability | Cell size | | |
|---|---|---|---|---|---|---|
| | number | (×10⁶ cells) | (%) | (μm) | PDT (h) | PDL |
| Naïve | PN9 | 6.87 ± 0.37 | 99.6 ± 0.1 | 12.9 ± 0.1 | 19.21 ± 0.44 | 3.52 ± 0.08 |
| MSC | PN10 | 6.43 ± 0.13 | 99.7 ± 0.1 | 13.7 ± 0.1 | 21.33 ± 0.18 | 3.42 ± 0.03 |
| | PN11 | 6.57 ± 0.40 | 99.6 ± 0.4 | 13.3 ± 0.1 | 21.75 ± 0.56 | 3.45 ± 0.09 |
| | PN12 | 7.38 ± 1.72 | 99.6 ± 0.1 | 13.6 ± 0.2 | 20.53 ± 0.31 | 3.41 ± 0.05 |
| | PN13 | 5.15 ± 0.31 | 99.4 ± 0.3 | 13.3 ± 0.1 | 23.24 ± 0.64 | 3.10 ± 0.09 |
| | PN14 | 5.58 ± 0.18 | 99.5 ± 0.1 | 13.5 ± 0.0 | 22.39 ± 0.32 | 3.22 ± 0.05 |
| | PN15 | 5.00 ± 0.25 | 99.1 ± 0.5 | 13.8 ± 0.2 | 23.55 ± 0.57 | 3.06 ± 0.07 |
| | PN16 | 5.51 ± 0.15 | 99.0 ± 0.3 | 13.9 ± 0.1 | 22.51 ± 0.28 | 3.20 ± 0.04 |
| | PN17 | 3.98 ± 0.23 | 99.1 ± 0.4 | 14.1 ± 0.2 | 26.39 ± 0.79 | 2.73 ± 0.08 |
| | PN18 | 6.36 ± 0.30 | 99.1 ± 0.2 | 14.9 ± 0.1 | 28.20 ± 0.54 | 3.40 ± 0.07 |
| | PN19 | 3.58 ± 0.06 | 97.7 ± 0.4 | 15.3 ± 0.1 | 37.23 ± 0.33 | 2.58 ± 0.02 |
| eMSC | PN9 | 13.51 ± 0.12 | 99.60 ± 0.14 | 13.10 ± 0.00 | 21.78 ± 0.07 | 4.27 ± 0.01 |
| (w/CBh) | PN10 | 9.14 ± 0.55 | 99.65 ± 0.21 | 13.10 ± 0.00 | 20.52 ± 0.48 | 3.71 ± 0.09 |
| | PN11 | 10.70 ± 0.65 | 99.53 ± 0.12 | 13.67 ± 0.06 | 23.15 ± 0.50 | 3.93 ± 0.09 |
| | PN12 | 6.88 ± 0.19 | 99.60 ± 0.10 | 12.90 ± 0.10 | 22.75 ± 0.28 | 3.30 ± 0.04 |
| | PN13 | 5.96 ± 0.04 | 99.53 ± 0.15 | 13.13 ± 0.15 | 22.66 ± 0.07 | 3.09 ± 0.01 |
| | PN14 | 5.82 ± 0.22 | 99.67 ± 0.06 | 13.23 ± 0.12 | 23.58 ± 0.42 | 3.05 ± 0.05 |
| | PN15 | 5.44 ± 0.41 | 99.73 ± 0.06 | 13.10 ± 0.17 | 23.70 ± 0.87 | 2.96 ± 0.11 |
| | PN16 | 5.90 ± 0.13 | 99.63 ± 0.21 | 13.30 ± 0.17 | 25.37 ± 0.26 | 3.08 ± 0.03 |
| | PN17 | 4.32 ± 0.14 | 99.33 ± 0.32 | 13.47 ± 0.12 | 26.68 ± 0.46 | 2.62 ± 0.05 |
| | PN18 | 8.27 ± 0.36 | 99.63 ± 0.06 | 13.37 ± 0.15 | 25.84 ± 0.46 | 3.56 ± 0.06 |
| | PN19 | 4.32 ± 0.10 | 99.10 ± 0.00 | 15.03 ± 0.15 | 38.11 ± 0.49 | 2.62 ± 0.03 |
| eMSC | PN9 | 11.75 ± 0.42 | 99.45 ± 0.07 | 13.35 ± 0.21 | 22.86 ± 0.29 | 4.07 ± 0.05 |
| (w/EF1A) | PN10 | 7.92 ± 0.57 | 99.70 ± 0.00 | 12.95 ± 0.07 | 21.73 ± 0.64 | 3.50 ± 0.10 |
| | PN11 | 10.68 ± 0.64 | 99.63 ± 0.12 | 13.60 ± 0.00 | 23.16 ± 0.51 | 3.93 ± 0.09 |
| | PN12 | 6.91 ± 0.63 | 99.83 ± 0.12 | 12.93 ± 0.06 | 22.71 ± 0.09 | 3.30 ± 0.01 |
| | PN13 | 5.53 ± 0.28 | 99.50 ± 0.10 | 13.30 ± 0.10 | 23.49 ± 0.57 | 2.98 ± 0.07 |
| | PN14 | 6.12 ± 0.52 | 99.67 ± 0.06 | 13.20 ± 0.00 | 23.02 ± 0.09 | 3.13 ± 0.01 |
| | PN15 | 4.98 ± 0.24 | 99.83 ± 0.06 | 13.20 ± 0.17 | 24.76 ± 0.60 | 2.83 ± 0.07 |
| | PN16 | 5.85 ± 0.28 | 99.80 ± 0.00 | 13.33 ± 0.06 | 25.48 ± 0.57 | 3.06 ± 0.07 |
| | PN17 | 4.61 ± 0.33 | 99.10 ± 0.17 | 13.47 ± 0.15 | 25.80 ± 1.01 | 2.72 ± 0.10 |
| | PN18 | 7.57 ± 0.36 | 99.47 ± 0.15 | 13.53 ± 0.15 | 26.81 ± 0.56 | 3.43 ± 0.07 |
| | PN19 | 3.85 ± 0.09 | 99.03 ± 0.06 | 15.30 ± 0.17 | 40.67 ± 0.55 | 2.46 ± 0.03 |

### Example 8: Analysis of Binding and Inhibitory Activity of Anti-CD40L Stefin A Protein Variant

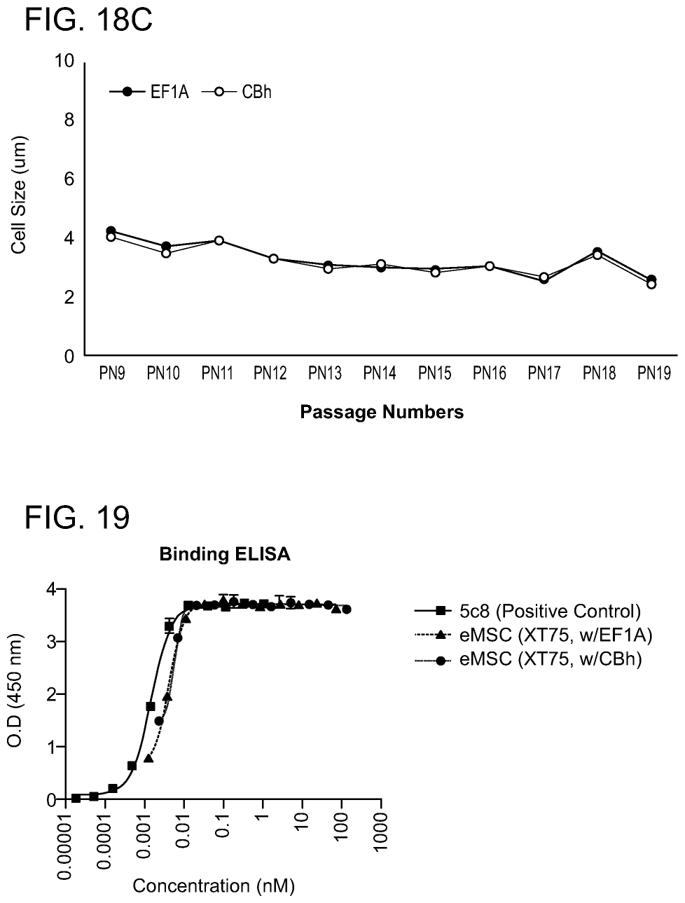

Whether the stefin A protein variant secreted from the constructed anti-CD40L stefin A protein variant gene-introduced cell line had the ability to bind to CD40L and to inhibit binding thereof was evaluated. The anti-CD40L stefin A protein variant gene-introduced cell lines including two promoters were constructed using a lentivirus in the same manner as in Example 7-6. Each cell line was inoculated into a T175 flask at a cell density of 4.0×10³ cells/cm² using a culture medium. After culture at 37° C. and 5% CO₂ for 18 to 24 hours, the culture medium was thoroughly replaced with a fresh culture medium every 2 days, followed by culture. After culture for 3 days, the cells were harvested, and 7.0×10⁶ cells were inoculated into a T175 flask, followed by culture at 37° C. and 5% CO₂ for 18 to 24 hours. The culture medium was thoroughly removed, and the remaining medium was completely removed through washing with 10 mL of MEM alpha. 30 mL of an MEM alpha medium was added to a T175 flask, followed by culture for 48 hours. After 48 hours, all of the cell culture fluid was harvested and then centrifuged to remove residual cells and impurities. The cell culture fluid was transferred to a Vivaspin 20 and centrifuged, the culture fluid was concentrated, and the stefin A protein variant was quantified through sandwich ELISA (LSBio, LS-F4620). For binding ELISA, a 96-well plate was coated with recombinant human CD40L (R&D Systems, 6420-CL-025/CF), after which each of the two concentrated culture fluids was subjected to serial dilution by ⅓ and allowed to react with CD40L. The unbound stefin A protein variant was removed through washing, after which the extent of binding between the stefin A protein variant and CD40L was detected based on a difference in absorbance resulting from reaction with a TMB substrate. Consequently, EC50 values of the stefin A protein variant secreted by the two gene-introduced cell lines were determined to be 0.0039 and 0.0053 nM through bELISA (FIG. 19).

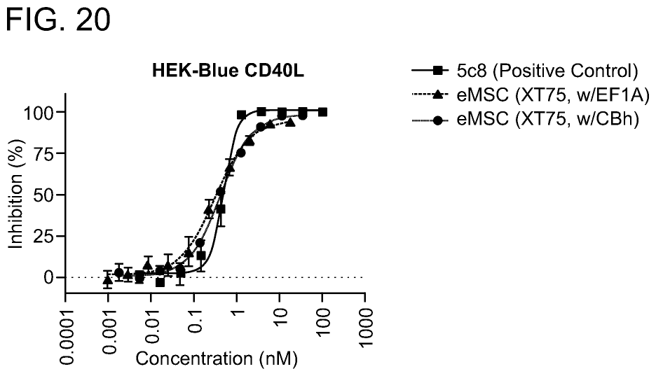

In order to confirm whether the stefin A protein variant secreted by the two gene-introduced cell lines had inhibitory activity on CD40L, a HEK-Blue CD40L cell line (InvivoGen, hkb-cd40) was used. The HEK-Blue CD40L cell line was inoculated at 2.0×10⁴ cells/well into a 96-well plate, followed by culture at 37° C. and 5% CO₂ for 18 to 24 hours. Thereafter, whether the HEK-Blue CD40L cell line was attached well was confirmed, and then 50 μL of the medium was removed. Each of the two concentrated culture fluids was subjected to serial dilution by ⅓, and mixed with a human Mega CD40L recombinant protein (Enzo Lifesciences, ALX-522-100-C010), and 50 μL thereof was placed in the 96-well plate inoculated with HEK-Blue CD40L cells, followed by reaction at 37° C. and 5% CO₂ for 20 to 22 hours. Thereafter, the HEK-Blue CD40L culture fluid was harvested and then allowed to react with a HEK-Blue solution at 37° C. for 3 hours. By measuring the absorbance of each well using a multiplate reader, whether the secreted stefin A protein variant had CD40L-binding inhibitory activity was analyzed. Based on the results of analysis, IC50 for hMega CD40L was 0.470 nM in a benchmark molecule, and IC50 values of the stefin A protein variant secreted by the two gene-introduced cell lines were 0.303 and 0.400 nM, showing similar activities. Therefore, it was concluded that the stefin A protein variant secreted by the gene-introduced cell line had CD40L-binding ability and activity inhibitory ability similar to the reference material (FIG. 20).

### Example 9: Confirmation of Immunosuppressive Activity of MSC into which Anti-CD40L Stefin A Protein Variant Gene was Introduced

### Example 9-1: Confirmation of Effect of Inhibiting T-Cell Activity

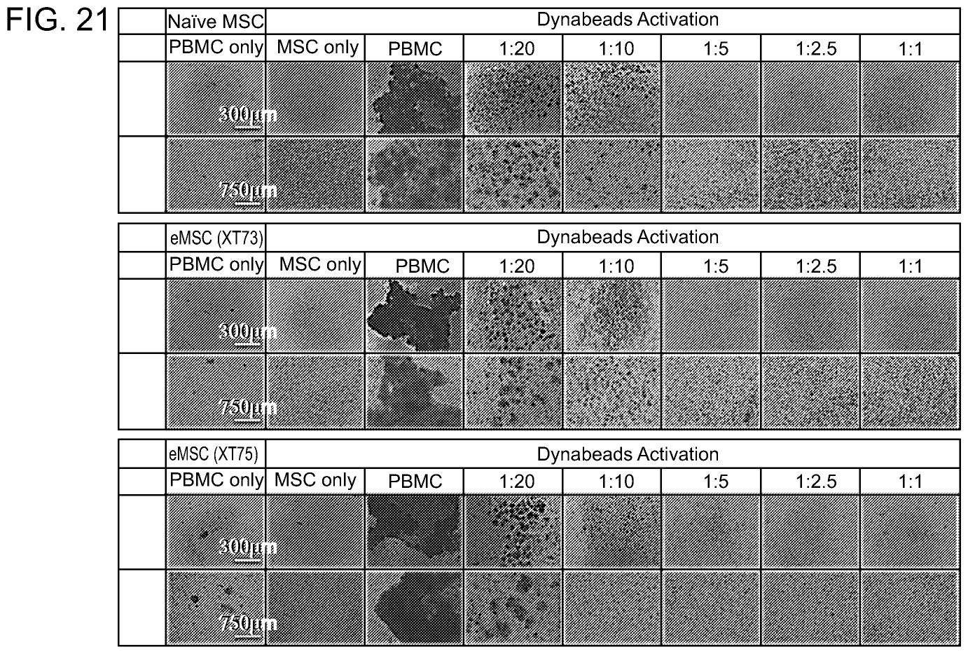

The stefin A protein variant secreted by the anti-CD40L stefin A protein variant gene-introduced cell line was confirmed to have an effect of inhibiting immune cell activity due to the antagonistic effect on CD40L. Each of the constructed stefin A protein variant gene (SEQ ID NO: 692 or 694)-introduced cell line and Naïve MSC was co-cultured with PBMC (Stem Cell Technologies, 70025), and the activation rate of T cells in PBMC was compared. In a 24-well plate, PBMC at 5.0×10⁵ cells/well and each of two anti-CD40L stefin A protein variant gene-introduced cell lines (SEQ ID NO: 692 or 694) and Naïve MSC were mixed in ratios of 1:20, 1:10, 1:5, 1:2.5, and 1:1, followed by cell inoculation. As such, PBMC was inoculated after staining with CFSE capable of confirming cell proliferation, and anti-CD3, CD28 Dynabeads (Gibco, 11161D), and IL-2 (Gibco, PHC0023) were added thereto, followed by culture for 7 days. On the 4^{th} day after inoculation, the culture medium was replaced with a medium containing anti-CD3, CD28 Dynabeads, and IL-2. Based on the results of observation of PBMC clustering capable of evaluating T-cell activity using a phase-contrast microscope, the largest amount of distinct clustering was confirmed when inducing the activation of PBMC alone with Dynabeads. It was observed that clustering of PBMC was decreased with an increase in the proportion of each of the two gene-introduced cell lines and Naïve MSC and was further decreased in the gene-introduced cell lines than in Naïve MSC (FIG. 21).

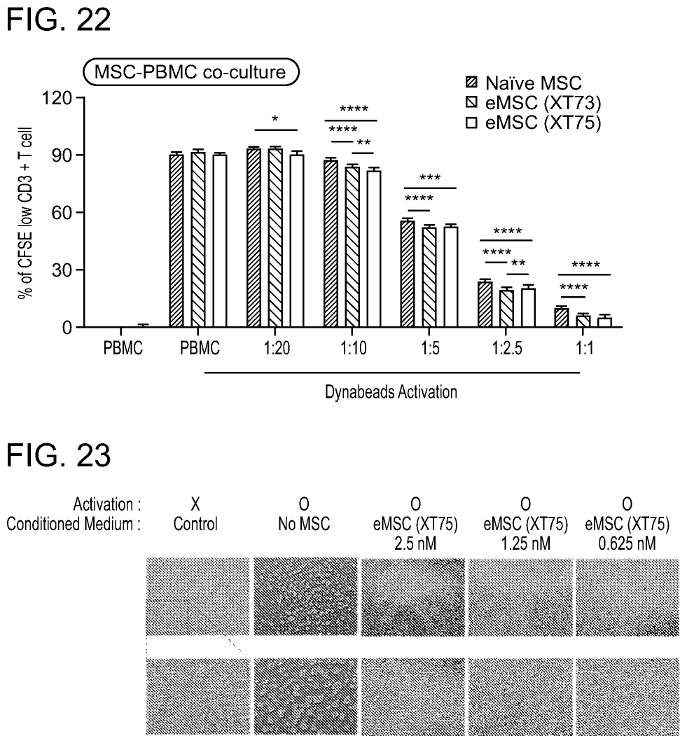

Only PBMC was harvested and the T-cell activity was compared through marker analysis using a flow cytometer. Consequently, CFSE low/CD3 positive cells, corresponding to the population of proliferating T cells, showed a tendency to decrease with an increase in the mixing ratio with each of the two gene-introduced cell lines and Naïve MSC. Moreover, it was confirmed that, when co-cultured with the two gene-introduced cell lines than when co-cultured with Naïve MSC, the T-cell activity was further inhibited (FIG. 22).

### Example 9-2: Confirmation of Effect of Inhibiting B-Cell Activity

The effect of inhibiting T-cell-mediated B-cell activity by the stefin A protein variant secreted from the anti-CD40L stefin A protein variant gene-introduced cell line was confirmed. A gene-introduced cell line into which the anti-CD40L stefin A protein variant gene (SEQ ID NO: 694) was introduced was constructed using a lentivirus and then inoculated into a T175 flask at a cell density of 4.0×10³ cells/cm² using a culture medium, followed by culture. The culture medium was thoroughly replaced with a fresh culture medium every 2 days, followed by culture for 3 days. Thereafter, the cells were harvested and 7.0×10⁶ cells were inoculated into a T175 flask, followed by culture at 37° C. and 5% CO₂ for 18 to 24 hours. Thereafter, the culture medium was thoroughly removed, and the remaining medium was completely removed through washing with MEM-alpha. 30 mL of an MEM alpha medium was added to a T175 flask, followed by culture for 48 hours. After 48 hours, all of the cell culture fluid was harvested and then centrifuged to remove residual cells and impurities. The cell culture fluid was transferred to a Vivaspin 20 and concentrated by centrifugation, and the stefin A protein variant was quantified through sandwich ELISA. The frozen B cells (Lonza, 4W-601) were thawed, stabilized in a T75 flask for one day, and then inoculated at 5.0×10⁵ cells/well into a 24-well plate. Here, MEGACD40L (Enzo Lifesciences, ALX-522-110-C010), IgM (Jackson Immuno Research Laboratories, 109-006-129), and IL-21 (Peprotech, 200-21) were added to the medium to induce activation of B cells, and simultaneously, the concentrated culture fluid was used together after serial dilution. After culture for 30 hours, B-cell clustering capable of evaluating B-cell activity was observed using a phase-contrast microscope. Consequently, when the cells were treated with MEGA CD40L, IgM, and IL-21 alone, clustering was clearly observed. However, clustering was decreased when the cells were additionally treated with the concentrated culture fluid, confirming that B-cell activity was inhibited (FIG. 23).

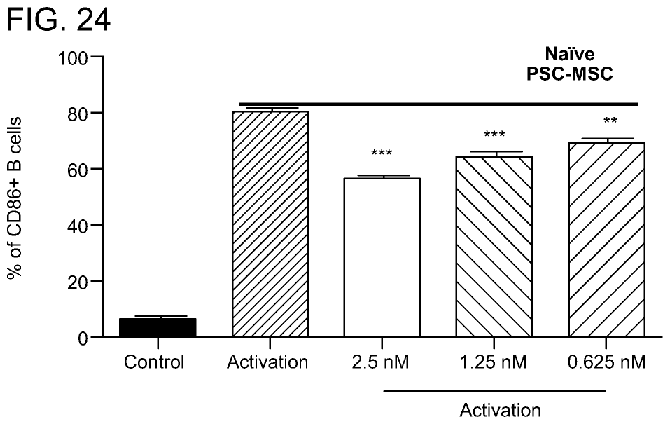

B cells were harvested and the B-cell activity was analyzed using a cell surface marker through flow cytometry. Based on the results of measurement of the population of CD19 positive/CD86 positive cells, which are markers of activated B cells, it was confirmed that the B-cell population, which increased by about 80% when the activity thereof was induced, was significantly decreased when the concentrated culture fluid was further added thereto. Moreover, as the concentrated culture fluid was diluted, the inhibitory effect was decreased, indicating concentration dependence. These results were not confirmed in the culture fluid of Naïve MSC into which the stefin A protein variant gene was not introduced. Therefore, it was concluded that the CD40L signal was suppressed by the stefin A protein variant secreted from the stefin A protein variant gene-introduced cell line, thereby inhibiting B-cell activation (FIG. 24).

### Example 9-3: Confirmation of Immunomodulatory Factor Expression of Anti-CD40L Stefin A Protein Variant Gene-Introduced MSC (eMSC)

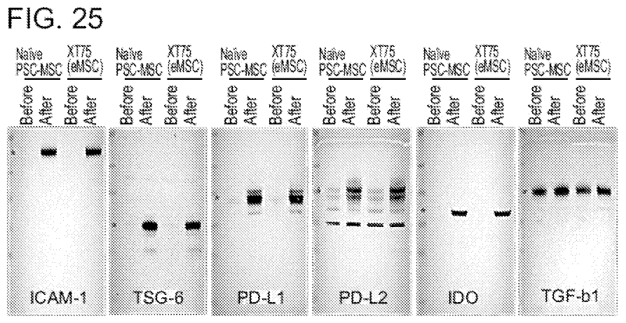

In order to evaluate whether the expression of immunomodulatory factors in an inflammatory environment varies depending on whether the stefin A protein variant gene was introduced, an inflammatory environment was induced in Naïve MSC and the stefin A protein variant gene (SEQ ID NO: 694)-introduced cell line. Naïve MSC of the same passage number as the frozen anti-CD40L stefin A protein variant gene-introduced cell line was thawed, and then inoculated into a T175 flask at a cell density of 4.0×10³ cells/cm² using a culture medium, followed by culture. The culture medium was thoroughly replaced with a fresh culture medium every 2 days, followed by culture for 3 days, after which the cells were harvested. Each of the two cell lines thus harvested was inoculated at 1.5×10⁶ cells in a 100 mm dish, followed by culture for 18 to 24 hours. Thereafter, the culture medium was thoroughly removed and then replaced with a culture medium containing 20 ng/mL of IFN-gamma (PEPROTECH, AF-300-02) and 10 ng/mL of TNF-alpha (PEPROTECH, AF-300-01A), followed by culture for 48 hours to induce an inflammatory environment. After 48 hours, the culture medium was thoroughly removed, washing was performed with PBS, and the cells were harvested with a scraper. The cell pellets were isolated and protein whole lysis was performed using RIPA buffer. The protein was quantified with BCA, loaded in the same amount on SDS-PAGE, and then transferred to a membrane, followed by reaction with TGF-beta1 (Abcam, ab179695), IDO (Abcam, ab76157), IL-10 (Abcam, ab133575), MCP-1 (Abcam, ab214819), and TSG-6, which were secretory factors induced in an inflammatory environment (Abcam, ab267469), and cell surface expression factors such as ICAM-1 (Abcam, ab282575), VCAM-1 (Abcam, ab174279), PD-L1 (Abcam, ab243877), and PD-L2 (Abcam, ab283344) antibodies, after which expression thereof was confirmed. Consequently, when Naïve MSC was treated with IFN-gamma and TNF-alpha to induce an inflammatory environment, expression of immunomodulatory secretion factors such as TGF-beta1, IDO, and TSG-6 was increased, and expression of cell surface expression factors such as ICAM-1, PD-L1, and PD-L2 was also increased (FIG. 25). When an inflammatory environment was induced in the stefin A protein variant gene-introduced cell line, in addition to Naïve MSC, expression of the immunomodulatory factors was also increased, confirming that there was no difference in expression between Naïve MSC and the stefin A protein variant gene-introduced cell line (FIG. 25).

### Example 10: Verification of Purity of Anti-CD40L Stefin A Protein Variant Gene-Introduced MSC (eMSC)

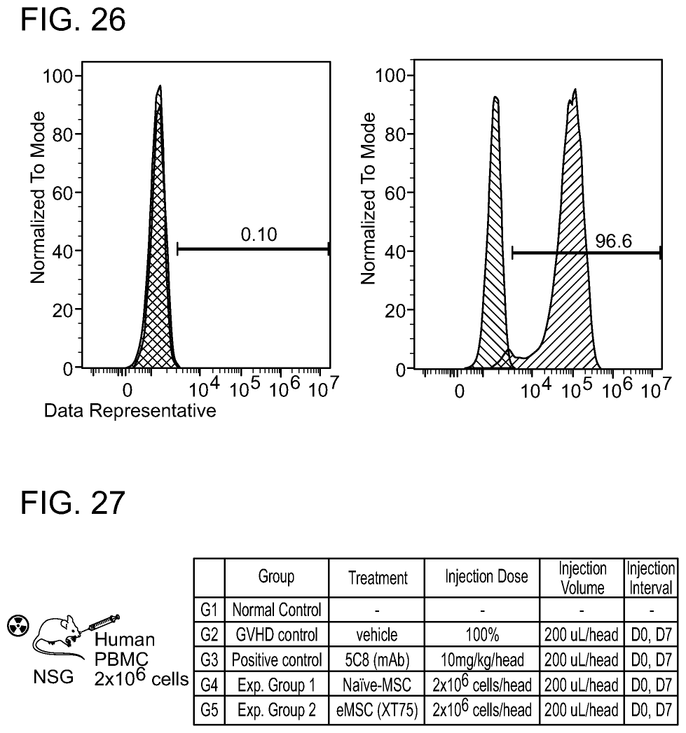

Purity was verified by analyzing the proportion of cells expressing the stefin A protein variant gene (SEQ ID NO: 694) transferred into the constructed stefin A protein variant gene-introduced mesenchymal stem cells (non-target cell proportion). When constructing the stefin A protein variant gene-introduced cell line, a signal peptide sequence (SEQ ID NO: 503) was added so that the stefin A protein variant could be secreted out of the cells. Accordingly, since the stefin A protein variant gene-introduced cell line secretes all of the stefin A protein variant out of the cells, it is necessary to suppress the secretion of the secreted protein in order to identify the cells into which the stefin A protein variant gene is transferred. The secreted protein was accumulated in the Golgi apparatus, which is an intracellular organelle, using Golgiplug (Brefeldin A; BD Pharmingen, 555029), and the proportion of cells expressing the stefin A protein variant therein was determined. The expression of the introduced stefin A protein variant after intracellular protein accumulation by inhibiting the secretion of the secreted protein through Golgiplug was confirmed. Naïve MSC of the same passage number as the frozen anti-CD40L stefin A protein variant gene-introduced cell line was thawed and then inoculated into a 100 mm dish at a cell density of 3.5×10⁴ cells/cm² using a culture medium, followed by culture. The next day, the culture medium was removed and then replaced with a culture medium containing 0.1 μg/mL of Golgiplug, followed by reaction at 37° C. and 5% CO₂ for 4 hours. Thereafter, the cells were harvested, followed by permeabilization and then reaction with an antibody (Novus Biologicals, NBP2-59470) capable of identifying the stefin A protein variant to stain the cells. Using a flow cytometer, the proportion of the cells expressing the stefin A protein variant was determined. Consequently, the proportion of the stefin A protein variant gene-transferred cells in the stefin A protein variant gene-introduced cell line was determined to be 96.6%. It was found that the proportion of the cells into which the stefin A protein variant was introduced in the constructed stefin A protein variant gene-introduced cell line was maintained very high (FIG. 26).

### Example 11: Therapeutic Effect of Anti-CD40L Stefin A Protein-Secreting Cell Line on GVHD Animal Model

### Example 11-1: Experimental Method

NOD.Cg-Prkdcscid Il2rgtm1Wjl/SzJ (NSG) 5- to 6-week-old mice were purchased from Jackson Laboratory and JABio, and after acclimatization for 1 to 2 weeks, 7-week-old mice were used for the experiment.

For xenotrasplantation GVHD induction, 4-5 NSG mice were irradiated with 1.5 Gy of radiation, and then injected intravenously (i.v.) with human PBMC (Lonza) at 2×10⁶ cells/head the next day and thus GVHD was induced.

The drug was administered intravenously to the cell-injected group at DO and D7 based on 24 hours after PBMC administration, and was administered intraperitoneally (i.p.) to a reference material 5c8 antibody-injected group at DO and D7 based on 24 hours after PBMC administration (FIG. 27).

The animals in this experiment were randomly grouped before irradiation depending on the body weight for the experiment, and analysis of variance (ANOVA) was performed to determine homogeneity between groups.

The animals were observed for general symptoms every day, and particularly observed before and after injection of cells and materials, and animals in severe pain or a moribund state during the observation period were euthanized after symptoms thereof were recorded. The body weight of the mice was measured twice a week, and the GVHD clinical score was measured every 3 days based on the drug administration day 0 and followed-up for 60 days.

### Example 11-2: Experiment Result

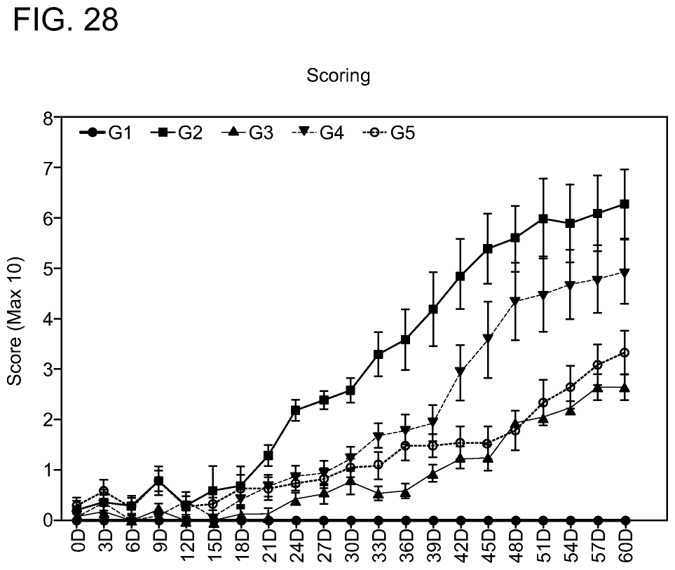

GVHD clinical scoring was given 0 to 2 scores each for five items of weight loss, posture, activity, fur texture, and skin integrity, and the summed values are shown in FIG. 28. The XT75 gene-expressing engineered MSC (eMSC) administration group showed low score values compared to the control, and statistical significance was confirmed through two-way ANOVA, and the interaction, column factor, and row factor all showed P<0.0001. Compared to the positive control administered with 5c8, the XT75-expressing eMSC administration group showed similar score values (FIG. 28).

### Example 12: Therapeutic Effect of Anti-CD40L Stefin A Protein Variant on Haploidentical Genotype GVHD Animal Model

### Example 12-1: Experimental Method

192 healthy female C57BL/6 mice (6 weeks old) were purchased from Janvier (France) and used for splenocyte extraction for GVHD induction. 12 healthy female B6D2F1 mice (6 weeks old) were purchased from Charles River and used for GVHD induction (syngeneic control). 70 healthy female B6D2F1 mice (6 weeks old) were purchased from Charles River and used as donor mice.

The stefin A protein variant specifically binding to mouse CD40L was expressed in the form of a trimeric in-line fusion (Table 17).

TABLE 17

| Stefin A | | SEQ |
|---|---|---|
| protein | | ID |
| variant | Sequence | NO: |
| XT54 | IPGGLSEAKPATPEIQEIVDKVKPQLEEKTGETYGKLEA | 739 |
| | VQYKTQVVFQYVFSDEDGTNYYIKVRAGDNKYMHLKVFK | |
| | SLFRWQRGNPEDLVLTGYQVDKNKDDELTGFGGGGSGGG | |
| | GSGGGGSGGGGSGGGGSGGGGSMIPGGLSEAKPATPEIQ | |
| | EIVDKVKPQLEEKTGETYGKLEAVQYKTQVVFQYVFSDE | |
| | DGTNYYIKVRAGDNKYMHLKVFKSLFRWQRGNPEDLVLT | |
| | GYQVDKNKDDELTGFGGGGSGGGGSGGGGSGGGGSGGGG | |
| | SGGGGSMIPGGLSEAKPATPEIQEIVDKVKPQLEEKTGE | |
| | TYGKLEAVQYKTQVVFQYVFSDEDGTNYYIKVRAGDNKY | |
| | MHLKVFKSLFRWQRGNPEDLVLTGYQVDKNKDDELTGFG | |
| | GGGSGGGGSGGGGSGGGGSGGGGSGGGGSMIPRGLSEAK | |
| | PATPEIQEIVDKVKPQLEEKTGETYGKLEAVQYKTQVLA | |
| | NFFQRRWPGSTNYYIKVRAGDNKYMHLKVFNGPWKFRNT | |
| | DRGADRVLTGYQVDKNKDDELTGFAAAGEQKLISEEDLG | |
| | AAHHHHHH-- | |
| XT55 | IPGGLSEAKPATPEIQEIVDKVKPQLEEKTGETYGKLEA | 740 |
| | VQYKTQVVFQYVFSDEDGTNYYIKVRAGDNKYMHLKVFK | |
| | SLFRWQRGNPEDLVLTGYQVDKNKDDELTGFAEAAAKEA | |
| | AAKEAAAKEAAAKEAAAKEAAAKMIPGGLSEAKPATPEI | |
| | QEIVDKVKPQLEEKTGETYGKLEAVQYKTQVVFQYVFSD | |
| | EDGTNYYIKVRAGDNKYMHLKVFKSLFRWQRGNPEDLVL | |
| | TGYQVDKNKDDELTGFAEAAAKEAAAKEAAAKEAAAKEA | |
| | AAKEAAAKMIPGGLSEAKPATPEIQEIVDKVKPQLEEKT | |
| | GETYGKLEAVQYKTQVVFQYVFSDEDGTNYYIKVRAGDN | |
| | KYMHLKVFKSLFRWQRGNPEDLVLTGYQVDKNKDDELTG | |
| | FAEAAAKEAAAKEAAAKEAAAKEAAAKEAAAKMIPRGLS | |
| | EAKPATPEIQEIVDKVKPQLEEKTGETYGKLEAVQYKTQ | |
| | VLANFFQRRWPGSTNYYIKVRAGDNKYMHLKVFNGPWKF | |
| | RNTDRGADRVLTGYQVDKNKDDELTGFAAAGEQKLISEE | |
| | DLGAAHHHHHH-- | |

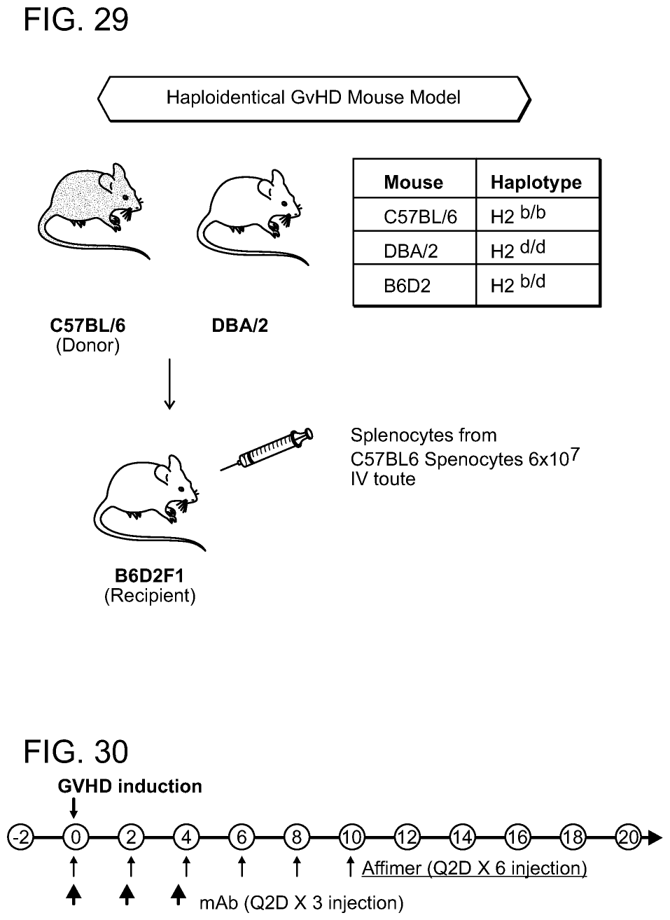

The spleens of C57BL/6 and B6D2F1 mice were excised, and splenocytes were extracted therefrom and prepared in an HBSS buffer. Red blood cells contained in the extracted splenocytes were prepared through dissolution in a red blood cell lysate (BD Pharma) and then washing. 64 B6D2F1 mice (G4-G11) were injected intravenously with 6×10⁷ cells/head of splenocytes isolated from C57BL/6 spleens. Three B6D2F1 mice (G3) were injected intravenously with 6×10⁷ cells/head of splenocytes isolated from the spleens of C57BL/6 mice. Three B6D2F1 mice (G2) were injected intravenously with 6×10⁷ cells/head of splenocytes isolated from the spleens of the same B6D2F1 mice (FIG. 29).

The animals for this experiment were randomly grouped depending on the body weight, and analysis of variance (ANOVA) for homogeneity between groups was performed to confirm that there was no statistical significance.

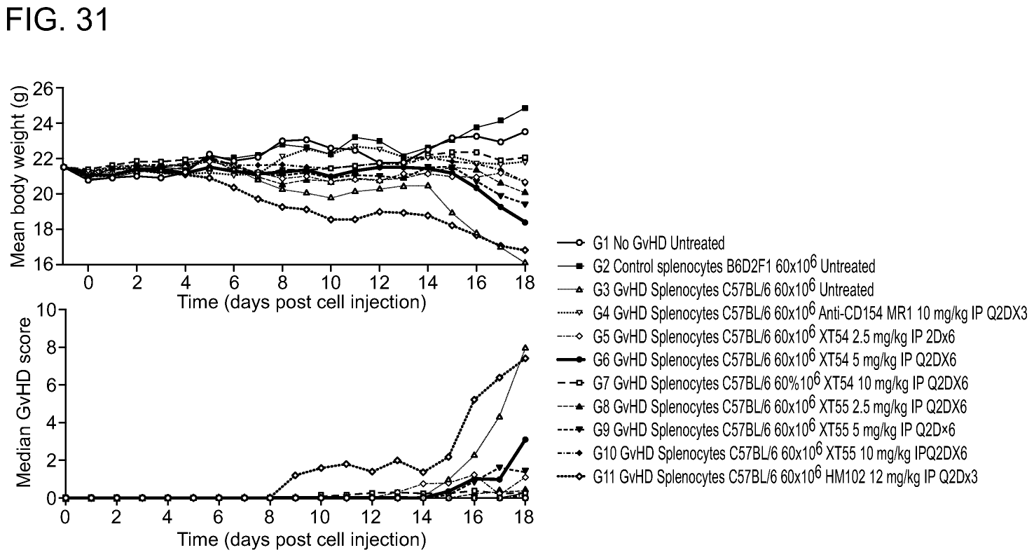

The test material was administered intraperitoneally. For intraperitoneal administration, the pH of the formulation was adjusted to 7.3-7.4, and the volume thereof was set to 20 mL/kg. The type and amount of administration material injected to the test animals are as shown in the following table. The first administration of the test material was carried out 1 hour after GVHD induction, and the administration interval depending on the administration material was set as follows. Administration was carried out six times at 2-day intervals after GVHD induction in the stefin A protein variant administration group except the antibody administration group, and the MR-1 antibody was administered three times at 2-day intervals after GVHD induction (FIG. 30, Table 18). Animal survival and behavior were observed daily and clinical measures were recorded daily. Body weight and clinical score were recorded daily. The clinical scoring of GVHD was evaluated by performing visual observation through the following evaluation formats and recording the sum of the observed scores. For statistical significance between groups, ANOVA was performed using GraphPad Prism. Statistical significance between groups was judged to be significant when p<0.05 or less.

TABLE 18

| | No. | | | Dose | Treatment |
|---|---|---|---|---|---|
| Group | Animals | Cells | Treatment | (mg/kg) | Schedule |
| 1 | 3 | NO | Untreated | — | — |
| 2 | 3 | syngenic control | Untreated | — | — |
| 3 | 3 | C57BL/6 | Untreated | — | — |
| | | splenocytes | | | |
| 4 | 8 | C57BL/6 | MR1 | 10 | Q2DX3 |
| | | splenocytes | | | |
| 5 | 8 | C57BL/6 | XT54 | 2.5 | Q2DX6 |
| | | splenocytes | | | |
| 6 | 8 | C57BL/6 | XT54 | 5 | Q2DX6 |
| | | splenocytes | | | |
| 7 | 8 | C57BL/6 | X154 | 10 | Q2DX6 |
| | | splenocytes | | | |
| 8 | 8 | C57BL/6 | XT55 | 2.5 | Q2DX6 |
| | | splenocytes | | | |
| 9 | 8 | C57BL/6 | XT55 | 5 | Q2DX6 |
| | | splenocytes | | | |
| 10 | 8 | C57BL/6 | XTS5 | 10 | Q2DX6 |
| | | splenocytes | | | |

### Example 12-2: Result

After random grouping, the mean body weight of the animal groups was 21.5 g, the body weight ranging from 18.8 g to 23.8 g. Based on the results of statistical analysis, there was no significant difference between groups. Changes in body weight of experimental animals were monitored throughout the study period. In order to confirm body weight changes, the mean body weight change (MBWC %) was compared based on D18. The mean body weight change in the G1 group was increased by 9.7%, and the mean body weight change in the G2 group was increased by 15.6%. In the G3 group, which is the GVHD control, the mean body weight change was decreased by 24.4%. The mean body weight changes of the drug administration groups, G4 to G11 groups, were as follows.

G4 group: 1.6% increase, G5 group: 4.1% decrease, G6 group: 14.6% decrease, G7 group: 2.8% increase, G8 group: 6.4% decrease, G9 group: 9.0% decrease, G10 group: 4.4% decrease, G11 Group: 21.7% decrease, indicating that the stefin A protein variant administration groups showed good mean body weight changes compared to the G3 group.

Changes in clinical scores for individual animals were recorded using a scorecard including body, skin, hair, and mobility criteria. The average GVHD scores of the G1 and G2 groups did not increase throughout the study period. The GVHD score of the G3 group started increasing from D15, and recorded an average value of 8.0 on D18. Similar to the G1 and G2 groups, the GVHD score did not increase in the G4 group throughout the study period, and the GVHD scores of the G5 to G7 groups started to increase between 9 and 14 days after GVHD induction, and showed 0.3 to 1.1 on D18, the end of the experiment. The clinical scores of the G8 to G10 groups started to increase between 7 and 10 days after GVHD induction, and showed a GVHD score of 0.3 to 1.4 on D18, the end of the experiment (FIG. 31).

In consideration thereof, it could be confirmed that the therapeutic effect by the stefin A protein variant was statistically significantly inhibited compared to the GVHD control.

As is apparent from the above description, a genetically modified cell according to the present invention is capable of expressing a CD40L binding agent, such as, e.g., a stefin A protein variant that specifically binds to CD40L and/or a fusion protein including the same, thus enabling secretion thereof, expression thereof on a cell membrane, and/or intracellular expression thereof. It was confirmed that a CD40L binding agent (e.g., the stefin A protein variant) expressed in the genetically modified cell and/or the fusion protein including the same can specifically bind to CD40L, thus reducing or inhibiting the activity of CD40L. Thereby, the genetically modified cell of the present invention can exhibit excellent immunomodulatory effects such as inhibition of T-cell activity and B-cell activity, and is thus useful for the prevention or treatment of immune diseases such as autoimmune diseases or inflammatory diseases.

Although specific embodiments of the present invention have been described illustratively, those skilled in the art will appreciate that the present invention may be embodied in other specific forms without changing the technical spirit or essential features thereof. Thus, the embodiments described above should be understood to be non-limiting and illustrative in every way.

1. A population of genetically modified mesenchymal stromal cells (MSCs), wherein the MSCs comprise an exogenous nucleic acid comprising a coding sequence that encodes a CD40L binding agent, wherein the CD40L binding agent comprises one or more binding domains from an antibody or antibody mimetic.

2. The population of genetically modified MSCs of claim 1, wherein the CD40L binding agent comprises a stefin A protein variant, Fab, Fab′, F(ab′)2, Fv, Fd, scFv, sdFv), VL, VH, Camel Ig, V-NAR, VHH, trispecific (Fab3), bispecific (Fab2), diabody ((VL-VH)2 or (VH-VL)2), triabody (trivalent), tetrabody (tetravalent), minibody ((scFv-CH3)2), bispecific single-chain Fv (Bis-scFv), a shark heavy-chain-only antibody (VNAR), a microprotein (cysteine knot protein, knottin), affibody, aptamer, avimer, nanobody, unibody, a single domain antibody, affilin, affitin, adnectin, atrimer, evasin, DARPin, anticalin, avimer, fynomer, versabody, repebody or a duocalin.

3. The population of genetically modified MSCs of claim 1, wherein the CD40L binding agent comprises a stefin A protein variant.

4. The population of genetically modified MSCs of claim 1, wherein the exogenous nucleic acid comprises a transcriptional regulatory sequence that is operably linked to the coding sequence, wherein the transcriptional regulatory sequence is a promoter selected from a CMV promoter, a EFS promoter, a CBh promoter, a MSCV promoter, a SFFV promoter, and a E1FA promoter.

5.-6. (canceled)

7. The population of genetically modified MSCs of claim 1, wherein the exogenous nucleic acid comprises, in order, a promoter, the coding sequence that encodes a CD40L binding agent, a IRES or 2A sequence, and an antibiotic selection gene.

8. (canceled)

9. The population of genetically modified MSCs of claim 1, wherein the MSCs are derived from induced pluripotent stem cells or embryonic stem cells.

10. The population of genetically modified MSCs of claim 1, wherein (i) the MSCs express at least one cell surface marker selected from CD29, CD44, CD73, CD90, and CD105; and/or (ii) the MSCs do not express at least one cell surface marker selected from among CD11b, CD14, CD34, CD45, CD79, HLA-DR, TRA-1-60, and TRA-1-81.

11. The population of genetically modified MSCs of claim 10, wherein at least 90% of expression of the cell surface marker is maintained in the population of MSCs after at least 15 passages.

12. (canceled)

13. The population of genetically modified MSCs of claim 1, wherein at least 95% of the MSCs are CD73+ and CD105+, and less than 1% express CD45, SSEA-3, TRA-1-60, TRA-1-81, and HLA-DR.

14. (canceled)

15. The population of genetically modified MSCs of claim 3, wherein the stefin A protein variant comprises an amino acid sequence represented below:
(i)
MIPGGLSEAKPATPEIQEIVDKVKPQLEEKTGETYGKLEAVQYKTQVV-(Xaa)n-
GTNYYIKVRAGDNKYMHLKVFKSL-(Xaa)m-EDLVLTGYQVDKNKDDELTGF;
(ii)
MIPGGLSEAKPATPEIQEIVDKVKPQLEEKTGETYGKLEAVQYKTQVD-(Xaa)n-
GTNYYIKVRAGDNKYMHLKVFKSL-(Xaa)m-EDLVLTGYQVDKNKDDELTGF;
or
(iii)
MIPGGLSEAKPATPEIQEIVDKVKPQLEEKTGETYGKLEAVQYKTQVLA-
(Xaa)n-GTNYYIKVRAGDNKYMHLKVFKSL-(Xaa)m-EDLVLTGYQVDKNKDDELTGF
wherein Xaa is an amino acid residue, and n and m are each independently an integer from 3 to 20.

16. The population of genetically modified MSCs of claim 3, wherein the stefin A protein variant comprises an amino acid sequence selected from the group consisting of SEQ ID NOs: 246 to 365.

17. The population of genetically modified MSCs of claim 15, wherein
(i)(Xaa)n comprises an amino acid sequence selected from the group consisting of SEQ ID NOs: 6 to 125; and/or
(ii) (Xaa)m comprises an amino acid sequence selected from the group consisting of SEQ ID NOs: 126 to 245.

18. The population of genetically modified MSCs of claim 3, wherein the stefin A protein variant further comprises a signal peptide.

19. The population of genetically modified MSCs of claim 3, wherein the CD40L binding agent comprises a trimer or tetramer of stefin A protein variants.

20. The population of genetically modified MSCs of claim 1, wherein at least 90% of the MSCs of the population comprise the exogenous nucleic acid.

21. The population of genetically modified MSCs of claim 1, wherein
(a) the CD40L binding agent is expressed on the surface of the MSCs; or
(b) the CD40L binding agent is secreted extracellularly, wherein:
(i) the population of genetically modified MSCs secrete the CD40L binding agent at an average level of 200 fg/cell/day or more; and or
(ii) the population of genetically modified MSCs secrete the CD40L binding agent at an average level of 200 to 1500 fg/cell/day.

22.-25. (canceled)

26. A method of producing a population of genetically modified mesenchymal stem cells (MSCs) of claim 1, comprising:
contacting a population of MSCs with a lentiviral vector comprising an exogenous nucleic acid comprising a coding sequence that encodes a CD40L binding agent, and
culturing the population of MSCs.

27.-35. (canceled)

36. A method of treating an immune disease, comprising administering the population of genetically modified MSCs of claim 1 to a subject in need thereof.

37. (canceled)

38. A population of genetically modified cells, wherein the cells comprise an exogenous nucleic acid comprising a coding sequence that encodes a CD40L binding agent, wherein the CD40L binding agent comprises one or more binding domains from an antibody or antibody mimetic.

39.-41. (canceled)

42. The population of genetically modified cells of claim 38, wherein the cells are selected from the group consisting of stem cells, immune cells, and somatic cells.

43.-68. (canceled)

69. A method of treating an immune disease, comprising administering the population of genetically modified cells of claim 38 to a subject in need thereof.

70. (canceled)
